(19) United States
(12) Patent Application Publication
(10) Pub. No.: US 2024/0074305 A1
(43) Pub. Date: Feb. 29, 2024

(54) TETRAHYDRONAPHTHALENE-BASED ORGANIC COMPOUND, MIXTURE, COMPOSITION, AND ORGANIC ELECTRONIC DEVICE

(71) Applicant: Shenzhen China Star Optoelectronics Semiconductor Display Technology Co., Ltd., Shenzhen (CN)

(72) Inventors: Xinlong SONG, Shenzhen (CN); Zhihua XIAO, Shenzhen (CN); Ruifeng HE, Shenzhen (CN); Jingyao SONG, Shenzhen (CN)

(21) Appl. No.: 18/083,480

(22) Filed: Dec. 17, 2022

(30) Foreign Application Priority Data
Aug. 5, 2022 (CN) 202210939527.5

(51) Int. Cl. H10K 85/60; C07D 209/86; C09K 11/02; C09K 11/06

(52) U.S. Cl. CPC H01L 51/006; C07D 209/86; C09K 11/025; C09K 11/06; H01L 51/0061; C09K 2211/1011; C09K 2211/1014; C09K 2211/1022; H01L 51/0058; H01L 51/0072; H01L 51/5012

(57) ABSTRACT

Provided is a tetrahydronaphthalene-based organic compound having a structure as shown in general formula (1), a mixture, a composition, and an organic electronic device. When the tetrahydronaphthalene organic compound provided in the present disclosure is used as a blue fluorescent luminescent material, not only luminescence efficiency and lifetime of the device be improved, but also dark blue luminescence of the device can be realized.

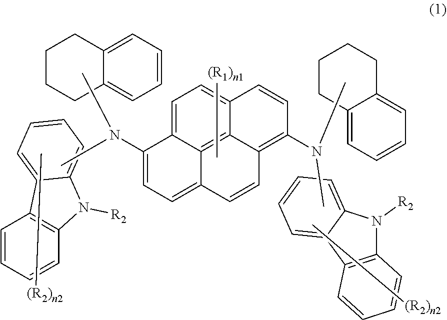

### CROSS-REFERENCE TO RELATED APPLICATION

The present disclosure claims priority to and the benefit of Chinese Patent Application No. 202210939527.5, filed on Aug. 5, 2022, the entire content of which is hereby incorporated by reference.

### TECHNICAL FIELD

The present disclosure relates to a technical field of display, and in particular, to a tetrahydronaphthalene-based organic compound, a mixture, a composition and an organic electronic device.

### BACKGROUND

Organic electronic devices, particularly organic light-emitting diode (OLED) devices, are widely used because of their self-lumination, high brightness, high efficiency, low driving voltage, wide viewing angle, high contrast, high responsiveness, etc. An OLED device generally includes an anode, a cathode, and an organic layer disposed between the anode and the cathode. The organic layer generally includes a hole injecting layer, a hole transporting layer, a light-emitting layer, an electron transporting layer, an electron injecting layer, etc. When a voltage is applied between the anode and the cathode of an OLED device, the anode injects holes into the organic layer, and the cathode injects electrons into the organic layer. When the holes meet the electrons, excitons are formed, and light is emitted when the excitons jump back to the ground state.

Currently, OLED using blue fluorescent materials have high reliability. However, the existing blue fluorescent materials have wide emission spectrums and poor color purity, which are not conducive to high-end display. Moreover, the synthesis of the existing blue fluorescent materials is complicated, which is not conducive to mass production. In addition, the efficiency and lifetime of the existing blue OLED devices need to be improved.

A light-emitting layer of an existing blue light OLED adopts a host-guest doping structure. Most of blue light host materials are anthracene-based fused ring derivatives, and most of blue light guest compounds are aryl vinyl amine compounds. These compounds have poor thermal stability and are easy to decompose, resulting in poor lifetime of OLED devices. At the same time, these compounds have poor color purity, and it is difficult to achieve dark blue luminescence. Therefore, there are problems in realizing full-color displays.

### SUMMARY

The present disclosure provides a tetrahydronaphthalene-based organic compound, a mixture, a composition, and an organic electronic device, which solves the technical problems of short lifetime and poor color purity of existing blue fluorescent materials.

Technical solutions of the present disclosure are as follows:

The present disclosure provides a tetrahydronaphthalene-based organic compound having a structure shown in general formula (1):

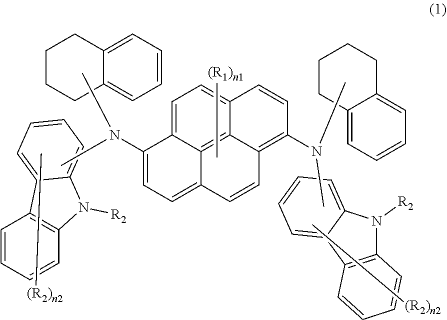

wherein, n1 is an integer selected from 1 to 8; n2 is an integer selected from 1 to 7;

R₁ and R₂, at each occurrence, is independently selected from the group consisting of H, deuterium, a linear alkyl group containing 1 to 20 carbon atoms, a linear alkoxy containing 1 to 20 carbon atoms, a linear thioalkoxy group containing carbon atoms, a branched or cyclic alkyl containing 3 to 20 carbon atoms, a branched or cyclic alkoxy containing 3 to 20 carbon atoms, a branched or cyclic thioalkoxy group containing 3 to 20 carbon atoms, a silyl group, a ketone group containing 1 to 20 carbon atoms, an alkoxycarbonyl group containing 2 to 20 carbon atoms, an aryloxycarbonyl group containing 7 to 20 carbon atoms, a substituted or unsubstituted aromatic or heteroaromatic group containing 5 to 60 ring atoms, an aryloxy or heteroaryloxy group containing 5 to 60 ring atoms, a cyano group, a carbamoyl group, a haloformyl group, a formyl group, an isocyano group, an isocyanate group, a thiocyanate group, an isothiocyanate group, a hydroxyl group, a nitro group, an amino group, —CF₃, —Cl, —Br, —F, —I, and combinations thereof.

Optionally, in some embodiments of the present disclosure, the tetrahydronaphthalene-based organic compound has a structure shown in general formula (2):

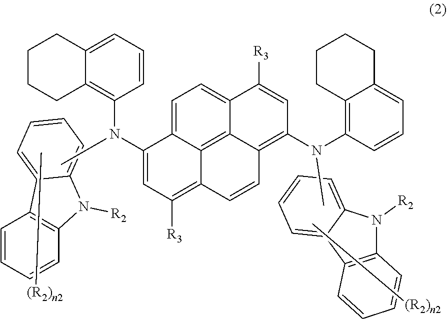

wherein, R₃, at each occurrence, is independently selected from the group consisting of methyl, ethyl, isopropyl and tert-butyl

Optionally, in some embodiments of the present disclosure, the tetrahydronaphthalene-based organic compound has a structure shown in general formulae from (3-1) to (3-4):

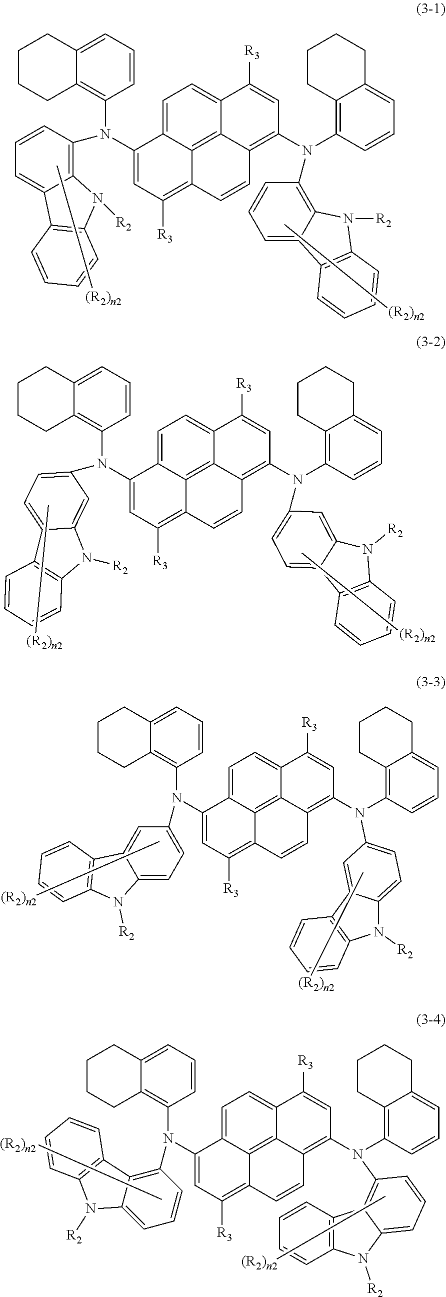

Optionally, in some embodiments of the present disclosure, adjacent R₁ form a ring with each other.

Optionally, in some embodiments of the present disclosure, R₁ and R₂, at each occurrence, is independently selected from the group consisting of a linear alkyl group having 1 to 10 carbon atoms, a branched or cyclic alkyl containing 3 to 10 carbon atoms, a substituted or unsubstituted aromatic group containing 5 to 30 ring atoms, or a substituted or unsubstituted heteroaromatic group containing 5 to 30 ring atoms, and a combination thereof.

Optionally, in some embodiments of the present disclosure, the tetrahydronaphthalene-based organic compound is selected from any one of the following structures:

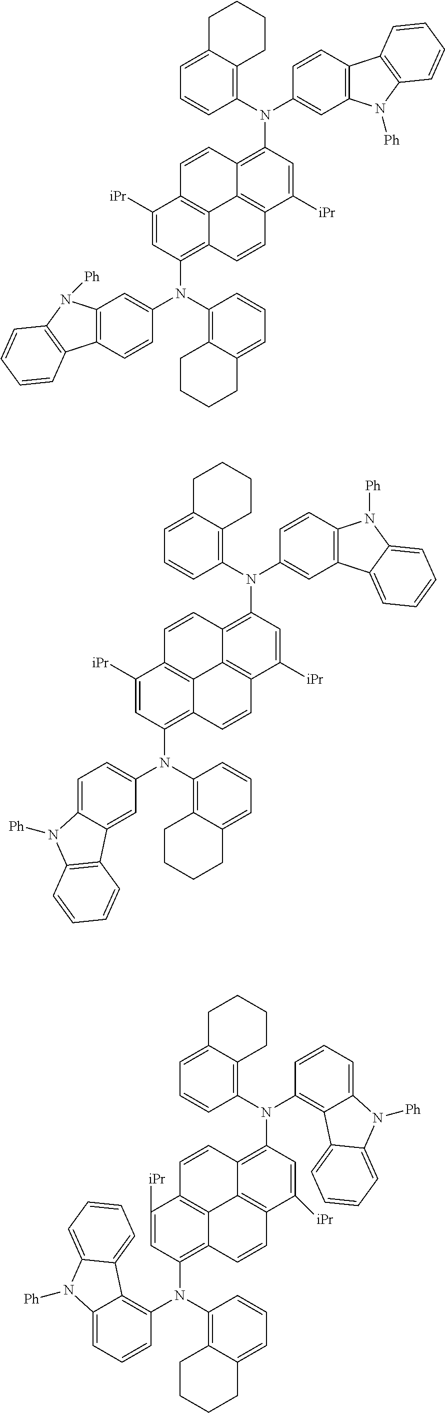 [SMILES: CC(C)c1cc(N(c2ccc3c4ccccc4n(-c4ccccc4)c3c2)c2cccc3c2CCCC3)c2ccc3c(C(C)C)cc(N(c4ccc5c6ccccc6n(-c6ccccc6)c5c4)c4cccc5c4CCCC5)c4ccc1c2c34 ; CC(C)c1cc(N(c2ccc3c(c2)c2ccccc2n3-c2ccccc2)c2cccc3c2CCCC3)c2ccc3c(C(C)C)cc(N(c4ccc5c(c4)c4ccccc4n5-c4ccccc4)c4cccc5c4CCCC5)c4ccc1c2c34 ; CC(C)c1cc(N(c2cccc3c2CCCC3)c2cccc3c2c2ccccc2n3-c2ccccc2)c2ccc3c(C(C)C)cc(N(c4cccc5c4CCCC5)c4cccc5c4c4ccccc4n5-c4ccccc4)c4ccc1c2c34]

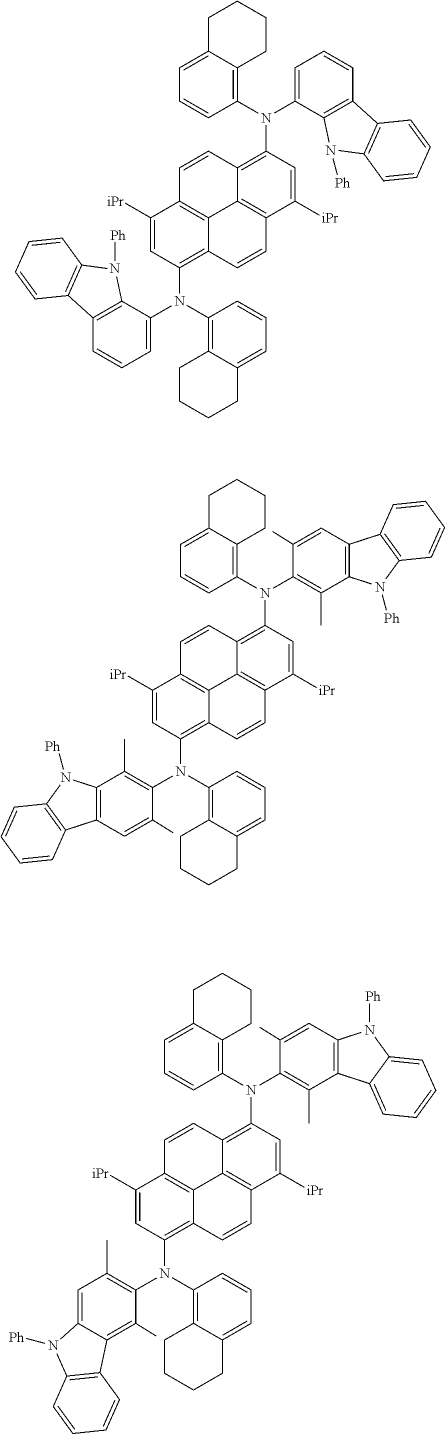 [SMILES: CC(C)c1cc(N(c2cccc3c2CCCC3)c2cccc3c4ccccc4n(-c4ccccc4)c23)c2ccc3c(C(C)C)cc(N(c4cccc5c4CCCC5)c4cccc5c6ccccc6n(-c6ccccc6)c45)c4ccc1c2c34 ; Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)cc5c6ccccc6n(-c6ccccc6)c5c4C)cc(C(C)C)c4ccc1c2c43 ; Cc1cc2c(c(C)c1N(c1cccc3c1CCCC3)c1cc(C(C)C)c3ccc4c(N(c5cccc6c5CCCC6)c5c(C)cc6c(c5C)c5ccccc5n6-c5ccccc5)cc(C(C)C)c5ccc1c3c54)c1ccccc1n2-c1ccccc1]

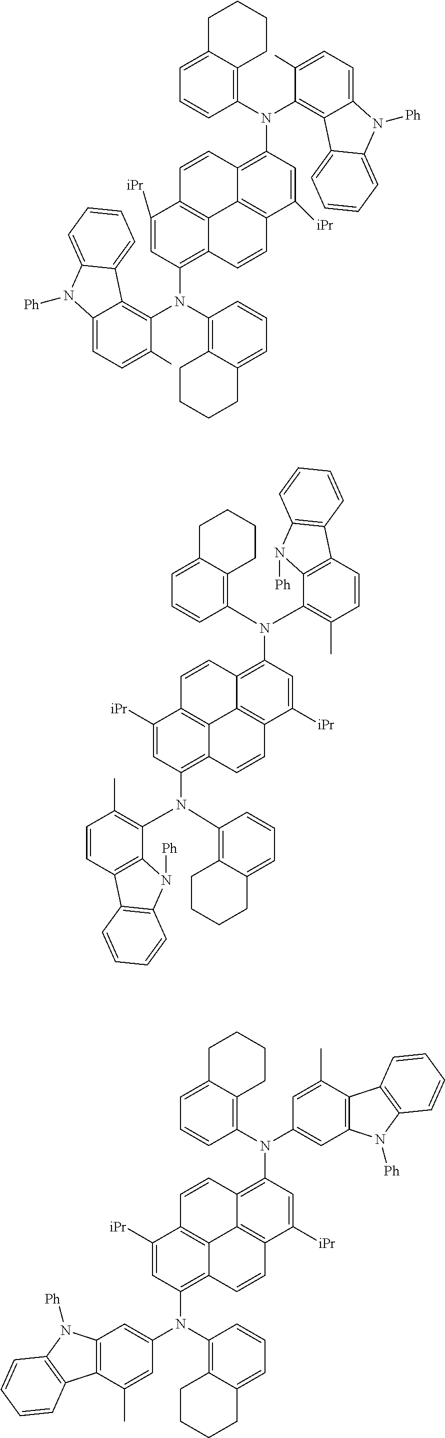 [SMILES: Cc1ccc2c(c1N(c1cccc3c1CCCC3)c1cc(C(C)C)c3ccc4c(N(c5cccc6c5CCCC6)c5c(C)ccc6c5c5ccccc5n6-c5ccccc5)cc(C(C)C)c5ccc1c3c54)c1ccccc1n2-c1ccccc1 ; Cc1ccc2c3ccccc3n(-c3ccccc3)c2c1N(c1cccc2c1CCCC2)c1cc(C(C)C)c2ccc3c(N(c4cccc5c4CCCC5)c4c(C)ccc5c6ccccc6n(-c6ccccc6)c45)cc(C(C)C)c4ccc1c2c43 ; Cc1cc(N(c2cccc3c2CCCC3)c2cc(C(C)C)c3ccc4c(N(c5cc(C)c6c7ccccc7n(-c7ccccc7)c6c5)c5cccc6c5CCCC6)cc(C(C)C)c5ccc2c3c54)cc2c1c1ccccc1n2-c1ccccc1]

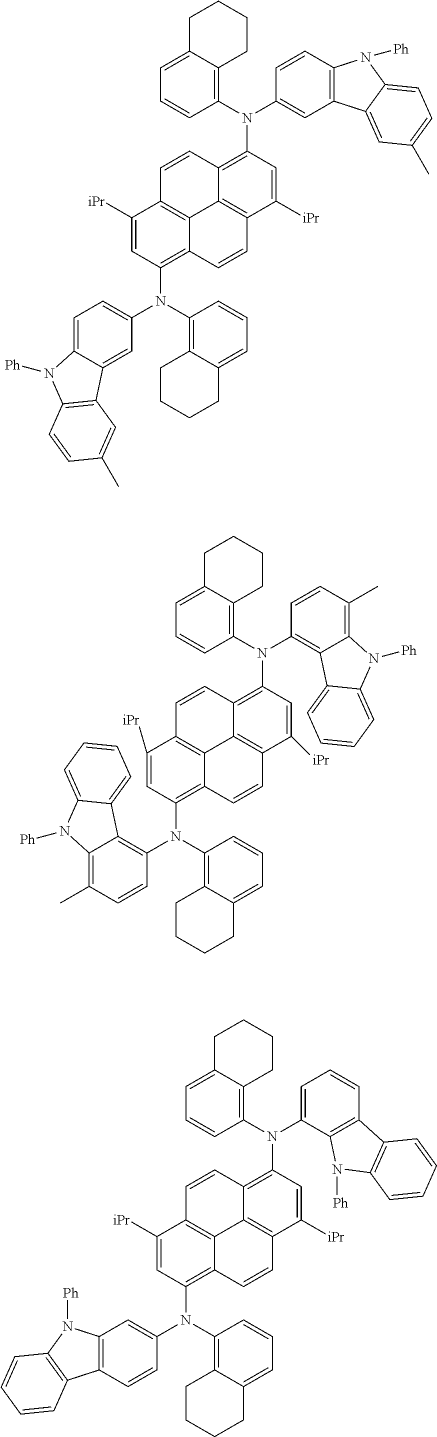 [SMILES: Cc1ccc2c(c1)c1cc(N(c3cccc4c3CCCC4)c3cc(C(C)C)c4ccc5c(N(c6ccc7c(c6)c6cc(C)ccc6n7-c6ccccc6)c6cccc7c6CCCC7)cc(C(C)C)c6ccc3c4c65)ccc1n2-c1ccccc1 ; Cc1ccc(N(c2cccc3c2CCCC3)c2cc(C(C)C)c3ccc4c(N(c5cccc6c5CCCC6)c5ccc(C)c6c5c5ccccc5n6-c5ccccc5)cc(C(C)C)c5ccc2c3c54)c2c3ccccc3n(-c3ccccc3)c12 ; CC(C)c1cc(N(c2ccc3c4ccccc4n(-c4ccccc4)c3c2)c2cccc3c2CCCC3)c2ccc3c(C(C)C)cc(N(c4cccc5c4CCCC5)c4cccc5c6ccccc6n(-c6ccccc6)c45)c4ccc1c2c34]

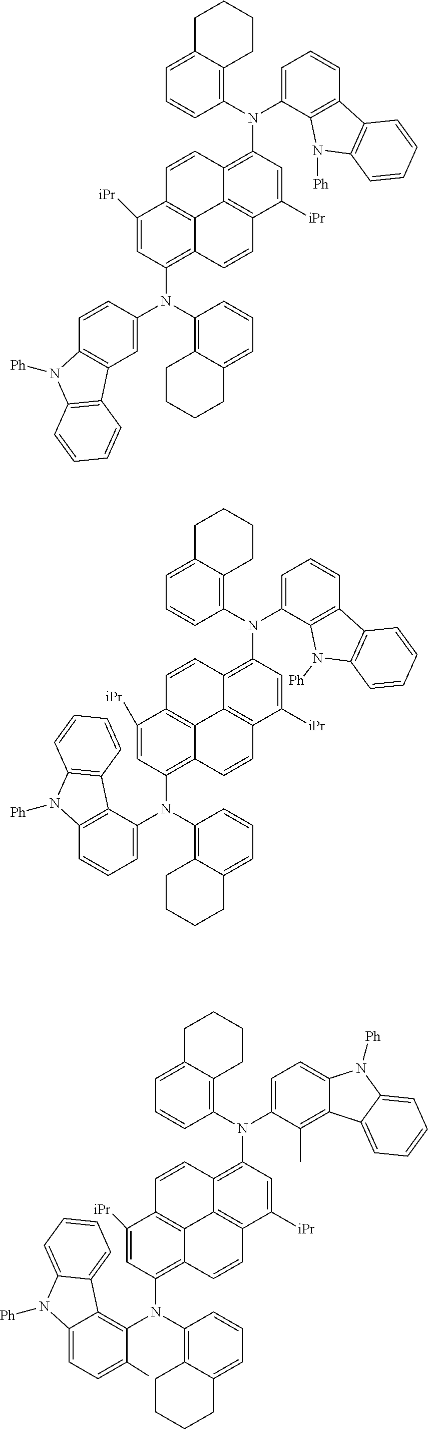 [SMILES: CC(C)c1cc(N(c2ccc3c(c2)c2ccccc2n3-c2ccccc2)c2cccc3c2CCCC3)c2ccc3c(C(C)C)cc(N(c4cccc5c4CCCC5)c4cccc5c6ccccc6n(-c6ccccc6)c45)c4ccc1c2c34 ; CC(C)c1cc(N(c2cccc3c2CCCC3)c2cccc3c2c2ccccc2n3-c2ccccc2)c2ccc3c(C(C)C)cc(N(c4cccc5c4CCCC5)c4cccc5c6ccccc6n(-c6ccccc6)c45)c4ccc1c2c34 ; Cc1ccc2c(c1N(c1cccc3c1CCCC3)c1cc(C(C)C)c3ccc4c(N(c5cccc6c5CCCC6)c5ccc6c(c5C)c5ccccc5n6-c5ccccc5)cc(C(C)C)c5ccc1c3c54)c1ccccc1n2-c1ccccc1]

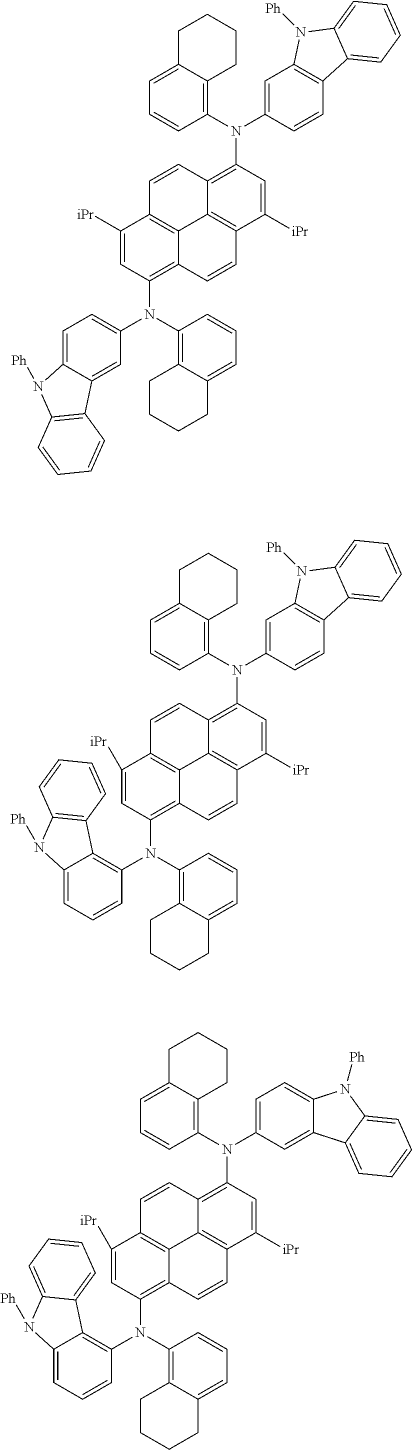 [SMILES: CC(C)c1cc(N(c2ccc3c(c2)c2ccccc2n3-c2ccccc2)c2cccc3c2CCCC3)c2ccc3c(C(C)C)cc(N(c4ccc5c6ccccc6n(-c6ccccc6)c5c4)c4cccc5c4CCCC5)c4ccc1c2c34 ; CC(C)c1cc(N(c2ccc3c4ccccc4n(-c4ccccc4)c3c2)c2cccc3c2CCCC3)c2ccc3c(C(C)C)cc(N(c4cccc5c4CCCC5)c4cccc5c4c4ccccc4n5-c4ccccc4)c4ccc1c2c34 ; CC(C)c1cc(N(c2ccc3c(c2)c2ccccc2n3-c2ccccc2)c2cccc3c2CCCC3)c2ccc3c(C(C)C)cc(N(c4cccc5c4CCCC5)c4cccc5c4c4ccccc4n5-c4ccccc4)c4ccc1c2c34]

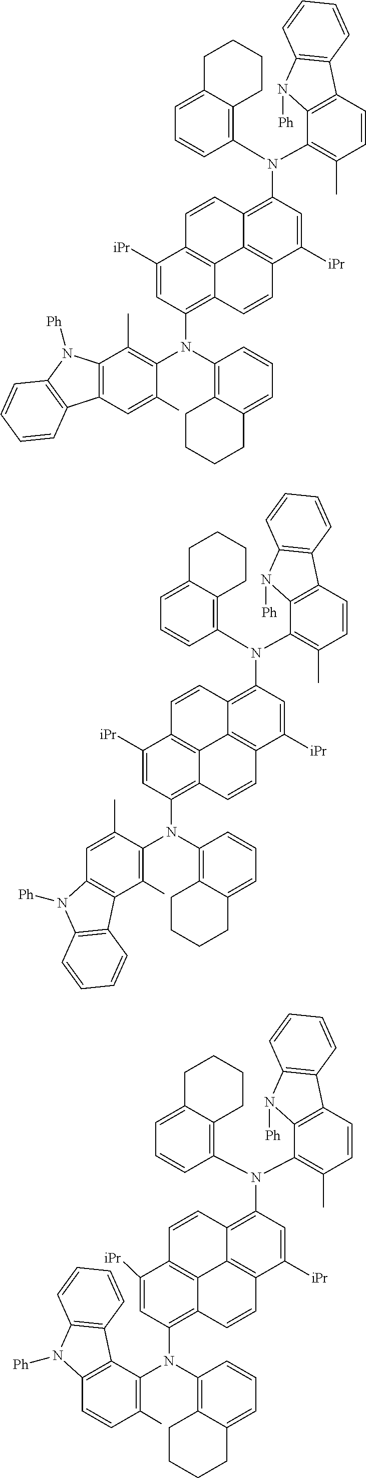 [SMILES: Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)ccc5c6ccccc6n(-c6ccccc6)c45)cc(C(C)C)c4ccc1c2c43 ; Cc1cc2c(c(C)c1N(c1cccc3c1CCCC3)c1cc(C(C)C)c3ccc4c(N(c5cccc6c5CCCC6)c5c(C)ccc6c7ccccc7n(-c7ccccc7)c56)cc(C(C)C)c5ccc1c3c54)c1ccccc1n2-c1ccccc1 ; Cc1ccc2c(c1N(c1cccc3c1CCCC3)c1cc(C(C)C)c3ccc4c(N(c5cccc6c5CCCC6)c5c(C)ccc6c7ccccc7n(-c7ccccc7)c56)cc(C(C)C)c5ccc1c3c54)c1ccccc1n2-c1ccccc1]

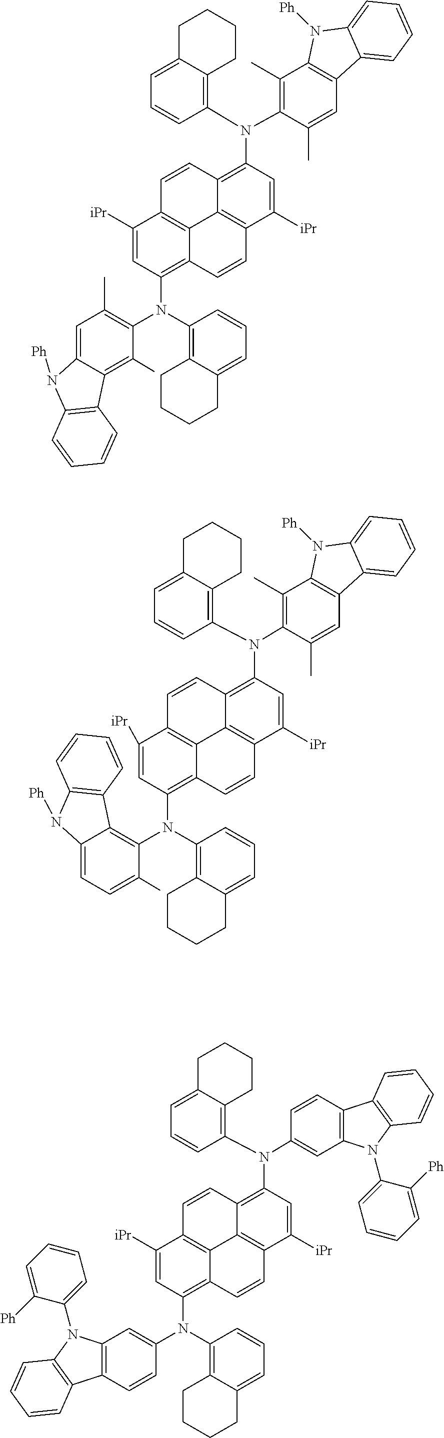 [SMILES: Cc1cc2c(c(C)c1N(c1cccc3c1CCCC3)c1cc(C(C)C)c3ccc4c(N(c5cccc6c5CCCC6)c5c(C)cc6c7ccccc7n(-c7ccccc7)c6c5C)cc(C(C)C)c5ccc1c3c54)c1ccccc1n2-c1ccccc1 ; Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)ccc5c4c4ccccc4n5-c4ccccc4)cc(C(C)C)c4ccc1c2c43 ; CC(C)c1cc(N(c2ccc3c4ccccc4n(-c4ccccc4-c4ccccc4)c3c2)c2cccc3c2CCCC3)c2ccc3c(C(C)C)cc(N(c4ccc5c6ccccc6n(-c6ccccc6-c6ccccc6)c5c4)c4cccc5c4CCCC5)c4ccc1c2c34]

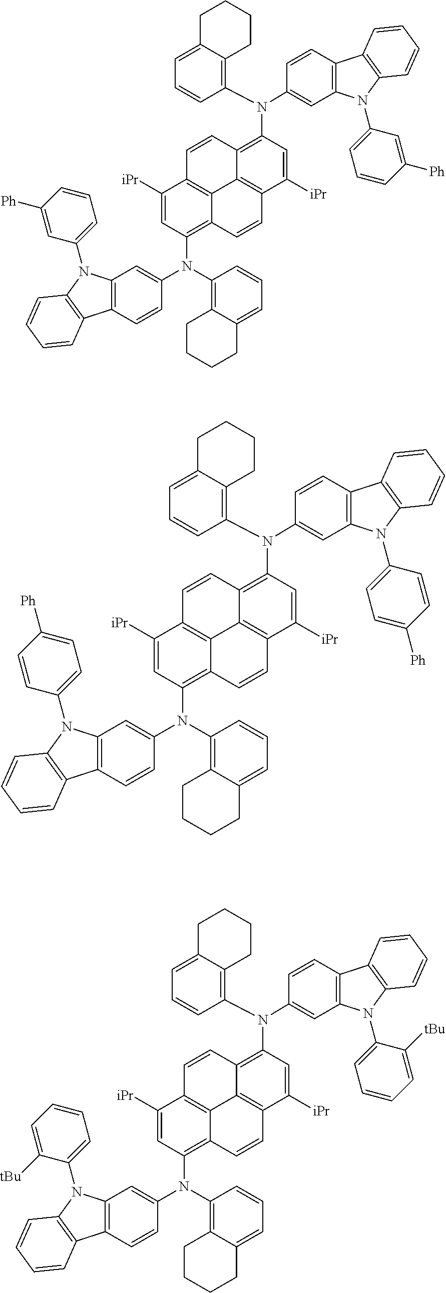 [SMILES: CC(C)c1cc(N(c2ccc3c4ccccc4n(-c4cccc(-c5ccccc5)c4)c3c2)c2cccc3c2CCCC3)c2ccc3c(C(C)C)cc(N(c4ccc5c6ccccc6n(-c6cccc(-c7ccccc7)c6)c5c4)c4cccc5c4CCCC5)c4ccc1c2c34 ; CC(C)c1cc(N(c2ccc3c4ccccc4n(-c4ccc(-c5ccccc5)cc4)c3c2)c2cccc3c2CCCC3)c2ccc3c(C(C)C)cc(N(c4ccc5c6ccccc6n(-c6ccc(-c7ccccc7)cc6)c5c4)c4cccc5c4CCCC5)c4ccc1c2c34 ; CC(C)c1cc(N(c2ccc3c4ccccc4n(-c4ccccc4C(C)(C)C)c3c2)c2cccc3c2CCCC3)c2ccc3c(C(C)C)cc(N(c4ccc5c6ccccc6n(-c6ccccc6C(C)(C)C)c5c4)c4cccc5c4CCCC5)c4ccc1c2c34]

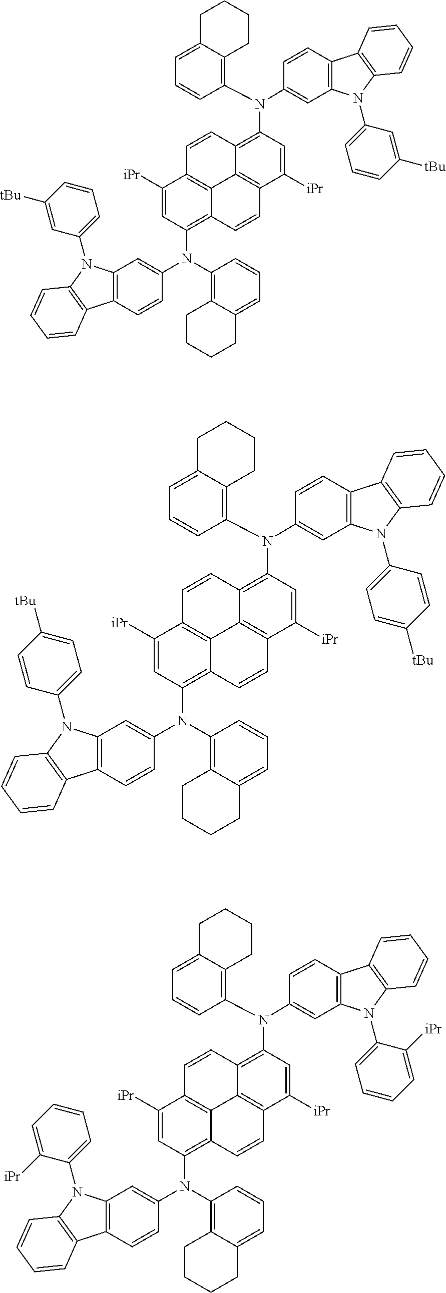 [SMILES: CC(C)c1cc(N(c2ccc3c4ccccc4n(-c4cccc(C(C)(C)C)c4)c3c2)c2cccc3c2CCCC3)c2ccc3c(C(C)C)cc(N(c4ccc5c6ccccc6n(-c6cccc(C(C)(C)C)c6)c5c4)c4cccc5c4CCCC5)c4ccc1c2c34 ; CC(C)c1cc(N(c2ccc3c4ccccc4n(-c4ccc(C(C)(C)C)cc4)c3c2)c2cccc3c2CCCC3)c2ccc3c(C(C)C)cc(N(c4ccc5c6ccccc6n(-c6ccc(C(C)(C)C)cc6)c5c4)c4cccc5c4CCCC5)c4ccc1c2c34 ; CC(C)c1ccccc1-n1c2ccccc2c2ccc(N(c3cccc4c3CCCC4)c3cc(C(C)C)c4ccc5c(N(c6ccc7c8ccccc8n(-c8ccccc8C(C)C)c7c6)c6cccc7c6CCCC7)cc(C(C)C)c6ccc3c4c65)cc21]

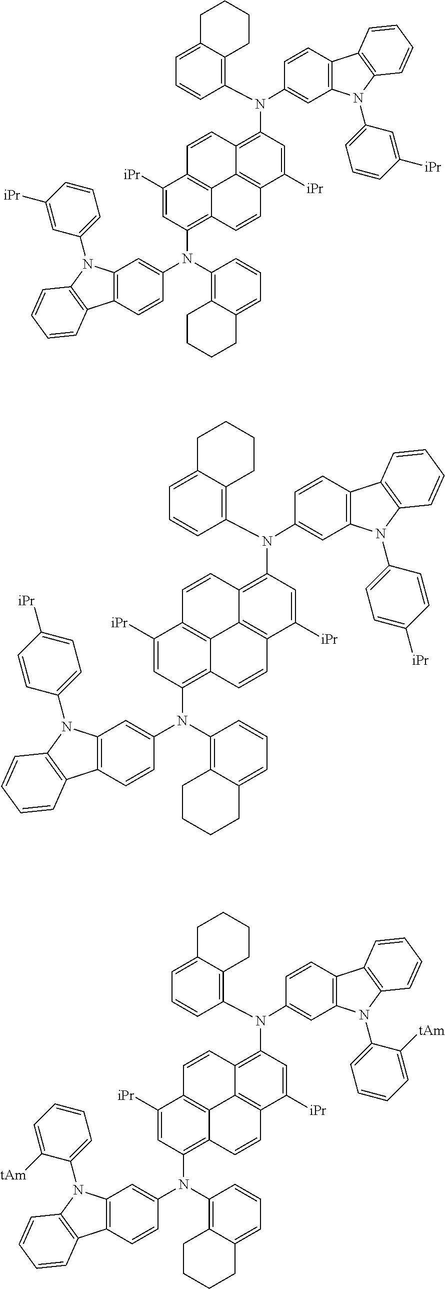

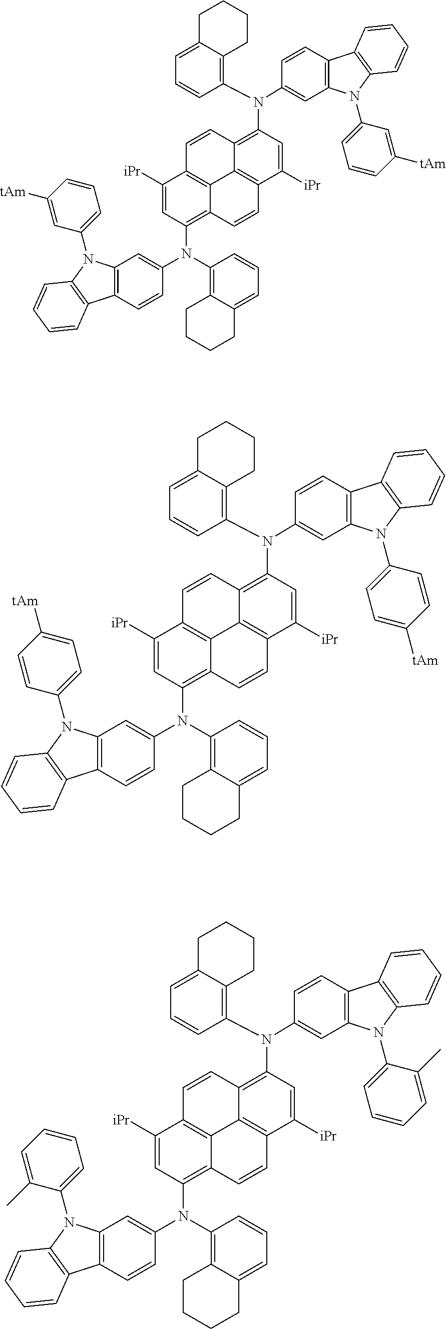

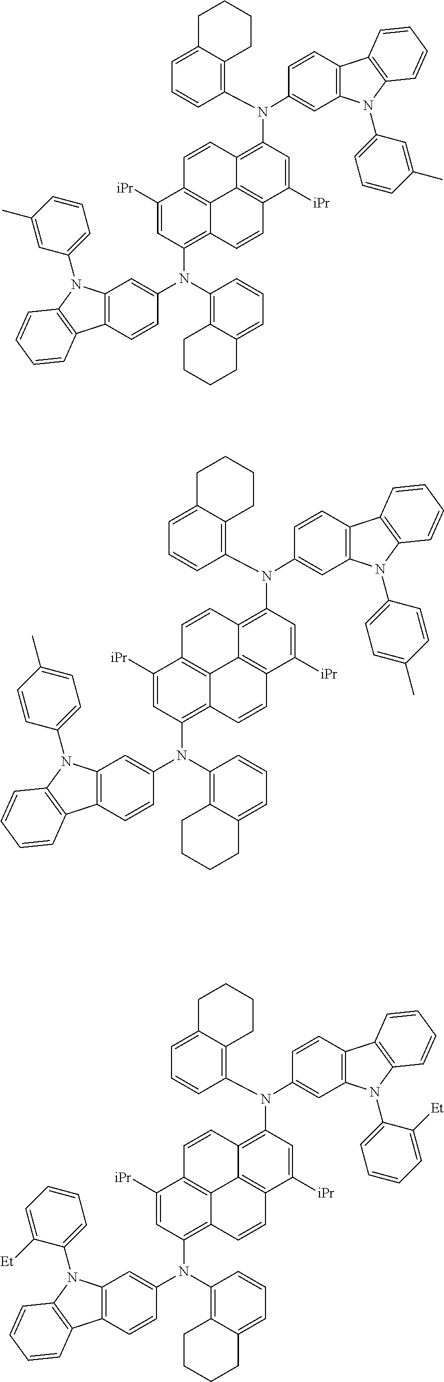

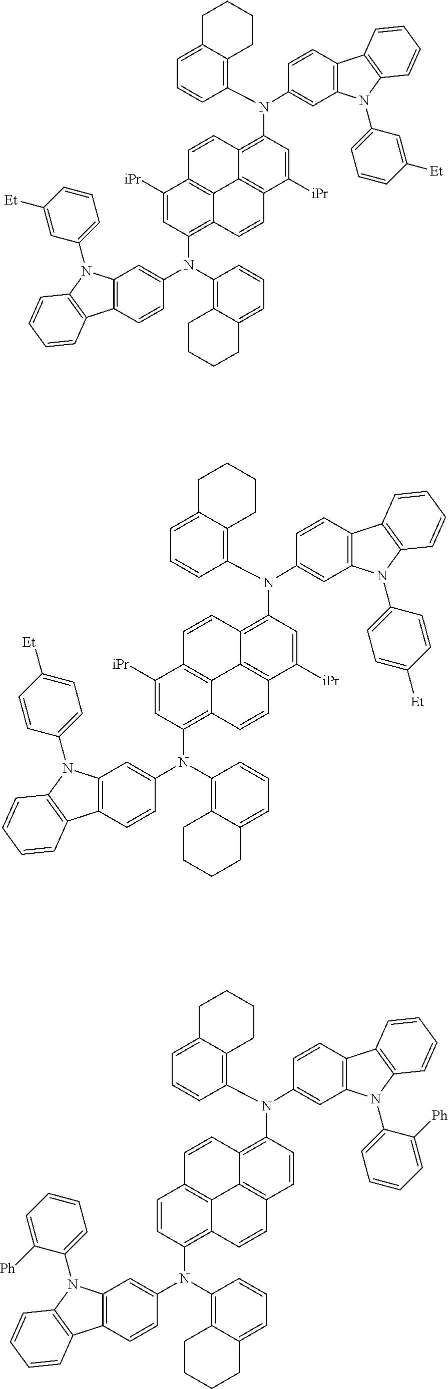 [SMILES: CCc1cccc(-n2c3ccccc3c3ccc(N(c4cccc5c4CCCC5)c4cc(C(C)C)c5ccc6c(N(c7ccc8c9ccccc9n(-c9cccc(CC)c9)c8c7)c7cccc8c7CCCC8)cc(C(C)C)c7ccc4c5c76)cc32)c1 ; CCc1ccc(-n2c3ccccc3c3ccc(N(c4cccc5c4CCCC5)c4cc(C(C)C)c5ccc6c(N(c7ccc8c9ccccc9n(-c9ccc(CC)cc9)c8c7)c7cccc8c7CCCC8)cc(C(C)C)c7ccc4c5c76)cc32)cc1 ; c1ccc(-c2ccccc2-n2c3ccccc3c3ccc(N(c4cccc5c4CCCC5)c4ccc5ccc6c(N(c7ccc8c9ccccc9n(-c9ccccc9-c9ccccc9)c8c7)c7cccc8c7CCCC8)ccc7ccc4c5c76)cc32)cc1]

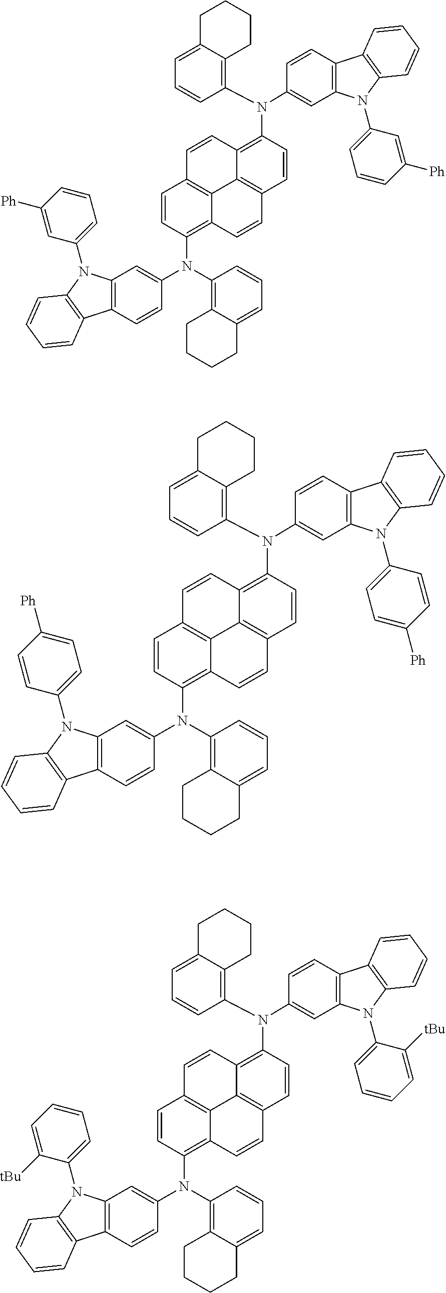 [SMILES: c1ccc(-c2cccc(-n3c4ccccc4c4ccc(N(c5cccc6c5CCCC6)c5ccc6ccc7c(N(c8ccc9c%10ccccc%10n(-c%10cccc(-c%11ccccc%11)c%10)c9c8)c8cccc9c8CCCC9)ccc8ccc5c6c87)cc43)c2)cc1 ; c1ccc(-c2ccc(-n3c4ccccc4c4ccc(N(c5cccc6c5CCCC6)c5ccc6ccc7c(N(c8ccc9c%10ccccc%10n(-c%10ccc(-c%11ccccc%11)cc%10)c9c8)c8cccc9c8CCCC9)ccc8ccc5c6c87)cc43)cc2)cc1 ; CC(C)(C)c1ccccc1-n1c2ccccc2c2ccc(N(c3cccc4c3CCCC4)c3ccc4ccc5c(N(c6ccc7c8ccccc8n(-c8ccccc8C(C)(C)C)c7c6)c6cccc7c6CCCC7)ccc6ccc3c4c65)cc21]

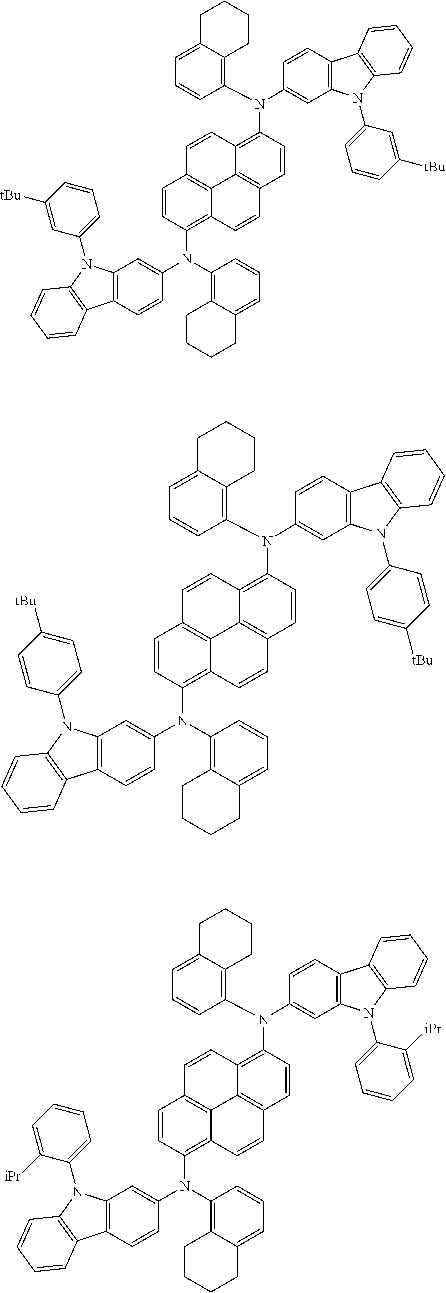 [SMILES: CC(C)(C)c1cccc(-n2c3ccccc3c3ccc(N(c4cccc5c4CCCC5)c4ccc5ccc6c(N(c7ccc8c9ccccc9n(-c9cccc(C(C)(C)C)c9)c8c7)c7cccc8c7CCCC8)ccc7ccc4c5c76)cc32)c1 ; CC(C)(C)c1ccc(-n2c3ccccc3c3ccc(N(c4cccc5c4CCCC5)c4ccc5ccc6c(N(c7ccc8c9ccccc9n(-c9ccc(C(C)(C)C)cc9)c8c7)c7cccc8c7CCCC8)ccc7ccc4c5c76)cc32)cc1 ; CC(C)c1ccccc1-n1c2ccccc2c2ccc(N(c3cccc4c3CCCC4)c3ccc4ccc5c(N(c6ccc7c8ccccc8n(-c8ccccc8C(C)C)c7c6)c6cccc7c6CCCC7)ccc6ccc3c4c65)cc21]

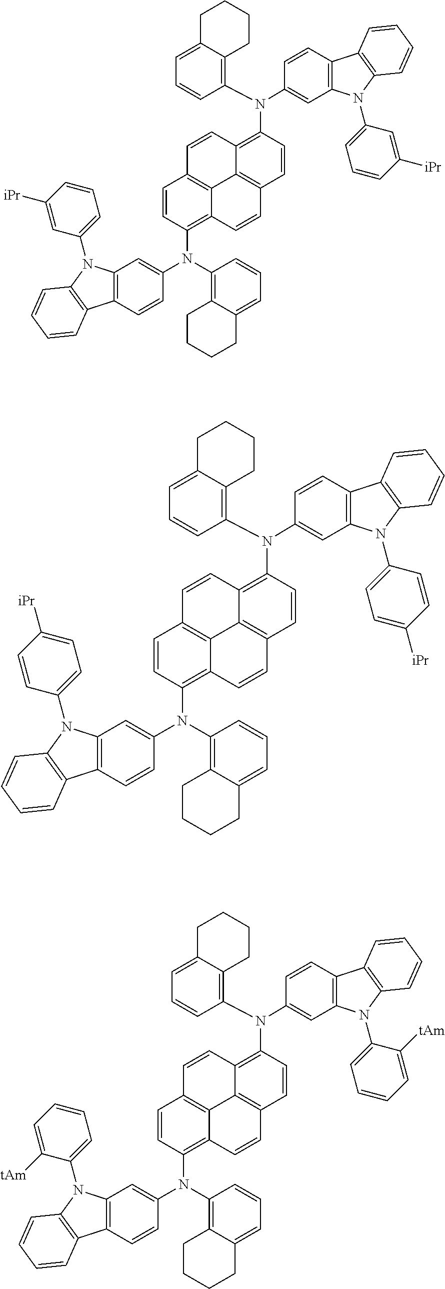

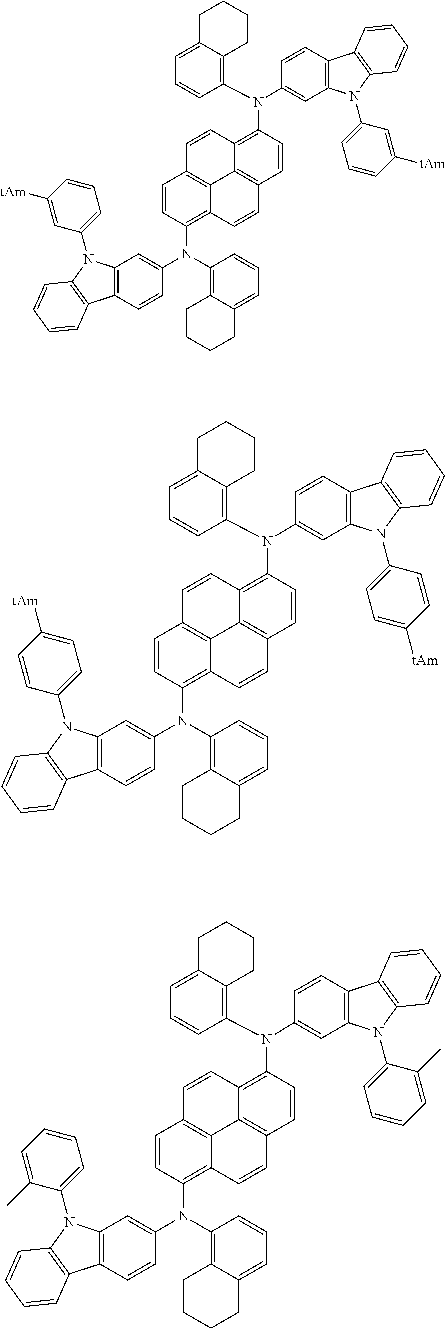

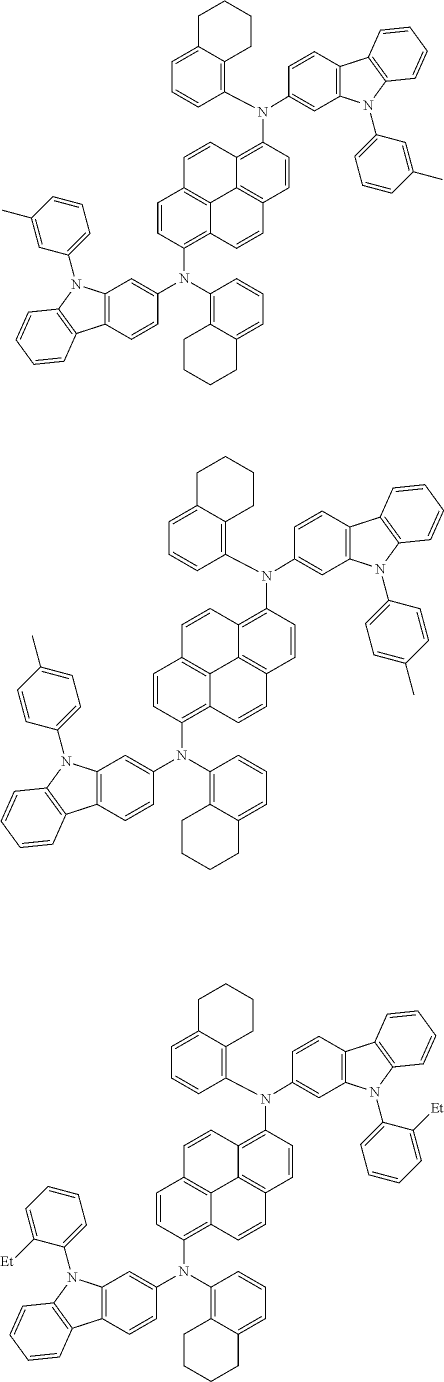

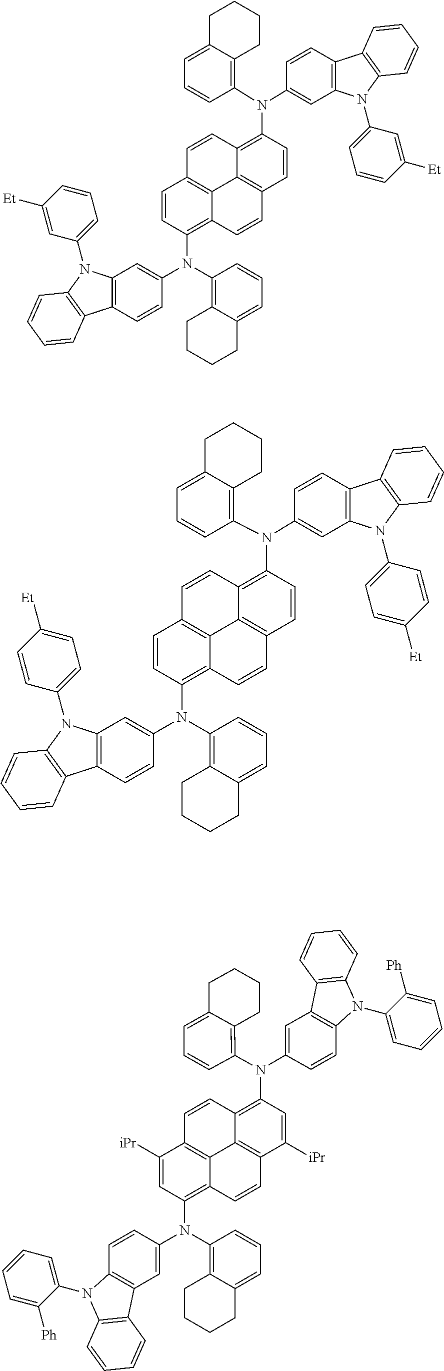 [SMILES: CCc1cccc(-n2c3ccccc3c3ccc(N(c4cccc5c4CCCC5)c4ccc5ccc6c(N(c7ccc8c9ccccc9n(-c9cccc(CC)c9)c8c7)c7cccc8c7CCCC8)ccc7ccc4c5c76)cc32)c1 ; CCc1ccc(-n2c3ccccc3c3ccc(N(c4cccc5c4CCCC5)c4ccc5ccc6c(N(c7ccc8c9ccccc9n(-c9ccc(CC)cc9)c8c7)c7cccc8c7CCCC8)ccc7ccc4c5c76)cc32)cc1 ; CC(C)c1cc(N(c2ccc3c(c2)c2ccccc2n3-c2ccccc2-c2ccccc2)c2cccc3c2CCCC3)c2ccc3c(C(C)C)cc(N(c4ccc5c(c4)c4ccccc4n5-c4ccccc4-c4ccccc4)c4cccc5c4CCCC5)c4ccc1c2c34]

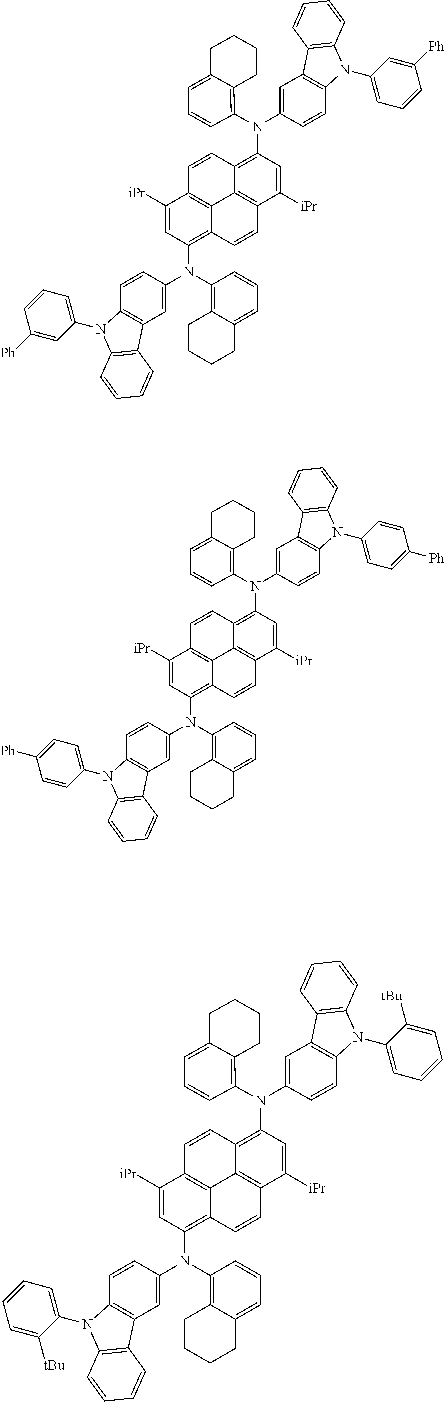 [SMILES: CC(C)c1cc(N(c2ccc3c(c2)c2ccccc2n3-c2cccc(-c3ccccc3)c2)c2cccc3c2CCCC3)c2ccc3c(C(C)C)cc(N(c4ccc5c(c4)c4ccccc4n5-c4cccc(-c5ccccc5)c4)c4cccc5c4CCCC5)c4ccc1c2c34 ; CC(C)c1cc(N(c2ccc3c(c2)c2ccccc2n3-c2ccc(-c3ccccc3)cc2)c2cccc3c2CCCC3)c2ccc3c(C(C)C)cc(N(c4ccc5c(c4)c4ccccc4n5-c4ccc(-c5ccccc5)cc4)c4cccc5c4CCCC5)c4ccc1c2c34 ; CC(C)c1cc(N(c2ccc3c(c2)c2ccccc2n3-c2ccccc2C(C)(C)C)c2cccc3c2CCCC3)c2ccc3c(C(C)C)cc(N(c4ccc5c(c4)c4ccccc4n5-c4ccccc4C(C)(C)C)c4cccc5c4CCCC5)c4ccc1c2c34]

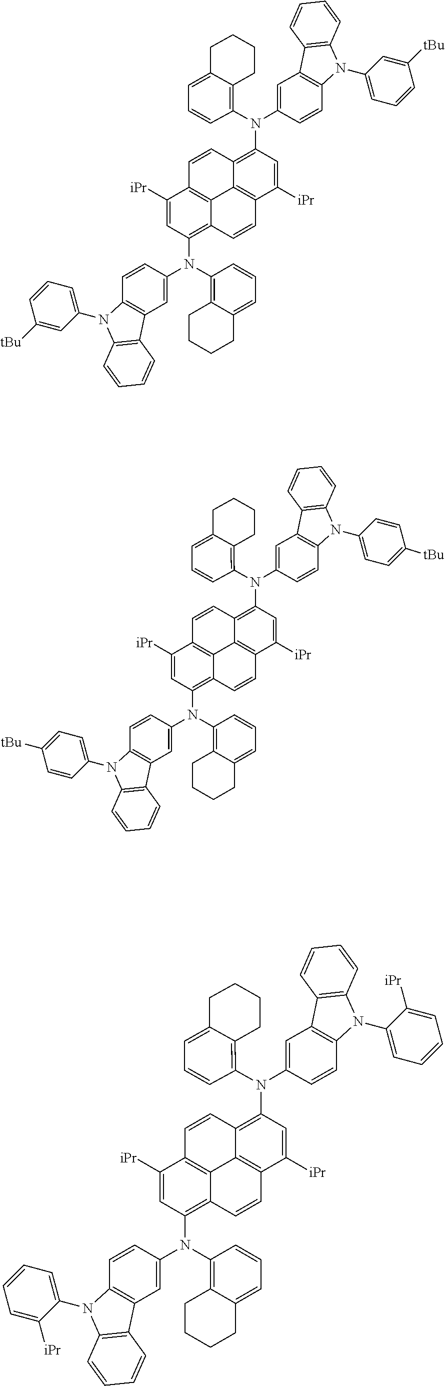 [SMILES: CC(C)c1cc(N(c2ccc3c(c2)c2ccccc2n3-c2cccc(C(C)(C)C)c2)c2cccc3c2CCCC3)c2ccc3c(C(C)C)cc(N(c4ccc5c(c4)c4ccccc4n5-c4cccc(C(C)(C)C)c4)c4cccc5c4CCCC5)c4ccc1c2c34 ; CC(C)c1cc(N(c2ccc3c(c2)c2ccccc2n3-c2ccc(C(C)(C)C)cc2)c2cccc3c2CCCC3)c2ccc3c(C(C)C)cc(N(c4ccc5c(c4)c4ccccc4n5-c4ccc(C(C)(C)C)cc4)c4cccc5c4CCCC5)c4ccc1c2c34 ; CC(C)c1ccccc1-n1c2ccccc2c2cc(N(c3cccc4c3CCCC4)c3cc(C(C)C)c4ccc5c(N(c6ccc7c(c6)c6ccccc6n7-c6ccccc6C(C)C)c6cccc7c6CCCC7)cc(C(C)C)c6ccc3c4c65)ccc21]

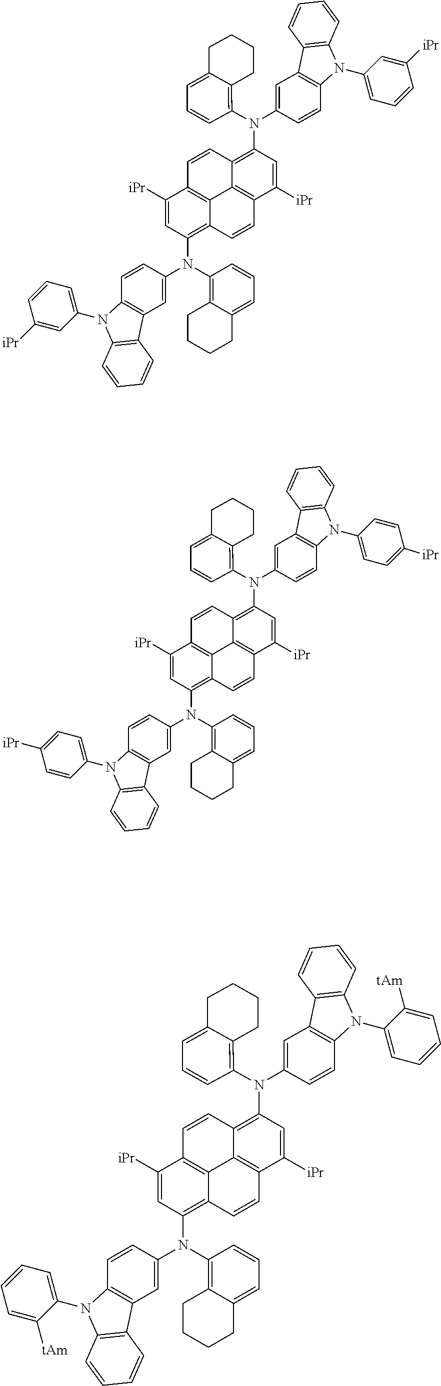

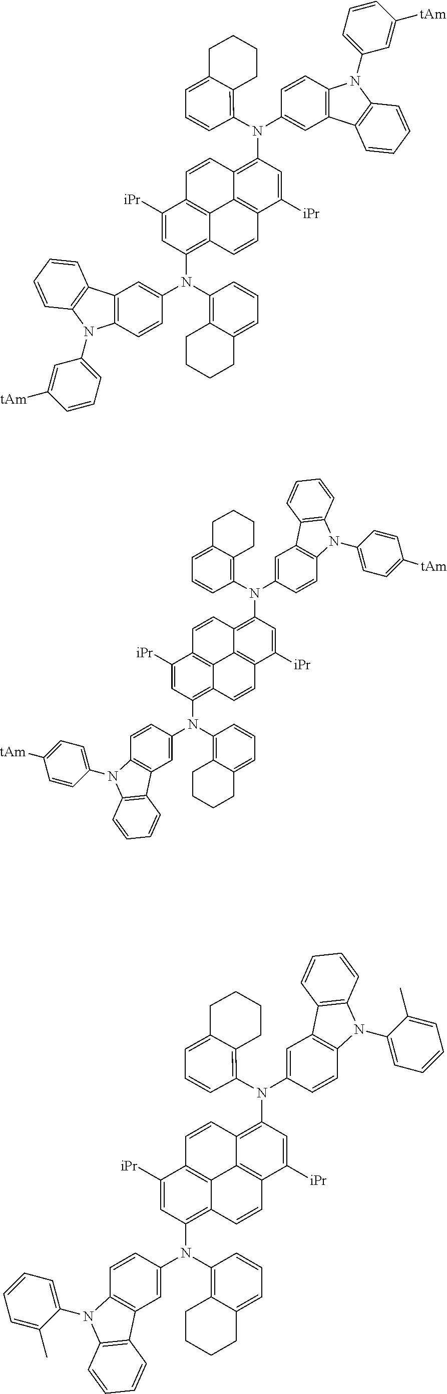

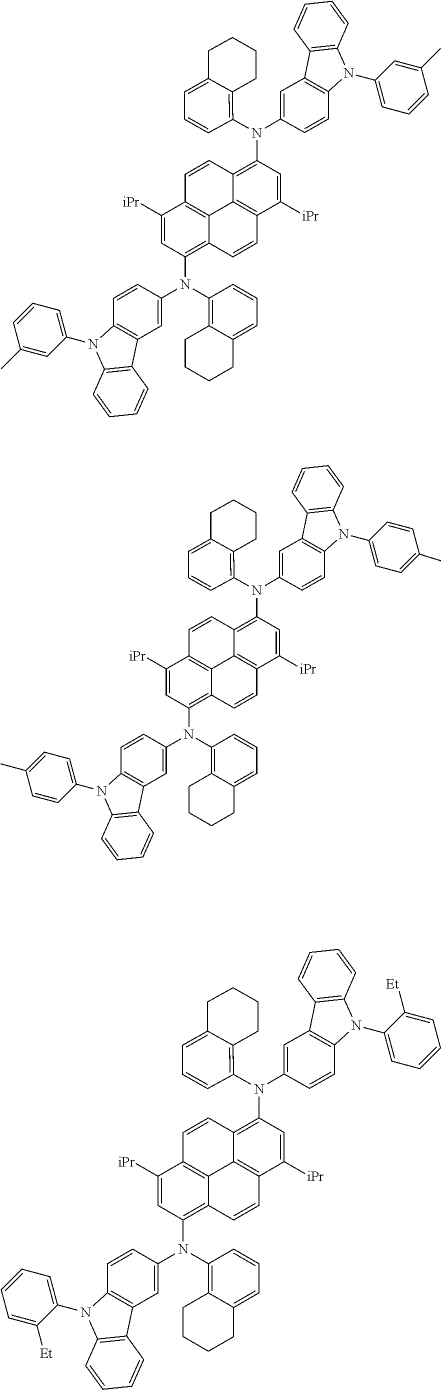

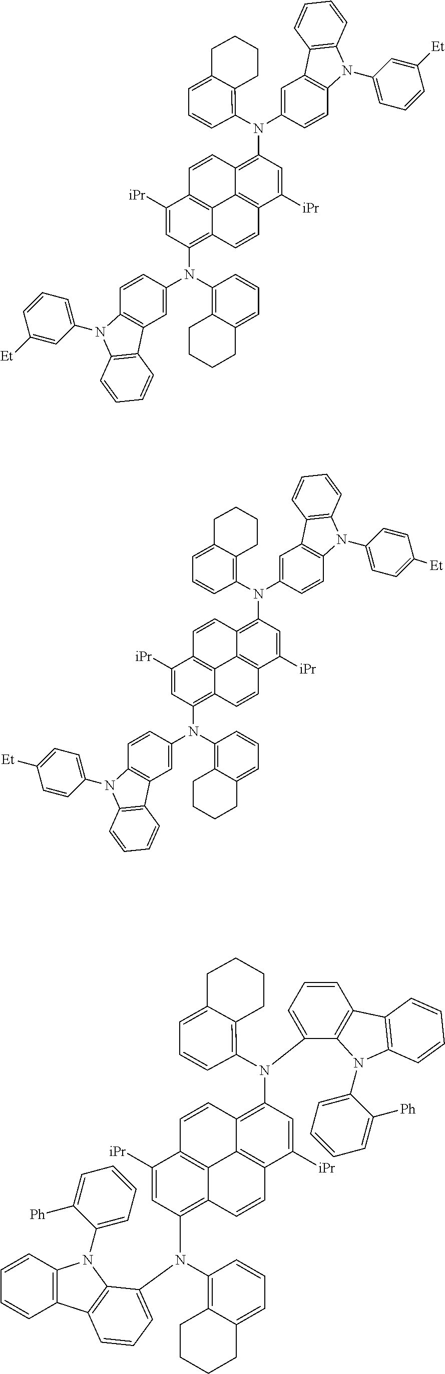 [SMILES: CCc1cccc(-n2c3ccccc3c3cc(N(c4cccc5c4CCCC5)c4cc(C(C)C)c5ccc6c(N(c7ccc8c(c7)c7ccccc7n8-c7cccc(CC)c7)c7cccc8c7CCCC8)cc(C(C)C)c7ccc4c5c76)ccc32)c1 ; CCc1ccc(-n2c3ccccc3c3cc(N(c4cccc5c4CCCC5)c4cc(C(C)C)c5ccc6c(N(c7ccc8c(c7)c7ccccc7n8-c7ccc(CC)cc7)c7cccc8c7CCCC8)cc(C(C)C)c7ccc4c5c76)ccc32)cc1 ; CC(C)c1cc(N(c2cccc3c2CCCC3)c2cccc3c4ccccc4n(-c4ccccc4-c4ccccc4)c23)c2ccc3c(C(C)C)cc(N(c4cccc5c4CCCC5)c4cccc5c6ccccc6n(-c6ccccc6-c6ccccc6)c45)c4ccc1c2c34]

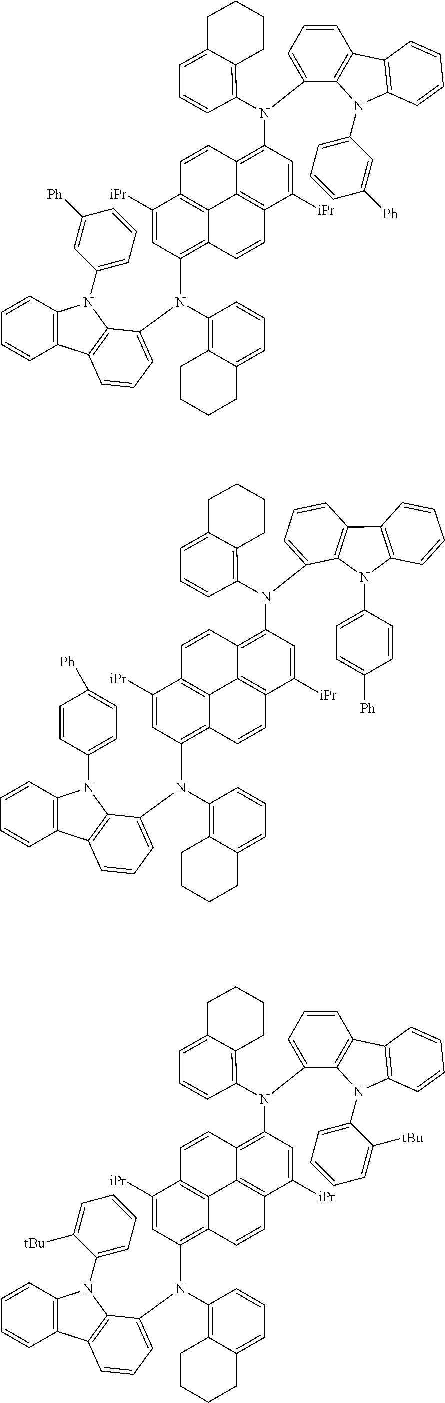 [SMILES: CC(C)c1cc(N(c2cccc3c2CCCC3)c2cccc3c4ccccc4n(-c4cccc(-c5ccccc5)c4)c23)c2ccc3c(C(C)C)cc(N(c4cccc5c4CCCC5)c4cccc5c6ccccc6n(-c6cccc(-c7ccccc7)c6)c45)c4ccc1c2c34 ; CC(C)c1cc(N(c2cccc3c2CCCC3)c2cccc3c4ccccc4n(-c4ccc(-c5ccccc5)cc4)c23)c2ccc3c(C(C)C)cc(N(c4cccc5c4CCCC5)c4cccc5c6ccccc6n(-c6ccc(-c7ccccc7)cc6)c45)c4ccc1c2c34 ; CC(C)c1cc(N(c2cccc3c2CCCC3)c2cccc3c4ccccc4n(-c4ccccc4C(C)(C)C)c23)c2ccc3c(C(C)C)cc(N(c4cccc5c4CCCC5)c4cccc5c6ccccc6n(-c6ccccc6C(C)(C)C)c45)c4ccc1c2c34]

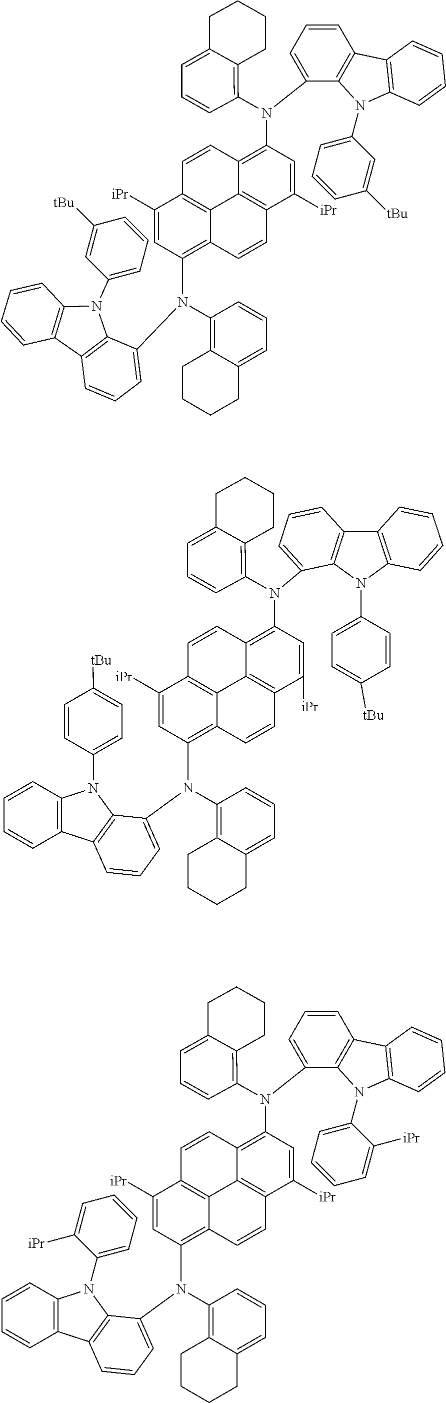 [SMILES: CC(C)c1cc(N(c2cccc3c2CCCC3)c2cccc3c4ccccc4n(-c4cccc(C(C)(C)C)c4)c23)c2ccc3c(C(C)C)cc(N(c4cccc5c4CCCC5)c4cccc5c6ccccc6n(-c6cccc(C(C)(C)C)c6)c45)c4ccc1c2c34 ; CC(C)c1cc(N(c2cccc3c2CCCC3)c2cccc3c4ccccc4n(-c4ccc(C(C)(C)C)cc4)c23)c2ccc3c(C(C)C)cc(N(c4cccc5c4CCCC5)c4cccc5c6ccccc6n(-c6ccc(C(C)(C)C)cc6)c45)c4ccc1c2c34 ; CC(C)c1ccccc1-n1c2ccccc2c2cccc(N(c3cccc4c3CCCC4)c3cc(C(C)C)c4ccc5c(N(c6cccc7c6CCCC7)c6cccc7c8ccccc8n(-c8ccccc8C(C)C)c67)cc(C(C)C)c6ccc3c4c65)c21]

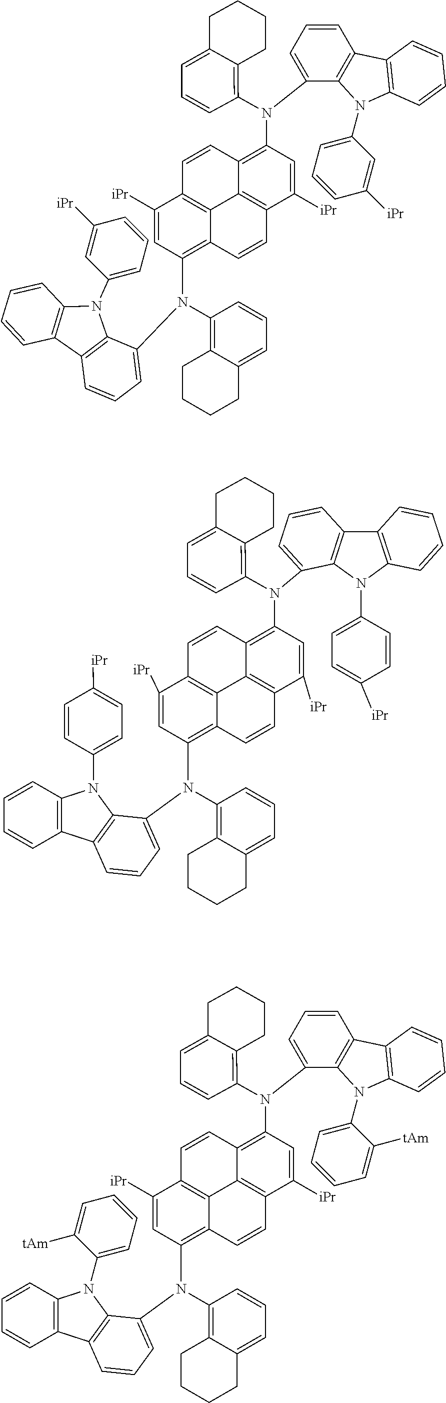

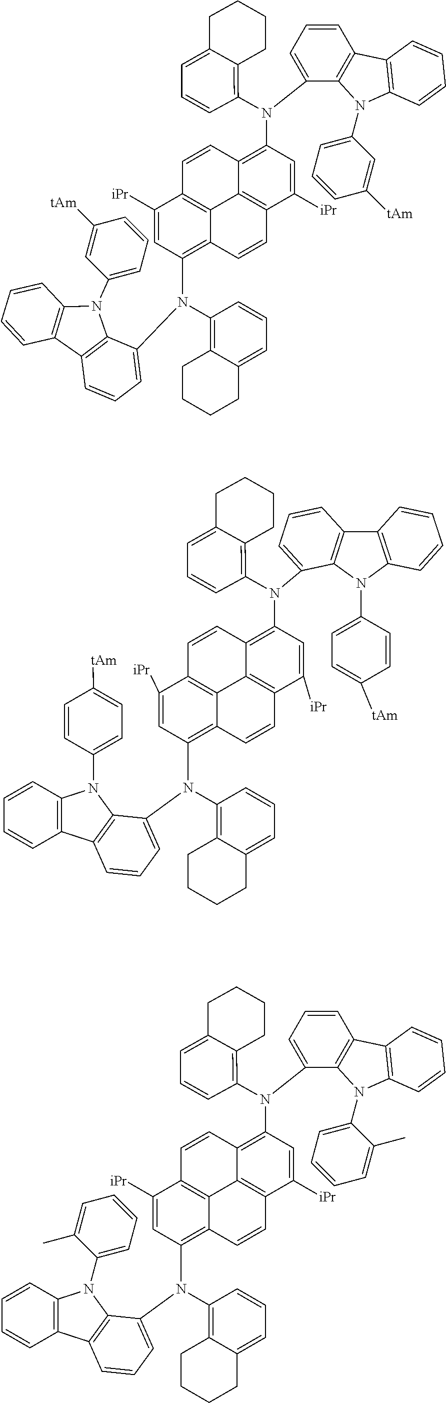

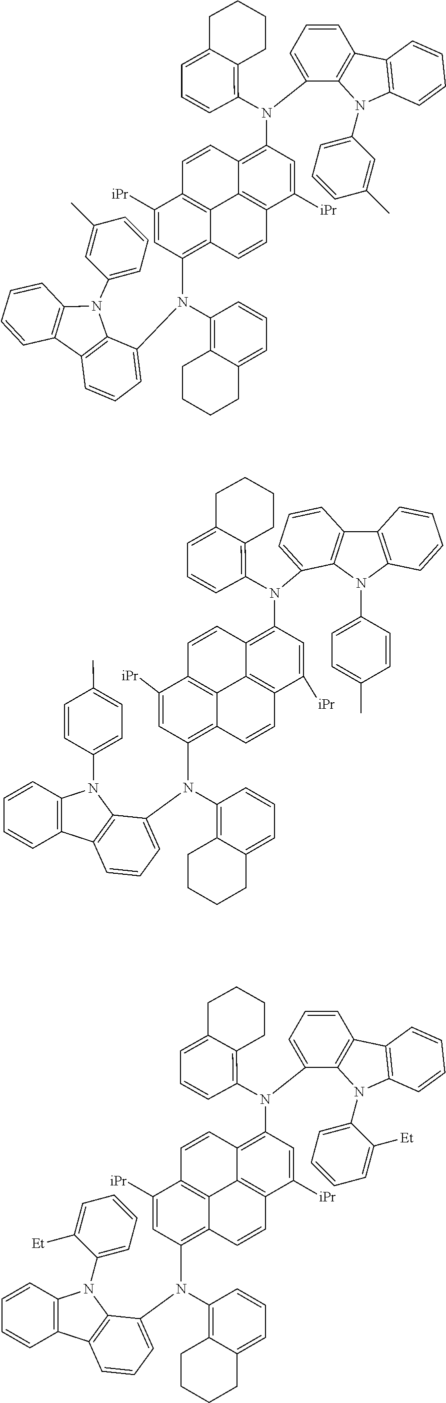 [SMILES: Cc1cccc(-n2c3ccccc3c3cccc(N(c4cccc5c4CCCC5)c4cc(C(C)C)c5ccc6c(N(c7cccc8c7CCCC8)c7cccc8c9ccccc9n(-c9cccc(C)c9)c78)cc(C(C)C)c7ccc4c5c76)c32)c1 ; Cc1ccc(-n2c3ccccc3c3cccc(N(c4cccc5c4CCCC5)c4cc(C(C)C)c5ccc6c(N(c7cccc8c7CCCC8)c7cccc8c9ccccc9n(-c9ccc(C)cc9)c78)cc(C(C)C)c7ccc4c5c76)c32)cc1 ; CCc1ccccc1-n1c2ccccc2c2cccc(N(c3cccc4c3CCCC4)c3cc(C(C)C)c4ccc5c(N(c6cccc7c6CCCC7)c6cccc7c8ccccc8n(-c8ccccc8CC)c67)cc(C(C)C)c6ccc3c4c65)c21]

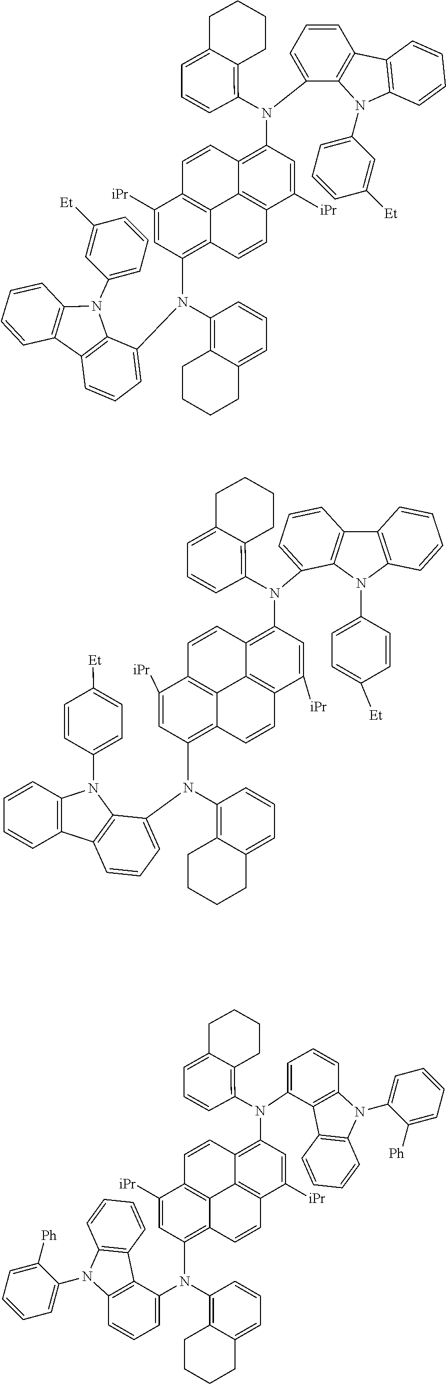 [SMILES: CCc1cccc(-n2c3ccccc3c3cccc(N(c4cccc5c4CCCC5)c4cc(C(C)C)c5ccc6c(N(c7cccc8c7CCCC8)c7cccc8c9ccccc9n(-c9cccc(CC)c9)c78)cc(C(C)C)c7ccc4c5c76)c32)c1 ; CCc1ccc(-n2c3ccccc3c3cccc(N(c4cccc5c4CCCC5)c4cc(C(C)C)c5ccc6c(N(c7cccc8c7CCCC8)c7cccc8c9ccccc9n(-c9ccc(CC)cc9)c78)cc(C(C)C)c7ccc4c5c76)c32)cc1 ; CC(C)c1cc(N(c2cccc3c2CCCC3)c2cccc3c2c2ccccc2n3-c2ccccc2-c2ccccc2)c2ccc3c(C(C)C)cc(N(c4cccc5c4CCCC5)c4cccc5c4c4ccccc4n5-c4ccccc4-c4ccccc4)c4ccc1c2c34]

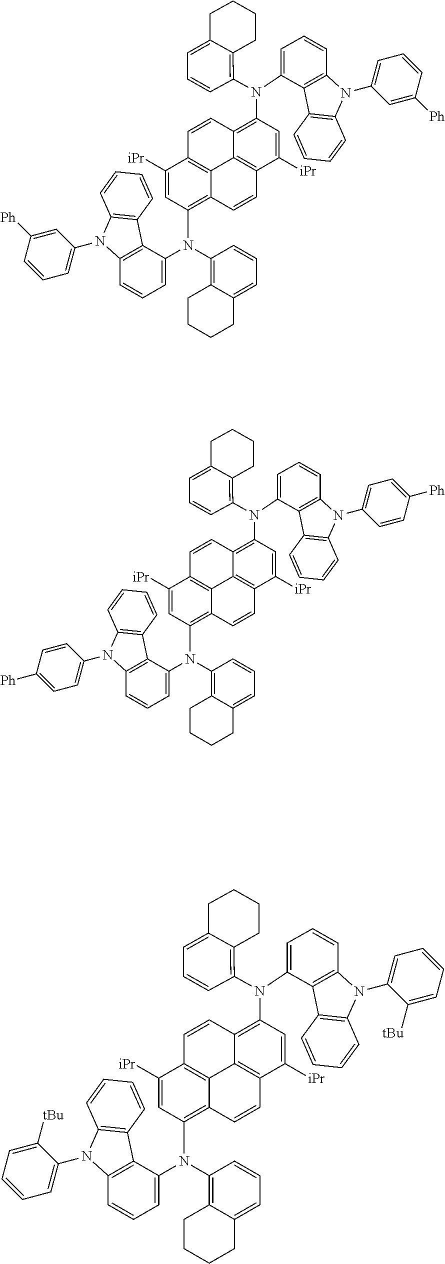 [SMILES: CC(C)c1cc(N(c2cccc3c2CCCC3)c2cccc3c2c2ccccc2n3-c2cccc(-c3ccccc3)c2)c2ccc3c(C(C)C)cc(N(c4cccc5c4CCCC5)c4cccc5c4c4ccccc4n5-c4cccc(-c5ccccc5)c4)c4ccc1c2c34 ; CC(C)c1cc(N(c2cccc3c2CCCC3)c2cccc3c2c2ccccc2n3-c2ccc(-c3ccccc3)cc2)c2ccc3c(C(C)C)cc(N(c4cccc5c4CCCC5)c4cccc5c4c4ccccc4n5-c4ccc(-c5ccccc5)cc4)c4ccc1c2c34 ; CC(C)c1cc(N(c2cccc3c2CCCC3)c2cccc3c2c2ccccc2n3-c2ccccc2C(C)(C)C)c2ccc3c(C(C)C)cc(N(c4cccc5c4CCCC5)c4cccc5c4c4ccccc4n5-c4ccccc4C(C)(C)C)c4ccc1c2c34]

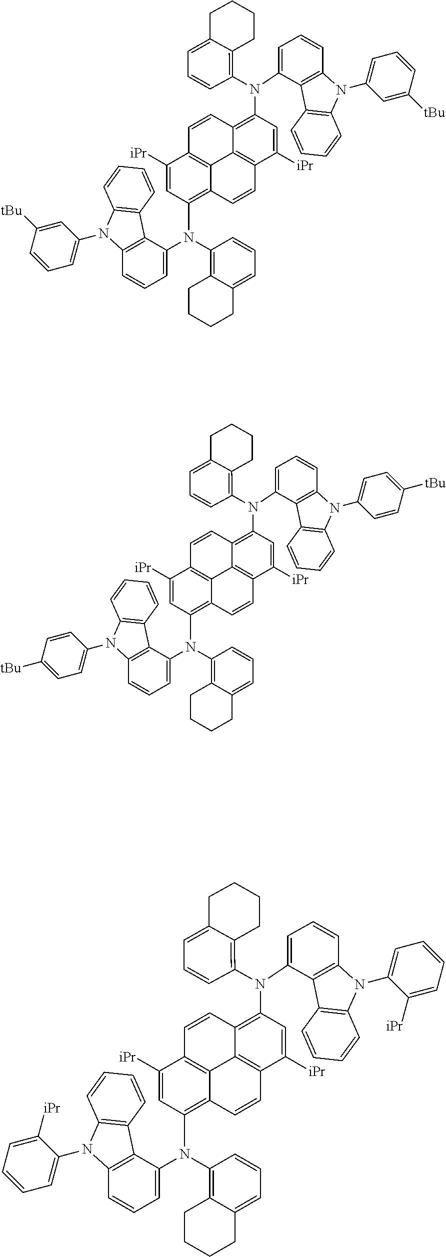 [SMILES: CC(C)c1cc(N(c2cccc3c2CCCC3)c2cccc3c2c2ccccc2n3-c2cccc(C(C)(C)C)c2)c2ccc3c(C(C)C)cc(N(c4cccc5c4CCCC5)c4cccc5c4c4ccccc4n5-c4cccc(C(C)(C)C)c4)c4ccc1c2c34 ; CC(C)c1cc(N(c2cccc3c2CCCC3)c2cccc3c2c2ccccc2n3-c2ccc(C(C)(C)C)cc2)c2ccc3c(C(C)C)cc(N(c4cccc5c4CCCC5)c4cccc5c4c4ccccc4n5-c4ccc(C(C)(C)C)cc4)c4ccc1c2c34 ; CC(C)c1ccccc1-n1c2ccccc2c2c(N(c3cccc4c3CCCC4)c3cc(C(C)C)c4ccc5c(N(c6cccc7c6CCCC7)c6cccc7c6c6ccccc6n7-c6ccccc6C(C)C)cc(C(C)C)c6ccc3c4c65)cccc21]

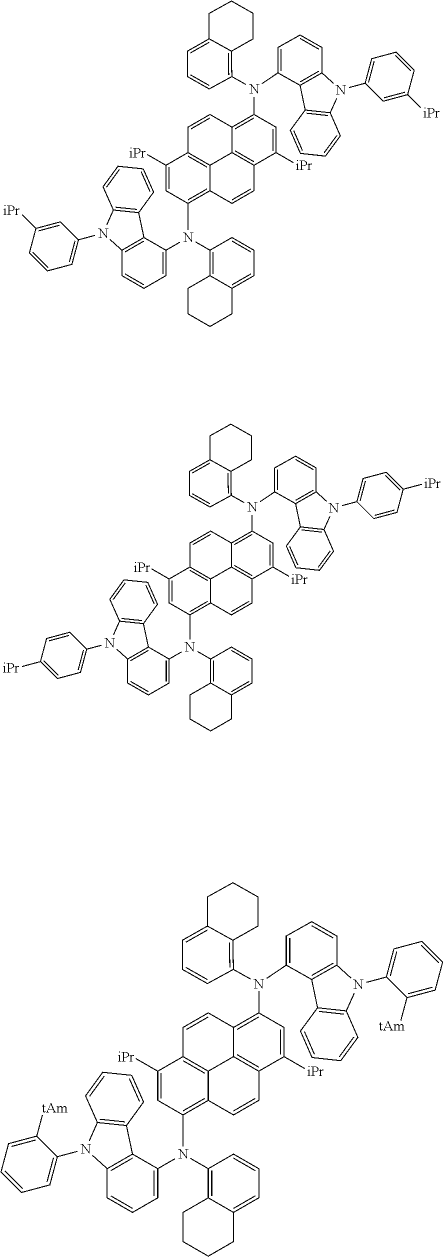

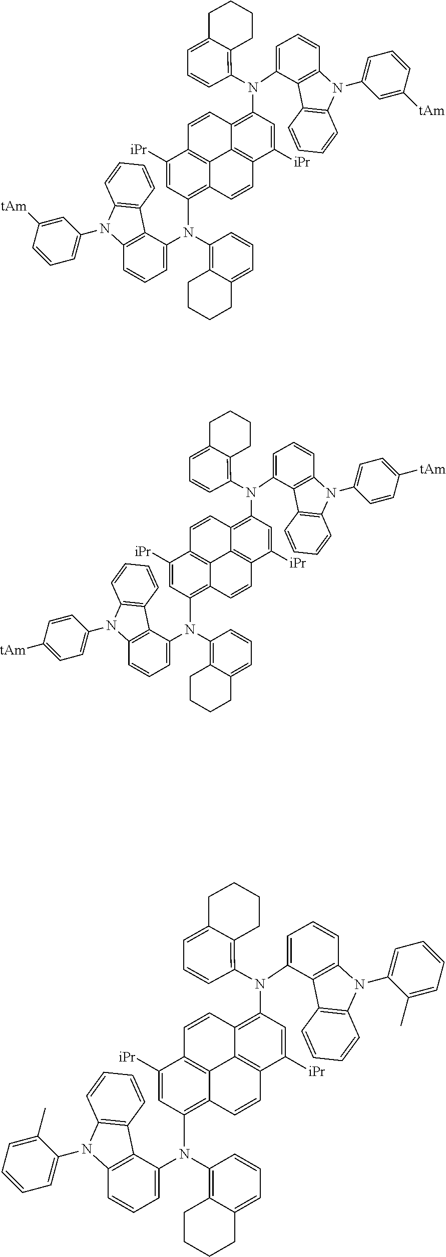

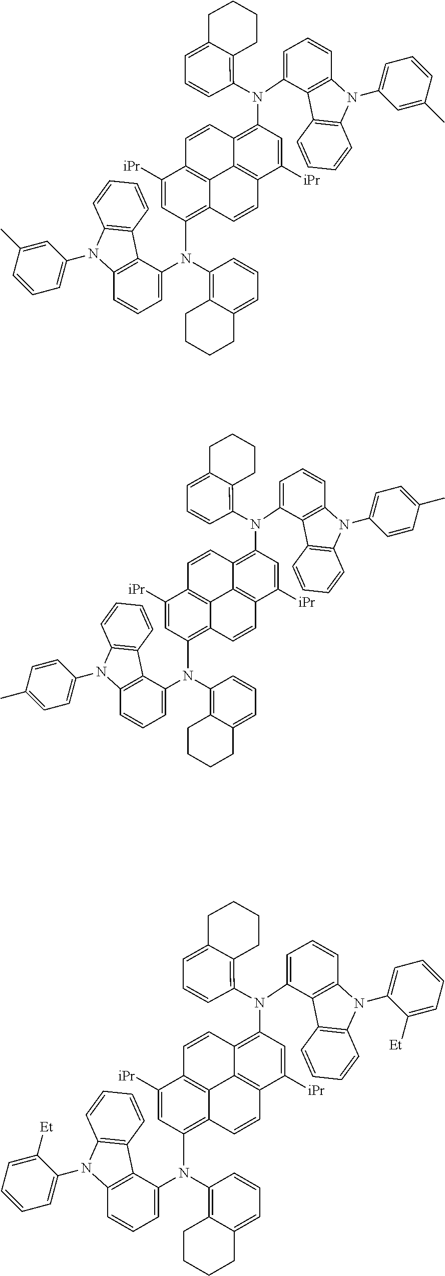

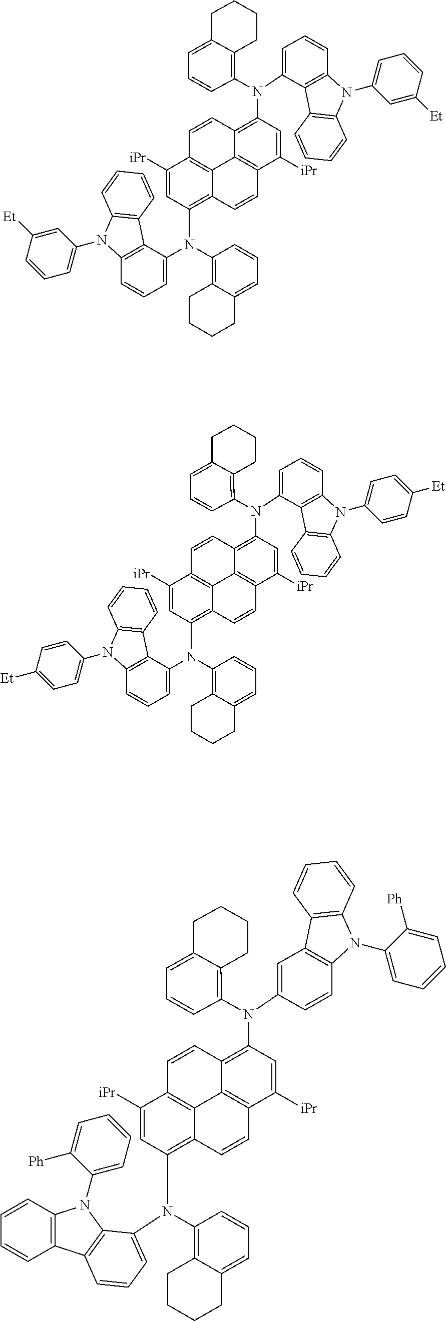 [SMILES: CCc1cccc(-n2c3ccccc3c3c(N(c4cccc5c4CCCC5)c4cc(C(C)C)c5ccc6c(N(c7cccc8c7CCCC8)c7cccc8c7c7ccccc7n8-c7cccc(CC)c7)cc(C(C)C)c7ccc4c5c76)cccc32)c1 ; CCc1ccc(-n2c3ccccc3c3c(N(c4cccc5c4CCCC5)c4cc(C(C)C)c5ccc6c(N(c7cccc8c7CCCC8)c7cccc8c7c7ccccc7n8-c7ccc(CC)cc7)cc(C(C)C)c7ccc4c5c76)cccc32)cc1 ; CC(C)c1cc(N(c2ccc3c(c2)c2ccccc2n3-c2ccccc2-c2ccccc2)c2cccc3c2CCCC3)c2ccc3c(C(C)C)cc(N(c4cccc5c4CCCC5)c4cccc5c6ccccc6n(-c6ccccc6-c6ccccc6)c45)c4ccc1c2c34]

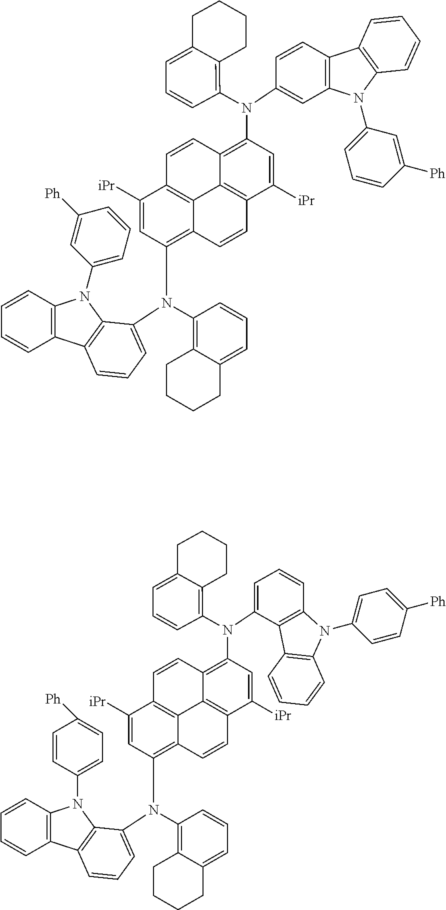 [SMILES: CC(C)c1cc(N(c2ccc3c4ccccc4n(-c4cccc(-c5ccccc5)c4)c3c2)c2cccc3c2CCCC3)c2ccc3c(C(C)C)cc(N(c4cccc5c4CCCC5)c4cccc5c6ccccc6n(-c6cccc(-c7ccccc7)c6)c45)c4ccc1c2c34 ; CC(C)c1cc(N(c2cccc3c2CCCC3)c2cccc3c2c2ccccc2n3-c2ccc(-c3ccccc3)cc2)c2ccc3c(C(C)C)cc(N(c4cccc5c4CCCC5)c4cccc5c6ccccc6n(-c6ccc(-c7ccccc7)cc6)c45)c4ccc1c2c34]

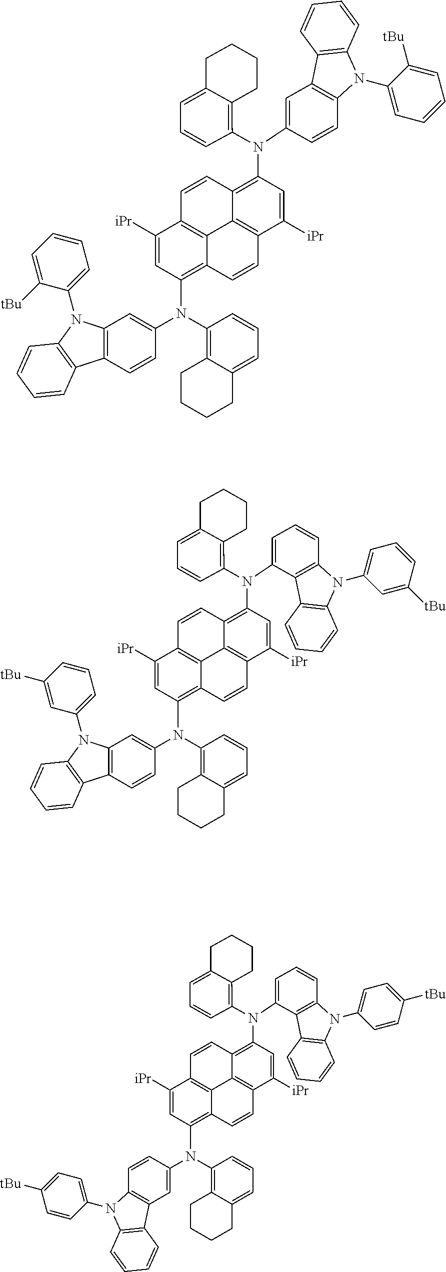 [SMILES: CC(C)c1cc(N(c2ccc3c(c2)c2ccccc2n3-c2ccccc2C(C)(C)C)c2cccc3c2CCCC3)c2ccc3c(C(C)C)cc(N(c4ccc5c6ccccc6n(-c6ccccc6C(C)(C)C)c5c4)c4cccc5c4CCCC5)c4ccc1c2c34 ; CC(C)c1cc(N(c2ccc3c4ccccc4n(-c4cccc(C(C)(C)C)c4)c3c2)c2cccc3c2CCCC3)c2ccc3c(C(C)C)cc(N(c4cccc5c4CCCC5)c4cccc5c4c4ccccc4n5-c4cccc(C(C)(C)C)c4)c4ccc1c2c34 ; CC(C)c1cc(N(c2ccc3c(c2)c2ccccc2n3-c2ccc(C(C)(C)C)cc2)c2cccc3c2CCCC3)c2ccc3c(C(C)C)cc(N(c4cccc5c4CCCC5)c4cccc5c4c4ccccc4n5-c4ccc(C(C)(C)C)cc4)c4ccc1c2c34]

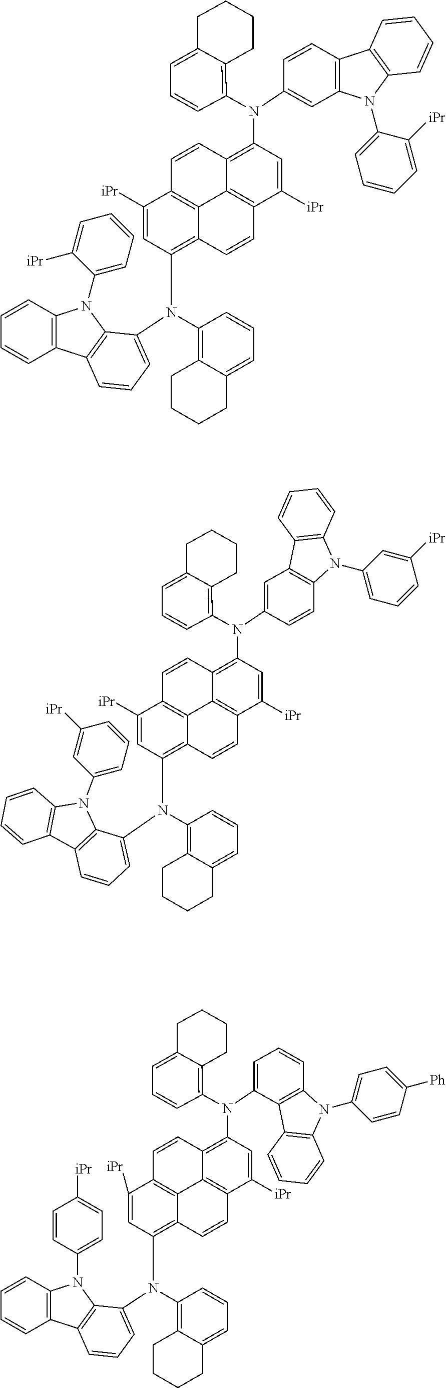 [SMILES: CC(C)c1ccccc1-n1c2ccccc2c2ccc(N(c3cccc4c3CCCC4)c3cc(C(C)C)c4ccc5c(N(c6cccc7c6CCCC7)c6cccc7c8ccccc8n(-c8ccccc8C(C)C)c67)cc(C(C)C)c6ccc3c4c65)cc21 ; CC(C)c1cccc(-n2c3ccccc3c3cc(N(c4cccc5c4CCCC5)c4cc(C(C)C)c5ccc6c(N(c7cccc8c7CCCC8)c7cccc8c9ccccc9n(-c9cccc(C(C)C)c9)c78)cc(C(C)C)c7ccc4c5c76)ccc32)c1 ; CC(C)c1ccc(-n2c3ccccc3c3cccc(N(c4cccc5c4CCCC5)c4cc(C(C)C)c5ccc6c(N(c7cccc8c7CCCC8)c7cccc8c7c7ccccc7n8-c7ccc(-c8ccccc8)cc7)cc(C(C)C)c7ccc4c5c76)c32)cc1]

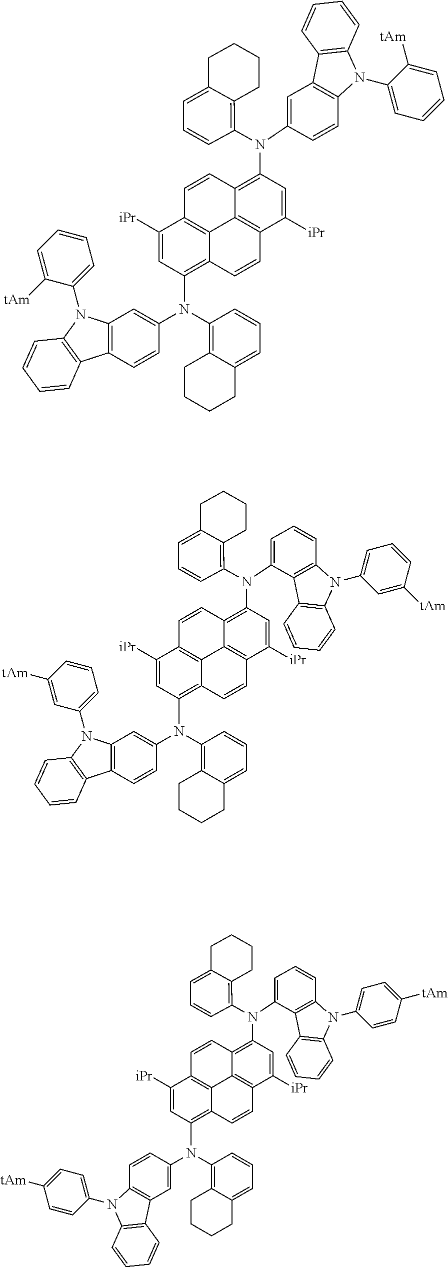

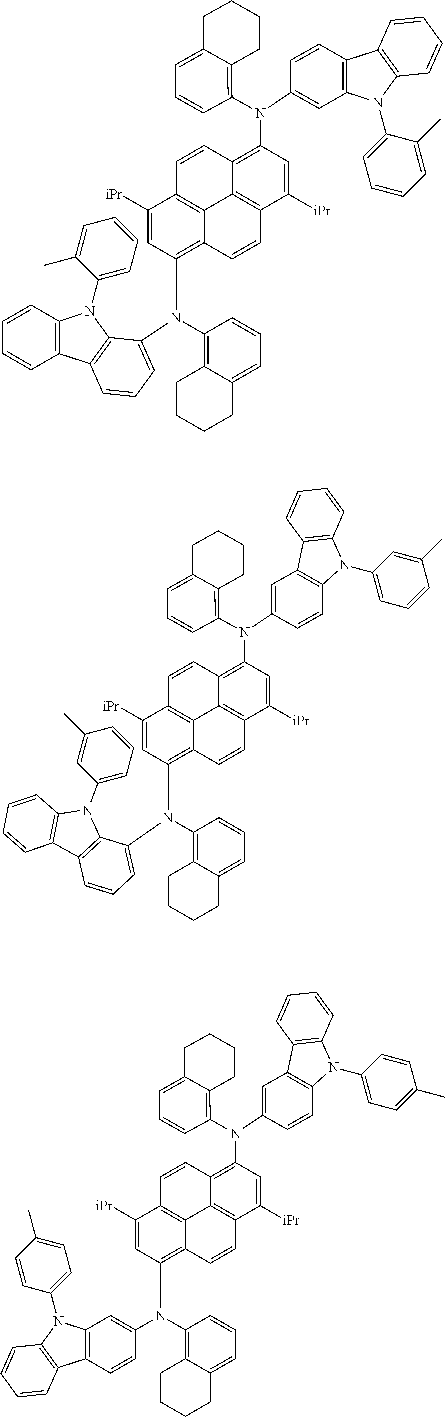 [SMILES: Cc1ccccc1-n1c2ccccc2c2ccc(N(c3cccc4c3CCCC4)c3cc(C(C)C)c4ccc5c(N(c6cccc7c6CCCC7)c6cccc7c8ccccc8n(-c8ccccc8C)c67)cc(C(C)C)c6ccc3c4c65)cc21 ; Cc1cccc(-n2c3ccccc3c3cc(N(c4cccc5c4CCCC5)c4cc(C(C)C)c5ccc6c(N(c7cccc8c7CCCC8)c7cccc8c9ccccc9n(-c9cccc(C)c9)c78)cc(C(C)C)c7ccc4c5c76)ccc32)c1 ; Cc1ccc(-n2c3ccccc3c3cc(N(c4cccc5c4CCCC5)c4cc(C(C)C)c5ccc6c(N(c7ccc8c9ccccc9n(-c9ccc(C)cc9)c8c7)c7cccc8c7CCCC8)cc(C(C)C)c7ccc4c5c76)ccc32)cc1]

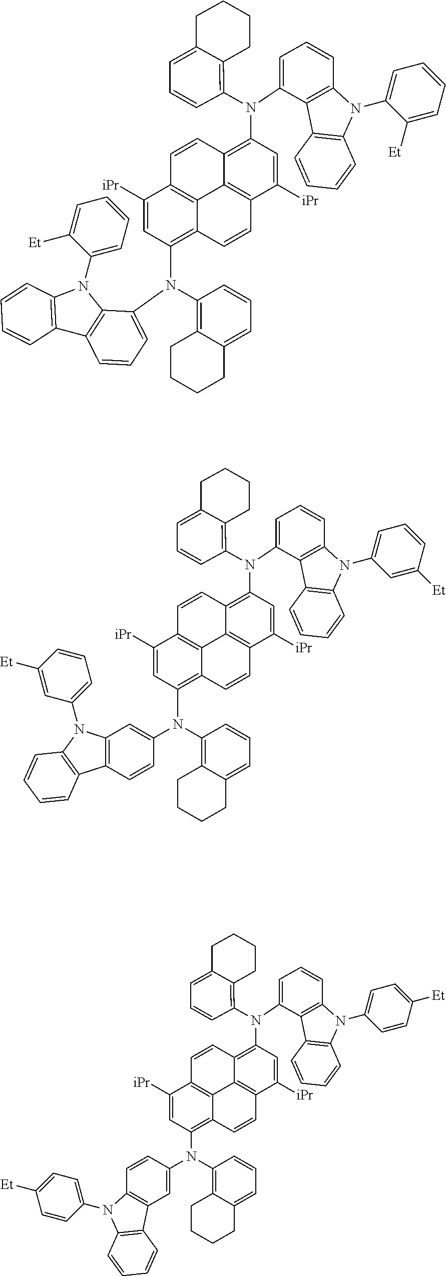 [SMILES: CCc1ccccc1-n1c2ccccc2c2c(N(c3cccc4c3CCCC4)c3cc(C(C)C)c4ccc5c(N(c6cccc7c6CCCC7)c6cccc7c8ccccc8n(-c8ccccc8CC)c67)cc(C(C)C)c6ccc3c4c65)cccc21 ; CCc1cccc(-n2c3ccccc3c3ccc(N(c4cccc5c4CCCC5)c4cc(C(C)C)c5ccc6c(N(c7cccc8c7CCCC8)c7cccc8c7c7ccccc7n8-c7cccc(CC)c7)cc(C(C)C)c7ccc4c5c76)cc32)c1 ; CCc1ccc(-n2c3ccccc3c3cc(N(c4cccc5c4CCCC5)c4cc(C(C)C)c5ccc6c(N(c7cccc8c7CCCC8)c7cccc8c7c7ccccc7n8-c7ccc(CC)cc7)cc(C(C)C)c7ccc4c5c76)ccc32)cc1]

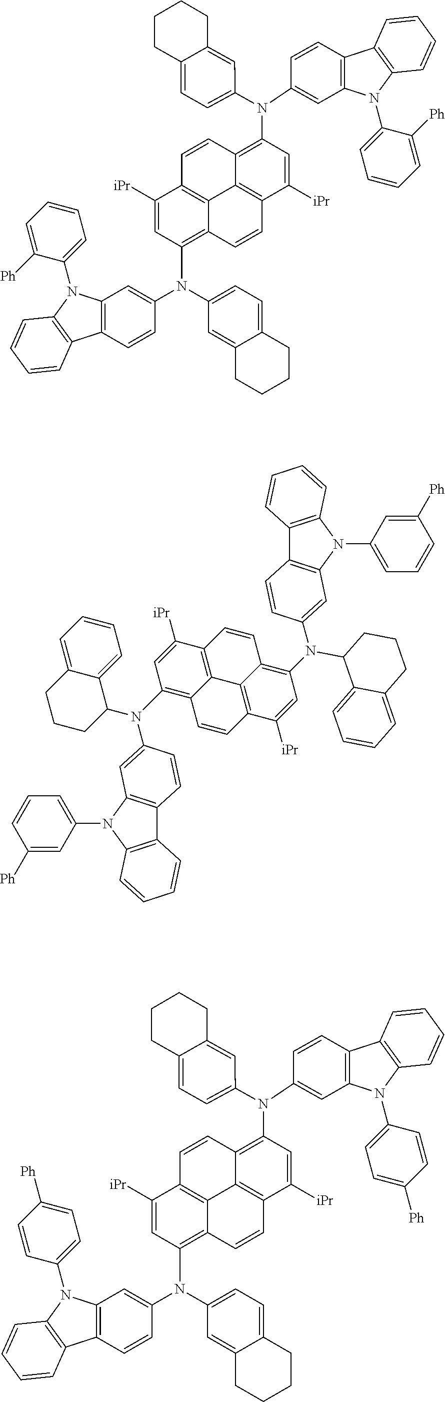 [SMILES: CC(C)c1cc(N(c2ccc3c(c2)CCCC3)c2ccc3c4ccccc4n(-c4ccccc4-c4ccccc4)c3c2)c2ccc3c(C(C)C)cc(N(c4ccc5c(c4)CCCC5)c4ccc5c6ccccc6n(-c6ccccc6-c6ccccc6)c5c4)c4ccc1c2c34 ; CC(C)c1cc(N(c2ccc3c4ccccc4n(-c4cccc(-c5ccccc5)c4)c3c2)C2CCCc3ccccc32)c2ccc3c(C(C)C)cc(N(c4ccc5c6ccccc6n(-c6cccc(-c7ccccc7)c6)c5c4)C4CCCc5ccccc54)c4ccc1c2c34 ; CC(C)c1cc(N(c2ccc3c(c2)CCCC3)c2ccc3c4ccccc4n(-c4ccc(-c5ccccc5)cc4)c3c2)c2ccc3c(C(C)C)cc(N(c4ccc5c(c4)CCCC5)c4ccc5c6ccccc6n(-c6ccc(-c7ccccc7)cc6)c5c4)c4ccc1c2c34]

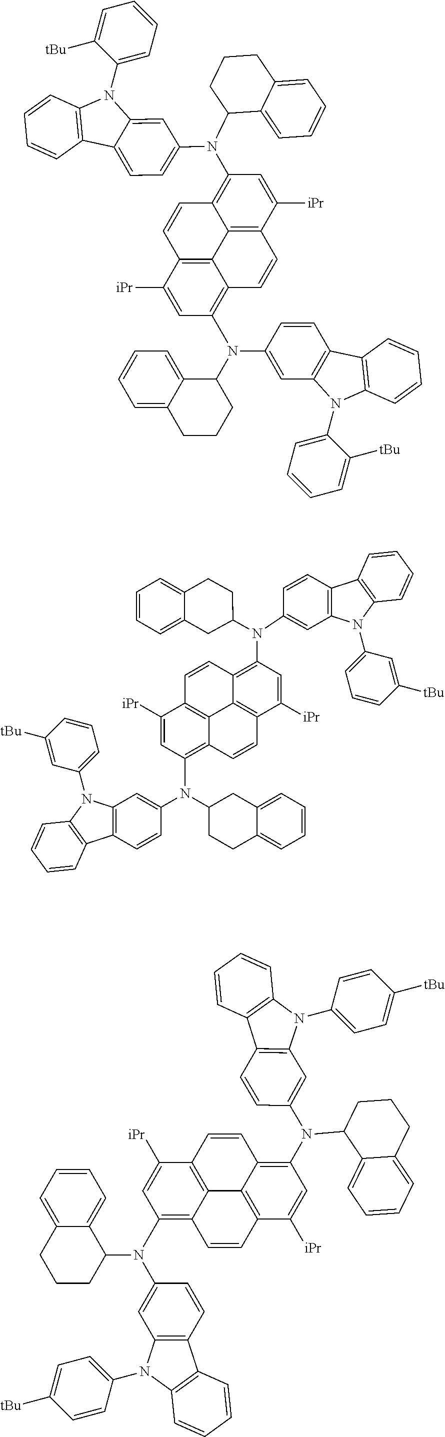 [SMILES: CC(C)c1cc(N(c2ccc3c4ccccc4n(-c4ccccc4C(C)(C)C)c3c2)C2CCCc3ccccc32)c2ccc3c(C(C)C)cc(N(c4ccc5c6ccccc6n(-c6ccccc6C(C)(C)C)c5c4)C4CCCc5ccccc54)c4ccc1c2c34 ; CC(C)c1cc(N(c2ccc3c4ccccc4n(-c4cccc(C(C)(C)C)c4)c3c2)C2CCc3ccccc3C2)c2ccc3c(C(C)C)cc(N(c4ccc5c6ccccc6n(-c6cccc(C(C)(C)C)c6)c5c4)C4CCc5ccccc5C4)c4ccc1c2c34 ; CC(C)c1cc(N(c2ccc3c4ccccc4n(-c4ccc(C(C)(C)C)cc4)c3c2)C2CCCc3ccccc32)c2ccc3c(C(C)C)cc(N(c4ccc5c6ccccc6n(-c6ccc(C(C)(C)C)cc6)c5c4)C4CCCc5ccccc54)c4ccc1c2c34]

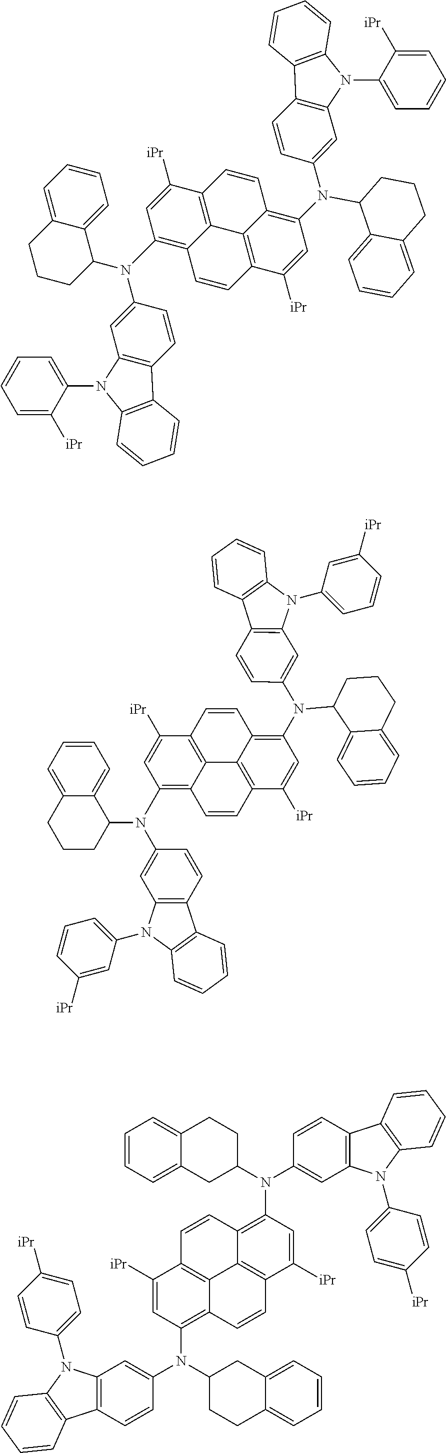 [SMILES: CC(C)c1ccccc1-n1c2ccccc2c2ccc(N(c3cc(C(C)C)c4ccc5c(N(c6ccc7c8ccccc8n(-c8ccccc8C(C)C)c7c6)C6CCCc7ccccc76)cc(C(C)C)c6ccc3c4c65)C3CCCc4ccccc43)cc21 ; CC(C)c1cccc(-n2c3ccccc3c3ccc(N(c4cc(C(C)C)c5ccc6c(N(c7ccc8c9ccccc9n(-c9cccc(C(C)C)c9)c8c7)C7CCCc8ccccc87)cc(C(C)C)c7ccc4c5c76)C4CCCc5ccccc54)cc32)c1 ; CC(C)c1ccc(-n2c3ccccc3c3ccc(N(c4cc(C(C)C)c5ccc6c(N(c7ccc8c9ccccc9n(-c9ccc(C(C)C)cc9)c8c7)C7CCc8ccccc8C7)cc(C(C)C)c7ccc4c5c76)C4CCc5ccccc5C4)cc32)cc1]

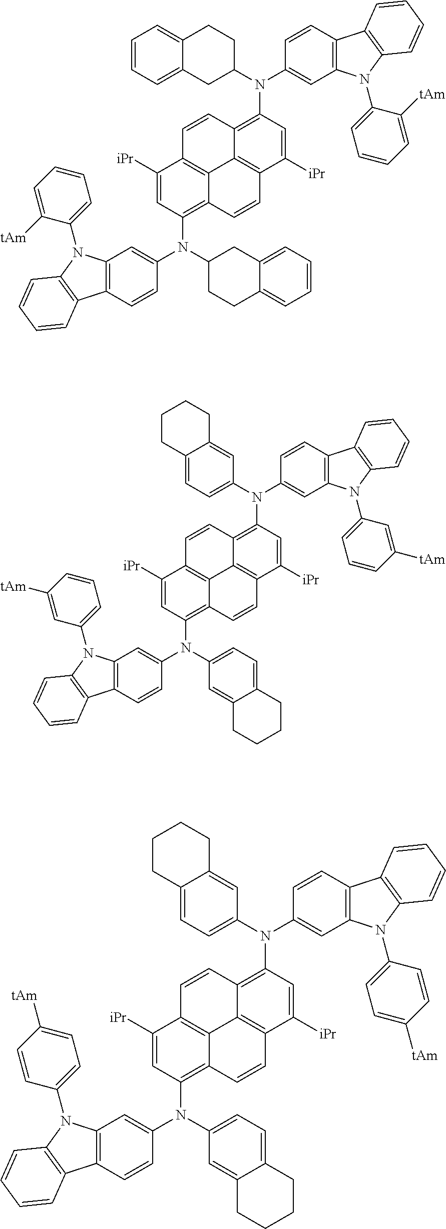

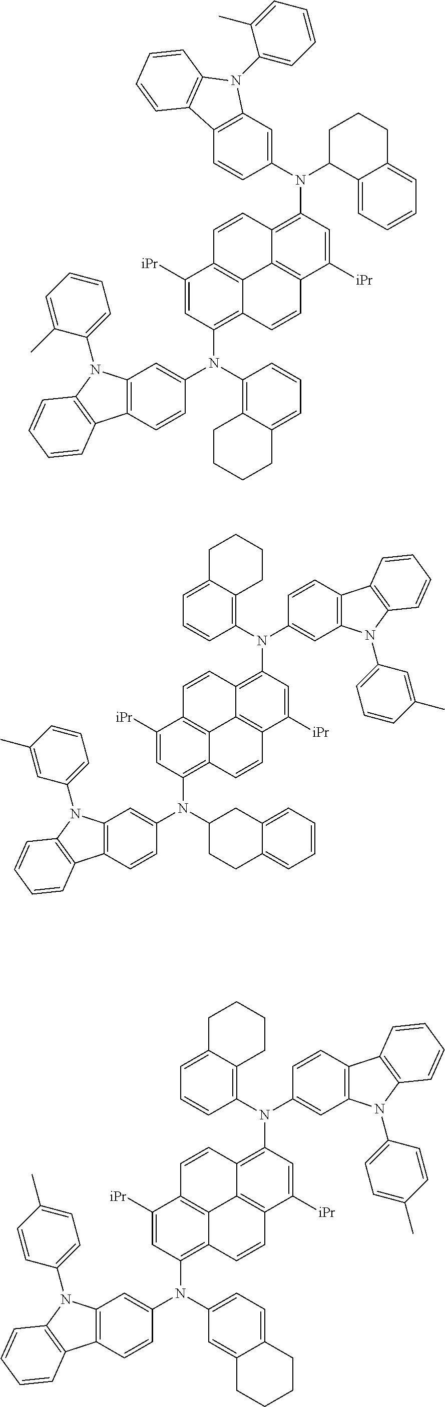

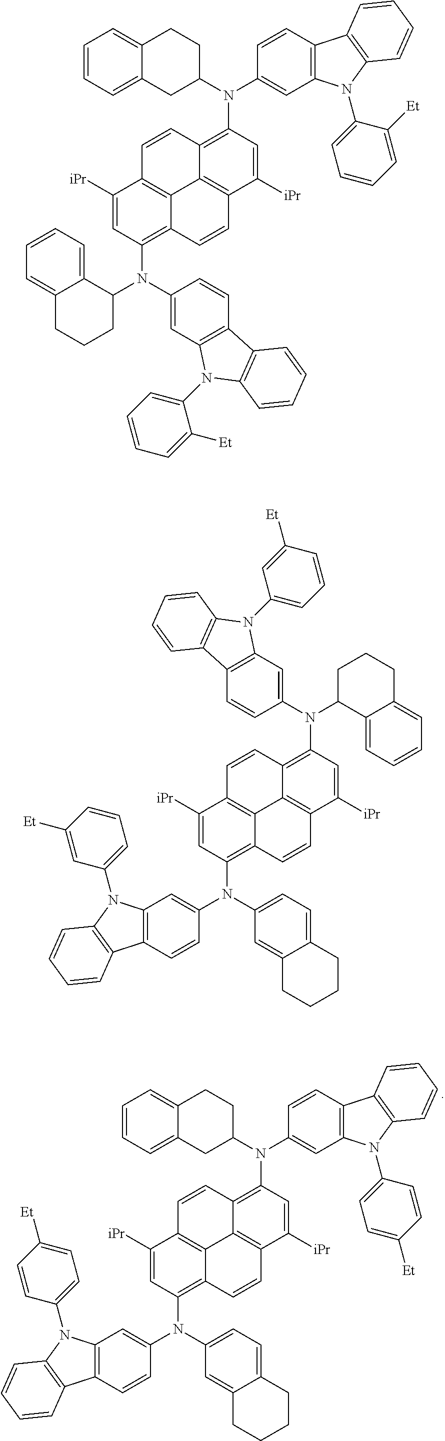 [SMILES: CCc1ccccc1-n1c2ccccc2c2ccc(N(c3cc(C(C)C)c4ccc5c(N(c6ccc7c8ccccc8n(-c8ccccc8CC)c7c6)C6CCCc7ccccc76)cc(C(C)C)c6ccc3c4c65)C3CCc4ccccc4C3)cc21 ; CCc1cccc(-n2c3ccccc3c3ccc(N(c4ccc5c(c4)CCCC5)c4cc(C(C)C)c5ccc6c(N(c7ccc8c9ccccc9n(-c9cccc(CC)c9)c8c7)C7CCCc8ccccc87)cc(C(C)C)c7ccc4c5c76)cc32)c1 ; CCc1ccc(-n2c3ccccc3c3ccc(N(c4ccc5c(c4)CCCC5)c4cc(C(C)C)c5ccc6c(N(c7ccc8c9ccccc9n(-c9ccc(CC)cc9)c8c7)C7CCc8ccccc8C7)cc(C(C)C)c7ccc4c5c76)cc32)cc1]

The present disclosure provides a mixture comprising any of the above mentioned tetrahydronaphthalene-based organic compounds and at least one organic functional material which is selected from the group consisting of a hole injecting material, a hole transporting material, an electron transporting material, an electron injecting material, an electron blocking material, a hole blocking material, a light emitter, a host material, and an organic dye.

The present disclosure provides a composition comprising any of the above mentioned tetrahydronaphthalene-based organic compounds or the above mentioned mixture, and at least one organic solvent.

The present disclosure provides an organic electronic device comprising any of the above mentioned tetrahydronaphthalene-based organic compounds or the above mentioned mixture.

Optionally, in some embodiments of the present disclosure, the organic electronic device comprises at least a light-emitting layer comprising any of the above mentioned tetrahydronaphthalene-based organic compounds or the above mentioned mixture.

Compared with the prior art, the present disclosure has the following beneficial effects:

When the tetrahydronaphthalene-based organic compound provided in the present disclosure is used as a blue fluorescent luminescent material, not only luminescence efficiency and lifetime of the device be improved, but also dark blue luminescence of the device can be realized due to the fact that the tetrahydronaphthalene-based organic compound provided in the present disclosure has an excellent conjugated system, and meanwhile, the tetrahydronaphthalene-based organic compound provided in the present disclosure has a fluorescence emission with a short wavelength, and the emission spectrum exhibits a narrow half-peak width.

### BRIEF DESCRIPTION OF THE DRAWINGS

In order to more clearly describe the technical solutions in embodiments of the present disclosure, hereinafter, the appended drawings used for describing the embodiments will be briefly introduced. Apparently, the appended drawings described below are only directed to some embodiments of the present disclosure, and for a person skilled in the art, without expenditure of creative labor, other drawings can be derived on the basis of these appended drawings.

FIG. 1 is a schematic structural diagram of an organic electronic device according to the present disclosure.

### DETAILED DESCRIPTION

Hereinafter, technical solutions in embodiments of the present disclosure will be clearly and completely described with reference to the accompanying drawings in embodiments of the present disclosure. Apparently, the described embodiments are part of, but not all of, the embodiments of the present disclosure. All the other embodiments, obtained by a person with ordinary skill in the art on the basis of the embodiments in the present disclosure without expenditure of creative labor, belong to the protection scope of the present disclosure. In addition, it should be understood that specific embodiments described herein are only used to illustrate and explain the present disclosure, and are not intended to limit the present disclosure. In the present disclosure, unless otherwise stated, in the description of the present disclosure, the term “comprising” means “including, but is not limited to”. The term “plurality” means “two or more”. Various embodiments of the present disclosure may exist in the form of a range. It should be understood that the description in the form of a range is merely for convenience and conciseness, and should not be construed as a hard limit on the scope of the present disclosure. Accordingly, it should be considered that the description of range should be considered to have specifically disclosed all possible subranges, as well as a single numerical value within the range. For example, it should be considered that the description of a range from 1 to 6 has specifically disclosed subranges, e.g., from 1 to 3, from 1 to 4, from 1 to 5, from 2 to 4, from 2 to 6, from 3 to 6, etc., as well as single numbers within the range, e.g., 1, 2, 3, 4, 5, and 6, which are applicable regardless of the range. Additionally, whenever a numerical range is indicated herein, it is meant to include any quoted numbers (fractions or integers) within the indicated range.

In the present disclosure, the composition, printing ink, or ink has the same meaning and they are interchangeable.

In the present disclosure, the aromatic group, aromatic, aromatic ring system have the same meaning and they are interchangeable.

In the present disclosure, heteroaromatic group, heteroaromatic, heteroaromatic ring system have the same meaning and they are interchangeable.

In the present disclosure, “substituted” means that hydrogen atom in the substituent is replaced by a substituent.

In the present disclosure, the same substituent may be independently selected from different groups. If a general formula contains a plurality of R₁, then R₁ may be independently selected from different groups. For example, in general formula

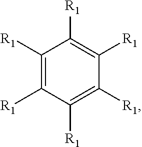

six R₁ on the benzene ring may be the same as or different from each other.

In the present disclosure, “substituted or unsubstituted” means that the defined group may or may not be substituted. When a group is substituted, it should be understood that it is optionally replaced by a group acceptable in the art, including, but is not limited to, an alkyl group containing 1 to 30 carbon atoms, a heterocyclic group containing 3 to 20 ring atoms, an aryl group containing 5 to 20 ring atoms, a heteroaryl group containing 5 to 20 ring atoms, a silyl group, a carbonyl group, an alkoxycarbonyl group, an aryloxycarbonyl group, a carbamoyl group, a haloformyl group, a formyl group, a —NRR′, a cyano group, an isocyano group, an isocyanate group, a thiocyanate group, an isothiocyanate group, a hydroxyl group, a trifluoromethyl group, a nitro group, or a halogen, and the above group may also be further substituted by a substituent acceptable in the art. It can be understood that R and R′ in —NRR′ are independently substituted by a group acceptable in the art, including, but is not limited to, —H, an alkyl group containing 1 to 6 carbon atoms, a cycloalkyl group containing 3 to 8 ring atoms, a heterocyclic group containing 3 to 8 ring atoms, an aryl group containing 5 to 20 ring atoms, or a heteroaryl group containing 5 to 10 ring atoms; an alkyl group containing 1 to 6 carbon atoms, a cycloalkyl group containing 3 to 8 ring atoms, a heterocyclic group containing 3 to 8 ring atoms, an aryl group containing 5 to 20 ring atoms, or a heteroaryl group containing 5 to 10 ring atoms, which may be optionally further substituted by one or more of of the following groups: an alkyl group containing 1 to 6 carbon atoms, a cycloalkyl group containing 3 to 8 ring atoms, a heterocyclic group containing 3 to 8 ring atoms, a halogen, a hydroxyl group, a nitro group, or an amino group.

In the present disclosure, “the number of ring atoms” refers to the number of atoms constituting the ring itself of a compound (e.g., a monocyclic compound, a fused ring compound, a cross-linking compound, a carbocyclic compound, or a heterocyclic compound), in which atoms are bonded to form a ring. When the ring is substituted by a substituent, the atoms contained in the substituent are not included in the ring-forming atoms. In the context, the “the number of ring atoms” is the same unless otherwise specified. For example, the number of ring atoms of a benzene ring is 6, the number of ring atoms of a naphthalene ring is 10, and the number of ring atoms of a thiophenyl group is 5.

In the present disclosure, “alkyl” may refer to a linear, branched and/or cyclic alkyl group. The number of carbon atoms of the alkyl group may be 1 to 50, 1 to 30, 1 to 20, 1 to 10, or 1 to 6. Non-limiting examples of the alkyl group include methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-tert-butylcyclohexyl, n-heptyl, 1-methylheptyl, 2,2-dimethylheptyl, 2-ethylheptyl, 2-butylheptyl, n-octyl, tert-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldodecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-heneicosyl, n-docosyl, n-tricosyl, n-tetracosyl, n-pentacosyl, n-hexacosyl, n-heptacosyl, n-octacosyl, n-nonacosyl, n-triacosyl, and the like.

“Aryl, aromatic, or aromatic group” refers to a hydrocarbyl group containing at least one aromatic ring. “Heteroaromatic or heteroaromatic group” refers to an aromatic hydrocarbyl group containing at least one heteroatom. The heteroatom is preferably selected from Si, N, P, O, S and/or Ge, particularly preferably from Si, N, P, O and/or S. A fused ring aromatic group refers to the ring of an aromatic group may have two or more rings, wherein two carbon atoms are shared by two adjacent rings, i.e., a fused ring. A fused heterocyclic aromatic group refers to a fused aromatic hydrocarbyl group containing at least one heteroatom. In the present disclosure, aromatic or heteroaromatic groups include not only aromatic ring systems, but also non-aromatic ring systems. In the context, rings such as pyridine, thiophene, pyrrole, pyrazole, triazole, imidazole, oxazole, oxadiazole, thiazole, tetrazole, pyrazine, pyridazine, pyrimidine, triazine, carbene, etc. are considered as aromatic or heterocyclic aromatic rings. For the purpose of the present disclosure, fused aromatic or fused heterocyclic aromatic rings include aromatic or heteroaromatic systems, and those in which a plurality of aryl grous or heteroaryl groups may be interrupted by adding short non-aromatic units (<10% by molar of non-H atoms, specially less than 5% of non-H atoms, such as C, N or O atoms). Therefore, compounds such as 9,9′-spirobifluorene, 9,9-diarylfluorene, triarylamine, diaryl ether are also considered to be aromatic ring for the purpose of the present disclosure.

In a certain preferred embodiment, the aromatic group is selected from the group consisting of benzene, naphthalene, anthracene, fluoranthene, phenanthrene, benzophenanthrene, perylene, tetracene, pyrene, benzopyrene, acenaphthene, fluorene, and derivatives thereof. The heteroaromatic group is selected from the group consisting of triazine, pyridine, pyrimidine, imidazole, furan, thiophene, benzofuran, benzothiophene, indole, carbazole, pyrroloimidazole, pyrrolopyrrole, thienopyrrole, thienothiophene, furopyrrole, furofuran, thienofuran, benzisoxazole, benzisothiazole, benzimidazole, quinoline, isoquinoline, odiazonaphthalene, quinoxaline, phenanthridine, perimidine, quinazoline, quinazolinone, dibenzothiophene, dibenzofuran, carbazole and derivatives thereof.

In the present disclosure, when the linking position position is not specified in the group, it means that any linking position in the group can be used as a linking position.

In the present disclosure, when the fused position position is not specified in the group, it means that an optional fused position in the group can be used as a fused position, and preferably two or more positions in the ortho position.

In the present disclosure, a single bond to which a substituent is attached runs through the corresponding ring, indicating that the substituent may be attached to an optional position of the ring, for example, R shown in

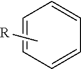

is attached to any substitutable position of the benzene ring,

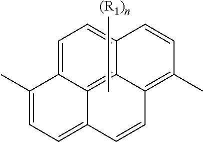

means that one or more R₁ may be attached to any substitutable position of the four benzene rings.

In the present disclosure, the energy level structure of an organic material: triplet energy level ET1, highest occupied molecular orbital (HOMO) energy level, and lowest unoccupied molecular orbital (LUMO) energy level play a key role. Determination of these energy levels is described below. HOMO and LUMO energy levels can be measured by photoelectric effects, such as XPS (X-ray photoelectron spectroscopy) and UPS (ultraviolet photoelectron spectrometer) or by cyclic voltammetry (hereinafter referred to as CV). Recently, quantum chemical methods, such as density functional theory (DFT), have also become effective methods for calculating molecular orbital energy levels. The triplet energy level ET1 of organic materials can be measured by low temperature time-resolved luminescence spectroscopy, or calculated by quantum simulation. It should be noted that the absolute values of HOMO, LUMO, ET1 depend on the measurement or calculation method used, and even for the same method, different evaluation methods, such as onset and peak points on the CV curve, may give different HOMO/LUMO values. Therefore, a reasonable and meaningful comparison should be made using the same measurement method and the same evaluation method. In the description of embodiments of the present disclosure, the values of HOMO, LUMO, ET1 are simulated based on Time-dependent DFT, but it does not affect the application of other measurement or calculation methods.

Technical solutions of the present disclosure are as follows:

A tetrahydronaphthalene-based organic compound having a structure shown in general formula (1):

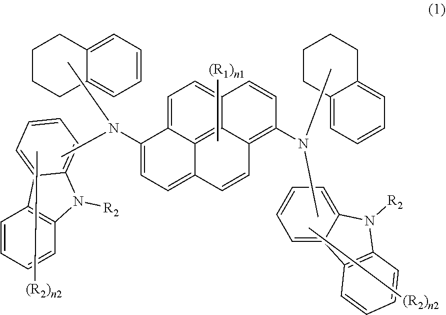

wherein, n1 is an integer selected from 1 to 8; n2 is an integer selected from 1 to 7.

R₁ and R₂, at each occurrence, is independently selected from the group consisting of H, deuterium, a linear alkyl group containing 1 to 20 carbon atoms, a linear alkoxy containing 1 to 20 carbon atoms, a linear thioalkoxy group containing 1 to 20 carbon atoms, a branched or cyclic alkyl containing 3 to 20 carbon atoms, a branched or cyclic alkoxy containing 3 to 20 carbon atoms, a branched or cyclic thioalkoxy group containing 3 to 20 carbon atoms, a silyl group, a ketone group containing 1 to 20 carbon atoms, an alkoxycarbonyl group containing 2 to 20 carbon atoms, an aryloxycarbonyl group containing 7 to 20 carbon atoms, a substituted or unsubstituted aromatic or heteroaromatic group containing 5 to 60 ring atoms, an aryloxy or heteroaryloxy group containing 5 to 60 ring atoms, a cyano group, a carbamoyl group, a haloformyl group, a formyl group, an isocyano group, an isocyanate group, a thiocyanate group, an isothiocyanate group, a hydroxyl group, a nitro group, an amino group, —CF₃, —Cl, —Br, —F, —I, and combinations thereof.

In some embodiments, n1 and n2 are each independently selected from 0 or 1 or 2.

In some embodiments, R₁ and R₂, at each occurrence is independently selected from the group consisting of —H, deuterium, a linear alkyl group containing 1 to 10 carbon atoms, a branched or cyclic alkyl containing 3 to 10 carbon atoms, a substituted or unsubstituted aromatic group containing 5 to 30 ring atoms, a substituted or unsubstituted heteroaromatic group containing 5 to 30 ring atoms, and combinations thereof.

In some embodiments, R₁ and R₂, at each occurrence, is independently selected from the group consisting of H, deuterium, a linear alkyl group containing 1 to 8 carbon atoms or a branched or cyclic alkyl containing 3 to 8 carbon atoms, phenyl, pyridyl, pyrimidinyl, naphthyl, and combinations thereof.

Further, in some embodiments, R₁ and R₂, at each occurrence, is independently selected from the group consisting of —H, deuterium, methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-tert-butylcyclohexyl, n-heptyl, 1-methylheptyl, 2,2-dimethylheptyl, 2-ethylheptyl, 2-butylheptyl, n-octyl, tert-octyl, 2-ethyloctyl, 2-butyloctyl, 2-hexyloctyl, 3,7-dimethyloctyl, cyclooctyl, n-nonyl, n-decyl, adamantly or 2-(2-methyl) butyl, phenyl, pyridyl, pyrimidinyl, and naphthyl.

In some embodiments, adjacent R₁ (if any) form a ring with each other.

In some embodiments, R₂, at each occurrence, is independently selected from the same group.

In some embodiments, the tetrahydronaphthalene-based organic compound has a structure shown in general formula (2):

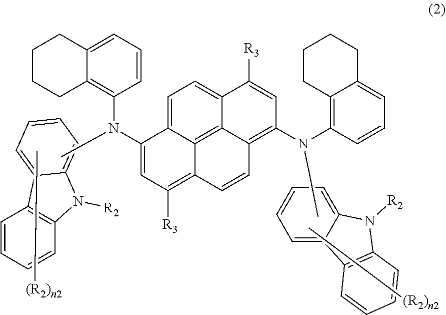

wherein, R₃, at each occurrence, is independently selected from the group consisting of methyl, ethyl, isopropyl and tert-butyl.

In some embodiments, the tetrahydronaphthalene-based organic compound has a structure shown in general formulae from (3-1) to (3-4):

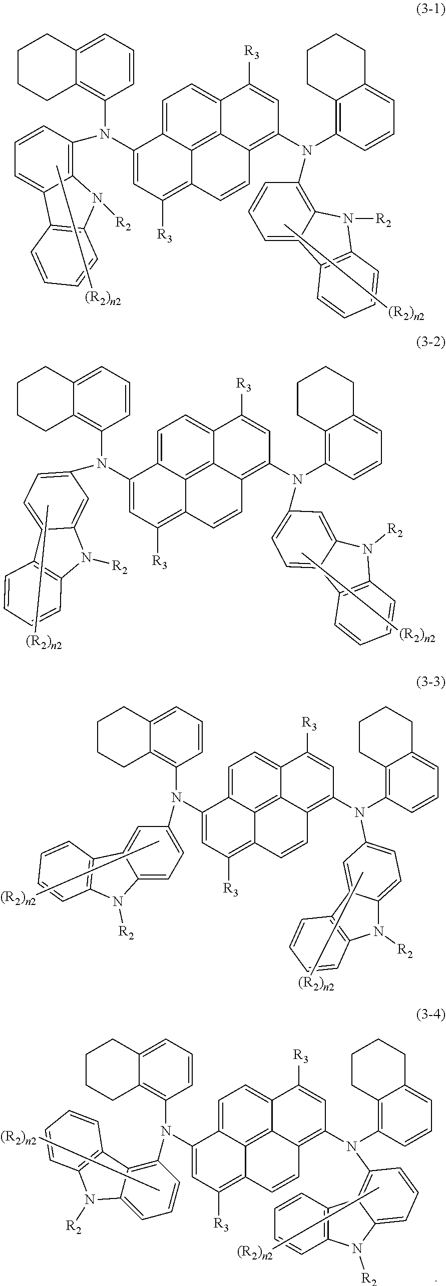

In some embodiments, R₂, at each occurrence, in general formulae from (3-1) to (3-4) is independently selected from deuterium, a linear alkyl group containing 1 to 10 carbon atoms, a branched or cyclic alkyl containing 3 to 10 carbon atoms, an aromatic group containing 5 to 20 ring atoms, a heteroaromatic group containing 5 to 20 ring atoms, or combination thereof.

In some embodiments, R₂, at each occurrence, in general formulae from (3-1) to (3-4) is independently selected from deuterium, a linear alkyl group containing 1 to 8 carbon atoms, or a branched or cyclic alkyl containing 3 to 8 carbon atoms.

In some embodiments, R₂, at each occurrence, in general formulae from (3-1) to (3-4) is independently selected from deuterium, methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-tert-butylcyclohexyl, n-heptyl, 1-methylheptyl, 2,2-dimethylheptyl, 2-ethylheptyl, 2-butylheptyl, n-octyl, tert-octyl, 2-ethyloctyl, 2-butyloctyl, 2-hexyloctyl, 3,7-dimethyloctyl, cyclooctyl, n-nonyl, n-decyl, adamantly, or 2-(2-methyl) butyl.

In some embodiments, the tetrahydronaphthalene-based organic compound is selected from any one of the following structures:

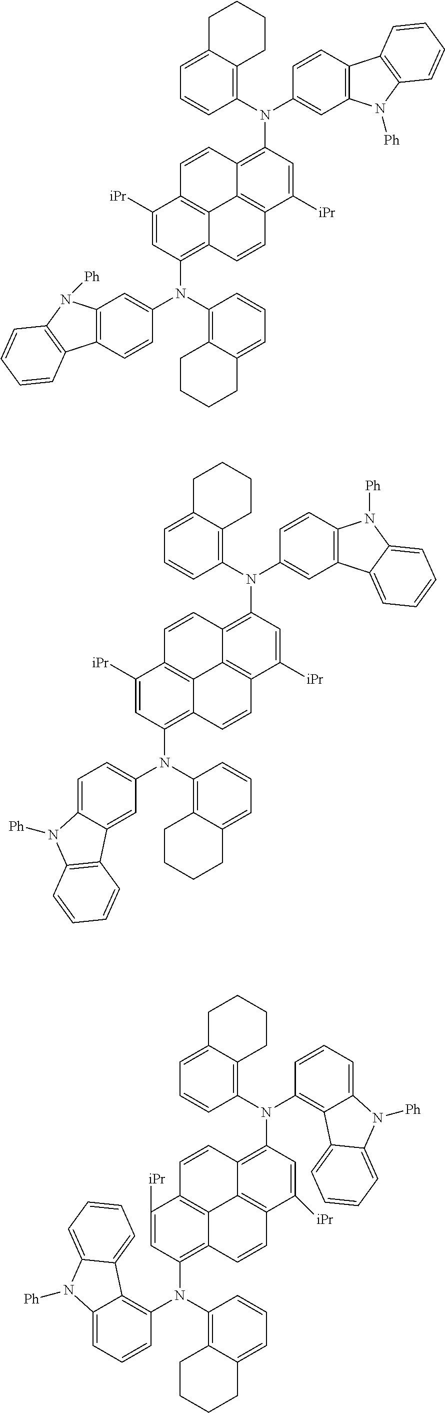 [SMILES: CC(C)c1cc(N(c2ccc3c4ccccc4n(-c4ccccc4)c3c2)c2cccc3c2CCCC3)c2ccc3c(C(C)C)cc(N(c4ccc5c6ccccc6n(-c6ccccc6)c5c4)c4cccc5c4CCCC5)c4ccc1c2c34 ; CC(C)c1cc(N(c2ccc3c(c2)c2ccccc2n3-c2ccccc2)c2cccc3c2CCCC3)c2ccc3c(C(C)C)cc(N(c4ccc5c(c4)c4ccccc4n5-c4ccccc4)c4cccc5c4CCCC5)c4ccc1c2c34 ; CC(C)c1cc(N(c2cccc3c2CCCC3)c2cccc3c2c2ccccc2n3-c2ccccc2)c2ccc3c(C(C)C)cc(N(c4cccc5c4CCCC5)c4cccc5c4c4ccccc4n5-c4ccccc4)c4ccc1c2c34]

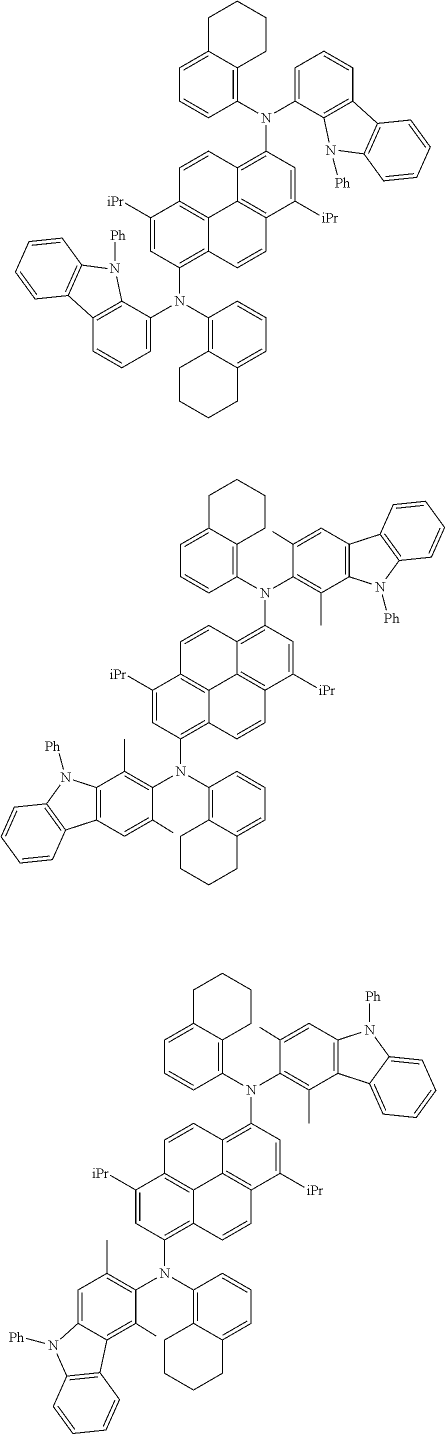 [SMILES: CC(C)c1cc(N(c2cccc3c2CCCC3)c2cccc3c4ccccc4n(-c4ccccc4)c23)c2ccc3c(C(C)C)cc(N(c4cccc5c4CCCC5)c4cccc5c6ccccc6n(-c6ccccc6)c45)c4ccc1c2c34 ; Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)cc5c6ccccc6n(-c6ccccc6)c5c4C)cc(C(C)C)c4ccc1c2c43 ; Cc1cc2c(c(C)c1N(c1cccc3c1CCCC3)c1cc(C(C)C)c3ccc4c(N(c5cccc6c5CCCC6)c5c(C)cc6c(c5C)c5ccccc5n6-c5ccccc5)cc(C(C)C)c5ccc1c3c54)c1ccccc1n2-c1ccccc1]

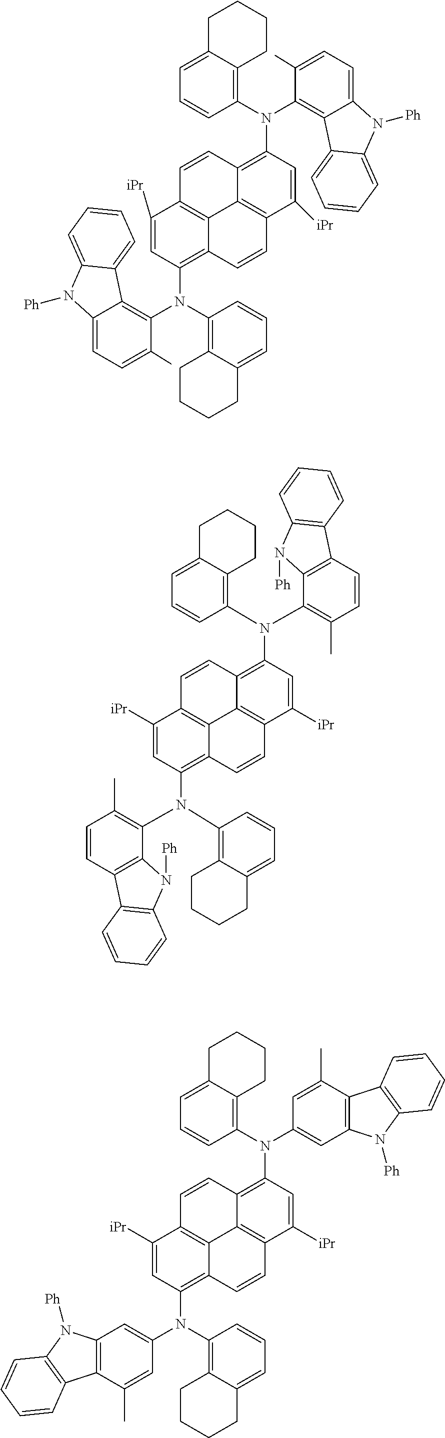 [SMILES: Cc1ccc2c(c1N(c1cccc3c1CCCC3)c1cc(C(C)C)c3ccc4c(N(c5cccc6c5CCCC6)c5c(C)ccc6c5c5ccccc5n6-c5ccccc5)cc(C(C)C)c5ccc1c3c54)c1ccccc1n2-c1ccccc1 ; Cc1ccc2c3ccccc3n(-c3ccccc3)c2c1N(c1cccc2c1CCCC2)c1cc(C(C)C)c2ccc3c(N(c4cccc5c4CCCC5)c4c(C)ccc5c6ccccc6n(-c6ccccc6)c45)cc(C(C)C)c4ccc1c2c43 ; Cc1cc(N(c2cccc3c2CCCC3)c2cc(C(C)C)c3ccc4c(N(c5cc(C)c6c7ccccc7n(-c7ccccc7)c6c5)c5cccc6c5CCCC6)cc(C(C)C)c5ccc2c3c54)cc2c1c1ccccc1n2-c1ccccc1]

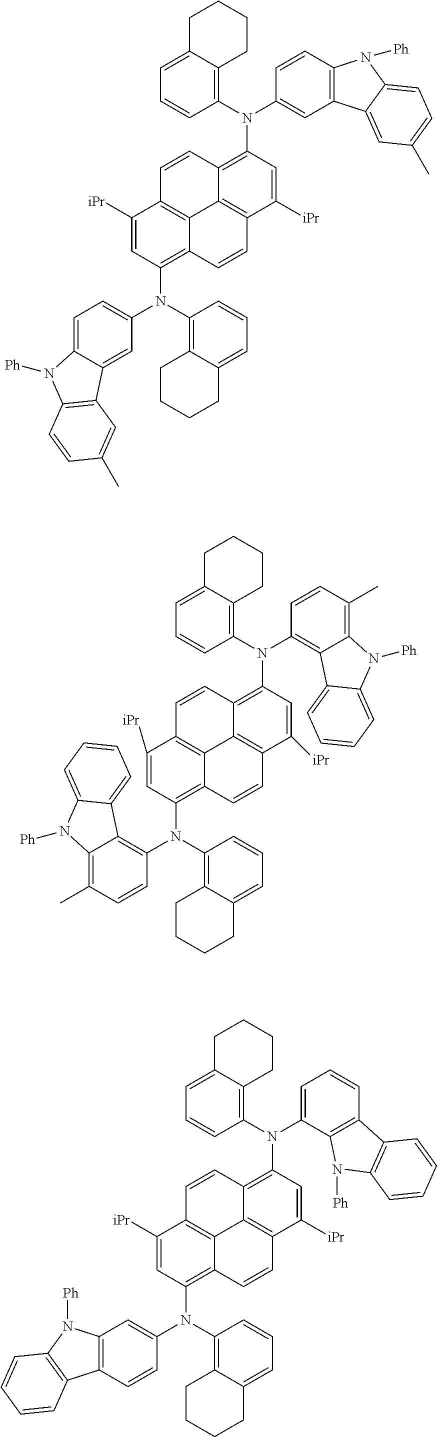 [SMILES: Cc1ccc2c(c1)c1cc(N(c3cccc4c3CCCC4)c3cc(C(C)C)c4ccc5c(N(c6ccc7c(c6)c6cc(C)ccc6n7-c6ccccc6)c6cccc7c6CCCC7)cc(C(C)C)c6ccc3c4c65)ccc1n2-c1ccccc1 ; Cc1ccc(N(c2cccc3c2CCCC3)c2cc(C(C)C)c3ccc4c(N(c5cccc6c5CCCC6)c5ccc(C)c6c5c5ccccc5n6-c5ccccc5)cc(C(C)C)c5ccc2c3c54)c2c3ccccc3n(-c3ccccc3)c12 ; CC(C)c1cc(N(c2ccc3c4ccccc4n(-c4ccccc4)c3c2)c2cccc3c2CCCC3)c2ccc3c(C(C)C)cc(N(c4cccc5c4CCCC5)c4cccc5c6ccccc6n(-c6ccccc6)c45)c4ccc1c2c34]

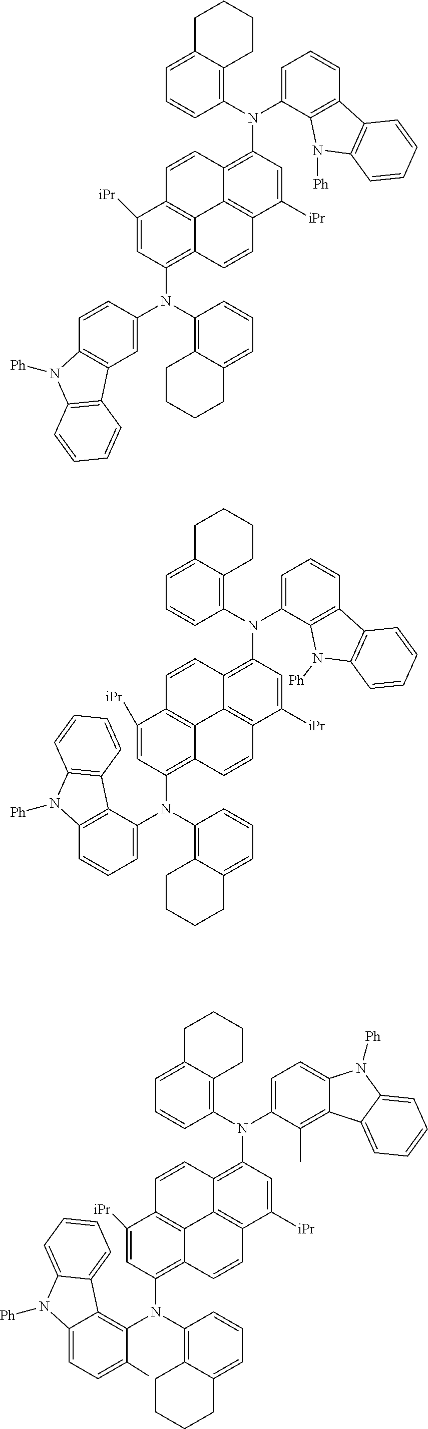 [SMILES: CC(C)c1cc(N(c2ccc3c(c2)c2ccccc2n3-c2ccccc2)c2cccc3c2CCCC3)c2ccc3c(C(C)C)cc(N(c4cccc5c4CCCC5)c4cccc5c6ccccc6n(-c6ccccc6)c45)c4ccc1c2c34 ; CC(C)c1cc(N(c2cccc3c2CCCC3)c2cccc3c2c2ccccc2n3-c2ccccc2)c2ccc3c(C(C)C)cc(N(c4cccc5c4CCCC5)c4cccc5c6ccccc6n(-c6ccccc6)c45)c4ccc1c2c34 ; Cc1ccc2c(c1N(c1cccc3c1CCCC3)c1cc(C(C)C)c3ccc4c(N(c5cccc6c5CCCC6)c5ccc6c(c5C)c5ccccc5n6-c5ccccc5)cc(C(C)C)c5ccc1c3c54)c1ccccc1n2-c1ccccc1]

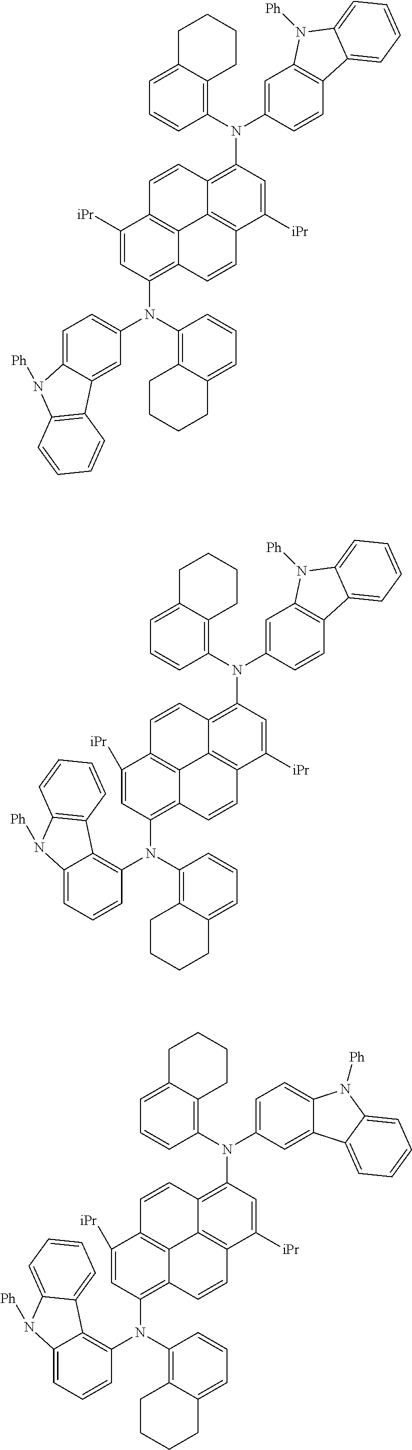 [SMILES: CC(C)c1cc(N(c2ccc3c(c2)c2ccccc2n3-c2ccccc2)c2cccc3c2CCCC3)c2ccc3c(C(C)C)cc(N(c4ccc5c6ccccc6n(-c6ccccc6)c5c4)c4cccc5c4CCCC5)c4ccc1c2c34 ; CC(C)c1cc(N(c2ccc3c4ccccc4n(-c4ccccc4)c3c2)c2cccc3c2CCCC3)c2ccc3c(C(C)C)cc(N(c4cccc5c4CCCC5)c4cccc5c4c4ccccc4n5-c4ccccc4)c4ccc1c2c34 ; CC(C)c1cc(N(c2ccc3c(c2)c2ccccc2n3-c2ccccc2)c2cccc3c2CCCC3)c2ccc3c(C(C)C)cc(N(c4cccc5c4CCCC5)c4cccc5c4c4ccccc4n5-c4ccccc4)c4ccc1c2c34]

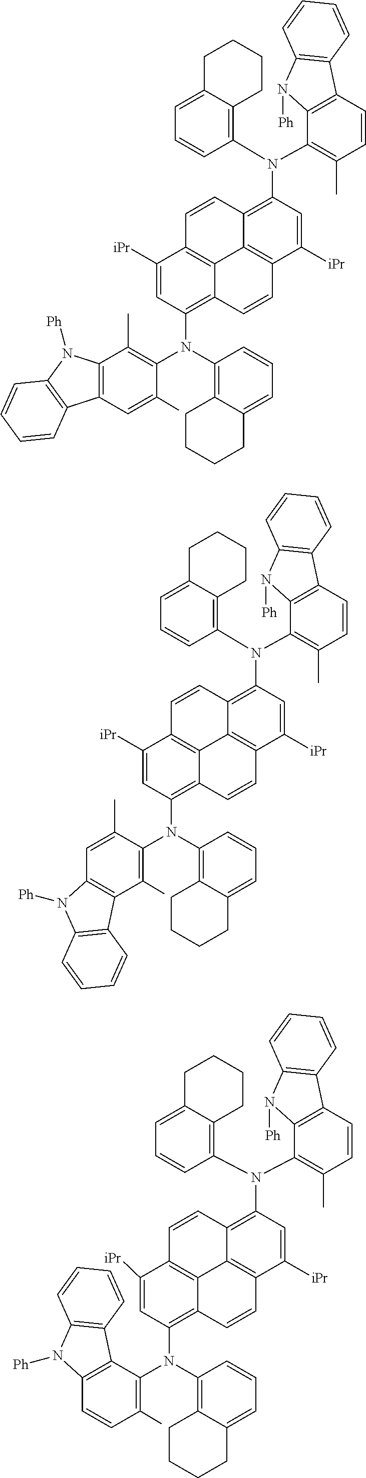 [SMILES: Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)ccc5c6ccccc6n(-c6ccccc6)c45)cc(C(C)C)c4ccc1c2c43 ; Cc1cc2c(c(C)c1N(c1cccc3c1CCCC3)c1cc(C(C)C)c3ccc4c(N(c5cccc6c5CCCC6)c5c(C)ccc6c7ccccc7n(-c7ccccc7)c56)cc(C(C)C)c5ccc1c3c54)c1ccccc1n2-c1ccccc1 ; Cc1ccc2c(c1N(c1cccc3c1CCCC3)c1cc(C(C)C)c3ccc4c(N(c5cccc6c5CCCC6)c5c(C)ccc6c7ccccc7n(-c7ccccc7)c56)cc(C(C)C)c5ccc1c3c54)c1ccccc1n2-c1ccccc1]

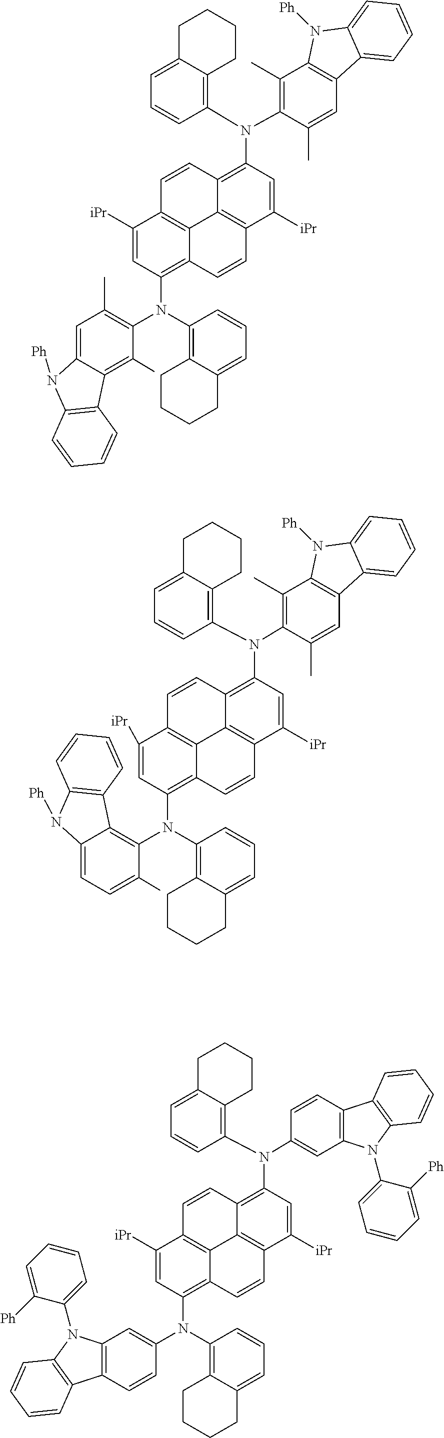 [SMILES: Cc1cc2c(c(C)c1N(c1cccc3c1CCCC3)c1cc(C(C)C)c3ccc4c(N(c5cccc6c5CCCC6)c5c(C)cc6c7ccccc7n(-c7ccccc7)c6c5C)cc(C(C)C)c5ccc1c3c54)c1ccccc1n2-c1ccccc1 ; Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)ccc5c4c4ccccc4n5-c4ccccc4)cc(C(C)C)c4ccc1c2c43 ; CC(C)c1cc(N(c2ccc3c4ccccc4n(-c4ccccc4-c4ccccc4)c3c2)c2cccc3c2CCCC3)c2ccc3c(C(C)C)cc(N(c4ccc5c6ccccc6n(-c6ccccc6-c6ccccc6)c5c4)c4cccc5c4CCCC5)c4ccc1c2c34]

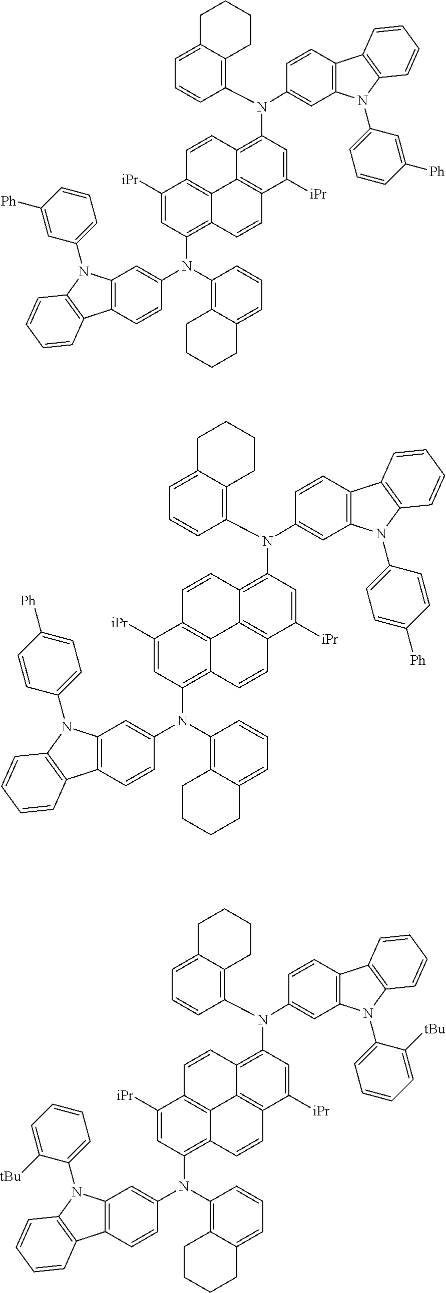 [SMILES: CC(C)c1cc(N(c2ccc3c4ccccc4n(-c4cccc(-c5ccccc5)c4)c3c2)c2cccc3c2CCCC3)c2ccc3c(C(C)C)cc(N(c4ccc5c6ccccc6n(-c6cccc(-c7ccccc7)c6)c5c4)c4cccc5c4CCCC5)c4ccc1c2c34 ; CC(C)c1cc(N(c2ccc3c4ccccc4n(-c4ccc(-c5ccccc5)cc4)c3c2)c2cccc3c2CCCC3)c2ccc3c(C(C)C)cc(N(c4ccc5c6ccccc6n(-c6ccc(-c7ccccc7)cc6)c5c4)c4cccc5c4CCCC5)c4ccc1c2c34 ; CC(C)c1cc(N(c2ccc3c4ccccc4n(-c4ccccc4C(C)(C)C)c3c2)c2cccc3c2CCCC3)c2ccc3c(C(C)C)cc(N(c4ccc5c6ccccc6n(-c6ccccc6C(C)(C)C)c5c4)c4cccc5c4CCCC5)c4ccc1c2c34]

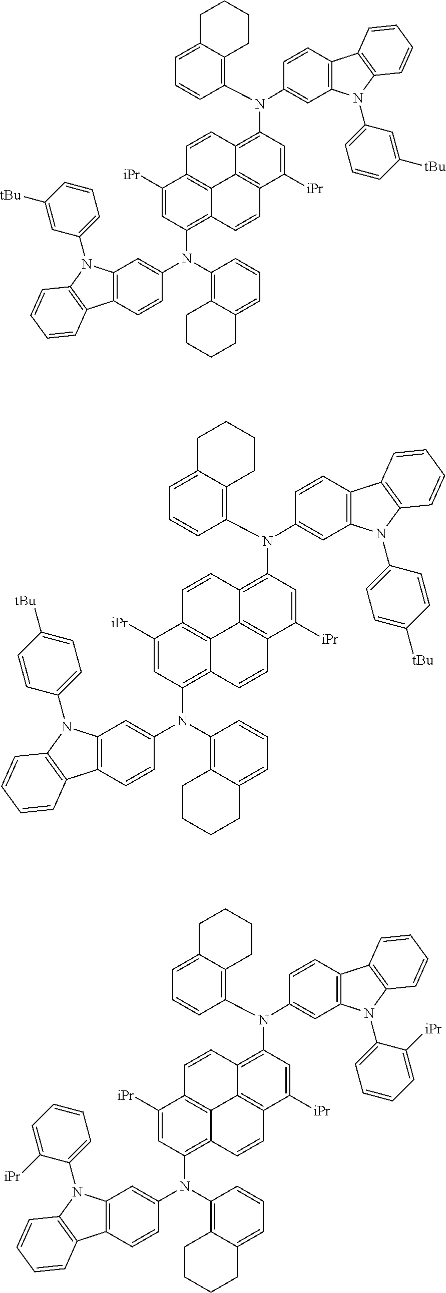 [SMILES: CC(C)c1cc(N(c2ccc3c4ccccc4n(-c4cccc(C(C)(C)C)c4)c3c2)c2cccc3c2CCCC3)c2ccc3c(C(C)C)cc(N(c4ccc5c6ccccc6n(-c6cccc(C(C)(C)C)c6)c5c4)c4cccc5c4CCCC5)c4ccc1c2c34 ; CC(C)c1cc(N(c2ccc3c4ccccc4n(-c4ccc(C(C)(C)C)cc4)c3c2)c2cccc3c2CCCC3)c2ccc3c(C(C)C)cc(N(c4ccc5c6ccccc6n(-c6ccc(C(C)(C)C)cc6)c5c4)c4cccc5c4CCCC5)c4ccc1c2c34 ; CC(C)c1ccccc1-n1c2ccccc2c2ccc(N(c3cccc4c3CCCC4)c3cc(C(C)C)c4ccc5c(N(c6ccc7c8ccccc8n(-c8ccccc8C(C)C)c7c6)c6cccc7c6CCCC7)cc(C(C)C)c6ccc3c4c65)cc21]

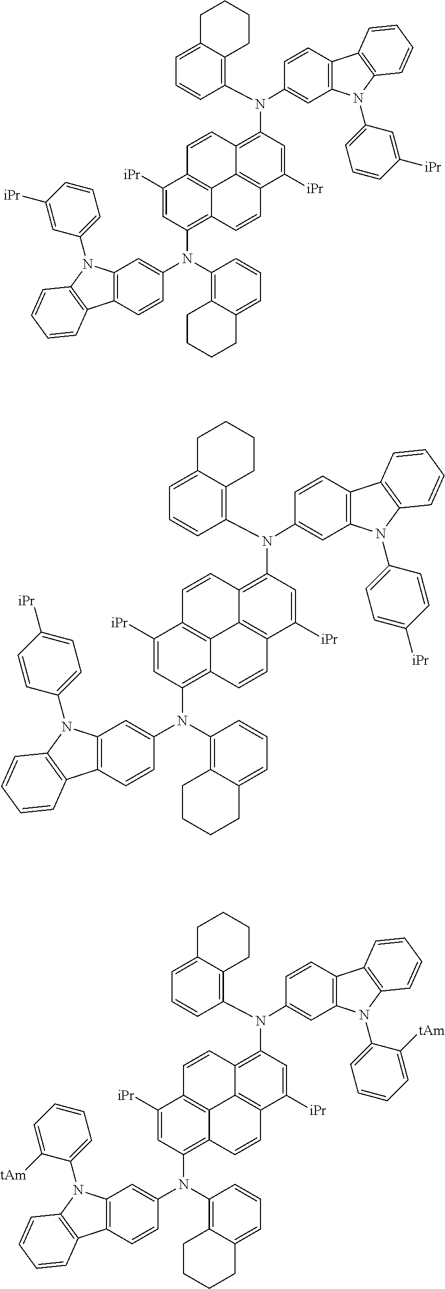

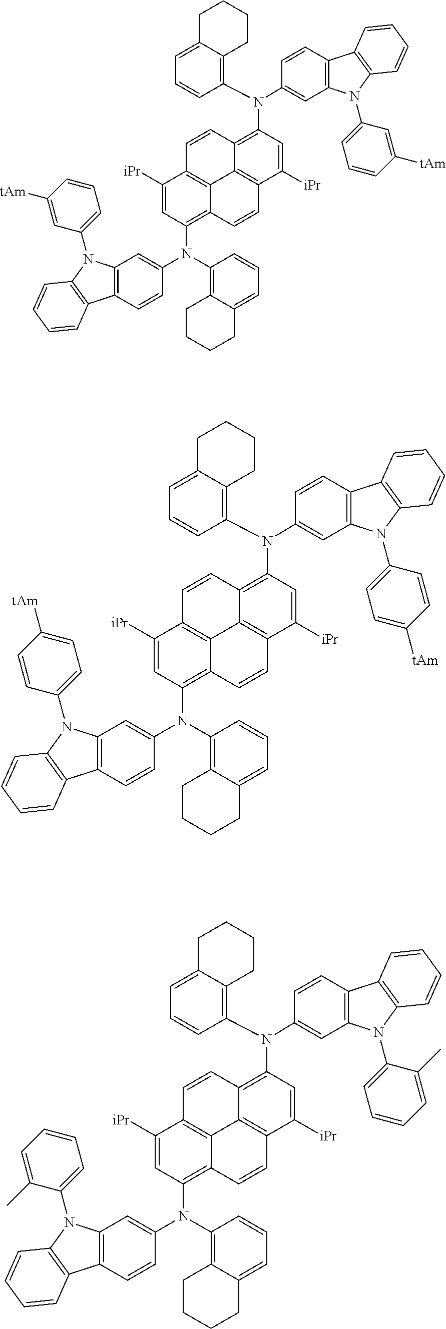

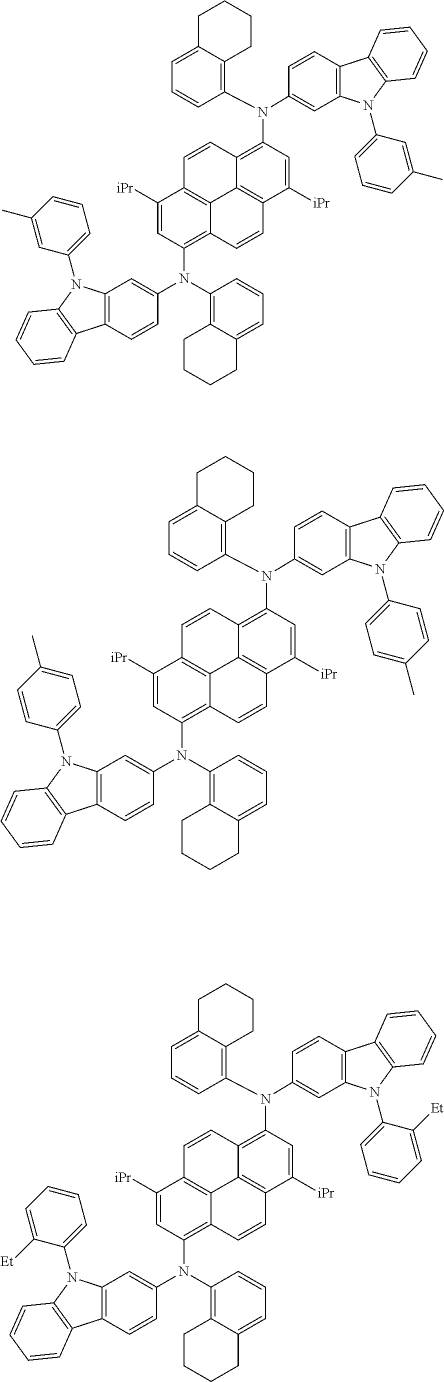

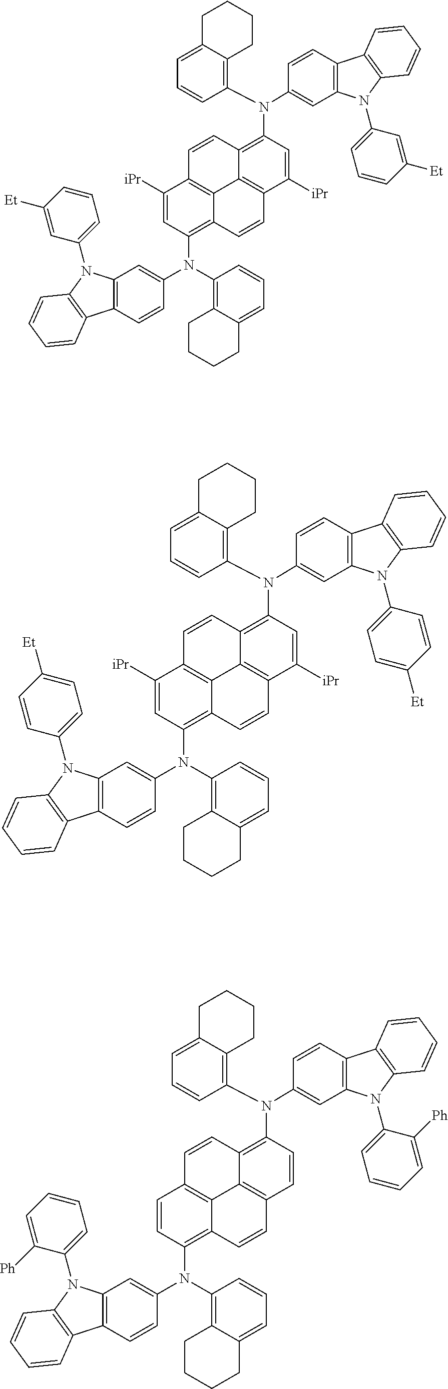 [SMILES: CCc1cccc(-n2c3ccccc3c3ccc(N(c4cccc5c4CCCC5)c4cc(C(C)C)c5ccc6c(N(c7ccc8c9ccccc9n(-c9cccc(CC)c9)c8c7)c7cccc8c7CCCC8)cc(C(C)C)c7ccc4c5c76)cc32)c1 ; CCc1ccc(-n2c3ccccc3c3ccc(N(c4cccc5c4CCCC5)c4cc(C(C)C)c5ccc6c(N(c7ccc8c9ccccc9n(-c9ccc(CC)cc9)c8c7)c7cccc8c7CCCC8)cc(C(C)C)c7ccc4c5c76)cc32)cc1 ; c1ccc(-c2ccccc2-n2c3ccccc3c3ccc(N(c4cccc5c4CCCC5)c4ccc5ccc6c(N(c7ccc8c9ccccc9n(-c9ccccc9-c9ccccc9)c8c7)c7cccc8c7CCCC8)ccc7ccc4c5c76)cc32)cc1]

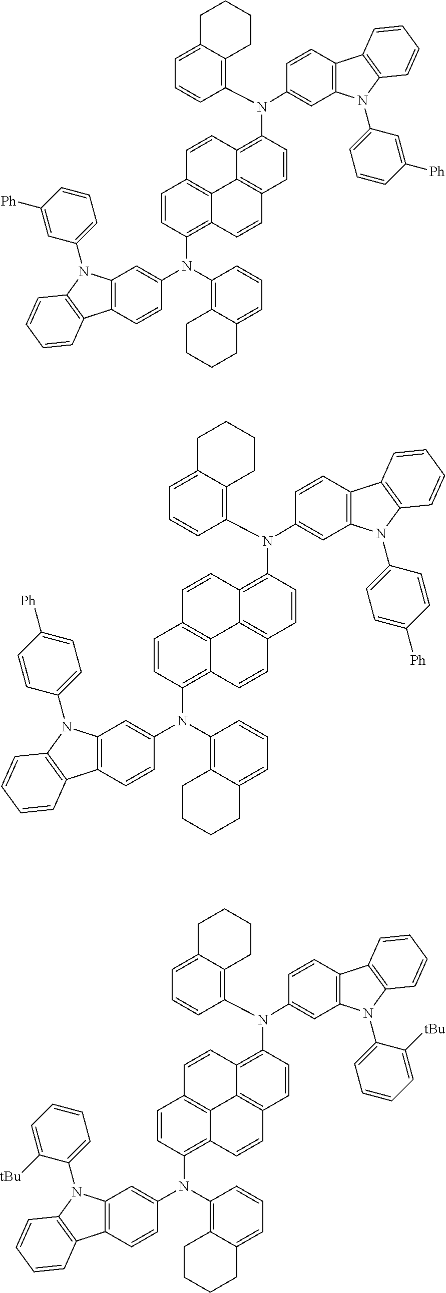 [SMILES: c1ccc(-c2cccc(-n3c4ccccc4c4ccc(N(c5cccc6c5CCCC6)c5ccc6ccc7c(N(c8ccc9c%10ccccc%10n(-c%10cccc(-c%11ccccc%11)c%10)c9c8)c8cccc9c8CCCC9)ccc8ccc5c6c87)cc43)c2)cc1 ; c1ccc(-c2ccc(-n3c4ccccc4c4ccc(N(c5cccc6c5CCCC6)c5ccc6ccc7c(N(c8ccc9c%10ccccc%10n(-c%10ccc(-c%11ccccc%11)cc%10)c9c8)c8cccc9c8CCCC9)ccc8ccc5c6c87)cc43)cc2)cc1 ; CC(C)(C)c1ccccc1-n1c2ccccc2c2ccc(N(c3cccc4c3CCCC4)c3ccc4ccc5c(N(c6ccc7c8ccccc8n(-c8ccccc8C(C)(C)C)c7c6)c6cccc7c6CCCC7)ccc6ccc3c4c65)cc21]

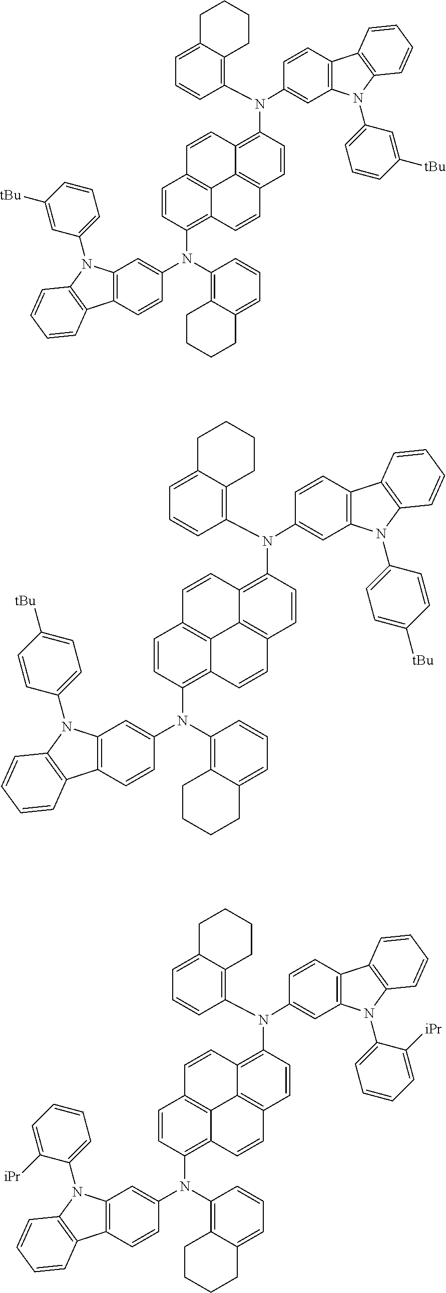 [SMILES: CC(C)(C)c1cccc(-n2c3ccccc3c3ccc(N(c4cccc5c4CCCC5)c4ccc5ccc6c(N(c7ccc8c9ccccc9n(-c9cccc(C(C)(C)C)c9)c8c7)c7cccc8c7CCCC8)ccc7ccc4c5c76)cc32)c1 ; CC(C)(C)c1ccc(-n2c3ccccc3c3ccc(N(c4cccc5c4CCCC5)c4ccc5ccc6c(N(c7ccc8c9ccccc9n(-c9ccc(C(C)(C)C)cc9)c8c7)c7cccc8c7CCCC8)ccc7ccc4c5c76)cc32)cc1 ; CC(C)c1ccccc1-n1c2ccccc2c2ccc(N(c3cccc4c3CCCC4)c3ccc4ccc5c(N(c6ccc7c8ccccc8n(-c8ccccc8C(C)C)c7c6)c6cccc7c6CCCC7)ccc6ccc3c4c65)cc21]

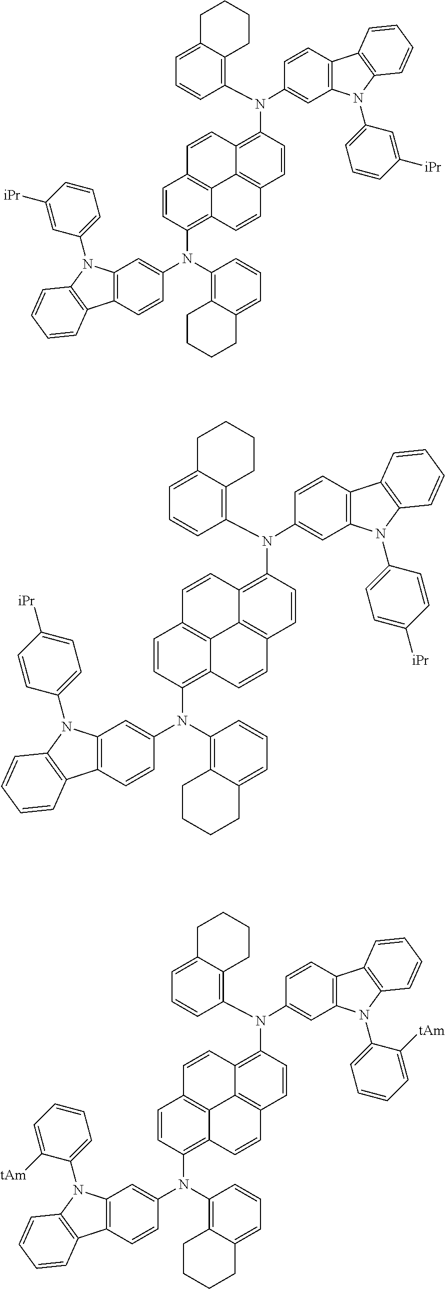

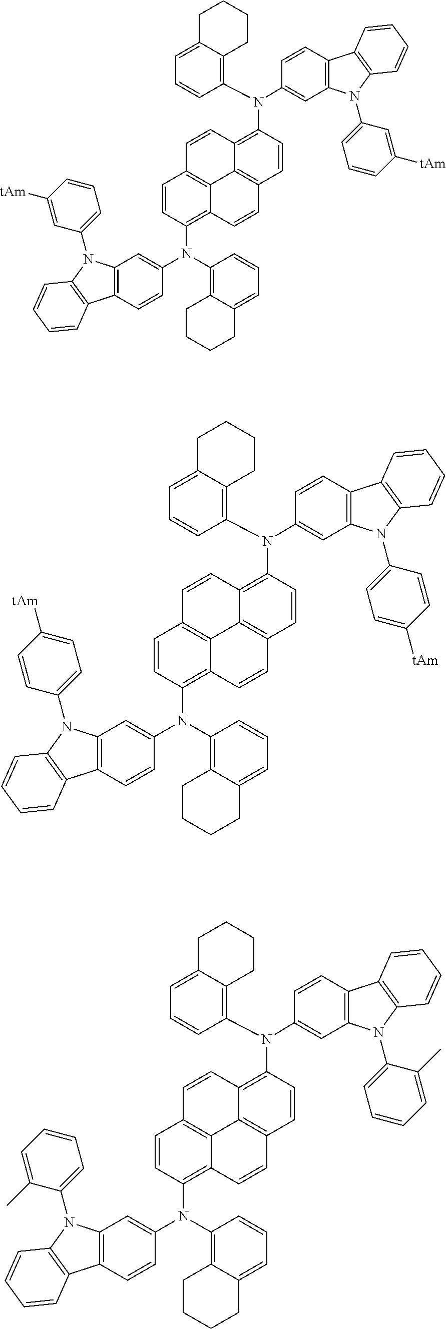

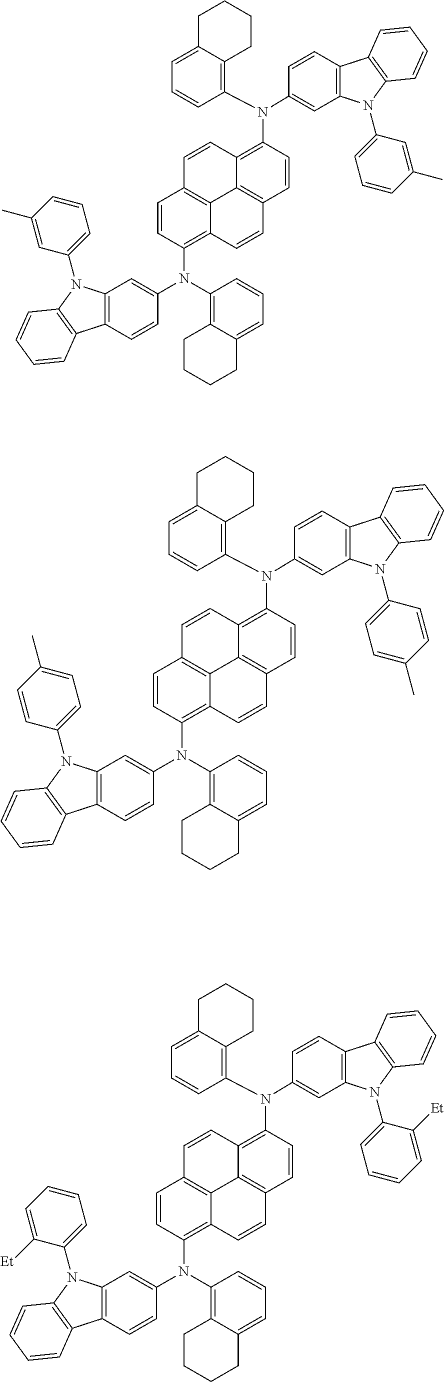

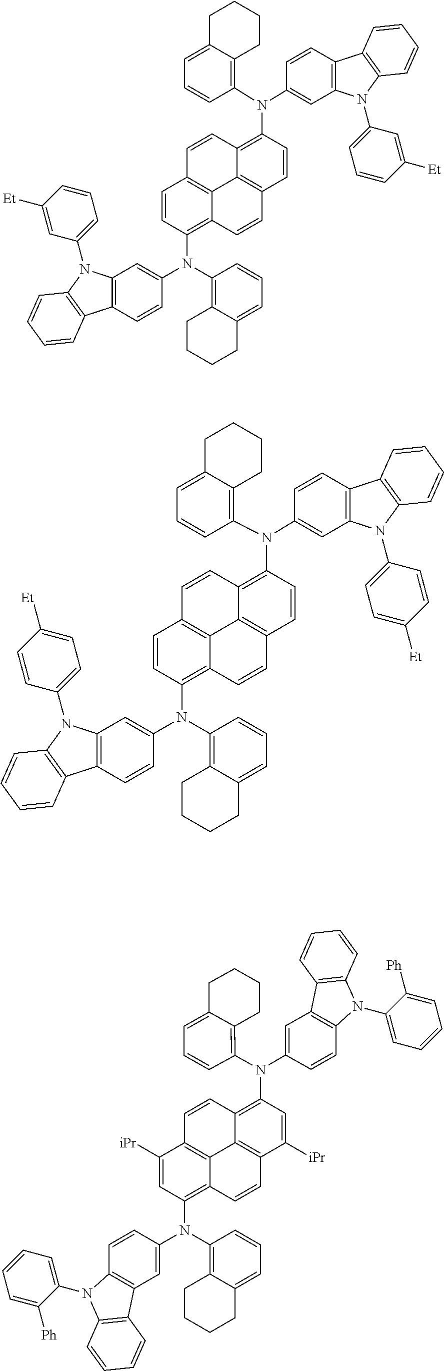 [SMILES: CCc1cccc(-n2c3ccccc3c3ccc(N(c4cccc5c4CCCC5)c4ccc5ccc6c(N(c7ccc8c9ccccc9n(-c9cccc(CC)c9)c8c7)c7cccc8c7CCCC8)ccc7ccc4c5c76)cc32)c1 ; CCc1ccc(-n2c3ccccc3c3ccc(N(c4cccc5c4CCCC5)c4ccc5ccc6c(N(c7ccc8c9ccccc9n(-c9ccc(CC)cc9)c8c7)c7cccc8c7CCCC8)ccc7ccc4c5c76)cc32)cc1 ; CC(C)c1cc(N(c2ccc3c(c2)c2ccccc2n3-c2ccccc2-c2ccccc2)c2cccc3c2CCCC3)c2ccc3c(C(C)C)cc(N(c4ccc5c(c4)c4ccccc4n5-c4ccccc4-c4ccccc4)c4cccc5c4CCCC5)c4ccc1c2c34]

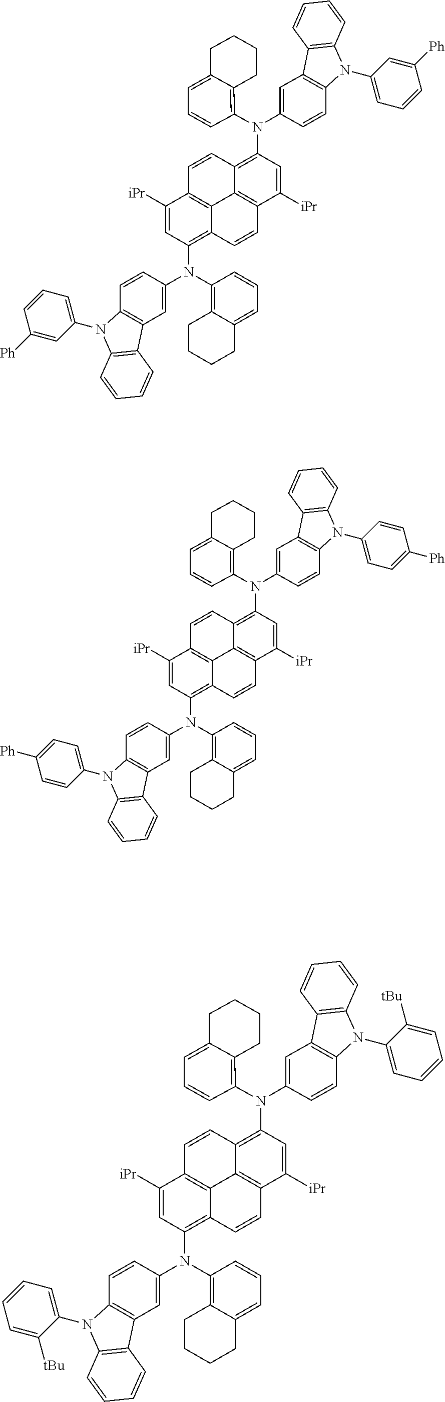 [SMILES: CC(C)c1cc(N(c2ccc3c(c2)c2ccccc2n3-c2cccc(-c3ccccc3)c2)c2cccc3c2CCCC3)c2ccc3c(C(C)C)cc(N(c4ccc5c(c4)c4ccccc4n5-c4cccc(-c5ccccc5)c4)c4cccc5c4CCCC5)c4ccc1c2c34 ; CC(C)c1cc(N(c2ccc3c(c2)c2ccccc2n3-c2ccc(-c3ccccc3)cc2)c2cccc3c2CCCC3)c2ccc3c(C(C)C)cc(N(c4ccc5c(c4)c4ccccc4n5-c4ccc(-c5ccccc5)cc4)c4cccc5c4CCCC5)c4ccc1c2c34 ; CC(C)c1cc(N(c2ccc3c(c2)c2ccccc2n3-c2ccccc2C(C)(C)C)c2cccc3c2CCCC3)c2ccc3c(C(C)C)cc(N(c4ccc5c(c4)c4ccccc4n5-c4ccccc4C(C)(C)C)c4cccc5c4CCCC5)c4ccc1c2c34]

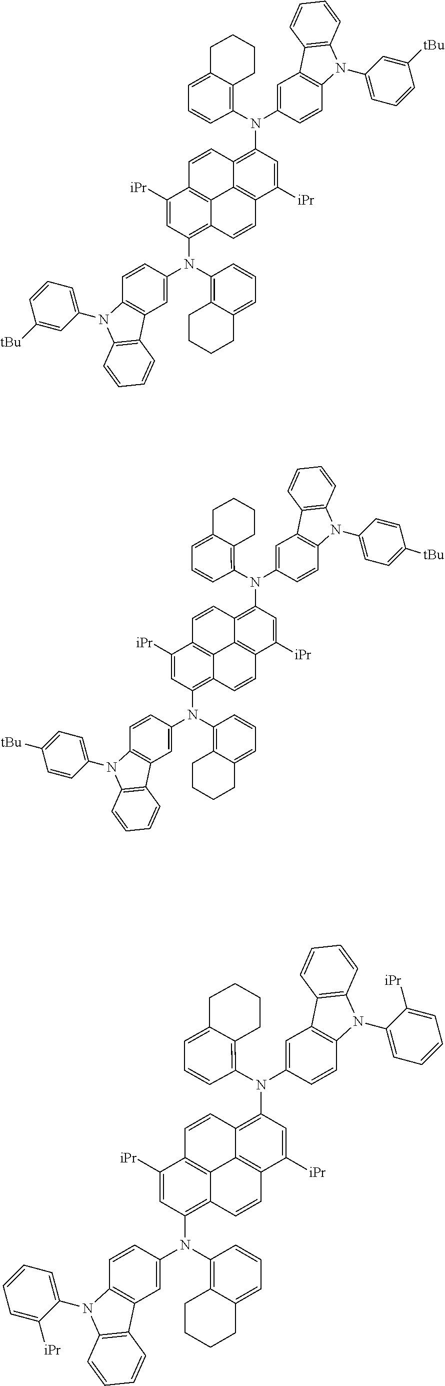 [SMILES: CC(C)c1cc(N(c2ccc3c(c2)c2ccccc2n3-c2cccc(C(C)(C)C)c2)c2cccc3c2CCCC3)c2ccc3c(C(C)C)cc(N(c4ccc5c(c4)c4ccccc4n5-c4cccc(C(C)(C)C)c4)c4cccc5c4CCCC5)c4ccc1c2c34 ; CC(C)c1cc(N(c2ccc3c(c2)c2ccccc2n3-c2ccc(C(C)(C)C)cc2)c2cccc3c2CCCC3)c2ccc3c(C(C)C)cc(N(c4ccc5c(c4)c4ccccc4n5-c4ccc(C(C)(C)C)cc4)c4cccc5c4CCCC5)c4ccc1c2c34 ; CC(C)c1ccccc1-n1c2ccccc2c2cc(N(c3cccc4c3CCCC4)c3cc(C(C)C)c4ccc5c(N(c6ccc7c(c6)c6ccccc6n7-c6ccccc6C(C)C)c6cccc7c6CCCC7)cc(C(C)C)c6ccc3c4c65)ccc21]

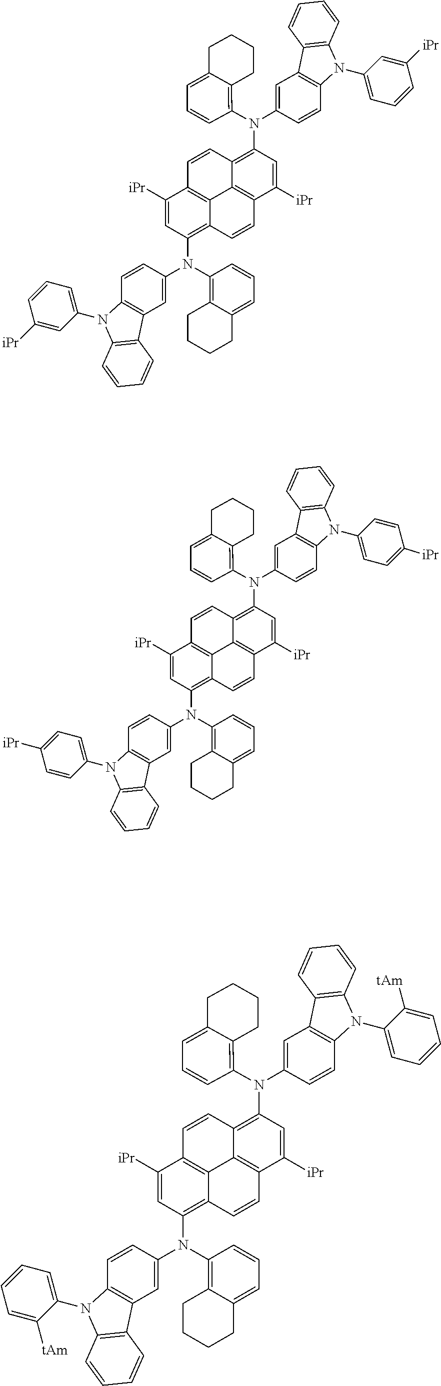

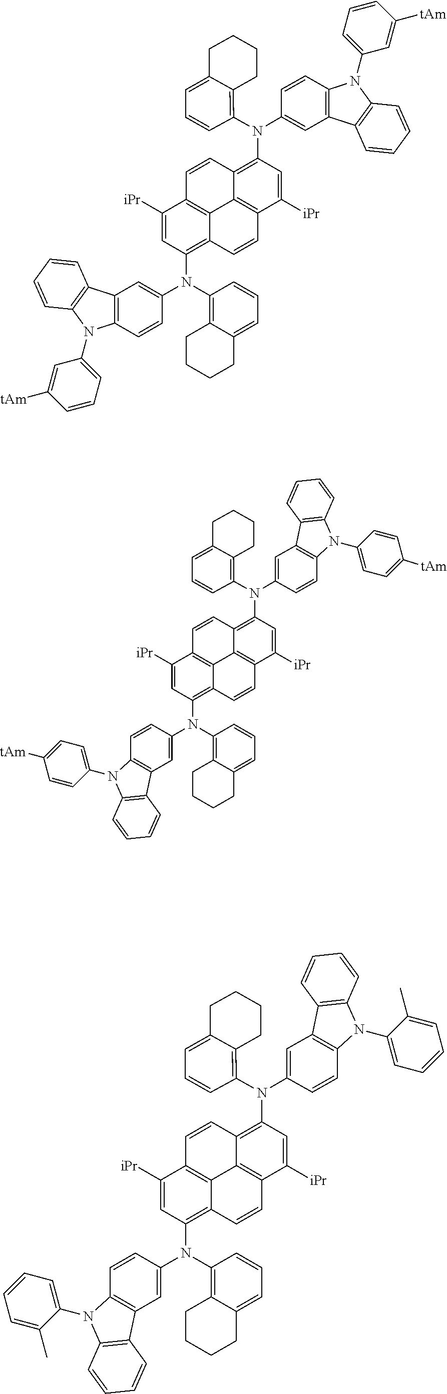

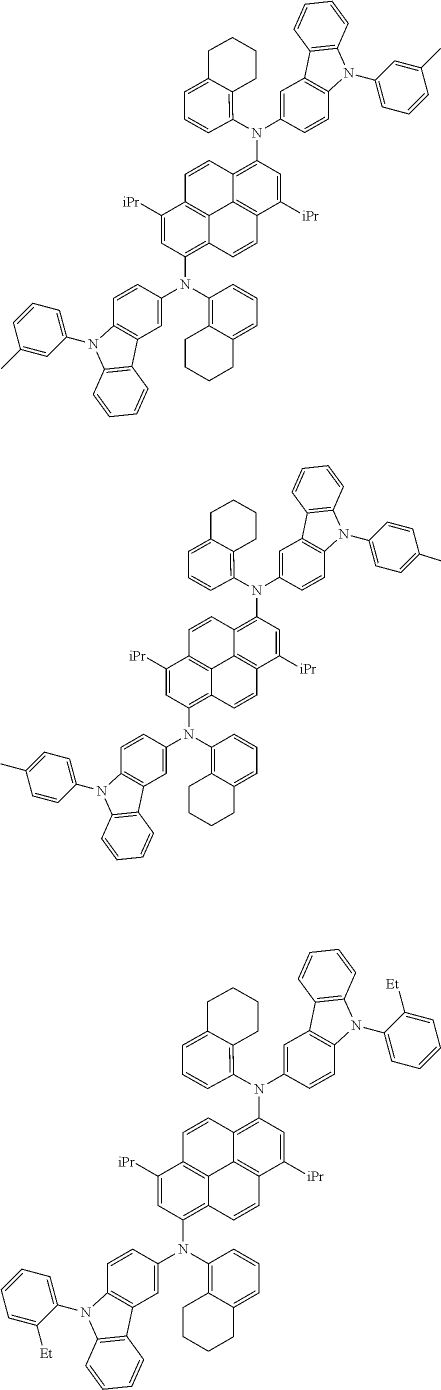

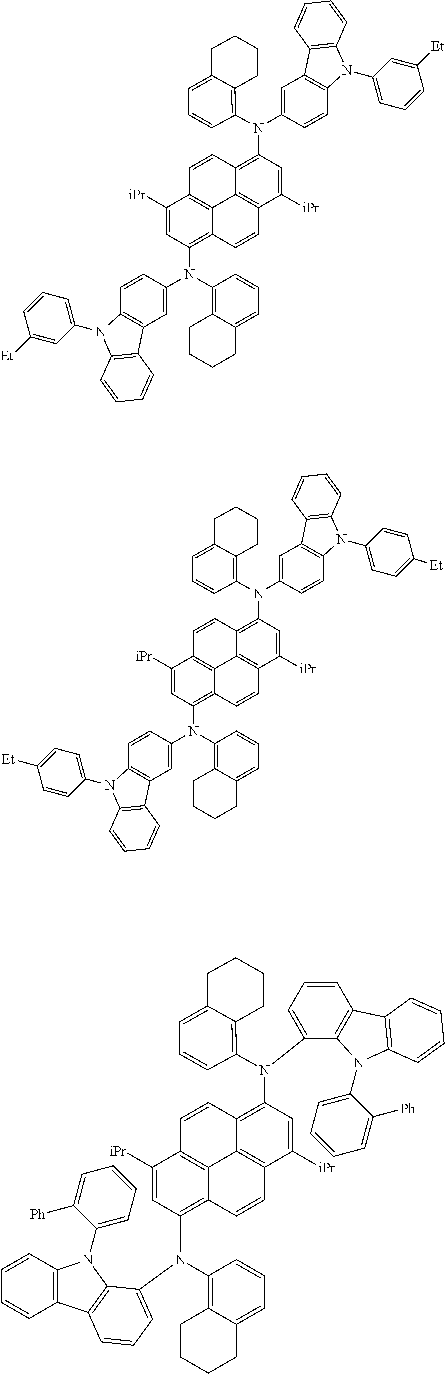 [SMILES: CCc1cccc(-n2c3ccccc3c3cc(N(c4cccc5c4CCCC5)c4cc(C(C)C)c5ccc6c(N(c7ccc8c(c7)c7ccccc7n8-c7cccc(CC)c7)c7cccc8c7CCCC8)cc(C(C)C)c7ccc4c5c76)ccc32)c1 ; CCc1ccc(-n2c3ccccc3c3cc(N(c4cccc5c4CCCC5)c4cc(C(C)C)c5ccc6c(N(c7ccc8c(c7)c7ccccc7n8-c7ccc(CC)cc7)c7cccc8c7CCCC8)cc(C(C)C)c7ccc4c5c76)ccc32)cc1 ; CC(C)c1cc(N(c2cccc3c2CCCC3)c2cccc3c4ccccc4n(-c4ccccc4-c4ccccc4)c23)c2ccc3c(C(C)C)cc(N(c4cccc5c4CCCC5)c4cccc5c6ccccc6n(-c6ccccc6-c6ccccc6)c45)c4ccc1c2c34]

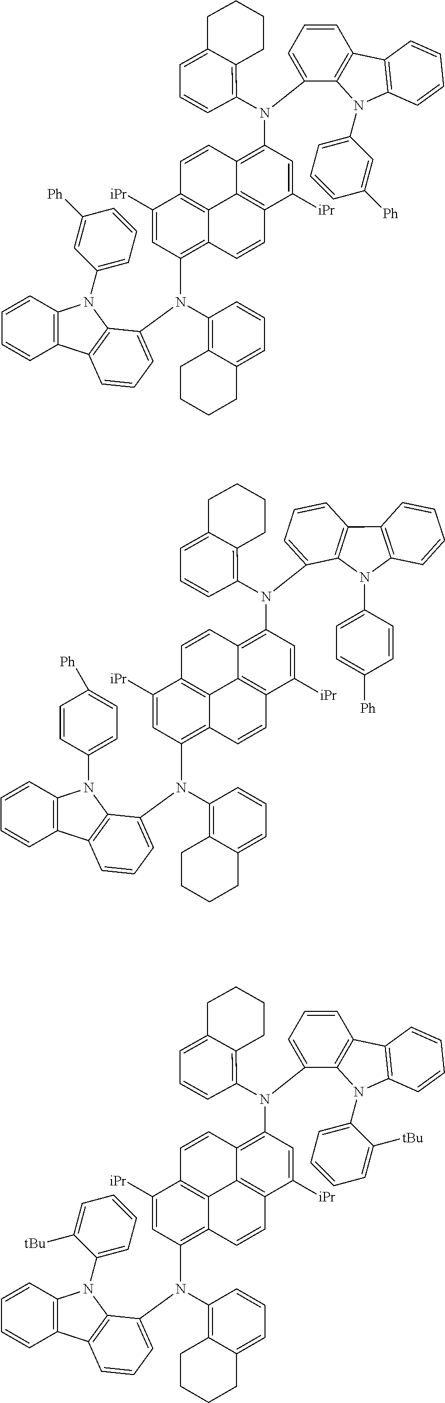 [SMILES: CC(C)c1cc(N(c2cccc3c2CCCC3)c2cccc3c4ccccc4n(-c4cccc(-c5ccccc5)c4)c23)c2ccc3c(C(C)C)cc(N(c4cccc5c4CCCC5)c4cccc5c6ccccc6n(-c6cccc(-c7ccccc7)c6)c45)c4ccc1c2c34 ; CC(C)c1cc(N(c2cccc3c2CCCC3)c2cccc3c4ccccc4n(-c4ccc(-c5ccccc5)cc4)c23)c2ccc3c(C(C)C)cc(N(c4cccc5c4CCCC5)c4cccc5c6ccccc6n(-c6ccc(-c7ccccc7)cc6)c45)c4ccc1c2c34 ; CC(C)c1cc(N(c2cccc3c2CCCC3)c2cccc3c4ccccc4n(-c4ccccc4C(C)(C)C)c23)c2ccc3c(C(C)C)cc(N(c4cccc5c4CCCC5)c4cccc5c6ccccc6n(-c6ccccc6C(C)(C)C)c45)c4ccc1c2c34]

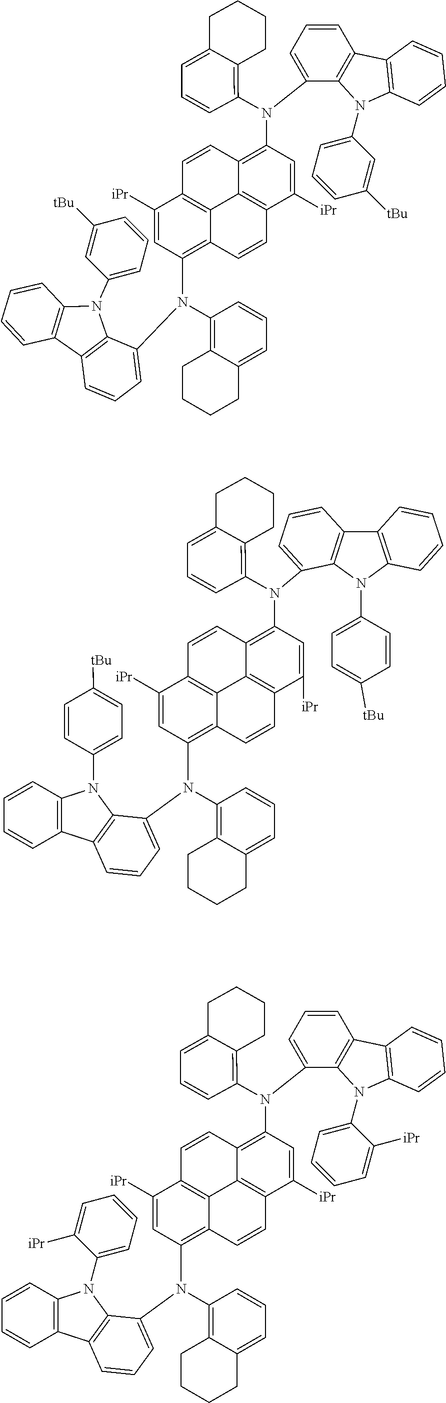 [SMILES: CC(C)c1cc(N(c2cccc3c2CCCC3)c2cccc3c4ccccc4n(-c4cccc(C(C)(C)C)c4)c23)c2ccc3c(C(C)C)cc(N(c4cccc5c4CCCC5)c4cccc5c6ccccc6n(-c6cccc(C(C)(C)C)c6)c45)c4ccc1c2c34 ; CC(C)c1cc(N(c2cccc3c2CCCC3)c2cccc3c4ccccc4n(-c4ccc(C(C)(C)C)cc4)c23)c2ccc3c(C(C)C)cc(N(c4cccc5c4CCCC5)c4cccc5c6ccccc6n(-c6ccc(C(C)(C)C)cc6)c45)c4ccc1c2c34 ; CC(C)c1ccccc1-n1c2ccccc2c2cccc(N(c3cccc4c3CCCC4)c3cc(C(C)C)c4ccc5c(N(c6cccc7c6CCCC7)c6cccc7c8ccccc8n(-c8ccccc8C(C)C)c67)cc(C(C)C)c6ccc3c4c65)c21]

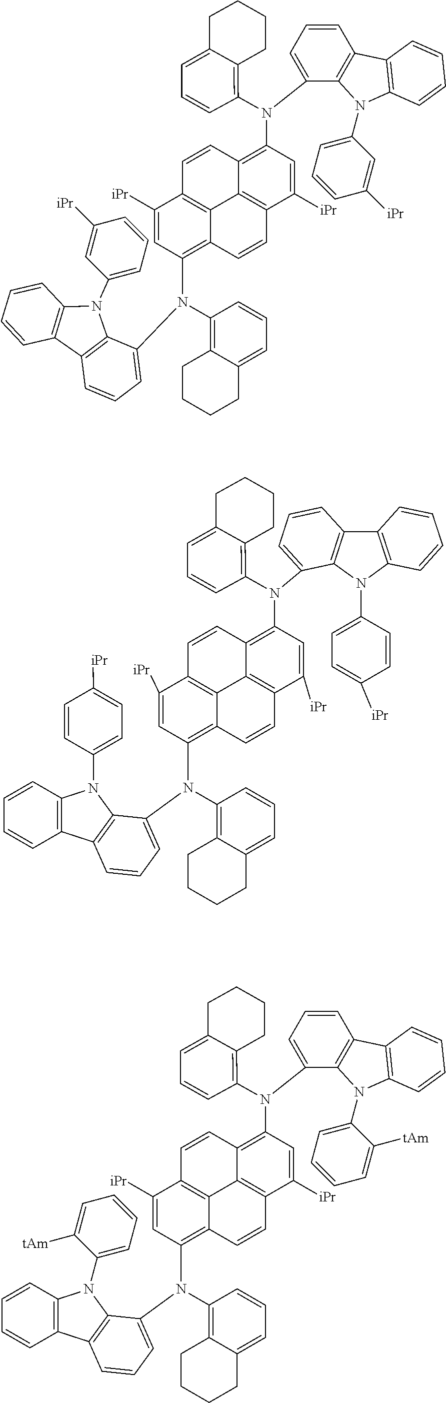

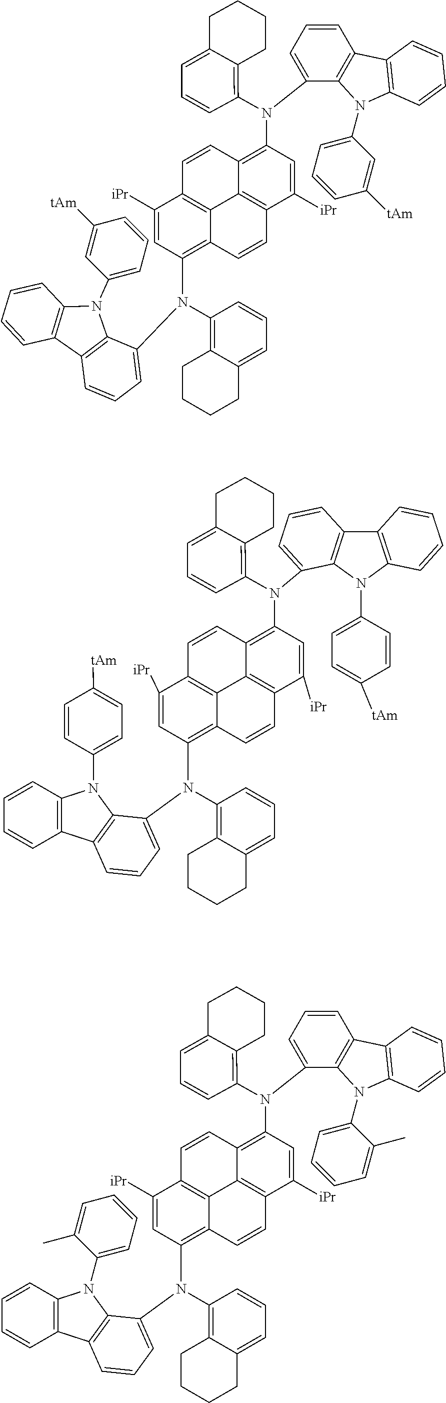

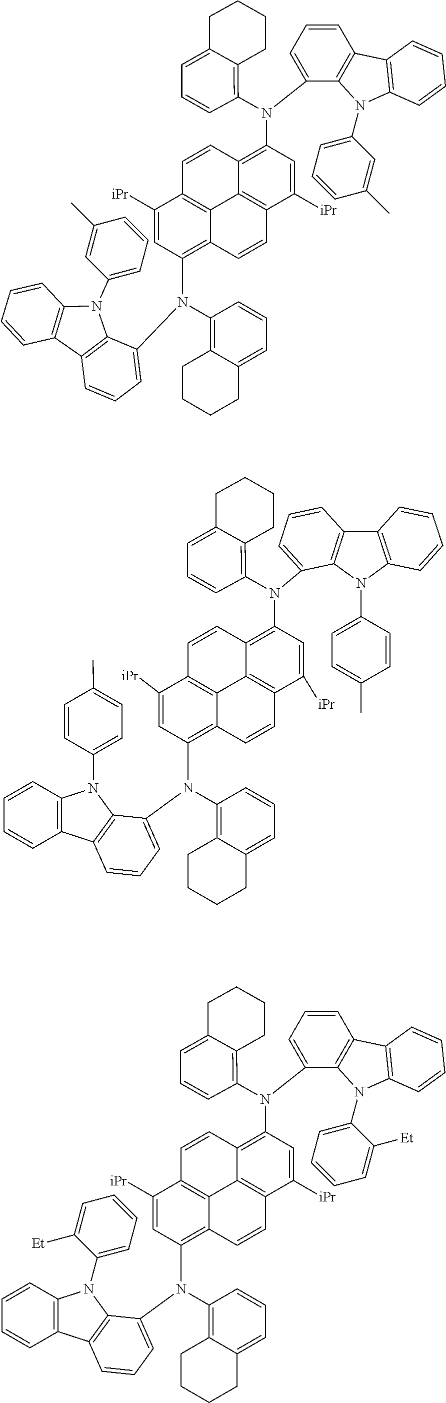 [SMILES: Cc1cccc(-n2c3ccccc3c3cccc(N(c4cccc5c4CCCC5)c4cc(C(C)C)c5ccc6c(N(c7cccc8c7CCCC8)c7cccc8c9ccccc9n(-c9cccc(C)c9)c78)cc(C(C)C)c7ccc4c5c76)c32)c1 ; Cc1ccc(-n2c3ccccc3c3cccc(N(c4cccc5c4CCCC5)c4cc(C(C)C)c5ccc6c(N(c7cccc8c7CCCC8)c7cccc8c9ccccc9n(-c9ccc(C)cc9)c78)cc(C(C)C)c7ccc4c5c76)c32)cc1 ; CCc1ccccc1-n1c2ccccc2c2cccc(N(c3cccc4c3CCCC4)c3cc(C(C)C)c4ccc5c(N(c6cccc7c6CCCC7)c6cccc7c8ccccc8n(-c8ccccc8CC)c67)cc(C(C)C)c6ccc3c4c65)c21]

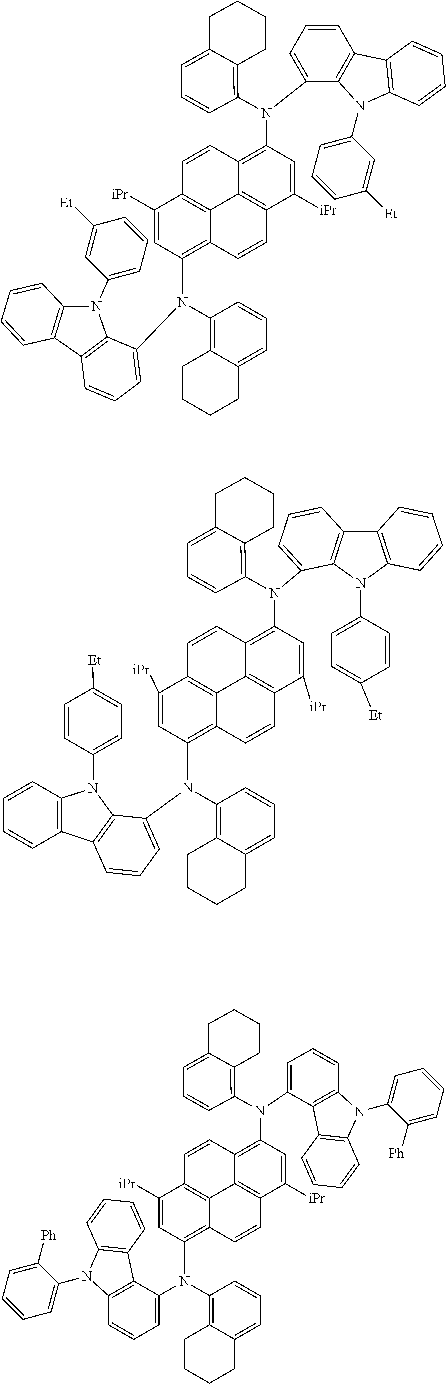 [SMILES: CCc1cccc(-n2c3ccccc3c3cccc(N(c4cccc5c4CCCC5)c4cc(C(C)C)c5ccc6c(N(c7cccc8c7CCCC8)c7cccc8c9ccccc9n(-c9cccc(CC)c9)c78)cc(C(C)C)c7ccc4c5c76)c32)c1 ; CCc1ccc(-n2c3ccccc3c3cccc(N(c4cccc5c4CCCC5)c4cc(C(C)C)c5ccc6c(N(c7cccc8c7CCCC8)c7cccc8c9ccccc9n(-c9ccc(CC)cc9)c78)cc(C(C)C)c7ccc4c5c76)c32)cc1 ; CC(C)c1cc(N(c2cccc3c2CCCC3)c2cccc3c2c2ccccc2n3-c2ccccc2-c2ccccc2)c2ccc3c(C(C)C)cc(N(c4cccc5c4CCCC5)c4cccc5c4c4ccccc4n5-c4ccccc4-c4ccccc4)c4ccc1c2c34]

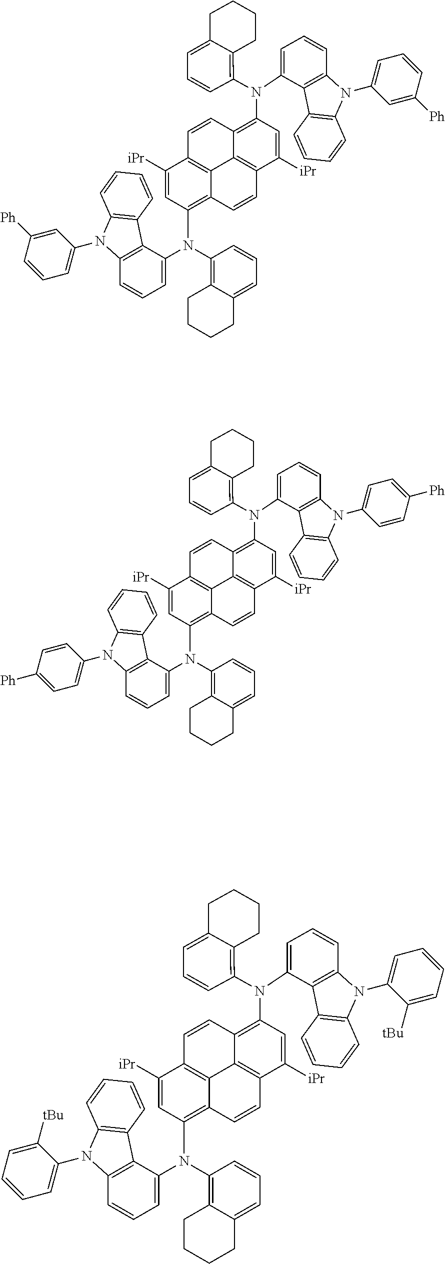 [SMILES: CC(C)c1cc(N(c2cccc3c2CCCC3)c2cccc3c2c2ccccc2n3-c2cccc(-c3ccccc3)c2)c2ccc3c(C(C)C)cc(N(c4cccc5c4CCCC5)c4cccc5c4c4ccccc4n5-c4cccc(-c5ccccc5)c4)c4ccc1c2c34 ; CC(C)c1cc(N(c2cccc3c2CCCC3)c2cccc3c2c2ccccc2n3-c2ccc(-c3ccccc3)cc2)c2ccc3c(C(C)C)cc(N(c4cccc5c4CCCC5)c4cccc5c4c4ccccc4n5-c4ccc(-c5ccccc5)cc4)c4ccc1c2c34 ; CC(C)c1cc(N(c2cccc3c2CCCC3)c2cccc3c2c2ccccc2n3-c2ccccc2C(C)(C)C)c2ccc3c(C(C)C)cc(N(c4cccc5c4CCCC5)c4cccc5c4c4ccccc4n5-c4ccccc4C(C)(C)C)c4ccc1c2c34]

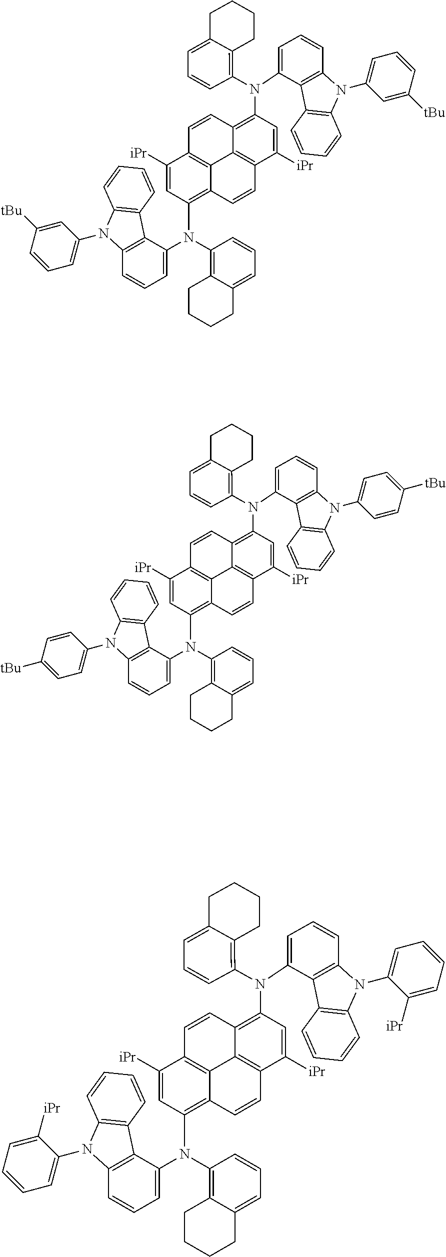 [SMILES: CC(C)c1cc(N(c2cccc3c2CCCC3)c2cccc3c2c2ccccc2n3-c2cccc(C(C)(C)C)c2)c2ccc3c(C(C)C)cc(N(c4cccc5c4CCCC5)c4cccc5c4c4ccccc4n5-c4cccc(C(C)(C)C)c4)c4ccc1c2c34 ; CC(C)c1cc(N(c2cccc3c2CCCC3)c2cccc3c2c2ccccc2n3-c2ccc(C(C)(C)C)cc2)c2ccc3c(C(C)C)cc(N(c4cccc5c4CCCC5)c4cccc5c4c4ccccc4n5-c4ccc(C(C)(C)C)cc4)c4ccc1c2c34 ; CC(C)c1ccccc1-n1c2ccccc2c2c(N(c3cccc4c3CCCC4)c3cc(C(C)C)c4ccc5c(N(c6cccc7c6CCCC7)c6cccc7c6c6ccccc6n7-c6ccccc6C(C)C)cc(C(C)C)c6ccc3c4c65)cccc21]

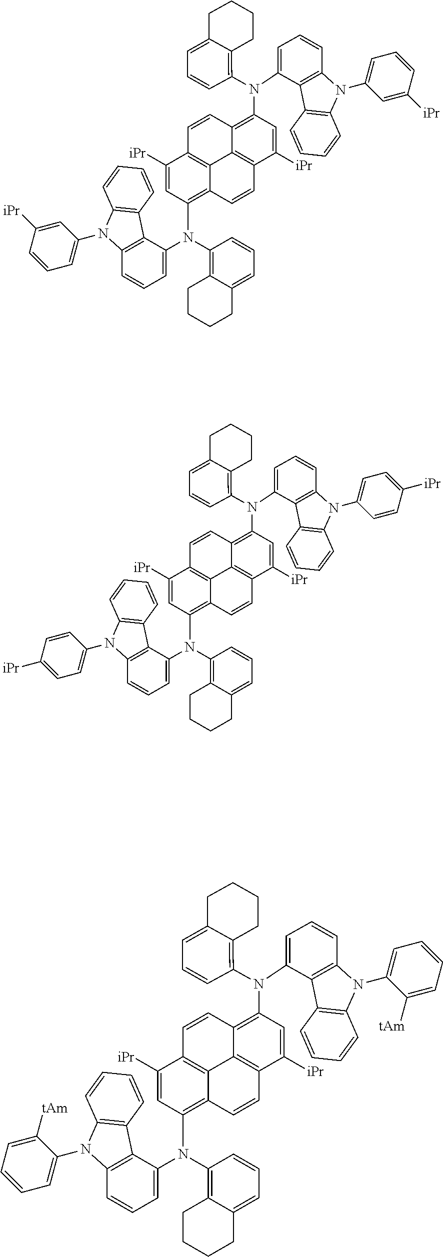

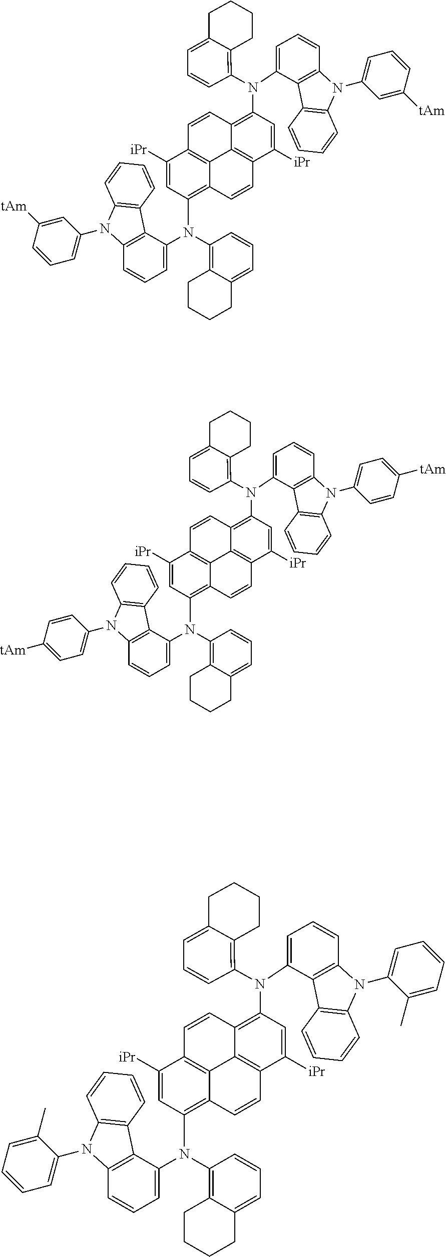

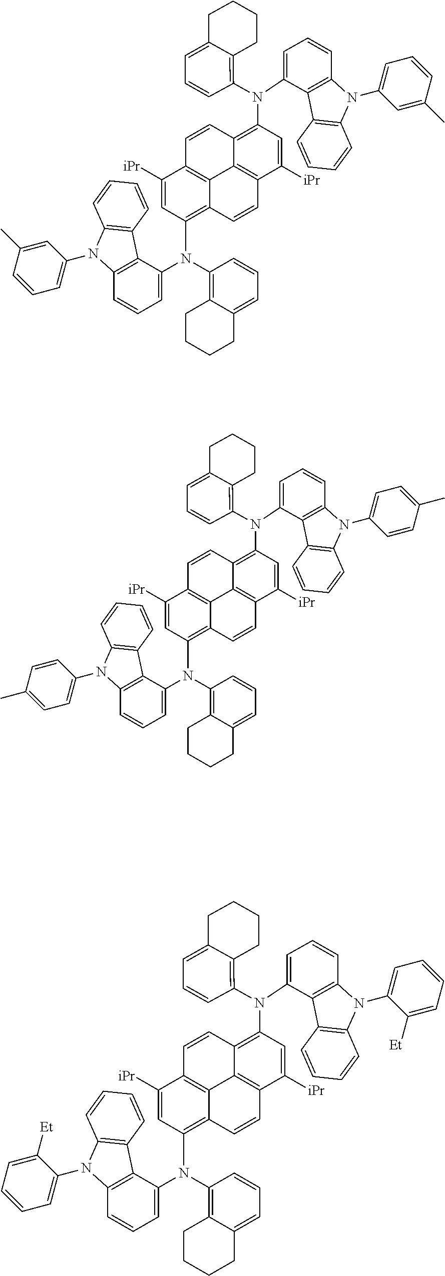

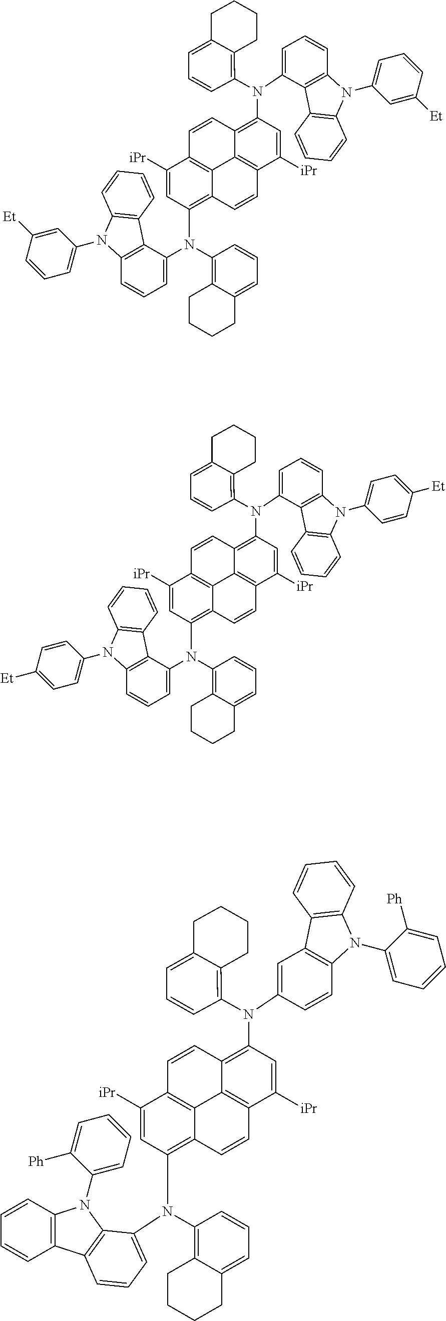 [SMILES: CCc1cccc(-n2c3ccccc3c3c(N(c4cccc5c4CCCC5)c4cc(C(C)C)c5ccc6c(N(c7cccc8c7CCCC8)c7cccc8c7c7ccccc7n8-c7cccc(CC)c7)cc(C(C)C)c7ccc4c5c76)cccc32)c1 ; CCc1ccc(-n2c3ccccc3c3c(N(c4cccc5c4CCCC5)c4cc(C(C)C)c5ccc6c(N(c7cccc8c7CCCC8)c7cccc8c7c7ccccc7n8-c7ccc(CC)cc7)cc(C(C)C)c7ccc4c5c76)cccc32)cc1 ; CC(C)c1cc(N(c2ccc3c(c2)c2ccccc2n3-c2ccccc2-c2ccccc2)c2cccc3c2CCCC3)c2ccc3c(C(C)C)cc(N(c4cccc5c4CCCC5)c4cccc5c6ccccc6n(-c6ccccc6-c6ccccc6)c45)c4ccc1c2c34]

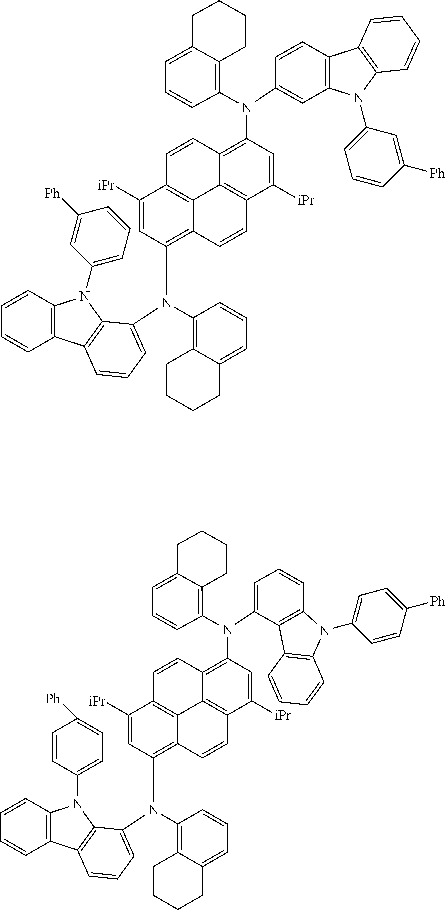 [SMILES: CC(C)c1cc(N(c2ccc3c4ccccc4n(-c4cccc(-c5ccccc5)c4)c3c2)c2cccc3c2CCCC3)c2ccc3c(C(C)C)cc(N(c4cccc5c4CCCC5)c4cccc5c6ccccc6n(-c6cccc(-c7ccccc7)c6)c45)c4ccc1c2c34 ; CC(C)c1cc(N(c2cccc3c2CCCC3)c2cccc3c2c2ccccc2n3-c2ccc(-c3ccccc3)cc2)c2ccc3c(C(C)C)cc(N(c4cccc5c4CCCC5)c4cccc5c6ccccc6n(-c6ccc(-c7ccccc7)cc6)c45)c4ccc1c2c34]

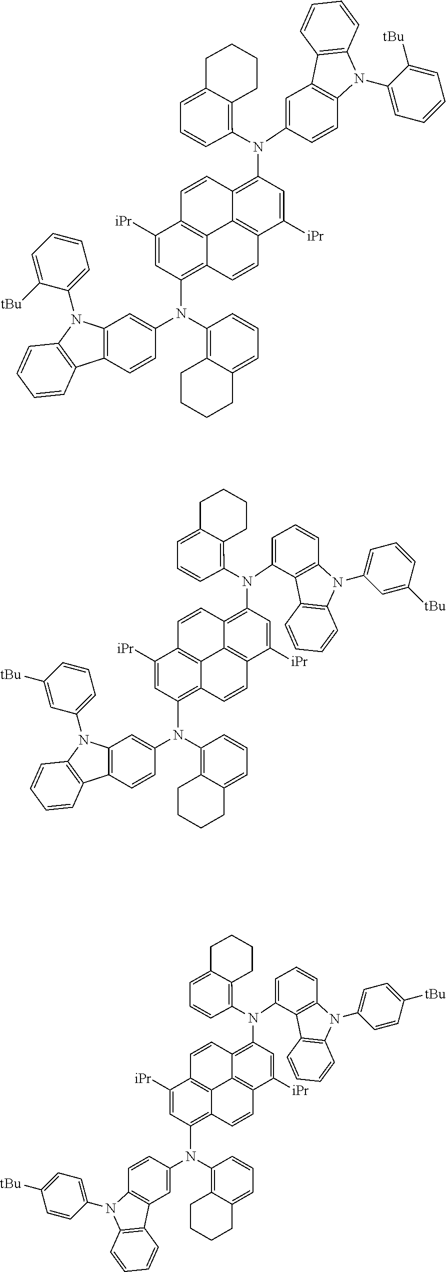 [SMILES: CC(C)c1cc(N(c2ccc3c(c2)c2ccccc2n3-c2ccccc2C(C)(C)C)c2cccc3c2CCCC3)c2ccc3c(C(C)C)cc(N(c4ccc5c6ccccc6n(-c6ccccc6C(C)(C)C)c5c4)c4cccc5c4CCCC5)c4ccc1c2c34 ; CC(C)c1cc(N(c2ccc3c4ccccc4n(-c4cccc(C(C)(C)C)c4)c3c2)c2cccc3c2CCCC3)c2ccc3c(C(C)C)cc(N(c4cccc5c4CCCC5)c4cccc5c4c4ccccc4n5-c4cccc(C(C)(C)C)c4)c4ccc1c2c34 ; CC(C)c1cc(N(c2ccc3c(c2)c2ccccc2n3-c2ccc(C(C)(C)C)cc2)c2cccc3c2CCCC3)c2ccc3c(C(C)C)cc(N(c4cccc5c4CCCC5)c4cccc5c4c4ccccc4n5-c4ccc(C(C)(C)C)cc4)c4ccc1c2c34]

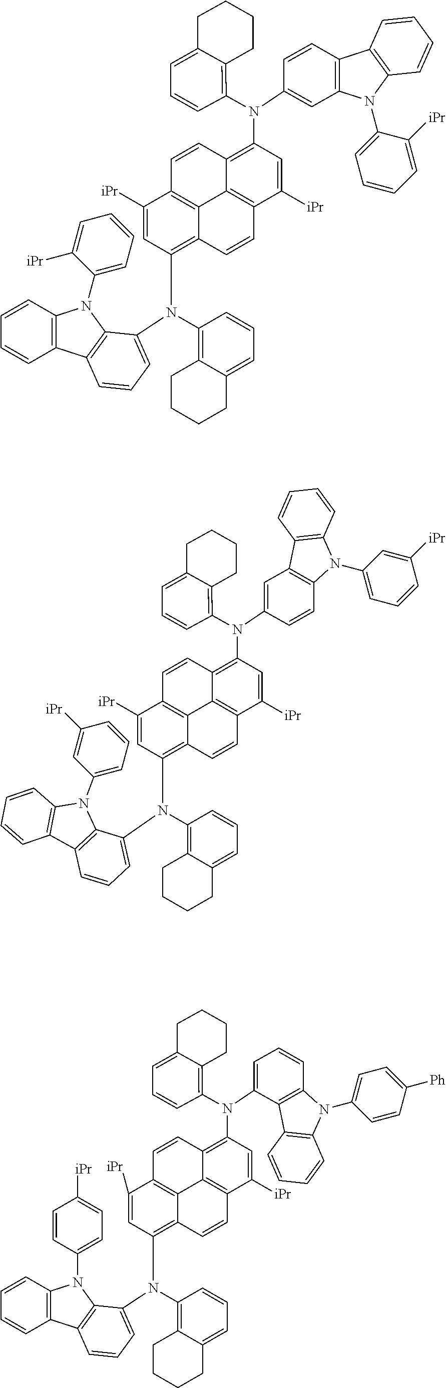 [SMILES: CC(C)c1ccccc1-n1c2ccccc2c2ccc(N(c3cccc4c3CCCC4)c3cc(C(C)C)c4ccc5c(N(c6cccc7c6CCCC7)c6cccc7c8ccccc8n(-c8ccccc8C(C)C)c67)cc(C(C)C)c6ccc3c4c65)cc21 ; CC(C)c1cccc(-n2c3ccccc3c3cc(N(c4cccc5c4CCCC5)c4cc(C(C)C)c5ccc6c(N(c7cccc8c7CCCC8)c7cccc8c9ccccc9n(-c9cccc(C(C)C)c9)c78)cc(C(C)C)c7ccc4c5c76)ccc32)c1 ; CC(C)c1ccc(-n2c3ccccc3c3cccc(N(c4cccc5c4CCCC5)c4cc(C(C)C)c5ccc6c(N(c7cccc8c7CCCC8)c7cccc8c7c7ccccc7n8-c7ccc(-c8ccccc8)cc7)cc(C(C)C)c7ccc4c5c76)c32)cc1]

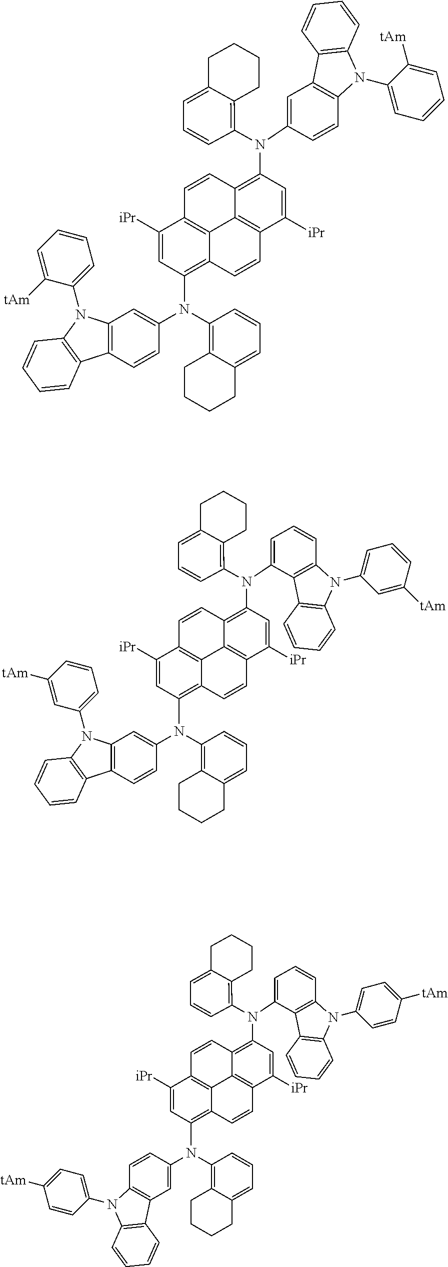

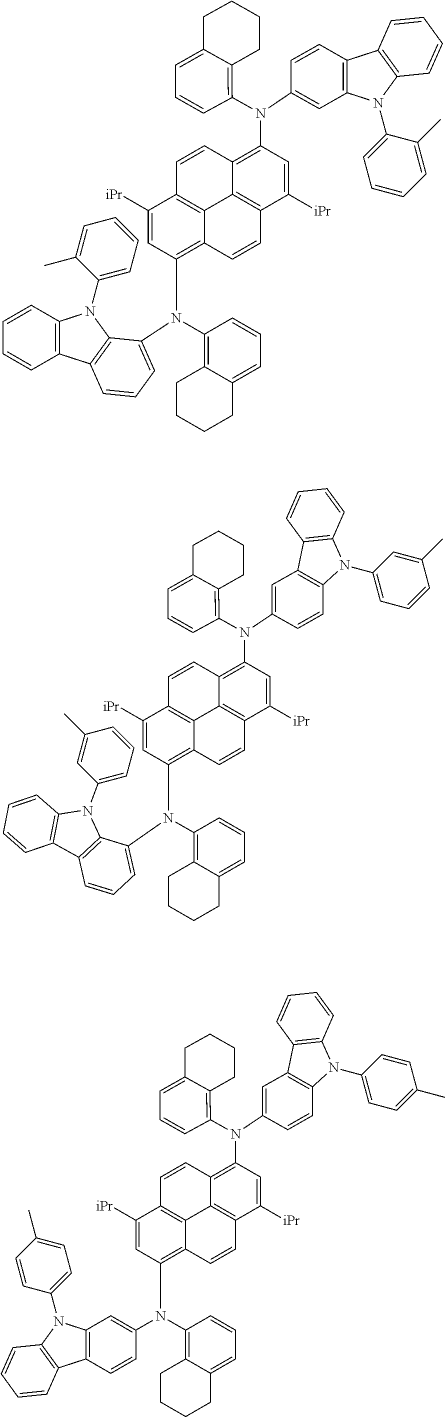 [SMILES: Cc1ccccc1-n1c2ccccc2c2ccc(N(c3cccc4c3CCCC4)c3cc(C(C)C)c4ccc5c(N(c6cccc7c6CCCC7)c6cccc7c8ccccc8n(-c8ccccc8C)c67)cc(C(C)C)c6ccc3c4c65)cc21 ; Cc1cccc(-n2c3ccccc3c3cc(N(c4cccc5c4CCCC5)c4cc(C(C)C)c5ccc6c(N(c7cccc8c7CCCC8)c7cccc8c9ccccc9n(-c9cccc(C)c9)c78)cc(C(C)C)c7ccc4c5c76)ccc32)c1 ; Cc1ccc(-n2c3ccccc3c3cc(N(c4cccc5c4CCCC5)c4cc(C(C)C)c5ccc6c(N(c7ccc8c9ccccc9n(-c9ccc(C)cc9)c8c7)c7cccc8c7CCCC8)cc(C(C)C)c7ccc4c5c76)ccc32)cc1]

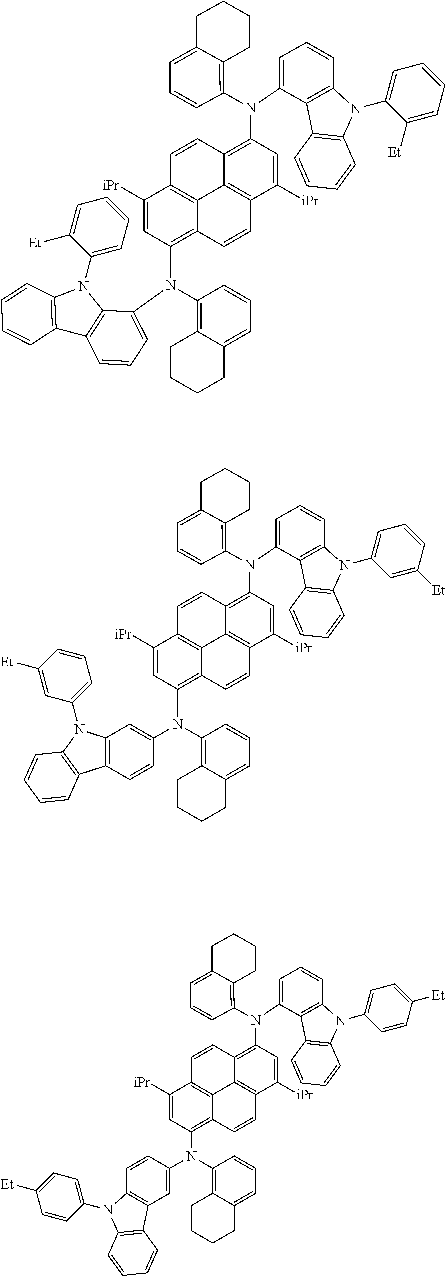 [SMILES: CCc1ccccc1-n1c2ccccc2c2c(N(c3cccc4c3CCCC4)c3cc(C(C)C)c4ccc5c(N(c6cccc7c6CCCC7)c6cccc7c8ccccc8n(-c8ccccc8CC)c67)cc(C(C)C)c6ccc3c4c65)cccc21 ; CCc1cccc(-n2c3ccccc3c3ccc(N(c4cccc5c4CCCC5)c4cc(C(C)C)c5ccc6c(N(c7cccc8c7CCCC8)c7cccc8c7c7ccccc7n8-c7cccc(CC)c7)cc(C(C)C)c7ccc4c5c76)cc32)c1 ; CCc1ccc(-n2c3ccccc3c3cc(N(c4cccc5c4CCCC5)c4cc(C(C)C)c5ccc6c(N(c7cccc8c7CCCC8)c7cccc8c7c7ccccc7n8-c7ccc(CC)cc7)cc(C(C)C)c7ccc4c5c76)ccc32)cc1]

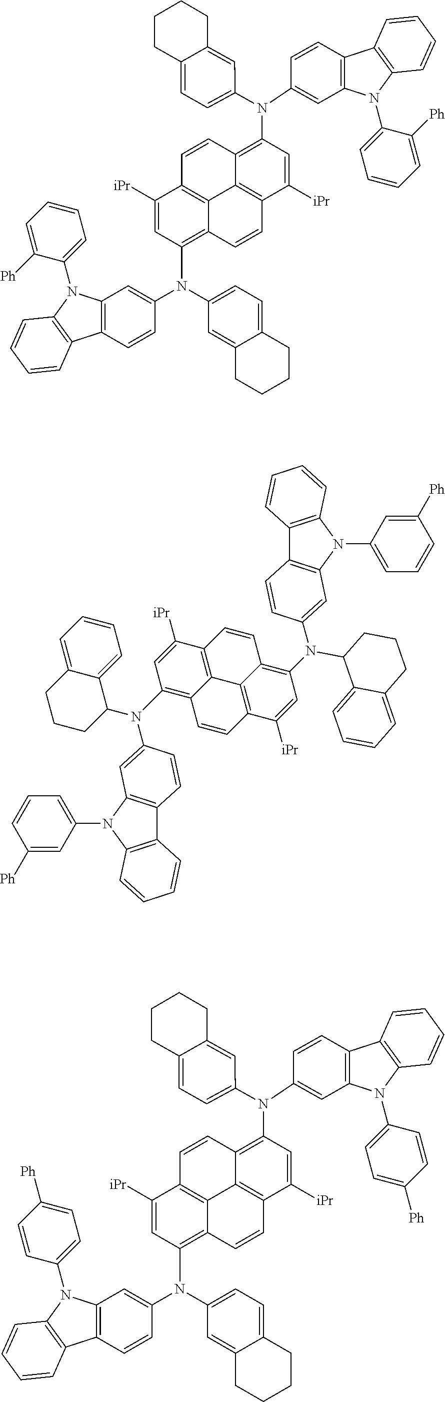 [SMILES: CC(C)c1cc(N(c2ccc3c(c2)CCCC3)c2ccc3c4ccccc4n(-c4ccccc4-c4ccccc4)c3c2)c2ccc3c(C(C)C)cc(N(c4ccc5c(c4)CCCC5)c4ccc5c6ccccc6n(-c6ccccc6-c6ccccc6)c5c4)c4ccc1c2c34 ; CC(C)c1cc(N(c2ccc3c4ccccc4n(-c4cccc(-c5ccccc5)c4)c3c2)C2CCCc3ccccc32)c2ccc3c(C(C)C)cc(N(c4ccc5c6ccccc6n(-c6cccc(-c7ccccc7)c6)c5c4)C4CCCc5ccccc54)c4ccc1c2c34 ; CC(C)c1cc(N(c2ccc3c(c2)CCCC3)c2ccc3c4ccccc4n(-c4ccc(-c5ccccc5)cc4)c3c2)c2ccc3c(C(C)C)cc(N(c4ccc5c(c4)CCCC5)c4ccc5c6ccccc6n(-c6ccc(-c7ccccc7)cc6)c5c4)c4ccc1c2c34]

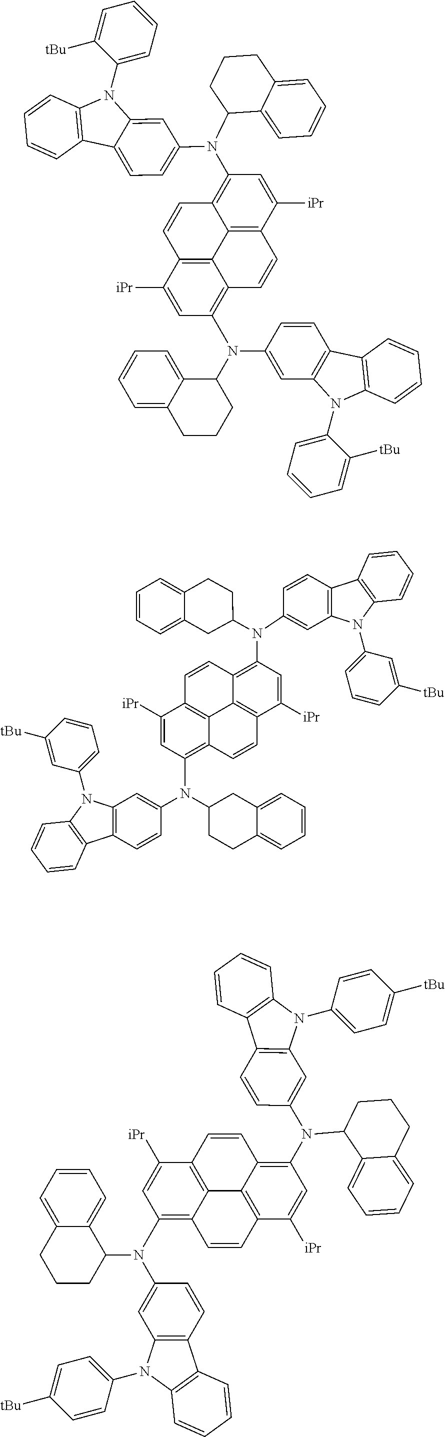 [SMILES: CC(C)c1cc(N(c2ccc3c4ccccc4n(-c4ccccc4C(C)(C)C)c3c2)C2CCCc3ccccc32)c2ccc3c(C(C)C)cc(N(c4ccc5c6ccccc6n(-c6ccccc6C(C)(C)C)c5c4)C4CCCc5ccccc54)c4ccc1c2c34 ; CC(C)c1cc(N(c2ccc3c4ccccc4n(-c4cccc(C(C)(C)C)c4)c3c2)C2CCc3ccccc3C2)c2ccc3c(C(C)C)cc(N(c4ccc5c6ccccc6n(-c6cccc(C(C)(C)C)c6)c5c4)C4CCc5ccccc5C4)c4ccc1c2c34 ; CC(C)c1cc(N(c2ccc3c4ccccc4n(-c4ccc(C(C)(C)C)cc4)c3c2)C2CCCc3ccccc32)c2ccc3c(C(C)C)cc(N(c4ccc5c6ccccc6n(-c6ccc(C(C)(C)C)cc6)c5c4)C4CCCc5ccccc54)c4ccc1c2c34]

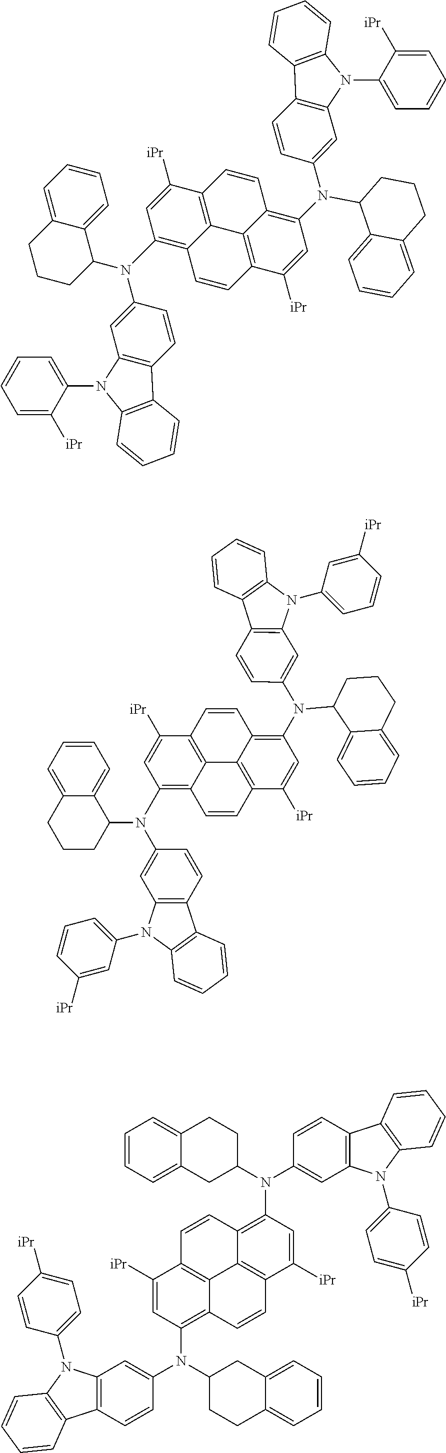 [SMILES: CC(C)c1ccccc1-n1c2ccccc2c2ccc(N(c3cc(C(C)C)c4ccc5c(N(c6ccc7c8ccccc8n(-c8ccccc8C(C)C)c7c6)C6CCCc7ccccc76)cc(C(C)C)c6ccc3c4c65)C3CCCc4ccccc43)cc21 ; CC(C)c1cccc(-n2c3ccccc3c3ccc(N(c4cc(C(C)C)c5ccc6c(N(c7ccc8c9ccccc9n(-c9cccc(C(C)C)c9)c8c7)C7CCCc8ccccc87)cc(C(C)C)c7ccc4c5c76)C4CCCc5ccccc54)cc32)c1 ; CC(C)c1ccc(-n2c3ccccc3c3ccc(N(c4cc(C(C)C)c5ccc6c(N(c7ccc8c9ccccc9n(-c9ccc(C(C)C)cc9)c8c7)C7CCc8ccccc8C7)cc(C(C)C)c7ccc4c5c76)C4CCc5ccccc5C4)cc32)cc1]

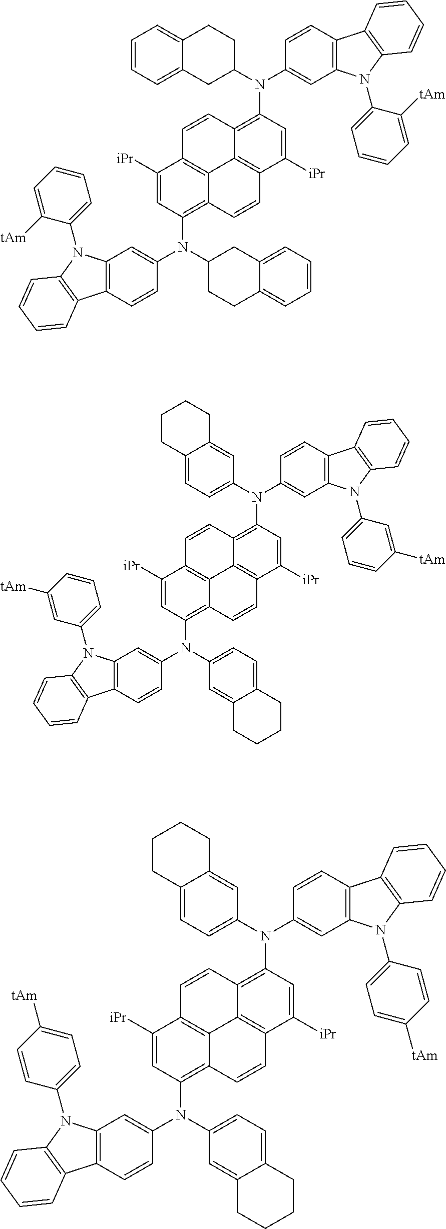

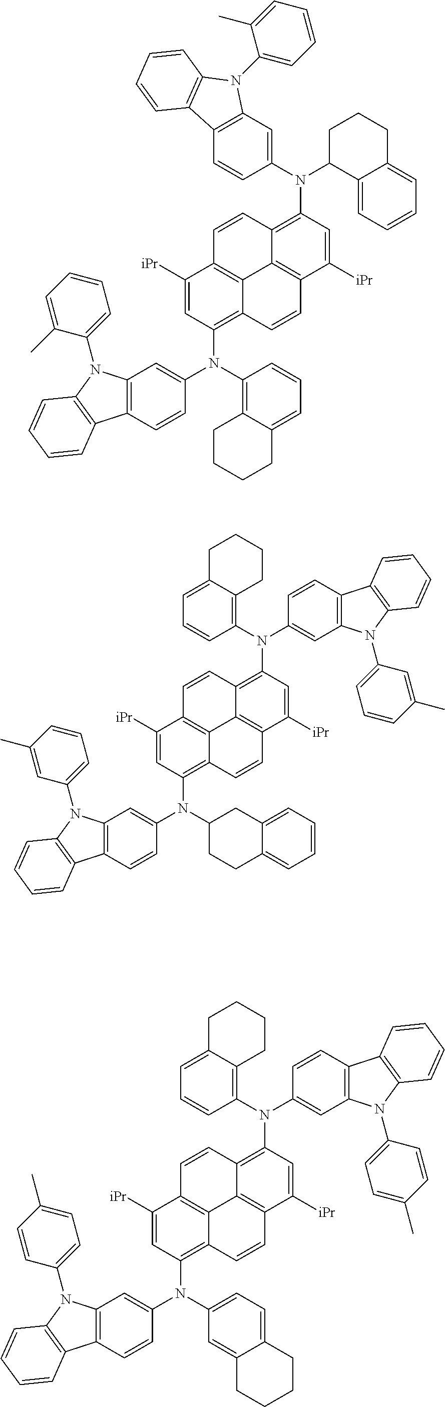

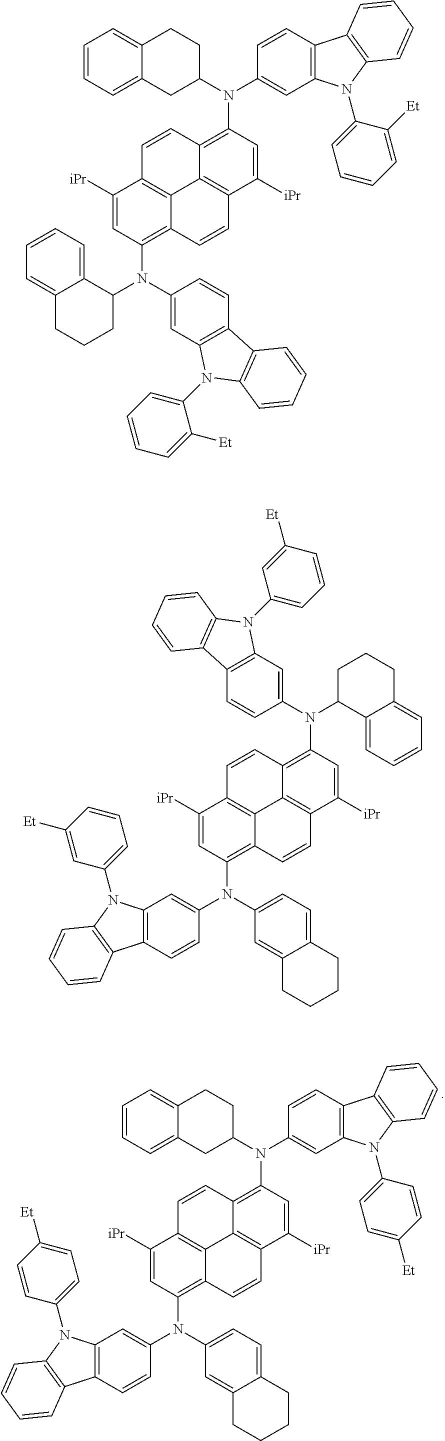 [SMILES: CCc1ccccc1-n1c2ccccc2c2ccc(N(c3cc(C(C)C)c4ccc5c(N(c6ccc7c8ccccc8n(-c8ccccc8CC)c7c6)C6CCCc7ccccc76)cc(C(C)C)c6ccc3c4c65)C3CCc4ccccc4C3)cc21 ; CCc1cccc(-n2c3ccccc3c3ccc(N(c4ccc5c(c4)CCCC5)c4cc(C(C)C)c5ccc6c(N(c7ccc8c9ccccc9n(-c9cccc(CC)c9)c8c7)C7CCCc8ccccc87)cc(C(C)C)c7ccc4c5c76)cc32)c1 ; CCc1ccc(-n2c3ccccc3c3ccc(N(c4ccc5c(c4)CCCC5)c4cc(C(C)C)c5ccc6c(N(c7ccc8c9ccccc9n(-c9ccc(CC)cc9)c8c7)C7CCc8ccccc8C7)cc(C(C)C)c7ccc4c5c76)cc32)cc1]

The tetrahydronaphthalene-based organic compounds provided herein can be used as a functional material in a functional layer of an organic electronic device. The functional layer includes, but is not limited to, one or more of a hole injecting layer (HIL), a hole transporting layer (HTL), an electron transporting layer (ETL), an electron injectinglayer (EIL), an electron barrier layer (EBL), a hole blocking layer (HBL), and a light-emitting layer (EML).

In some embodiments, the tetrahydronaphthalene-based organic compounds provided herein are used in a light-emitting layer.

Accordingly, the present disclosure also provides a blue light-emitting material which is a tetrahydronaphthalene-based organic compound as described above.

The present disclosure further provides a mixture comprising at least one of the tetrahydronaphthalene-based organic compounds described above, and at least one organic functional material selected from a hole injecting material, a hole transporting material, an electron transporting material, an electron injecting material, an electron blocking material, a hole blocking material, a light-emitting material, a host material, or an organic dye.

The present disclosure further provides a composition comprising at least one tetrahydronaphthalene-based organic compound as described above or a mixture as described above, and at least one organic solvent. The at least one organic solvent is selected from one or more of an aromatic compound, a heteroaromatic compound, an ester compound, an aromatic ketone compound, an aromatic ether compound, an aliphatic ketone compound, an aliphatic ether compound, an alicyclic compound, an alkene compound, a borate compound and a phosphate compound.

In a preferable embodiment, according to the formulation of the present disclosure, the at least one organic solvent is selected from aromatic or heteroaromatic based solvents.

Specifically, examples of the aromatic or heteroaromatic based solvents suitable for the present disclosure include, but are not limited to, p-diisopropylbenzene, pentyl benzene, tetrahydronaphthalene, cyclohexylbenzene, chloronaphthalene, 1,4-dimethylnaphthalene 3-isopropylbiphenyl, p-methylisopropylbenzene, dipentylbenzene, tripentylbenzene, pentyltoluene, o-diethylbezene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, cyclohexylbenzene, benzylbutylbenzene, dimethylnaphthalene, 3-isopropylbiphenyl, p-methylisopropylbenzene, 1-methylnaphthalene, 1,2,4-trichlorobenzene, 4,4-difluorodiphenylmethane, 1,2-dimethoxy-4-(1-propenyl)benzene, diphenylmethane, 2-phenylpyridine, 3-phenylpyridine, N-methyldiphenylamine, 4-isopropylbiphenyl, α,α-dichlorodiphenylmethane, 4-(3-phenylpropyl)pyridine, benzyl benzoate, 1,1-bis(3,4-dimethylphenyl) ethane, 2-isopropylnaphthalene, quinoline, isoquinoline, methyl 2-furancarboxylate, and ethyl 2-furancarboxylate.

Examples of the aromatic ketone based solvents suitable for the present disclosure include, but are not limited to, 1-tetralone, 2-tetralone, 2-(phenylepoxy)tetralone, 6-(methyloxy)tetralone, acetophenone, propiophenone, benzophenone, and derivatives thereof. The derivatives are exemplified as 4-methylacetophenone, 3-methylacetophenone, 2-methylacetophenone, 4-methylpropiophenone, 3-methylpropiophenone, 2-methylpropiophenone.

Examples of the aromatic ether based solvents suitable for the present disclosure include, but are not limited to, 3-phenoxytoluene, butoxybenzene, p-anisaldehyde dimethyl acetal, tetrahydro-2-phenoxy-2H-pyran, 1,2dimethoxy-4-(1-propenyl)benzene, 1,4-benzodioxane, 1,3dipropylbenzene, 2,5-dimethoxytoluene, 4-ethylphenetole, 1,3-dipropoxybenzene, 1,2,4-trimethoxybenzene, 4-(1-propenyl)-1,2-dimethoxybenzene, 1,3-dimethoxybenzene, gly-cidyl phenyl ether, dibenzyl ether, 4-tert-butylanisole, trans1,2-dimethoxybenzene, anisole, p-propenyl 1-methoxynaphthalene, diphenyl ether, 2-phenoxymethyl ether, 2-phenoxytetrahydrofuran, and ethyl-2-naphthyl ether.

In some preferred embodiments, according to the compositions of the present disclosure, the at least one organic solvent may be selected from the group consisting of aliphatic ketones, such as 2-nonanone, 3-nonanone, 5-nonanone, 2-decanone, 2,5-hexanedione, 2,6,8-trimethyl-4-nonanone, fenchone, phorone, isophorone, 6-undecanone, and the like; and aliphatic ethers, such as pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, and tetraethylene glycol dimethyl ether.

In other preferred embodiments, according to the compositions of the present disclosure, the at least one organic solvent may be selected from the ester based solvents such as alkyl caprylate, alkyl sebacate, alkyl stearate, alkyl benzoate, alkyl phenylacetate, alkyl cinnamate, alkyl oxalate, alkyl maleate, alkyl lactone, alkyl oleate, etc., in particular, octyl octanoate, diethyl sebacate, diallyl phthalate, and isononyl isononanoate.

The aforementioned solvent may be used alone or used as a mixture of two or more organic solvents.

In some preferred embodiments, one composition according to the present disclosure includes at least one tetrahydronaphthalene-based organic compound or polymer or mixture as described above, and at least one organic solvent, and may further comprise another organic solvent. Examples of the another organic solvent include, but are not limited to, one or more of methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, and decalin.

In the present disclosure, the boiling point of the organic solvent is greater than or equal to 150° C., preferably greater than or equal to 180° C., preferably greater than or equal to 200° C.; more preferably greater than or equal to 250° C.; most preferably greater than or equal to 275° C., or greater than or equal to 300° C. Boiling points in these ranges are beneficial for preventing the clogging of the nozzle of the inkjet printing head. A film comprising the functional material can be made by evaporating organic solvent from the solvent mixture.

In a preferred embodiment, the composition according to the present disclosure is a solution.

In another preferred embodiment, the composition according to the present disclosure is a suspension.

The composition in embodiments of the present disclosure may include 0.01 wt % to 20 wt %, preferably 0.1 wt % to 15 wt %, more preferably 0.2 wt % to 5 wt %, and most preferably 0.25 wt % to 3 wt % of the organic compound or mixture according to the present disclosure.

The present disclosure further relates to the use of the composition as a coating or printing ink in the preparation of organic electronic devices, particularly by the printing or coating method.

The appropriate printing technology or coating technology includes, but is not limited to, inkjet printing, typography, screen printing, dip coating, spin coating, blade coating, roller printing, twist roller printing, lithography, flexography, rotary printing, spray coating, brush coating or transfer printing, slot die coating, specially gravure printing, nozzle printing and inkjet printing. The solution or the suspension may further comprise one or more components, such as surfactant, lubricant, wetting agent, dispersant, hydrophobic agent, binder to adjust the viscosity and the film forming property and to improve the adhesion property.

The present disclosure further provides an use of the tetrahydronaphthalene-based organic compound, mixture or composition as described above in organic electronic devices. The organic electronic devices may be selected from, but are not limited to, organic light-emitting diode (OLED), organic photovoltaic cell (OPV), organic light-emitting electrochemical cell (OLEEC), organic field effect transistor (OFET), organic light-emitting field effect transistor, organic laser, organic spintronic device, organic sensor, and organic plasmon emitting diode (OPED). In an embodiment of the present disclosure, the organic electronic device is an OLED device, and preferably, the tetrahydronaphthalene-based organic compound is used in the light-emitting layer of the OLED device.

The present disclosure further relates to an organic electronic device comprising at least one functional layer, wherein the functional layer comprises the tetrahydronaphthalene-based organic compound, the mixture as described above or is prepared from the above-mentioned composition. Further, the organic electronic device includes a cathode, an anode and at least a functional layer, wherein the functional layer comprises the polycyclic compound, the mixture as described above or is prepared from the above-mentioned composition.

The functional layer may be a hole injecting layer, a hole transporting layer, a light-emitting layer, an electron blocking layer, an electron injectinglayer, an electron transporting layer, or a hole blocking layer. Preferably, the functional layer is a light-emitting layer.

In the above light-emitting device, particularly in the OLED, a substrate, an anode, at least one light-emitting layer and a cathode are included.

The substrate may be transparent or opaque. The substrate may be rigid or elastic. A material of the substrate may be plastic, metal, semiconductor wafer or glass. Preferably, the substrate has a smooth surface. In at least one preferred embodiment, the substrate is a flexible substrate, the material of which is a polymer thin film or plastic, and the substrate has a glass transition temperature Tg of greater than or equal to 150° C. Preferably, the substrate has a glass transition temperature Tg of greater than or equal to 200° C. More preferably, the substrate has a glass transition temperature Tg of greater than or equal to 250° C. Most preferably, the substrate has a glass transition temperature Tg of greater than or equal to 300° C. The flexible substrate may be polyethylene terephthalate (PET) or polyethylene glycol (2,6-naphthalene) (PEN).

Materials of the anode are known in the art for anodes, such as conductive metals, conductive metal oxides or conductive polymers. The anodes can inject holes easily into the hole injecting layer, hole transporting layer or light-emitting layer. In at least one embodiment, the absolute value of the difference between the work function of the anode and the HOMO energy level or the valence band energy level of the emitter in the light-emitting layer or of the p-type semiconductor material in the the hole injecting layer, the hole transporting layer, and the electron blocking layer is less than 0.5 eV, preferably less than 0.3 eV, most preferably less than 0.2 eV. Examples of the anode material include, but are not limited to, at least one of Al, Cu, Au, Ag, Mg, Fe, Co, Ni, Mn, Pd, Pt, ITO, aluminum-doped zinc oxide (AZO), and the like. The anode material may be formed using an anode forming method known in the art, such as physical vapor deposition method which includes, for example, radio frequency magnetron sputtering, vacuum thermal evaporation, and e-beam

Materials of the cathode are known in the art for cathodes, for example, conductive metals or metal oxides. The cathode can inject electrons easily into the electron injectinglayer, the electron transporting layer, or the light-emitting layer. In at least one embodiment, the absolute value of the difference between the work function of the cathode and the LUMO energy level or the valence band energy level of the emitter in the light-emitting layer or of the n type semiconductor material as the electron injectinglayer or the electron transporting layer or the hole blocking layer is less than 0.5 eV, preferably less than 0.3 eV, and most preferably less than 0.2 eV. In principle, all materials that can be used as cathodes for OLED may be used as the cathode materials for the devices of the present disclosure. Examples of the cathode material include, but are not limited to, at least one of Al, Au, Ag, Ca, Ba, Mg, LiF/Al, MgAg alloy, BaF₂/Al, Cu, Fe, Co, Ni, Mn, Pd, Pt, ITO, etc. The cathode material may be formed using a cathode forming method known in the ar, such as physical vapor deposition method which includes radio frequency magnetron sputtering, vacuum thermal evaporation, and e-beam.

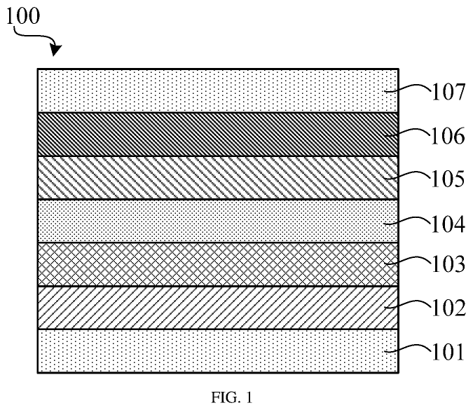

With reference to FIG. 1, in at least one embodiment, an organic electronic device 100 is provided, which may be an OLED, a OLEEC, or an organic light-emitting field effect transistor. Taking the organic electronic device 100 as an OLED device as an example, the OLED device 100 includes a substrate 101, an anode 102, a hole injecting layer 103, a hole transporting layer 104, a light-emitting layer 105, an electron transporting layer 106, and a cathode 107. The material of the light-emitting layer 105 comprises the tetrahydronaphthalene-based organic compound.

The OLED device 100 further includes other functional layers, such as a hole injecting layer, a hole transporting layer, an electron blocking layer, an electron injectinglayer, an electron transporting layer, or a hole blocking layer. The materials of the hole injecting layer, the hole transporting layer, the electron blocking layer, the electron injectinglayer, the electron transporting layer, and the hole blocking layer are materials known in the art to be applied to the hole injecting layer, the hole transporting layer, the electron blocking layer, the electron injectinglayer, the electron transporting layer, and the hole blocking layer, and details are not described herein.

The light-emitting wavelength range of the OLED device 100 is from 300 nm to 1000 nm, preferably from 350 nm to 900 nm, and more preferably from 400 nm to 800 nm.

The present disclosure also relates to an use of the organic electronic device in an electronic device. The electronic device includes, but is not limited to, a display equipment, a lighting equipment, a light source, and a sensor.

The present disclosure also relates to an electronic device including the organic electronic device, which may be, but is not limited to, a display equipment, a lighting equipment, a light source, and a sensor.

### EXAMPLES

The synthetic route and synthetic method of the tetrahydronaphthalene organic compound (hereinafter referred to as a compound) of the present disclosure will be described in detail by means of specific examples, which are only preferred examples of the present disclosure and are not limited to the present disclosure.

### Example 1

Synthesis route of compound 1 is as follows:

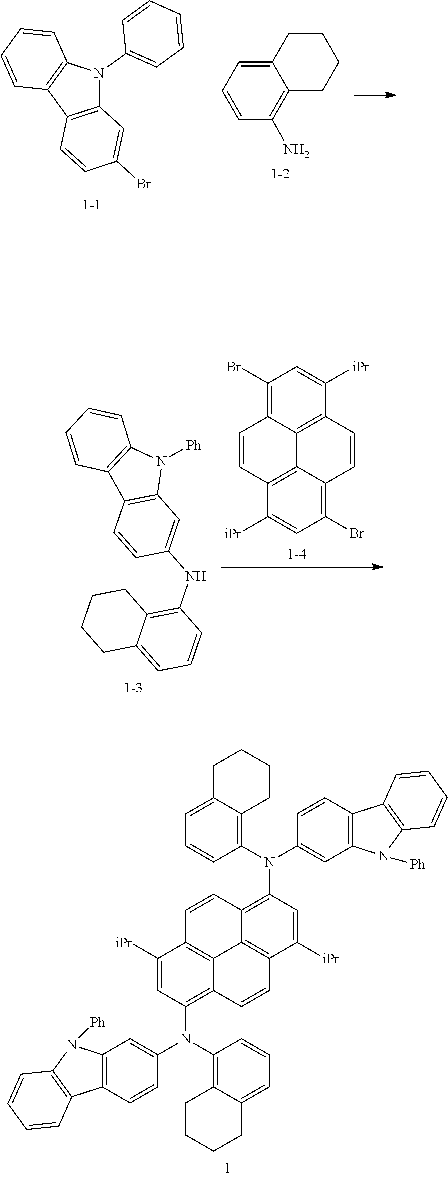 [SMILES: Brc1ccc2c3ccccc3n(-c3ccccc3)c2c1 ; Nc1cccc2c1CCCC2 ; c1ccc(-n2c3ccccc3c3ccc(Nc4cccc5c4CCCC5)cc32)cc1 ; CC(C)c1cc(Br)c2ccc3c(C(C)C)cc(Br)c4ccc1c2c43 ; CC(C)c1cc(N(c2ccc3c4ccccc4n(-c4ccccc4)c3c2)c2cccc3c2CCCC3)c2ccc3c(C(C)C)cc(N(c4ccc5c6ccccc6n(-c6ccccc6)c5c4)c4cccc5c4CCCC5)c4ccc1c2c34]

### 1) Synthesis of Intermediate Compound 1-3

Compound 1-1 (10 mmol), compound 1-2 (10 mmol), Pd(dba)₂ (palladium catalyst) (0.1 mmol), TTBP (tri-tert-butylphosphine) (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 9.13 mmol of intermediate compound 1-3, with a yield of 91.3%, and MS of 388.2.

### 2) Synthesis of Compound 1

Intermediate compound 1-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 1, with a yield of 62.2%, and MS of 1058.5.

### Example 2

Synthesis route of compound 2 is as follows:

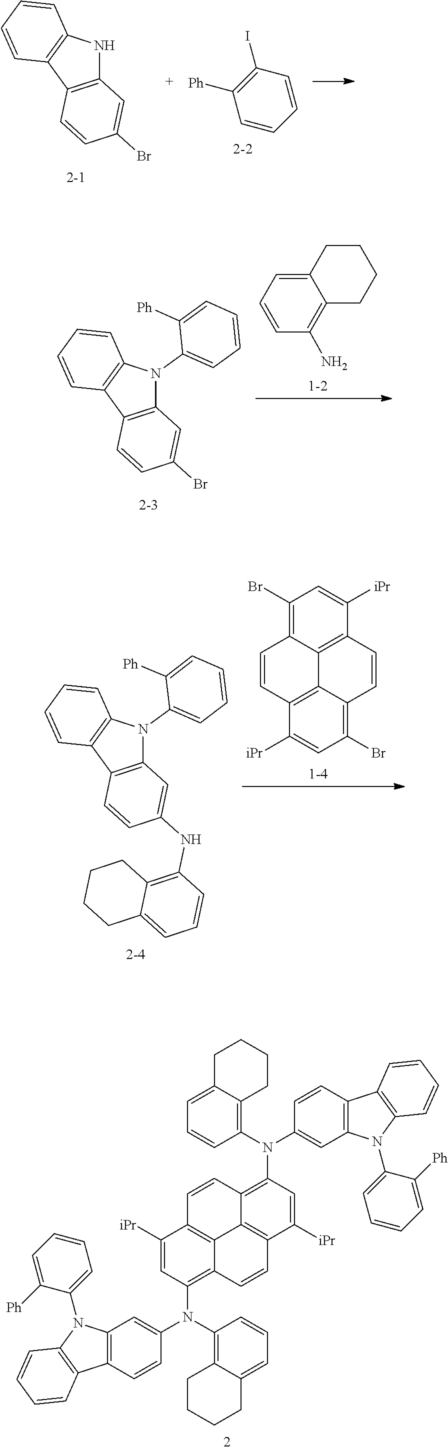

### 1) Synthesis of Intermediate Compound 2-3

Compound 2-1 (10 mmol), compound 2-2 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.17 mmol of intermediate compound 2-3, with a yield of 71.7%, and MS of 397.0.

### 2) Synthesis of Intermediate Compound 2-4

Compound 2-3 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.88 mmol of intermediate compound 2-4, with a yield of 68.8%, and MS of 464.2.

### 3) Synthesis of Compound 2

Compound 2-4 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 2, with a yield of 81.5%, and MS of 1210.6.

### Example 3

Synthesis route of compound 3 is as follows:

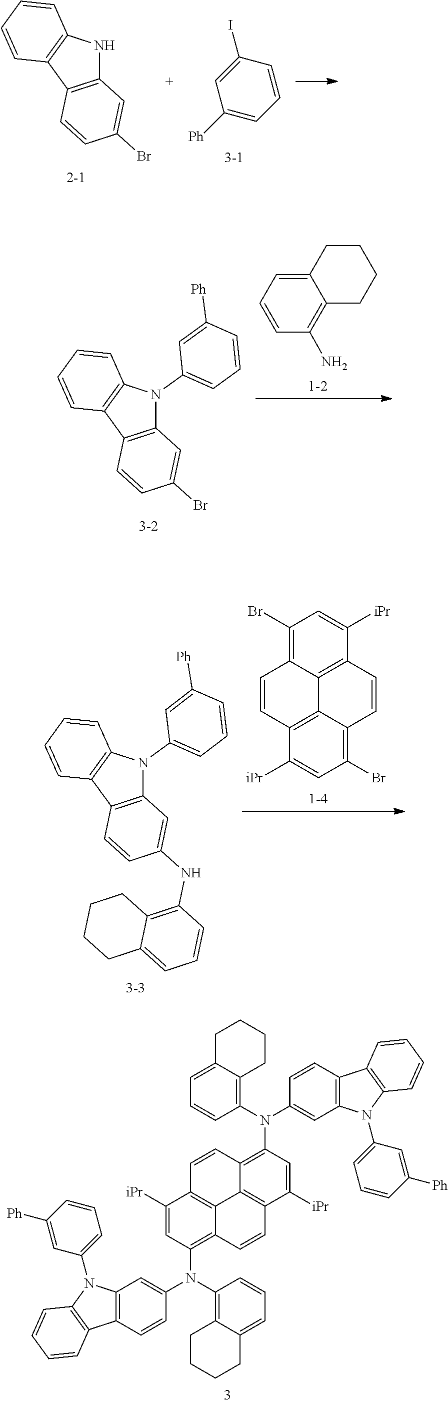

### 1) Synthesis of Intermediate Compound 3-2

Compound 2-1 (10 mmol), compound 3-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.05 mmol of intermediate compound 3-2, with a yield of 80.5%, and MS of 397.0.

### 2) Synthesis of Intermediate Compound 3-3

Compound 3-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 5.94 mmol of intermediate compound 3-3, with a yield of 59.4%, and MS of 464.2.

### 3) Synthesis of Compound 3

Compound 3-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 3, with a yield of 73.6%, and MS of 1210.6.

### Example 4

Synthesis route of compound 4 is as follows:

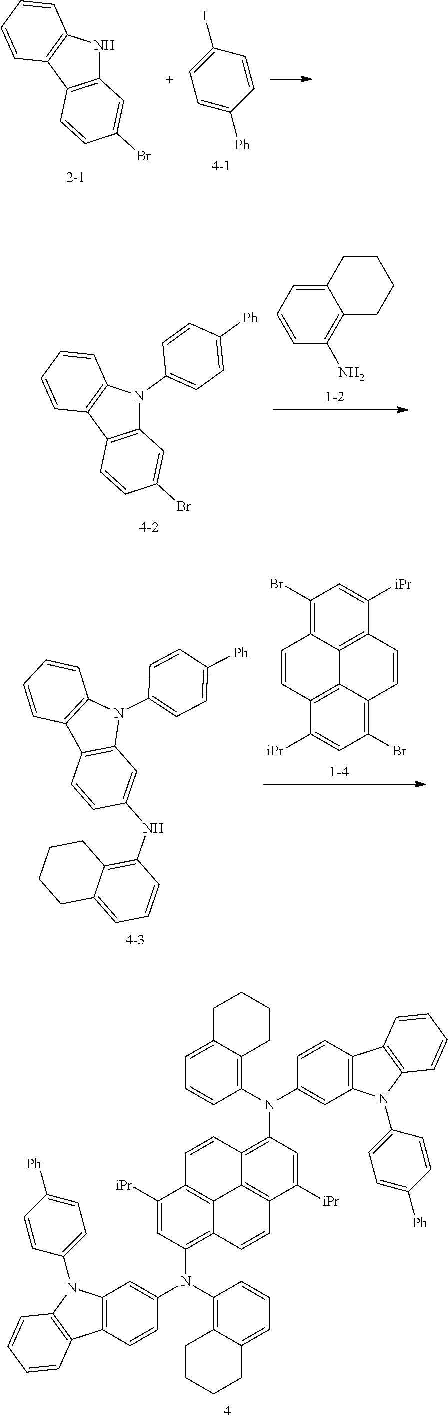

### 1) Synthesis of Intermediate Compound 4-2

Compound 2-1 (10 mmol), compound 4-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.67 mmol of intermediate compound 4-2, with a yield of 86.7%, and MS of 397.0.

### 2) Synthesis of Intermediate Compound 4-3

Compound 4-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.48 mmol of intermediate compound 4-3, with a yield of 64.8%, and MS of 464.2.

### 3) Synthesis of Compound 4

Compound 4-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 4, with a yield of 70.1%, and MS of 1210.6.

### Example 5

Synthesis route of compound 5 is as follows:

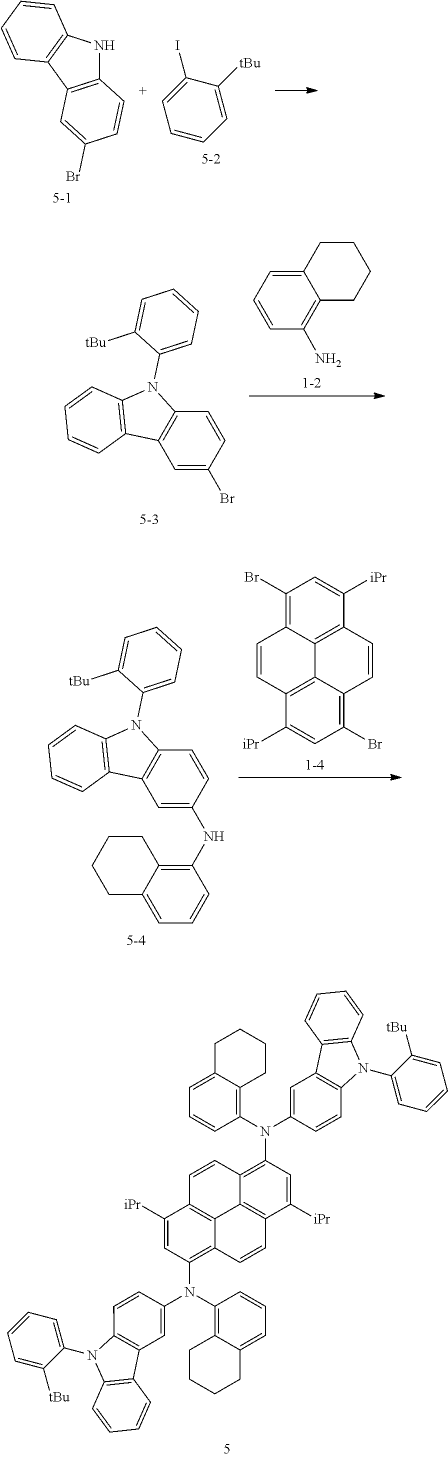

### 1) Synthesis of Intermediate Compound 5-3

Compound 5-1 (10 mmol), compound 5-2 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.31 mmol of intermediate compound 5-3, with a yield of 83.1%, and MS of 377.0.

### 2) Synthesis of Intermediate Compound 5-4

Compound 5-3 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.49 mmol of intermediate compound 5-4, with a yield of 74.9%, and MS of 444.2.

### 3) Synthesis of Compound 5

Compound 5-4 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 5, with a yield of 68.4%, and MS of 1170.6.

### Example 6

Synthesis route of compound 6 is as follows:

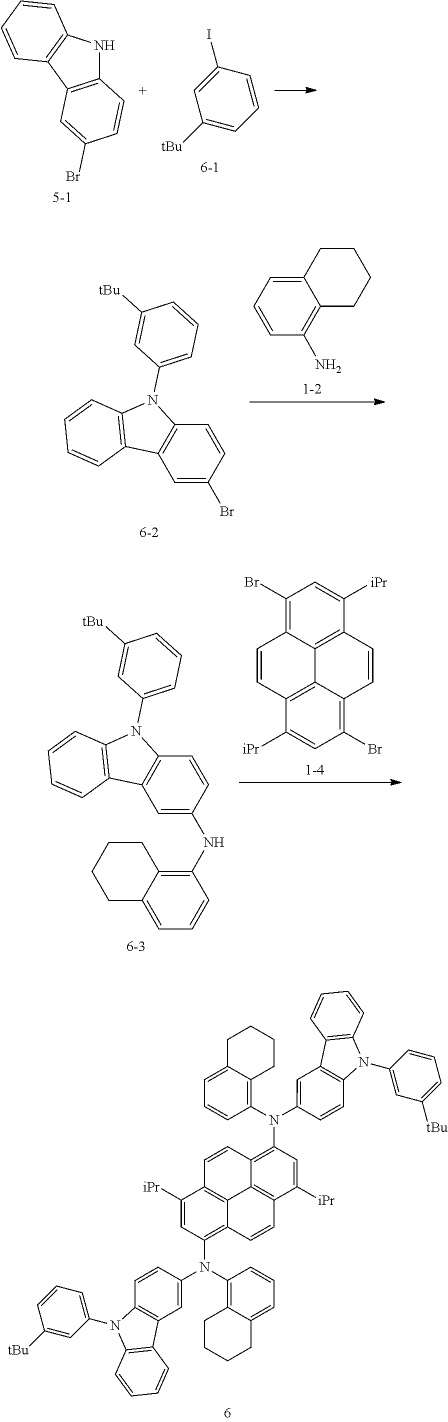

### 1) Synthesis of Intermediate Compound 6-2

Compound 5-1 (10 mmol), compound 6-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.84 mmol of intermediate compound 6-2, with a yield of 78.4%, and MS of 377.0.

### 2) Synthesis of Intermediate Compound 6-3

Compound 6-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.81 mmol of intermediate compound 6-3, with a yield of 68.1%, and MS of 444.2.

### 3) Synthesis of Compound 6

Compound 6-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6h in a nitrogen atmosphere. After completion of the reaction, it was cooled, and the solvent was removed by rotary evaporation, followed by extraction and washing with water for separation to obtain an organic phase, which was chromatographed to obtain compound 6, with a yield of 75.8%, and MS of 1170.6.

Example 7

Synthesis route of compound 7 is as follows:

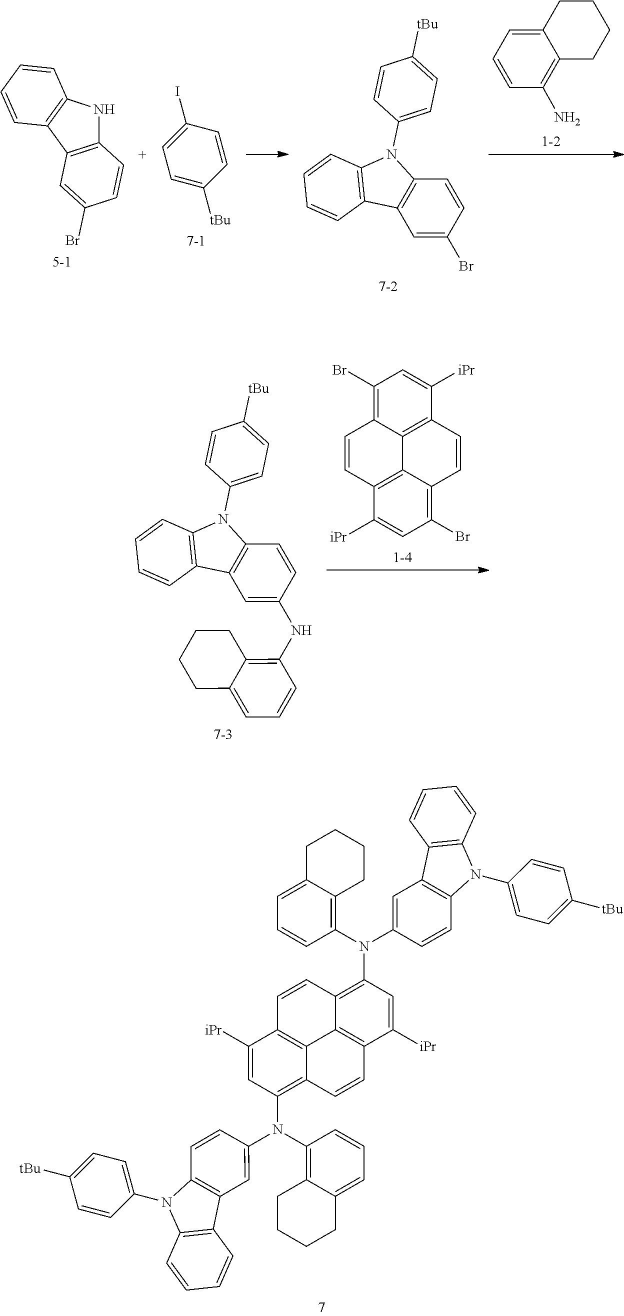

1) Synthesis of intermediate compound 7-2

Compound 5-1 (10 mmol), compound 7-1 (10 mmol), Pd(dba)2 (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.36 mmol of intermediate compound 7-2, with a yield of 73.6%, and MS of 377.0.

### 2) Synthesis of Intermediate Compound 7-3

Compound 7-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.17 mmol of intermediate compound 7-3, with a yield of 61.7%, and MS of 444.2.

### 3) Synthesis of Compound 7

Compound 7-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 7, with a yield of 55.3%, and MS of 1170.6.

### Example 8

Synthesis route of compound 8 is as follows:

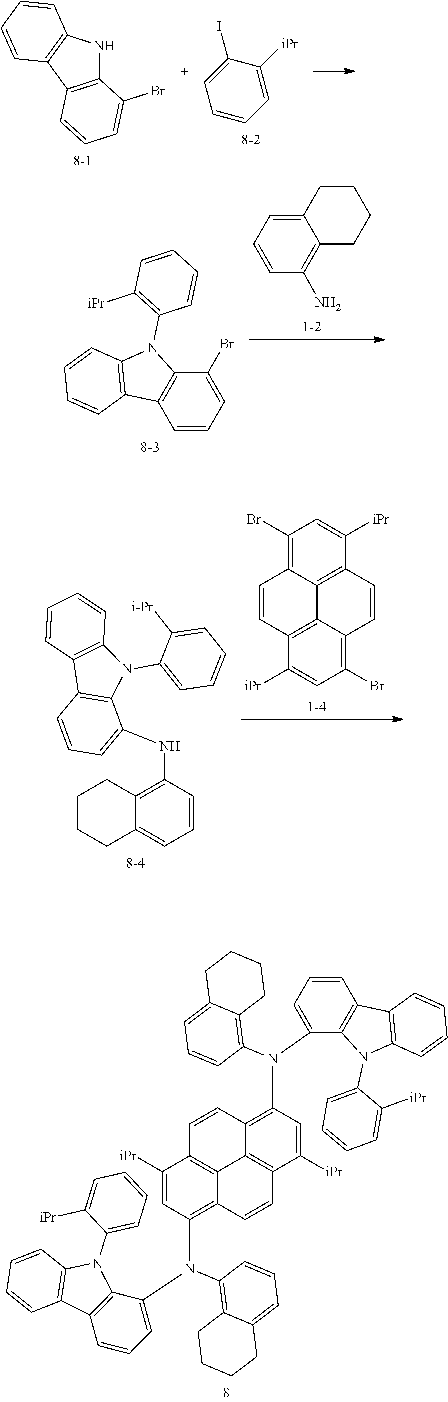

### 1) Synthesis of Intermediate Compound 8-3

Compound 8-1 (10 mmol), compound 8-2 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.59 mmol of intermediate compound 8-3, with a yield of 85.9%, and MS of 363.0.

### 2) Synthesis of Intermediate Compound 8-4

Compound 8-3 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 5.97 mmol of intermediate compound 8-4, with a yield of 59.7%, and MS of 430.2.

### 3) Synthesis of Compound 8

Compound 8-4 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 8, with a yield of 69.7%, and MS of 1142.6.

### Example 9

Synthesis route of compound 9 is as follows:

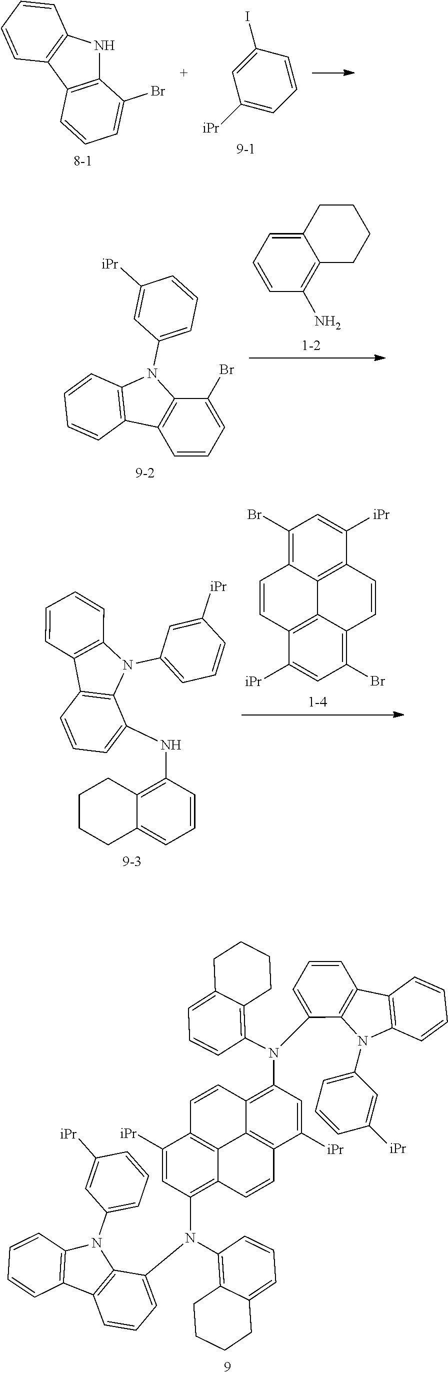

### 1) Synthesis of Intermediate Compound 9-2

Compound 8-1 (10 mmol), compound 9-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.88 mmol of intermediate compound 9-2, with a yield of 78.8%, and MS of 363.0.

### 2) Synthesis of Intermediate Compound 9-3

Compound 9-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.76 mmol of intermediate compound 9-3, with a yield of 67.6%, and MS of 430.2.

### 3) Synthesis of Compound 9

Compound 9-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 9, with a yield of 76.8%, and MS of 1142.6.

### Example 10

Synthesis route of compound 10 is as follows:

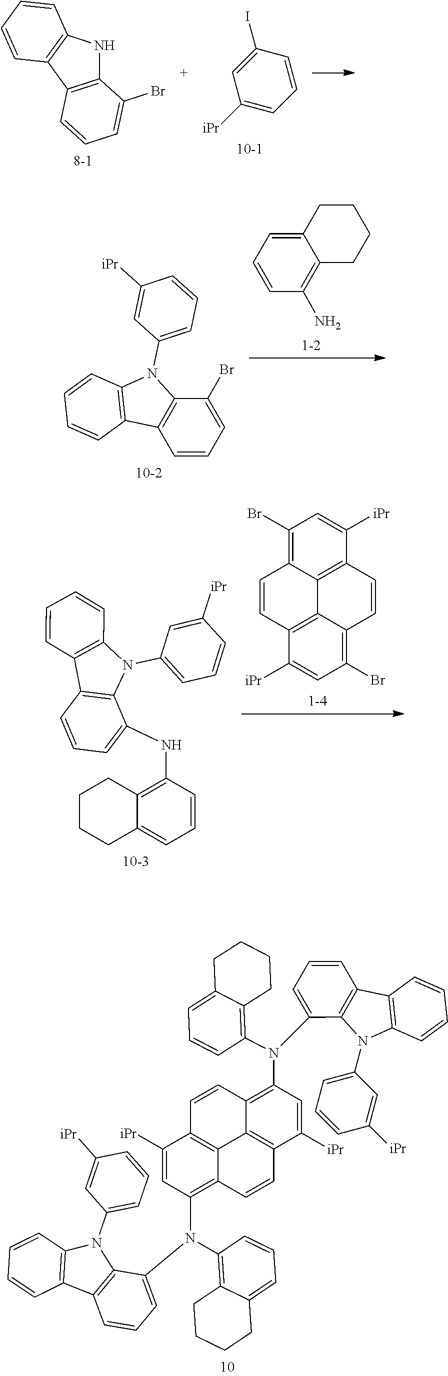

### 1) Synthesis of Intermediate Compound 10-2

Compound 8-1 (10 mmol), compound 10-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.35 mmol of intermediate compound 10-2, with a yield of 73.5%, and MS of 363.0.

### 2) Synthesis of Intermediate Compound 10-3

Compound 10-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.19 mmol of intermediate compound 10-3, with a yield of 61.9%, and MS of 430.2.

### 3) Synthesis of Compound 10

Compound 10-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 10, with a yield of 73.8%, and MS of 1142.6.

### Example 11

Synthesis route of compound 11 is as follows:

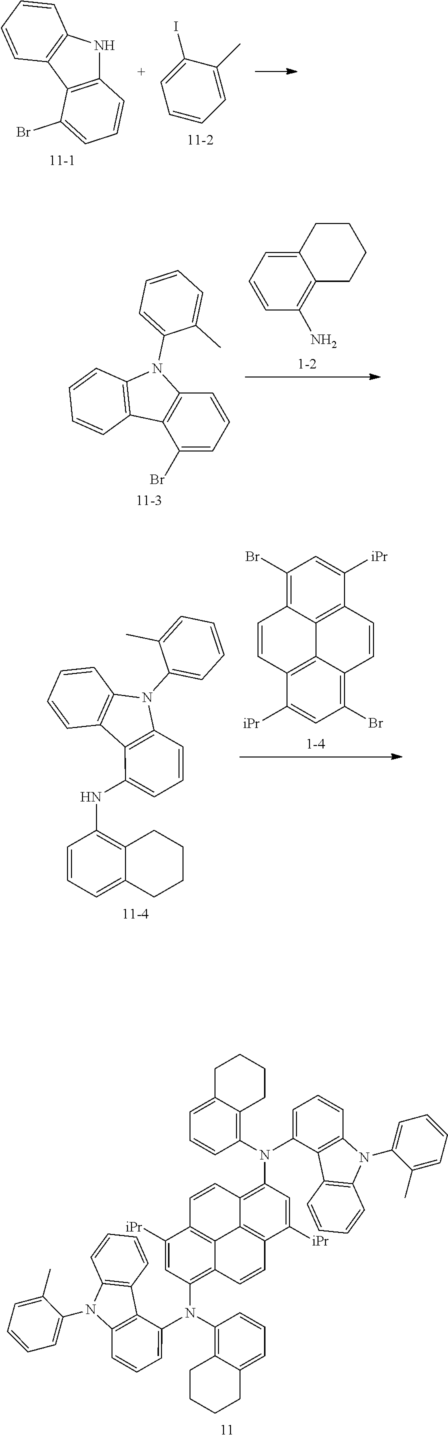

### 1) Synthesis of Intermediate Compound 11-3

Compound 11-1 (10 mmol), compound 11-2 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.36 mmol of intermediate compound 11-3, with a yield of 83.6%, and MS of 335.0.

### 2) Synthesis of Intermediate Compound 11-4

Compound 11-3 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 5.32 mmol of intermediate compound 11-4, with a yield of 53.2%, and MS of 402.2.

### 3) Synthesis of Compound 11

Compound 11-4 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 11, with a yield of 53.6%, and MS of 1086.6.

### Example 12

Synthesis route of compound 12 is as follows:

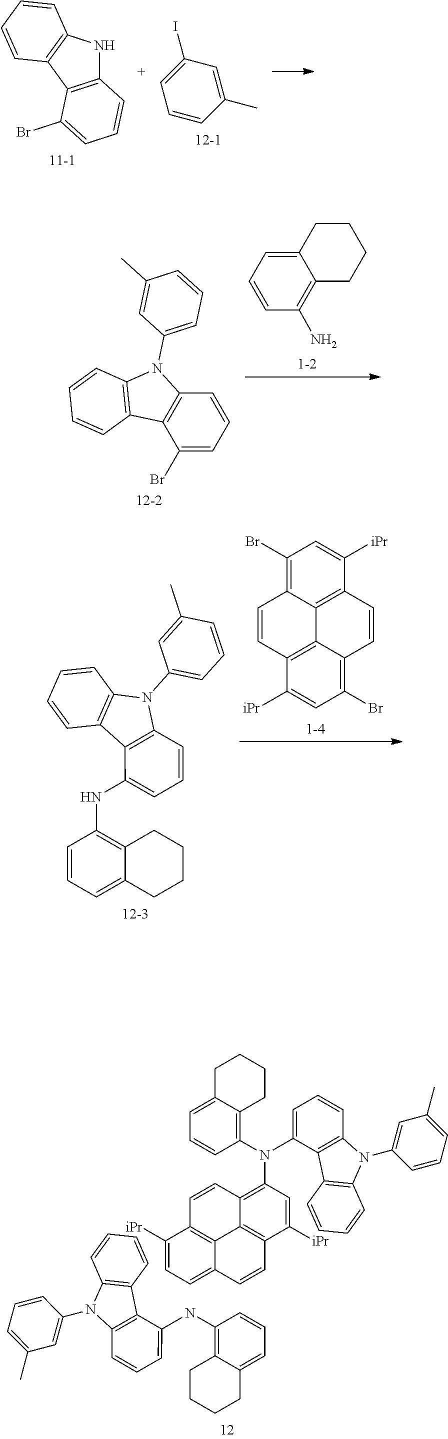

### 1) Synthesis of Intermediate Compound 12-2

Compound 11-1 (10 mmol), compound 12-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.49 mmol of intermediate compound 12-2, with a yield of 74.9%, and MS of 335.0.

### 2) Synthesis of Intermediate Compound 12-3

Compound 12-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.63 mmol of intermediate compound 12-3, with a yield of 76.3%, and MS of 402.2.

### 3) Synthesis of Compound 12

Compound 12-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 12, with a yield of 68.8%, and MS of 1086.6.

### Example 13

Synthesis route of compound 13 is as follows:

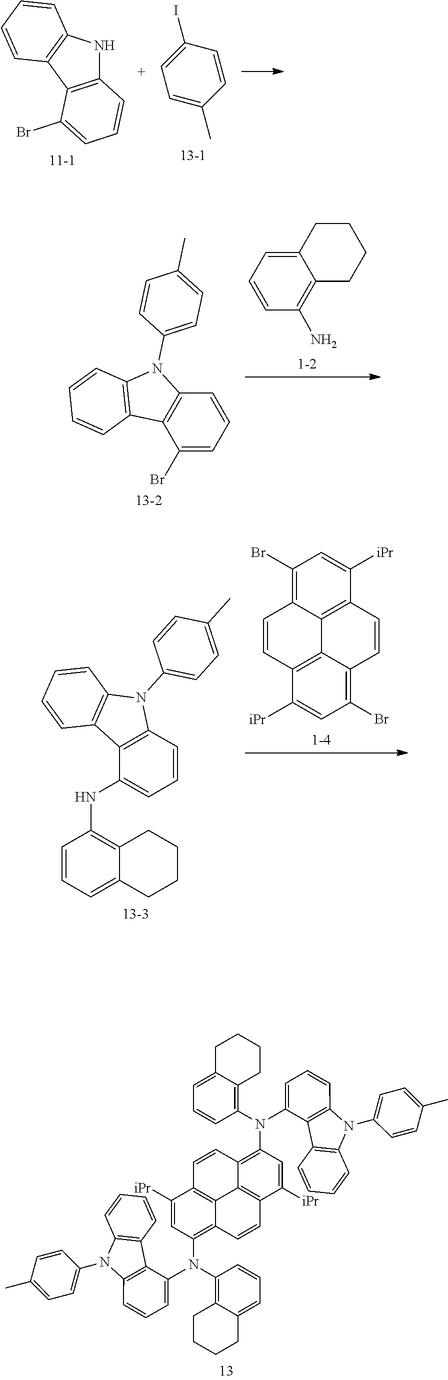

### 1) Synthesis of Intermediate Compound 13-2

Compound 11-1 (10 mmol), compound 13-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.96 mmol of intermediate compound 13-2, with a yield of 89.6%, and MS of 335.0.

### 2) Synthesis of Intermediate Compound 13-3

Compound 13-2 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.58 mmol of intermediate compound 12-3, with a yield of 65.8%, and MS of 402.2.

### 3) Synthesis of Compound 13

Compound 13-3 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 13, with a yield of 60.4%, and MS of 1086.6.

### Example 14

Synthesis route of compound 14 is as follows:

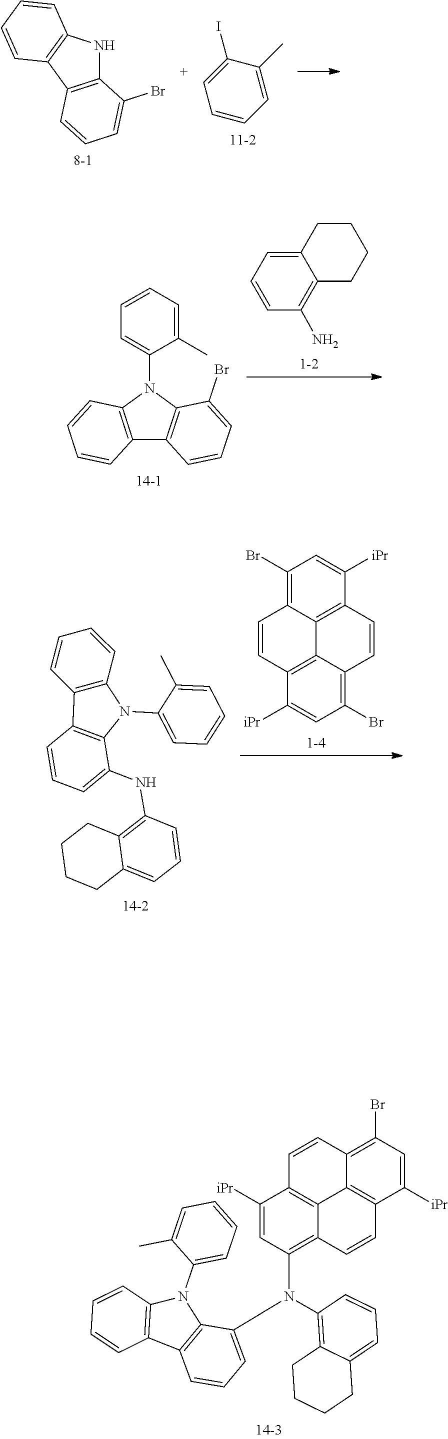

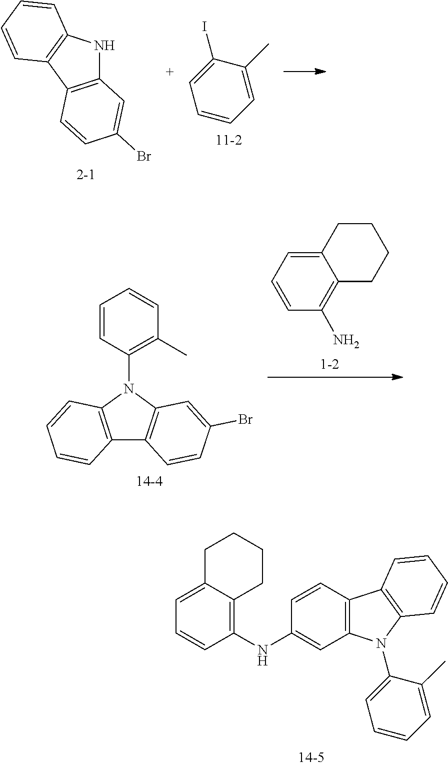

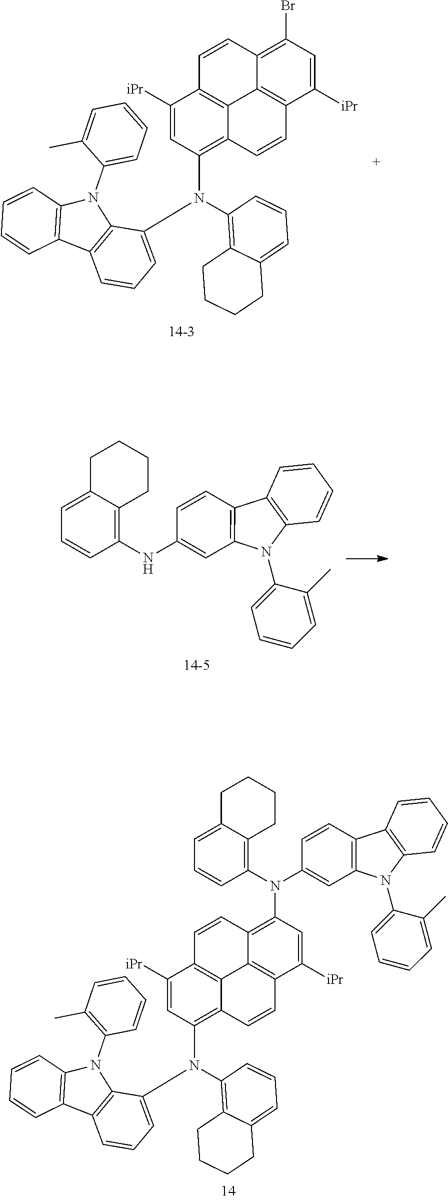 [SMILES: Cc1ccccc1-n1c2ccccc2c2cccc(N(c3cccc4c3CCCC4)c3cc(C(C)C)c4ccc5c(Br)cc(C(C)C)c6ccc3c4c56)c21 ; Cc1ccccc1-n1c2ccccc2c2ccc(Nc3cccc4c3CCCC4)cc21 ; Cc1ccccc1-n1c2ccccc2c2ccc(N(c3cccc4c3CCCC4)c3cc(C(C)C)c4ccc5c(N(c6cccc7c6CCCC7)c6cccc7c8ccccc8n(-c8ccccc8C)c67)cc(C(C)C)c6ccc3c4c65)cc21]

### 1) Synthesis of Intermediate Compound 14-1

Compound 8-1 (10 mmol), compound 11-2 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.33 mmol of intermediate compound 14-1, with a yield of 83.3%, and MS of 335.0.

### 2) Synthesis of Intermediate Compound 14-2

Compound 14-1 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.86 mmol of intermediate compound 14-2, with a yield of 78.6%, and MS of 402.2.

### 3) Synthesis of Intermediate Compound 14-3

Compound 14-2 (10 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.96 mmol of intermediate compound 14-3, with a yield of 79.6%, and MS of 764.2.

### 4) Synthesis of Intermediate Compound 14-4

Compound 2-1 (10 mmol), compound 11-2 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 7.48 mmol of intermediate compound 14-4, with a yield of 74.8%, and MS of 335.0.

### 5) Synthesis of Intermediate Compound 14-5

Compound 14-4 (10 mmol), compound 1-2 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.27 mmol of intermediate compound 14-5, with a yield of 62.7%, and MS of 402.2.

### Synthesis of Compound 14

Compound 14-3 (10 mmol), compound 14-5 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 14, with a yield of 38.2%, and MS of 1086.6.

### Example 15

Synthesis route of compound 15 is as follows:

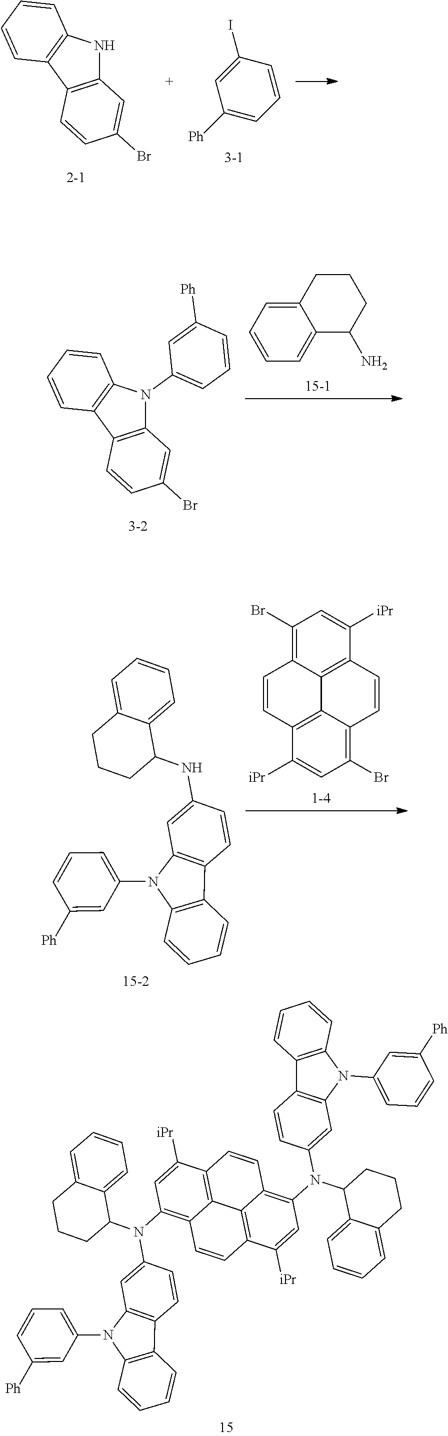

### 1) Synthesis of Intermediate Compound 3-2

Compound 2-1 (10 mmol), compound 3-1 (10 mmol), Pd(dba)₂ (0.1 mmol), TTBP (0.2 mmol) and sodium tert-butoxide (30 mmol) were dissolved in a toluene solvent and stirred at 60° C. for 6 h in a nitrogen atmosphere. After completion of the reaction, it was cooled, and the solvent was removed by rotary evaporation, followed by extraction and washing with water for separation to obtain an organic phase, which was chromatographed to obtain 8.05 mmol of intermediate compound 3-2, with a yield of 80.5%, and MS of 397.0.

### 2) Synthesis of Intermediate Compound 15-2

Compound 13-2 (10 mmol), compound 15-1 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain 6.84 mmol of intermediate compound 15-2, with a yield of 68.4%, and MS of 464.2.

### 3) Synthesis of Compound 15

Compound 15-2 (20 mmol), compound 1-4 (10 mmol), Pd(dba)₂ (0.1 mmol), TTBP (0.2 mmol) and sodium tert-butoxide (30 mmol) were dissolved in a toluene solvent and stirred at 100° C. for 6 h in a nitrogen atmosphere. After completion of the reaction, it was cooled, and the solvent was removed by rotary evaporation, followed by extraction and washing with water for separation to obtain an organic phase, which was chromatographed to obtain compound 15, with a yield of 54.9%, and MS of 1210.

In addition, comparative compound 1 is provided, and the chemical formula of the comparative compound 1 is as follows:

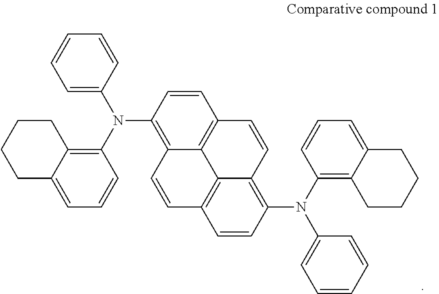 [SMILES: c1ccc(N(c2cccc3c2CCCC3)c2ccc3ccc4c(N(c5ccccc5)c5cccc6c5CCCC6)ccc5ccc2c3c54)cc1]

In the present disclosure, firstly, S1 (singlet energy levels) of the compounds 1 to 15 and the comparative compound 1 prepared in the aforementioned Examples 1 to 15 were calculated, respectively, as shown in table 1 below.

TABLE 1

| Material | S1(eV) |
|---|---|
| Compound 1 | 2.93 |
| Compound 2 | 2.91 |
| Compound 3 | 2.89 |
| Compound 4 | 2.92 |
| Compound 5 | 2.95 |
| Compound 6 | 2.91 |
| Compound 7 | 2.90 |
| Compound 8 | 2.97 |
| Compound 9 | 2.86 |
| Compound 10 | 2.93 |
| Compound 11 | 2.91 |
| Compound 12 | 2.93 |
| Compound 13 | 2.97 |
| Compound 14 | 2.91 |
| Compound 15 | 2.99 |
| Comparative compound 1 | 2.75 |

As can be seen from table 1, S1 of compounds 1 to 15 is significantly increased compared with that of the comparative compound 1, i.e., the light-emitting wavelengths of the compounds 1 to 15 are all blue shifted compared with that of the comparative compound 1. Therefore, under the same experimental conditions, when any one of the compounds 1 to 15 is applied to the OLED device and is used as the guest material in the light-emitting material, the light emitted by the OLED device will be bluer, thus improving the light-emitting efficiency of the OLED device.

Furthermore, the compounds 1 to 15 and the comparative compound 1 were used as light-emitting materials (guest materials) in the organic light-emitting layer of the OLED devices, respectively, and the OLED devices were prepared according to the following preparation method. The preparation method of the OLED device comprises steps as follows:

A. Providing an ITO conductive glass substrate and cleaning the substrate with a cleaning agent, followed by ultraviolet ozone treatment. The cleaning agent may include, but is not limited to, one or more of chloroform, acetone, or isopropanol.

B. Spin-coating PEDOT (polyethylenedioxythiophene) on the ITO conductive glass substrate, and treating on a hot plate at 180° C. for 10 minutes to obtain a hole injecting layer with a thickness of 40 nm.

C. Spin-coating a solution of TFB or PVK having a concentration of 5 mg/ml on the hole injecting layer with toluene as a solvent, and then treating on a hot plate at 180° C. for 60 minutes to obtain a hole transporting layer with a thickness of 20 nm.

D. Spin-coating an organic light-emitting material mixture on the hole transporting layer, followed by treating on a hot plate at 140° C. for 10 minutes to obtain an organic light-emitting layer with a thickness of 40 nm. In the organic light-emitting material mixture, the solvent is methyl benzoate, the host material is BH, the guest materials are the tetrahydronaphthalene-based organic compounds 1 to 15, and the comparative compound 1, respectively, and the weight ratio of the host material to the guest material is 95:5.

The chemical structure of BH in step D is as follows:

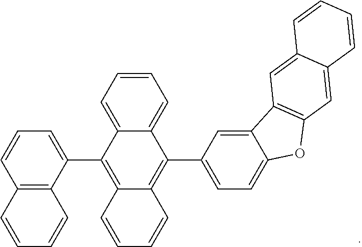 [SMILES: c1ccc2cc3c(cc2c1)oc1ccc(-c2c4ccccc4c(-c4cccc5ccccc45)c4ccccc24)cc13]

E. Transferring the substrate to a vacuum chamber, placing ET and Liq in different

evaporation units, co-depositing them respectively in a high vacuum at a ratio of 50% by weight, forming an electron transporting layer of 20 nm on the organic light-emitting layer, and then depositing a cathode with a thickness of 100 nm using Al as a cathode material, thereby obtaining the OLED device.

The chemical structure of the ET in step E is as follows:

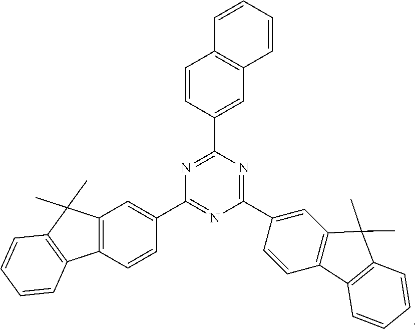 [SMILES: CC1(C)c2ccccc2-c2ccc(-c3nc(-c4ccc5c(c4)C(C)(C)c4ccccc4-5)nc(-c4ccc5ccccc5c4)n3)cc21]

The chemical structure of Liq in step E is as follows:

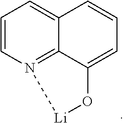

F. Encapsulating the OLED devices with ultraviolet curable resins.

Specifically, in the above preparation method, the compounds 1 to 15 and the comparative compound 1 were used as guest materials in the organic light-emitting layer, respectively, so as to correspondingly prepare devices OLED-1, OLED-2, OLED-3, OLED-4, OLED-5, OLED-6, OLED-7, OLED-8, OLED-9, OLED-10, OLED-11, OLED-12, OLED-13, OLED-14, OLED-15, and OLED-Refl. It can be understood that in the preparation method of the OLED device mentioned above, other experimental conditions are the same except that the guest materials are different.

Furthermore, the current-voltage-luminance (J-V) characteristics of the OLED devices are characterized, and the color coordinates, the light-emitting efficiency (CE@1knits), and the lifetime (LT90@1knits) corresponding to the OLED devices 1 to 15 and OLED-Ref1 are measured, as shown in FIG. 2.

TABLE 2

| | | Color | CE@1 knits | LT90 @ |
|---|---|---|---|---|
| Device | Guest material | coordinates | (cd/A) | 1 knits(h) |
| OLED-1 | Compound 1 | 0.144,0.089 | 9.1 | 486 |
| OLED-2 | Compound 2 | 0.145,0.088 | 8.9 | 483 |
| OLED-3 | Compound 3 | 0.145,0.089 | 8.9 | 489 |
| OLED-4 | Compound 4 | 0.145,0.084 | 8.7 | 490 |
| OLED-5 | Compound 5 | 0.146,0.087 | 8.6 | 476 |
| OLED-6 | Compound 6 | 0.145,0.085 | 8.6 | 481 |
| OLED-7 | Compound 7 | 0.146,0.088 | 8.5 | 470 |
| OLED-8 | Compound 8 | 0.145,0.088 | 8.6 | 469 |
| OLED-9 | Compound 9 | 0.145,0.085 | 8.6 | 475 |
| OLED-10 | Compound 10 | 0.146,0.085 | 8.5 | 478 |
| OLED-11 | Compound 11 | 0.146,0.085 | 8.4 | 465 |
| OLED-12 | Compound 12 | 0.146,0.083 | 8.5 | 471 |
| OLED-13 | Compound 13 | 0.147,0.081 | 8.3 | 467 |
| OLED-14 | Compound 14 | 0.145,0.083 | 8.0 | 450 |
| OLED-15 | Compound 15 | 0.143,0.081 | 9.2 | 503 |
| OLED-Ref1 | Comparative | 0.162,0.141 | 5.3 | 129 |
| | compound 1 | | | |

As can be seen from the table 2, color coordinates of the OLED devices prepared by using the compounds 1 to 15 as light-emitting materials in the organic light-emitting layer are better than those of the OLED devices prepared by using the comparative compound 1 as light-emitting material in the organic light-emitting layer.

In addition, the light-emitting efficiency of OLED devices prepared by using compounds 1 to 15 as light-emitting materials in the organic light-emitting layer is in a range from 8 cd/A to 9 cd/A. That is, they have more excellent light-emitting efficiency. This is because compounds 1 to 15 in the present disclosure are all tetrahydronaphthalene-based organic compounds. Since a phenyl carbazole derivative is introduced into the tetrahydronaphthalene-based organic compound, and the phenyl carbazole derivative makes the overall molecular conjugation of the compound higher compared with the benzene ring in the comparative compound 1. In addition, due to the presence of the phenyl carbazole derivative, stability of the tetrahydronaphthalene-based organic compound after film formation is also better than that of the comparative compound 1 after film formation. Furthermore, compared with an OLED device made of the comparative compound 1 as a light-emitting material, the lifetime of OLED devices in the present disclosure can be significantly improved due to the introduction of the phenyl carbazole derivative.

The tetrahydronaphthalene-based organic compounds, mixtures, compositions, and organic electronic devices provided herein have been described in detail above, and the principles and embodiments of the present disclosure are described by using specific examples herein. Descriptions of the above embodiments are merely intended to help understand the technical solutions and core ideas of the present disclosure. Meanwhile, for a person skilled in the art, there will be changes in both the specific embodiments and application scope in accordance with the teachings of the present disclosure. In view of the foregoing, contents of this specification should not be construed as a limitation on the present disclosure.

What is claimed is:

1. A tetrahydronaphthalene-based organic compound having a structure shown in general formula (1): 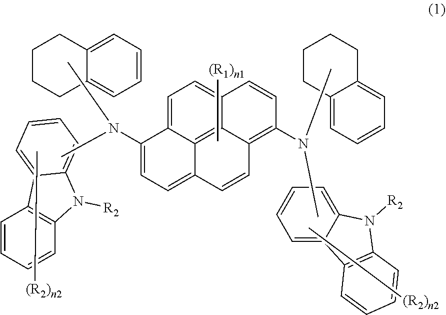
wherein, n1 is an integer selected from 1 to 8; n2 is an integer selected from 1 to 7;
R₁ and R₂ at each occurence is independently selected from the group consisting of -H, deuterium, a linear alkyl group containing 1 to 20 carbon atoms, a linear alkoxy containing 1 to 20 carbon atoms, a linear thioalkoxy group containing 1 to 20 carbon atoms, a branched or cyclic alkyl containing 3 to 20 carbon atoms, a branched or cyclic alkoxy containing 3 to 20 carbon atoms, a branched or cyclic thioalkoxy group containing 3 to 20 carbon atoms, a silyl group, a ketone group containing 1 to 20 carbon atoms, an alkoxycarbonyl group containing 2 to 20 carbon atoms, an aryloxycarbonyl group containing 7 to 20 carbon atoms, a substituted or unsubstituted aromatic or heteroaromatic group containing 5 to 60 ring atoms, an aryloxy or heteroaryloxy group containing 5 to 60 ring atoms, a cyano group, a carbamoyl group, a haloformyl group, a formyl group, an isocyano group, an isocyanate group, a thiocyanate group, an isothiocyanate group, a hydroxyl group, a nitro group, an amino group, —CF₃, —Cl, —Br, —F, —I, and combinations thereof.

2. The tetrahydronaphthalene-based organic compound according to claim 1, wherein the tetrahydronaphthalene-based organic compound has a structure shown in general formula (2): 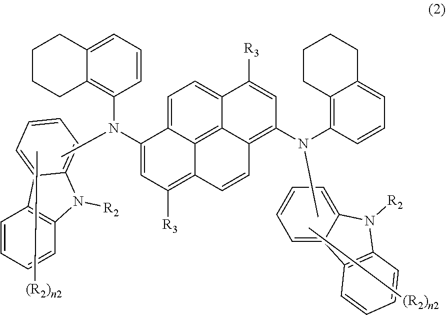
wherein, R₃, at each occurrence, is independently selected from the group consisting of methyl, ethyl, isopropyl and tert-butyl.

3. The tetrahydronaphthalene-based organic compound according to claim 2, wherein the tetrahydronaphthalene-based organic compound has a structure shown in general formulae from (3-1) to (3-4):

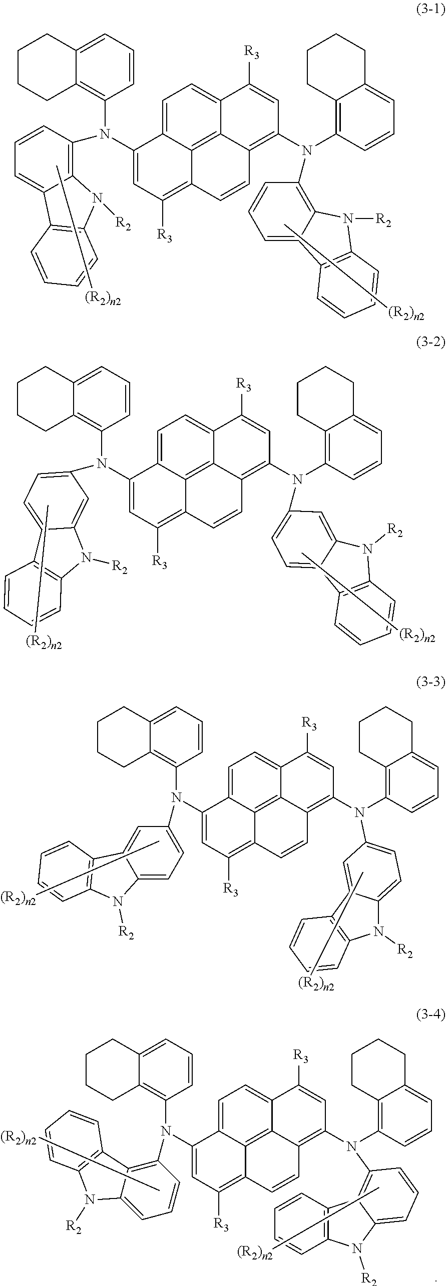

4. The tetrahydronaphthalene-based organic compound according to claim 1, wherein adjacent R₁ form a ring with each other.

5. The tetrahydronaphthalene-based organic compound according to claim 1, wherein R₁ and R₂, at each occurrence, is independently selected from the group consisting of a linear alkyl group containing 1 to 10 carbon atoms, a branched or cyclic alkyl containing 3 to 10 carbon atoms, a substituted or unsubstituted aromatic group containing 5 to 30 ring atoms, or a substituted or unsubstituted heteroaromatic group containing 5 to 30 ring atoms, and combinations thereof.

6. The tetrahydronaphthalene-based organic compound according to claim 1, wherein the tetrahydronaphthalene-based organic compound is selected from any one of the following structures:

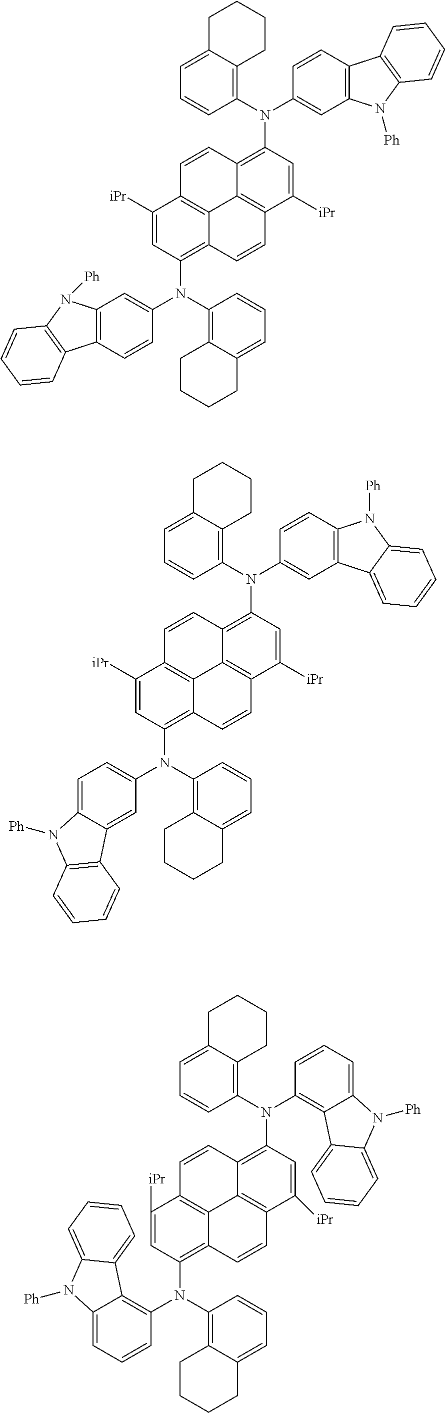 [SMILES: CC(C)c1cc(N(c2ccc3c4ccccc4n(-c4ccccc4)c3c2)c2cccc3c2CCCC3)c2ccc3c(C(C)C)cc(N(c4ccc5c6ccccc6n(-c6ccccc6)c5c4)c4cccc5c4CCCC5)c4ccc1c2c34 ; CC(C)c1cc(N(c2ccc3c(c2)c2ccccc2n3-c2ccccc2)c2cccc3c2CCCC3)c2ccc3c(C(C)C)cc(N(c4ccc5c(c4)c4ccccc4n5-c4ccccc4)c4cccc5c4CCCC5)c4ccc1c2c34 ; CC(C)c1cc(N(c2cccc3c2CCCC3)c2cccc3c2c2ccccc2n3-c2ccccc2)c2ccc3c(C(C)C)cc(N(c4cccc5c4CCCC5)c4cccc5c4c4ccccc4n5-c4ccccc4)c4ccc1c2c34]

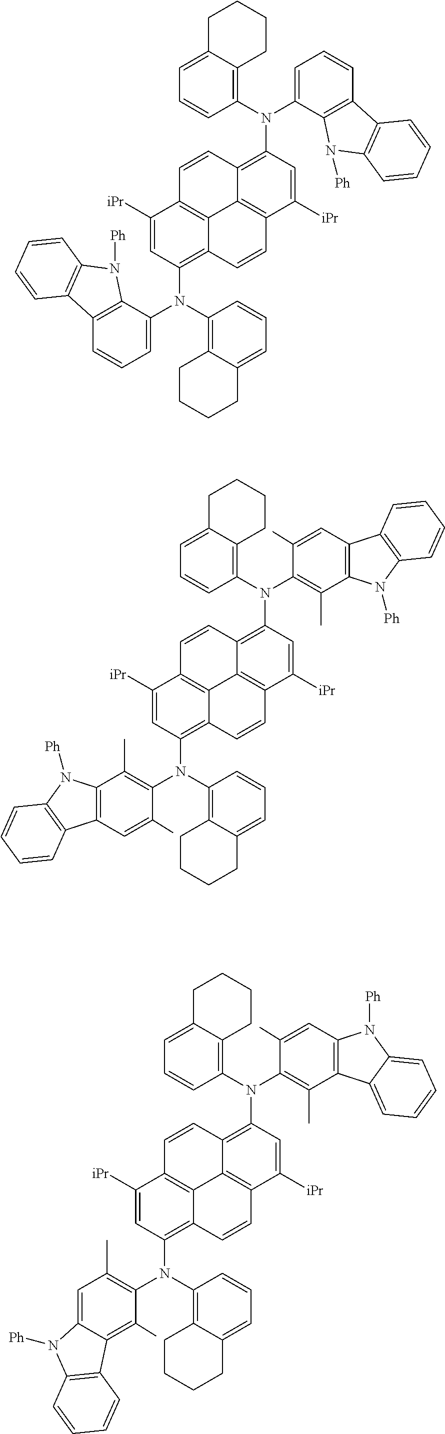 [SMILES: CC(C)c1cc(N(c2cccc3c2CCCC3)c2cccc3c4ccccc4n(-c4ccccc4)c23)c2ccc3c(C(C)C)cc(N(c4cccc5c4CCCC5)c4cccc5c6ccccc6n(-c6ccccc6)c45)c4ccc1c2c34 ; Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)cc5c6ccccc6n(-c6ccccc6)c5c4C)cc(C(C)C)c4ccc1c2c43 ; Cc1cc2c(c(C)c1N(c1cccc3c1CCCC3)c1cc(C(C)C)c3ccc4c(N(c5cccc6c5CCCC6)c5c(C)cc6c(c5C)c5ccccc5n6-c5ccccc5)cc(C(C)C)c5ccc1c3c54)c1ccccc1n2-c1ccccc1]

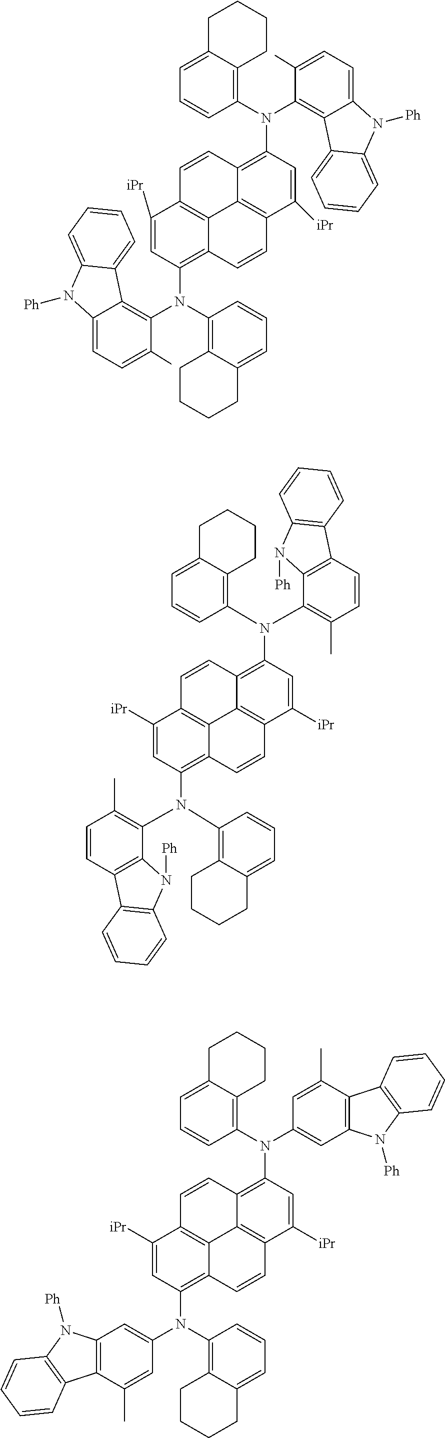 [SMILES: Cc1ccc2c(c1N(c1cccc3c1CCCC3)c1cc(C(C)C)c3ccc4c(N(c5cccc6c5CCCC6)c5c(C)ccc6c5c5ccccc5n6-c5ccccc5)cc(C(C)C)c5ccc1c3c54)c1ccccc1n2-c1ccccc1 ; Cc1ccc2c3ccccc3n(-c3ccccc3)c2c1N(c1cccc2c1CCCC2)c1cc(C(C)C)c2ccc3c(N(c4cccc5c4CCCC5)c4c(C)ccc5c6ccccc6n(-c6ccccc6)c45)cc(C(C)C)c4ccc1c2c43 ; Cc1cc(N(c2cccc3c2CCCC3)c2cc(C(C)C)c3ccc4c(N(c5cc(C)c6c7ccccc7n(-c7ccccc7)c6c5)c5cccc6c5CCCC6)cc(C(C)C)c5ccc2c3c54)cc2c1c1ccccc1n2-c1ccccc1]

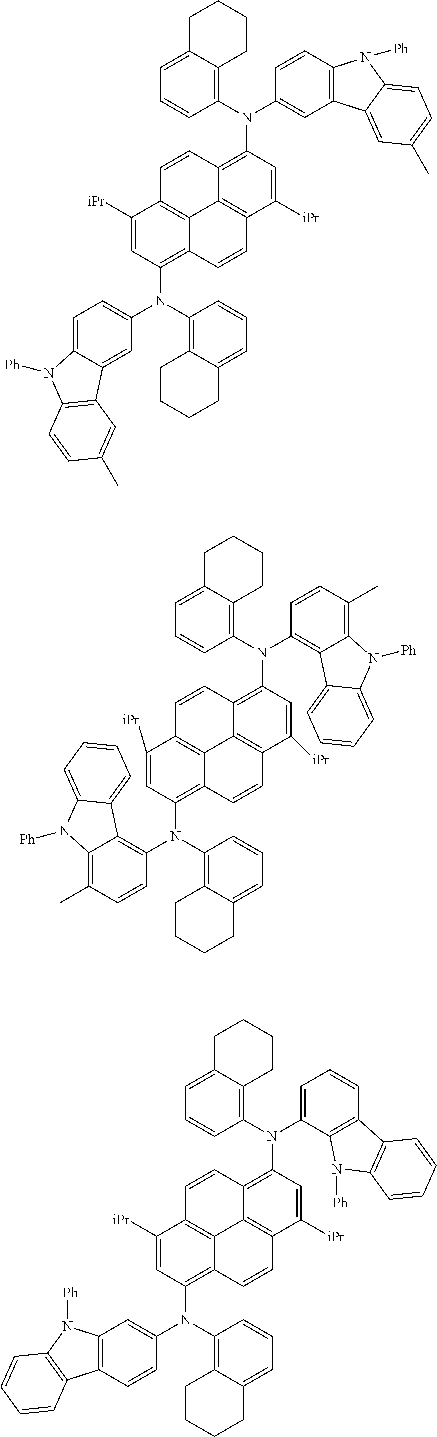 [SMILES: Cc1ccc2c(c1)c1cc(N(c3cccc4c3CCCC4)c3cc(C(C)C)c4ccc5c(N(c6ccc7c(c6)c6cc(C)ccc6n7-c6ccccc6)c6cccc7c6CCCC7)cc(C(C)C)c6ccc3c4c65)ccc1n2-c1ccccc1 ; Cc1ccc(N(c2cccc3c2CCCC3)c2cc(C(C)C)c3ccc4c(N(c5cccc6c5CCCC6)c5ccc(C)c6c5c5ccccc5n6-c5ccccc5)cc(C(C)C)c5ccc2c3c54)c2c3ccccc3n(-c3ccccc3)c12 ; CC(C)c1cc(N(c2ccc3c4ccccc4n(-c4ccccc4)c3c2)c2cccc3c2CCCC3)c2ccc3c(C(C)C)cc(N(c4cccc5c4CCCC5)c4cccc5c6ccccc6n(-c6ccccc6)c45)c4ccc1c2c34]

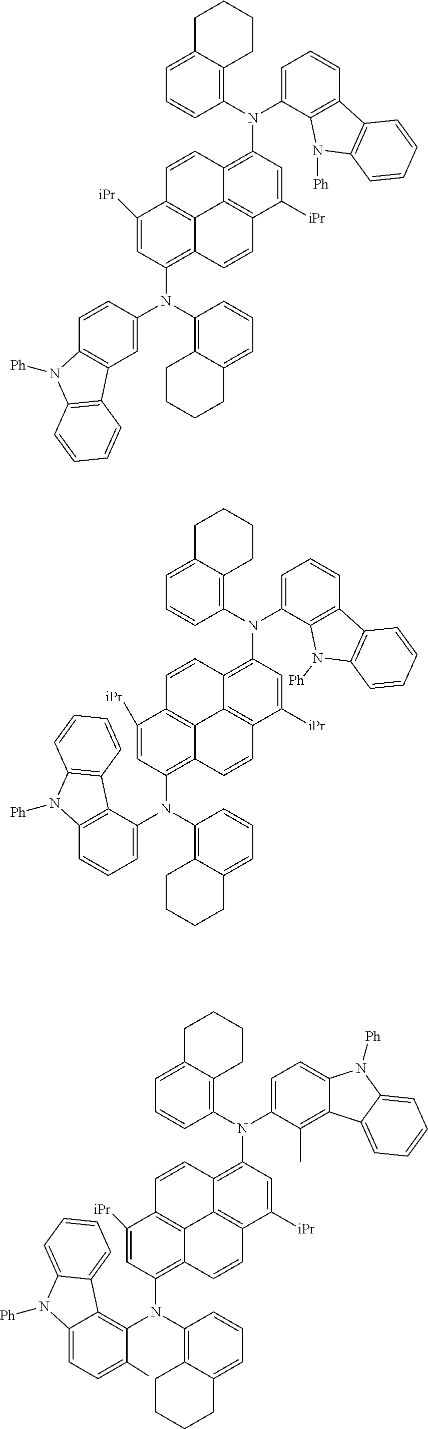 [SMILES: CC(C)c1cc(N(c2ccc3c(c2)c2ccccc2n3-c2ccccc2)c2cccc3c2CCCC3)c2ccc3c(C(C)C)cc(N(c4cccc5c4CCCC5)c4cccc5c6ccccc6n(-c6ccccc6)c45)c4ccc1c2c34 ; CC(C)c1cc(N(c2cccc3c2CCCC3)c2cccc3c2c2ccccc2n3-c2ccccc2)c2ccc3c(C(C)C)cc(N(c4cccc5c4CCCC5)c4cccc5c6ccccc6n(-c6ccccc6)c45)c4ccc1c2c34 ; Cc1ccc2c(c1N(c1cccc3c1CCCC3)c1cc(C(C)C)c3ccc4c(N(c5cccc6c5CCCC6)c5ccc6c(c5C)c5ccccc5n6-c5ccccc5)cc(C(C)C)c5ccc1c3c54)c1ccccc1n2-c1ccccc1]

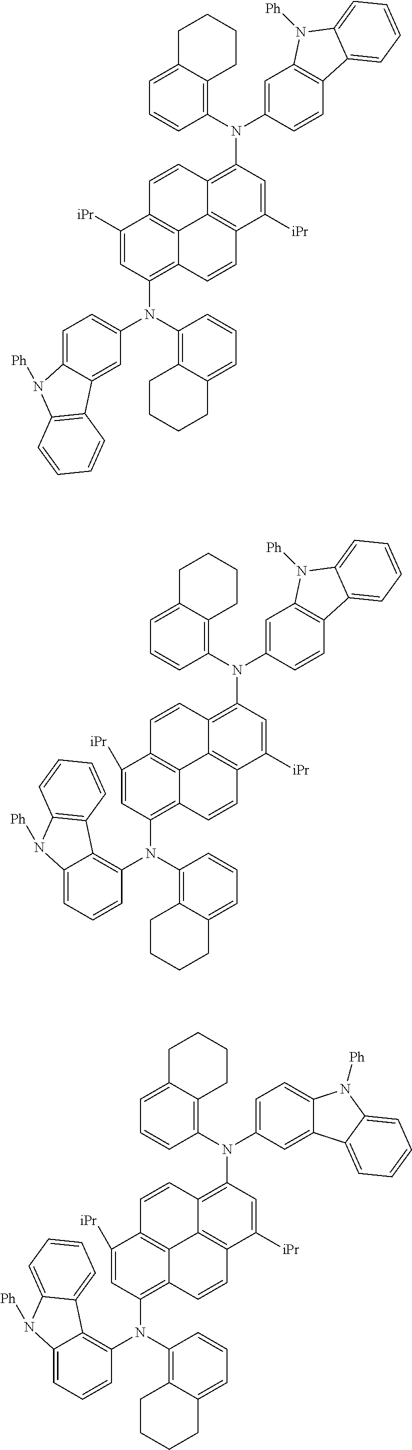 [SMILES: CC(C)c1cc(N(c2ccc3c(c2)c2ccccc2n3-c2ccccc2)c2cccc3c2CCCC3)c2ccc3c(C(C)C)cc(N(c4ccc5c6ccccc6n(-c6ccccc6)c5c4)c4cccc5c4CCCC5)c4ccc1c2c34 ; CC(C)c1cc(N(c2ccc3c4ccccc4n(-c4ccccc4)c3c2)c2cccc3c2CCCC3)c2ccc3c(C(C)C)cc(N(c4cccc5c4CCCC5)c4cccc5c4c4ccccc4n5-c4ccccc4)c4ccc1c2c34 ; CC(C)c1cc(N(c2ccc3c(c2)c2ccccc2n3-c2ccccc2)c2cccc3c2CCCC3)c2ccc3c(C(C)C)cc(N(c4cccc5c4CCCC5)c4cccc5c4c4ccccc4n5-c4ccccc4)c4ccc1c2c34]

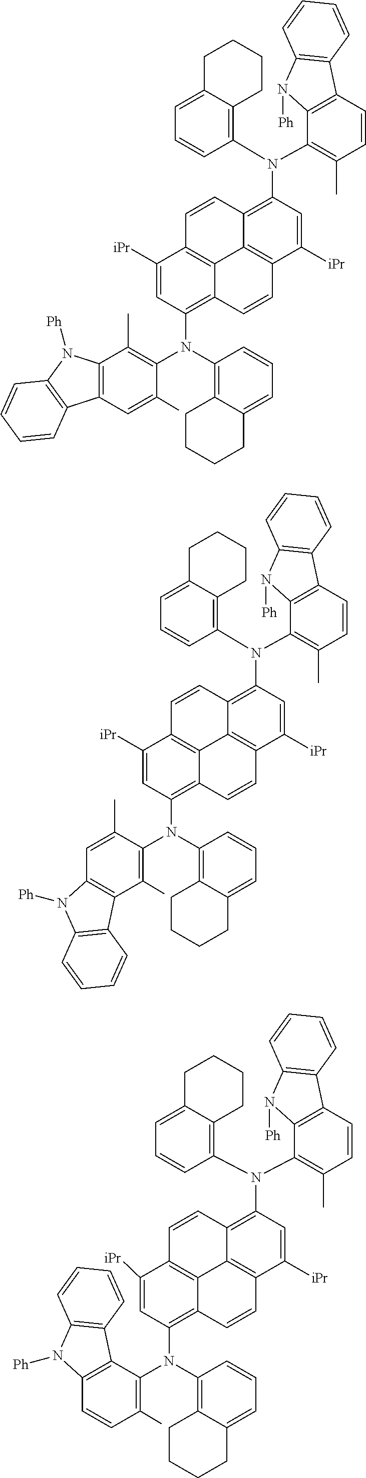 [SMILES: Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)ccc5c6ccccc6n(-c6ccccc6)c45)cc(C(C)C)c4ccc1c2c43 ; Cc1cc2c(c(C)c1N(c1cccc3c1CCCC3)c1cc(C(C)C)c3ccc4c(N(c5cccc6c5CCCC6)c5c(C)ccc6c7ccccc7n(-c7ccccc7)c56)cc(C(C)C)c5ccc1c3c54)c1ccccc1n2-c1ccccc1 ; Cc1ccc2c(c1N(c1cccc3c1CCCC3)c1cc(C(C)C)c3ccc4c(N(c5cccc6c5CCCC6)c5c(C)ccc6c7ccccc7n(-c7ccccc7)c56)cc(C(C)C)c5ccc1c3c54)c1ccccc1n2-c1ccccc1]

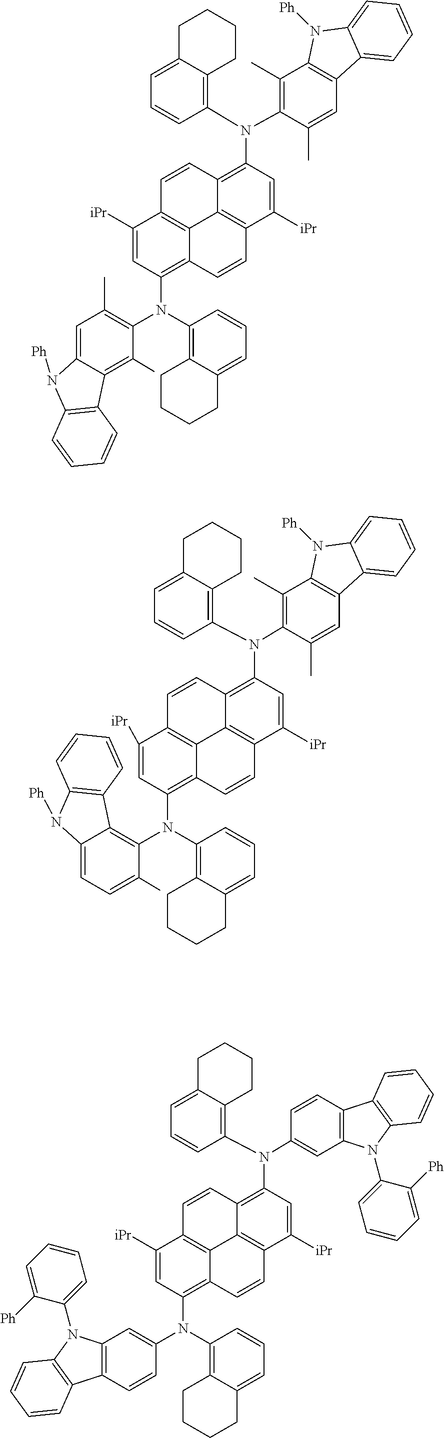 [SMILES: Cc1cc2c(c(C)c1N(c1cccc3c1CCCC3)c1cc(C(C)C)c3ccc4c(N(c5cccc6c5CCCC6)c5c(C)cc6c7ccccc7n(-c7ccccc7)c6c5C)cc(C(C)C)c5ccc1c3c54)c1ccccc1n2-c1ccccc1 ; Cc1cc2c3ccccc3n(-c3ccccc3)c2c(C)c1N(c1cccc2c1CCCC2)c1cc(C(C)C)c2ccc3c(N(c4cccc5c4CCCC5)c4c(C)ccc5c4c4ccccc4n5-c4ccccc4)cc(C(C)C)c4ccc1c2c43 ; CC(C)c1cc(N(c2ccc3c4ccccc4n(-c4ccccc4-c4ccccc4)c3c2)c2cccc3c2CCCC3)c2ccc3c(C(C)C)cc(N(c4ccc5c6ccccc6n(-c6ccccc6-c6ccccc6)c5c4)c4cccc5c4CCCC5)c4ccc1c2c34]

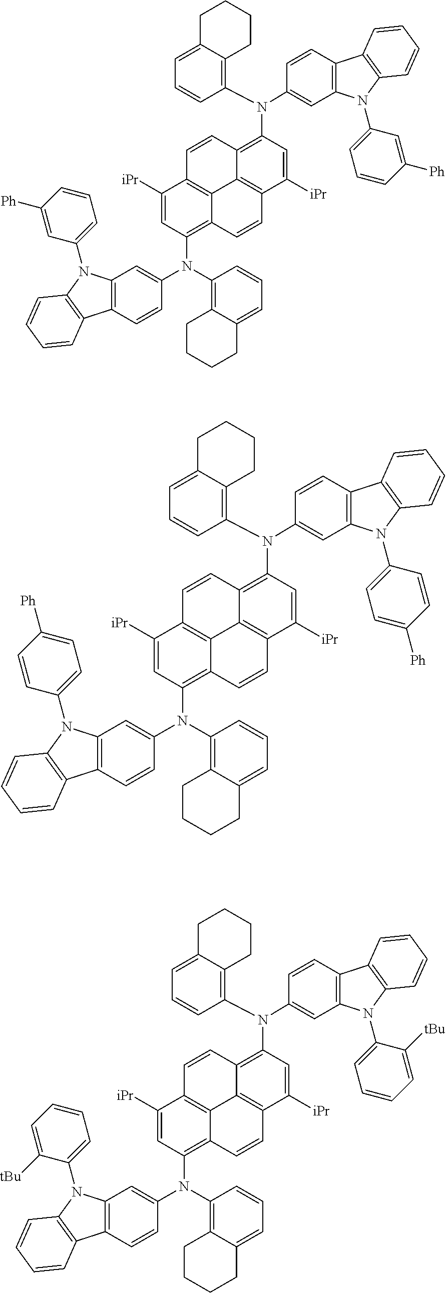 [SMILES: CC(C)c1cc(N(c2ccc3c4ccccc4n(-c4cccc(-c5ccccc5)c4)c3c2)c2cccc3c2CCCC3)c2ccc3c(C(C)C)cc(N(c4ccc5c6ccccc6n(-c6cccc(-c7ccccc7)c6)c5c4)c4cccc5c4CCCC5)c4ccc1c2c34 ; CC(C)c1cc(N(c2ccc3c4ccccc4n(-c4ccc(-c5ccccc5)cc4)c3c2)c2cccc3c2CCCC3)c2ccc3c(C(C)C)cc(N(c4ccc5c6ccccc6n(-c6ccc(-c7ccccc7)cc6)c5c4)c4cccc5c4CCCC5)c4ccc1c2c34 ; CC(C)c1cc(N(c2ccc3c4ccccc4n(-c4ccccc4C(C)(C)C)c3c2)c2cccc3c2CCCC3)c2ccc3c(C(C)C)cc(N(c4ccc5c6ccccc6n(-c6ccccc6C(C)(C)C)c5c4)c4cccc5c4CCCC5)c4ccc1c2c34]

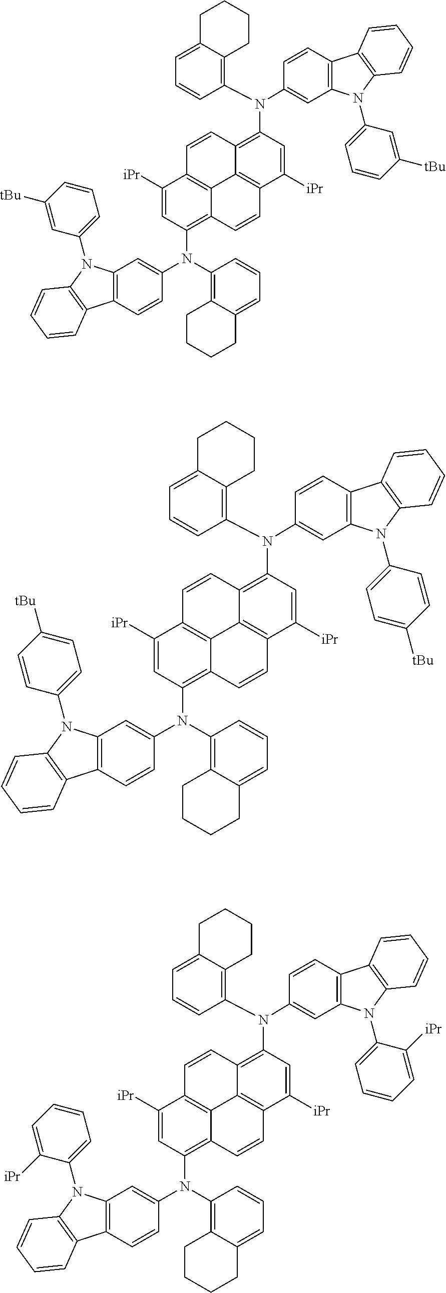 [SMILES: CC(C)c1cc(N(c2ccc3c4ccccc4n(-c4cccc(C(C)(C)C)c4)c3c2)c2cccc3c2CCCC3)c2ccc3c(C(C)C)cc(N(c4ccc5c6ccccc6n(-c6cccc(C(C)(C)C)c6)c5c4)c4cccc5c4CCCC5)c4ccc1c2c34 ; CC(C)c1cc(N(c2ccc3c4ccccc4n(-c4ccc(C(C)(C)C)cc4)c3c2)c2cccc3c2CCCC3)c2ccc3c(C(C)C)cc(N(c4ccc5c6ccccc6n(-c6ccc(C(C)(C)C)cc6)c5c4)c4cccc5c4CCCC5)c4ccc1c2c34 ; CC(C)c1ccccc1-n1c2ccccc2c2ccc(N(c3cccc4c3CCCC4)c3cc(C(C)C)c4ccc5c(N(c6ccc7c8ccccc8n(-c8ccccc8C(C)C)c7c6)c6cccc7c6CCCC7)cc(C(C)C)c6ccc3c4c65)cc21]

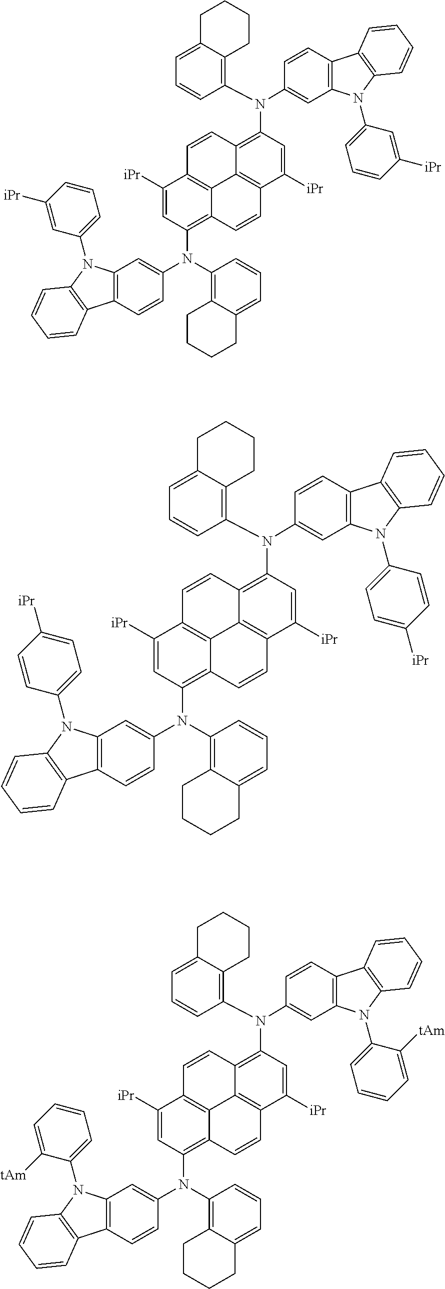

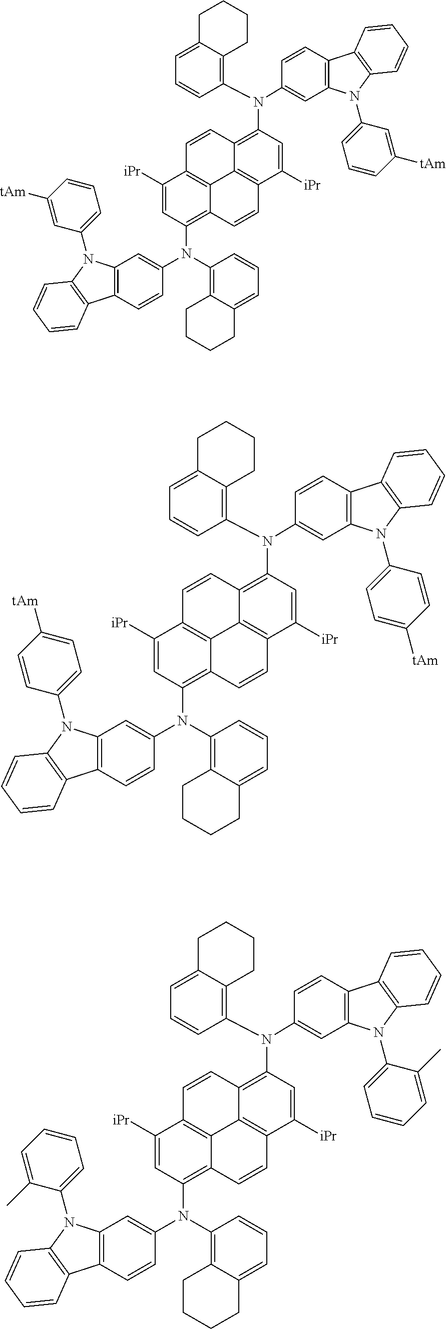

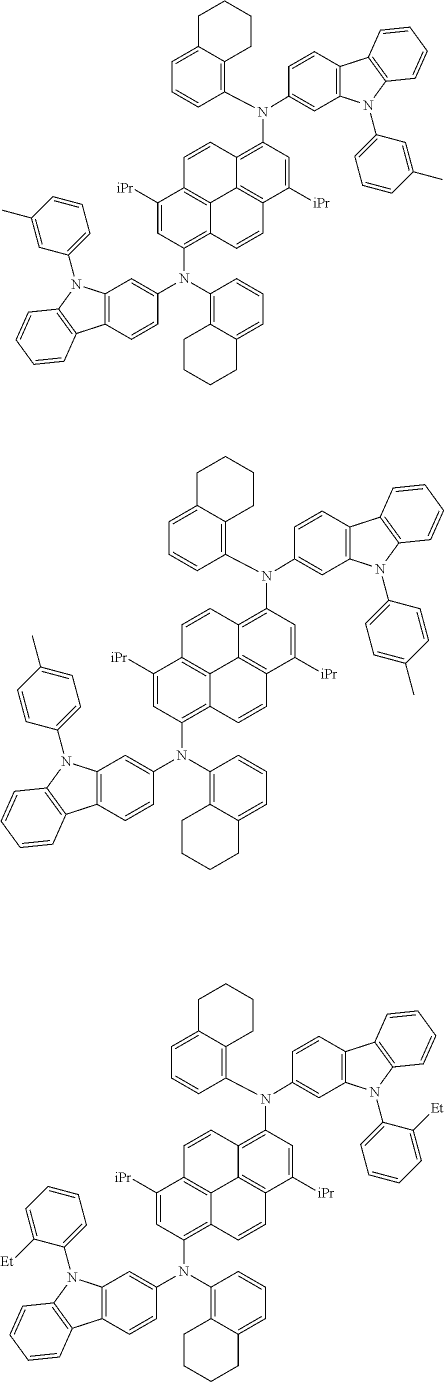

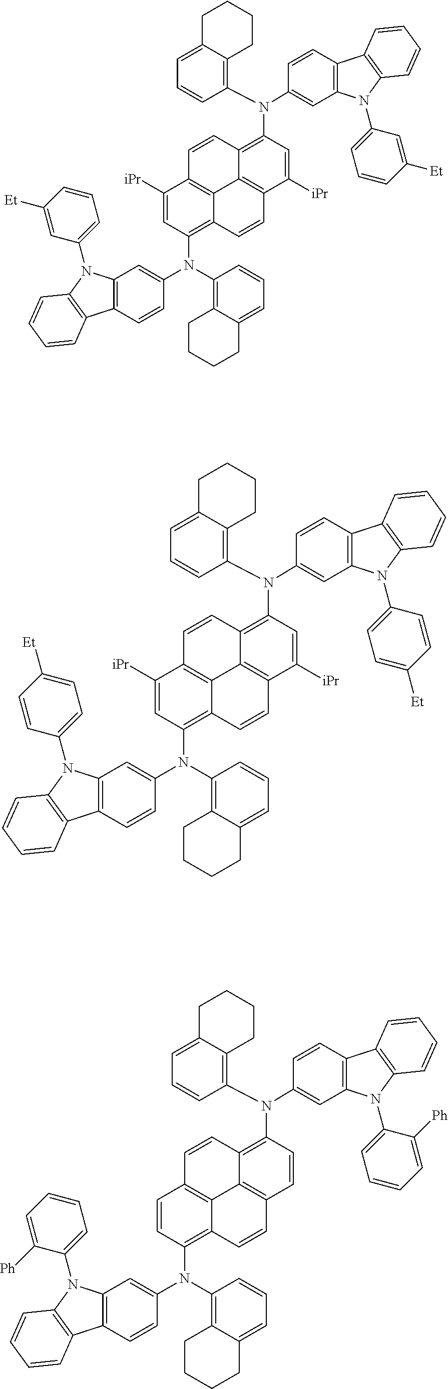 [SMILES: CCc1cccc(-n2c3ccccc3c3ccc(N(c4cccc5c4CCCC5)c4cc(C(C)C)c5ccc6c(N(c7ccc8c9ccccc9n(-c9cccc(CC)c9)c8c7)c7cccc8c7CCCC8)cc(C(C)C)c7ccc4c5c76)cc32)c1 ; CCc1ccc(-n2c3ccccc3c3ccc(N(c4cccc5c4CCCC5)c4cc(C(C)C)c5ccc6c(N(c7ccc8c9ccccc9n(-c9ccc(CC)cc9)c8c7)c7cccc8c7CCCC8)cc(C(C)C)c7ccc4c5c76)cc32)cc1 ; c1ccc(-c2ccccc2-n2c3ccccc3c3ccc(N(c4cccc5c4CCCC5)c4ccc5ccc6c(N(c7ccc8c9ccccc9n(-c9ccccc9-c9ccccc9)c8c7)c7cccc8c7CCCC8)ccc7ccc4c5c76)cc32)cc1]

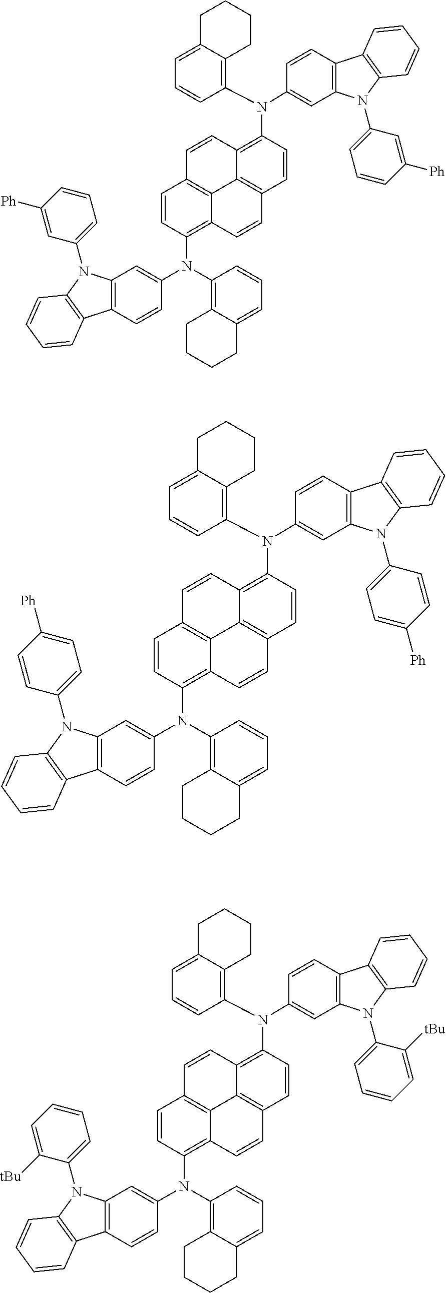 [SMILES: c1ccc(-c2cccc(-n3c4ccccc4c4ccc(N(c5cccc6c5CCCC6)c5ccc6ccc7c(N(c8ccc9c%10ccccc%10n(-c%10cccc(-c%11ccccc%11)c%10)c9c8)c8cccc9c8CCCC9)ccc8ccc5c6c87)cc43)c2)cc1 ; c1ccc(-c2ccc(-n3c4ccccc4c4ccc(N(c5cccc6c5CCCC6)c5ccc6ccc7c(N(c8ccc9c%10ccccc%10n(-c%10ccc(-c%11ccccc%11)cc%10)c9c8)c8cccc9c8CCCC9)ccc8ccc5c6c87)cc43)cc2)cc1 ; CC(C)(C)c1ccccc1-n1c2ccccc2c2ccc(N(c3cccc4c3CCCC4)c3ccc4ccc5c(N(c6ccc7c8ccccc8n(-c8ccccc8C(C)(C)C)c7c6)c6cccc7c6CCCC7)ccc6ccc3c4c65)cc21]

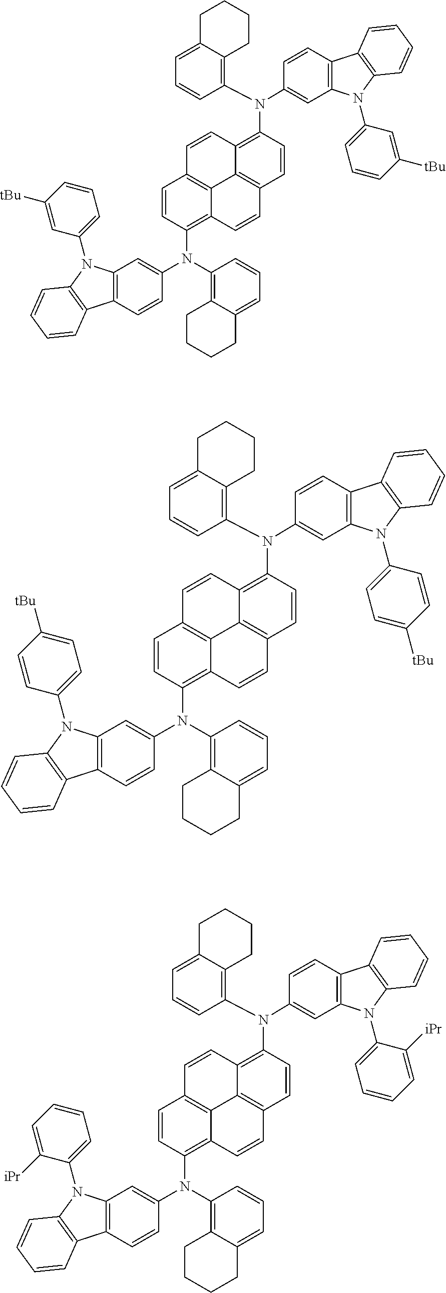 [SMILES: CC(C)(C)c1cccc(-n2c3ccccc3c3ccc(N(c4cccc5c4CCCC5)c4ccc5ccc6c(N(c7ccc8c9ccccc9n(-c9cccc(C(C)(C)C)c9)c8c7)c7cccc8c7CCCC8)ccc7ccc4c5c76)cc32)c1 ; CC(C)(C)c1ccc(-n2c3ccccc3c3ccc(N(c4cccc5c4CCCC5)c4ccc5ccc6c(N(c7ccc8c9ccccc9n(-c9ccc(C(C)(C)C)cc9)c8c7)c7cccc8c7CCCC8)ccc7ccc4c5c76)cc32)cc1 ; CC(C)c1ccccc1-n1c2ccccc2c2ccc(N(c3cccc4c3CCCC4)c3ccc4ccc5c(N(c6ccc7c8ccccc8n(-c8ccccc8C(C)C)c7c6)c6cccc7c6CCCC7)ccc6ccc3c4c65)cc21]

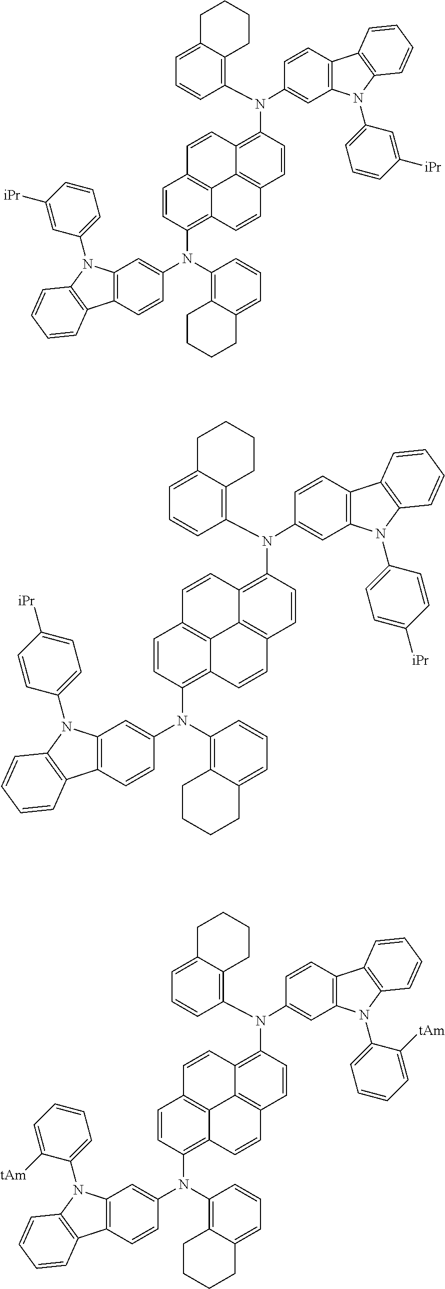

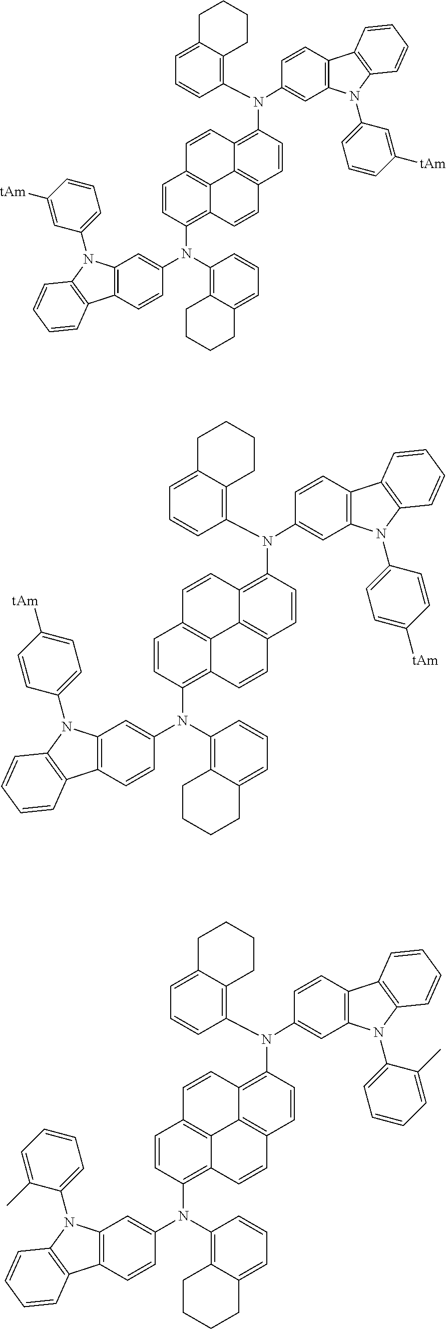

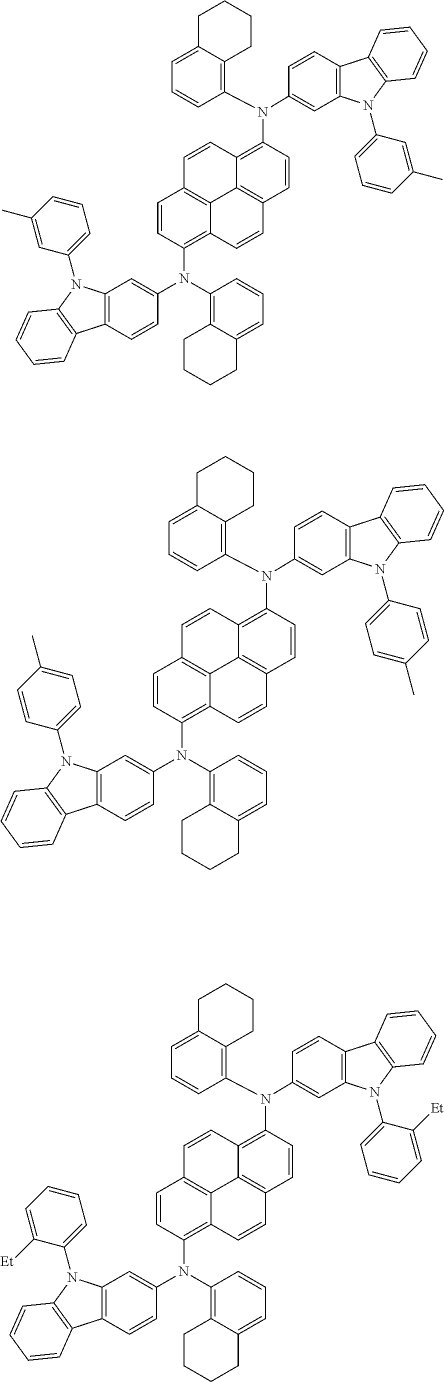

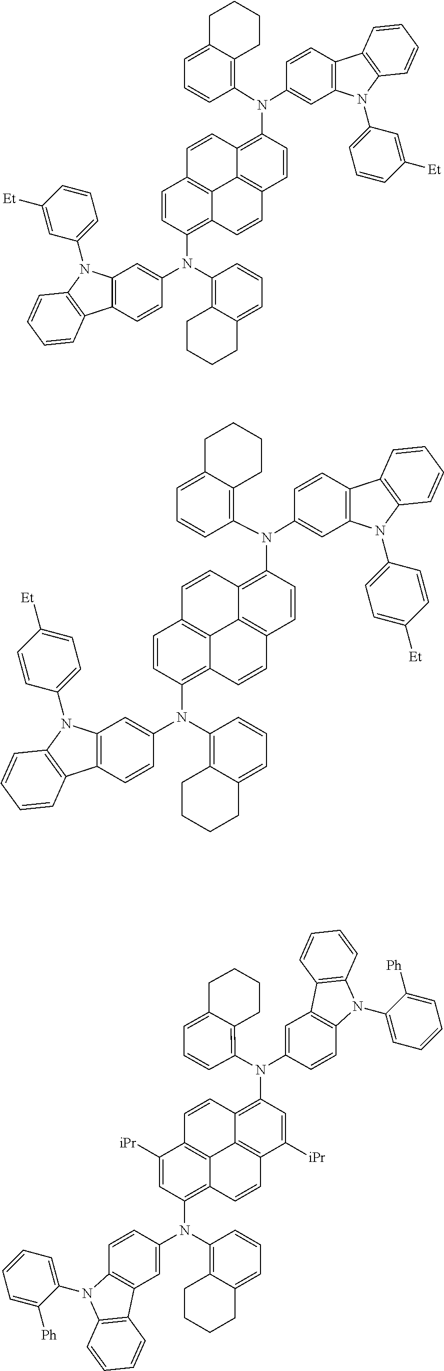 [SMILES: CCc1cccc(-n2c3ccccc3c3ccc(N(c4cccc5c4CCCC5)c4ccc5ccc6c(N(c7ccc8c9ccccc9n(-c9cccc(CC)c9)c8c7)c7cccc8c7CCCC8)ccc7ccc4c5c76)cc32)c1 ; CCc1ccc(-n2c3ccccc3c3ccc(N(c4cccc5c4CCCC5)c4ccc5ccc6c(N(c7ccc8c9ccccc9n(-c9ccc(CC)cc9)c8c7)c7cccc8c7CCCC8)ccc7ccc4c5c76)cc32)cc1 ; CC(C)c1cc(N(c2ccc3c(c2)c2ccccc2n3-c2ccccc2-c2ccccc2)c2cccc3c2CCCC3)c2ccc3c(C(C)C)cc(N(c4ccc5c(c4)c4ccccc4n5-c4ccccc4-c4ccccc4)c4cccc5c4CCCC5)c4ccc1c2c34]

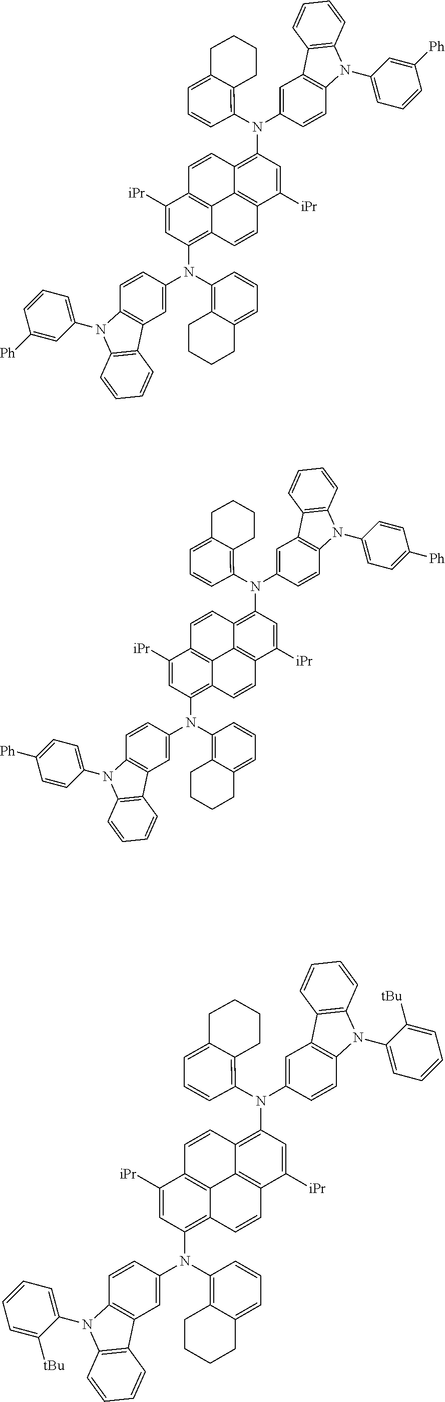 [SMILES: CC(C)c1cc(N(c2ccc3c(c2)c2ccccc2n3-c2cccc(-c3ccccc3)c2)c2cccc3c2CCCC3)c2ccc3c(C(C)C)cc(N(c4ccc5c(c4)c4ccccc4n5-c4cccc(-c5ccccc5)c4)c4cccc5c4CCCC5)c4ccc1c2c34 ; CC(C)c1cc(N(c2ccc3c(c2)c2ccccc2n3-c2ccc(-c3ccccc3)cc2)c2cccc3c2CCCC3)c2ccc3c(C(C)C)cc(N(c4ccc5c(c4)c4ccccc4n5-c4ccc(-c5ccccc5)cc4)c4cccc5c4CCCC5)c4ccc1c2c34 ; CC(C)c1cc(N(c2ccc3c(c2)c2ccccc2n3-c2ccccc2C(C)(C)C)c2cccc3c2CCCC3)c2ccc3c(C(C)C)cc(N(c4ccc5c(c4)c4ccccc4n5-c4ccccc4C(C)(C)C)c4cccc5c4CCCC5)c4ccc1c2c34]

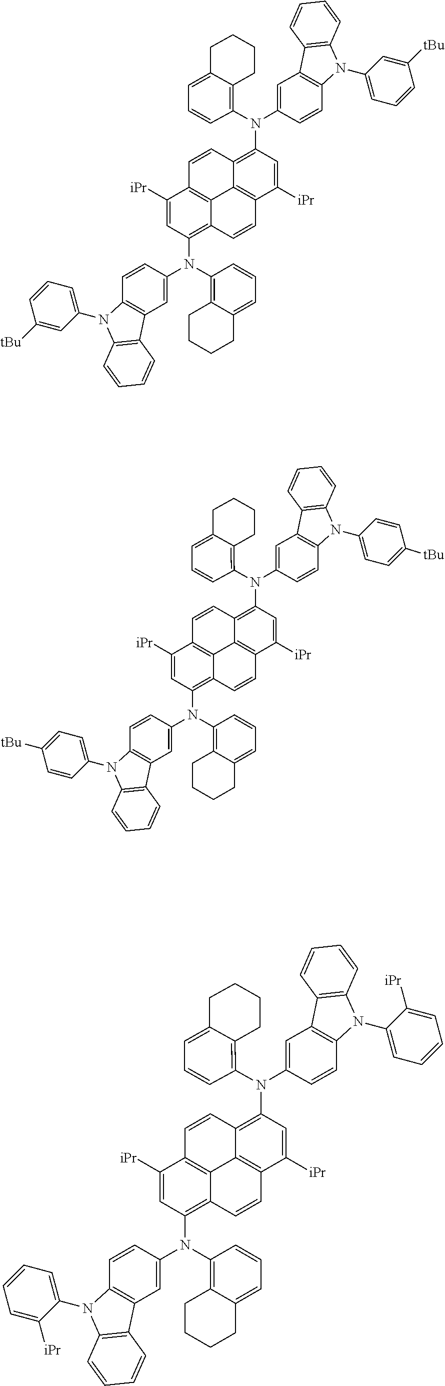 [SMILES: CC(C)c1cc(N(c2ccc3c(c2)c2ccccc2n3-c2cccc(C(C)(C)C)c2)c2cccc3c2CCCC3)c2ccc3c(C(C)C)cc(N(c4ccc5c(c4)c4ccccc4n5-c4cccc(C(C)(C)C)c4)c4cccc5c4CCCC5)c4ccc1c2c34 ; CC(C)c1cc(N(c2ccc3c(c2)c2ccccc2n3-c2ccc(C(C)(C)C)cc2)c2cccc3c2CCCC3)c2ccc3c(C(C)C)cc(N(c4ccc5c(c4)c4ccccc4n5-c4ccc(C(C)(C)C)cc4)c4cccc5c4CCCC5)c4ccc1c2c34 ; CC(C)c1ccccc1-n1c2ccccc2c2cc(N(c3cccc4c3CCCC4)c3cc(C(C)C)c4ccc5c(N(c6ccc7c(c6)c6ccccc6n7-c6ccccc6C(C)C)c6cccc7c6CCCC7)cc(C(C)C)c6ccc3c4c65)ccc21]

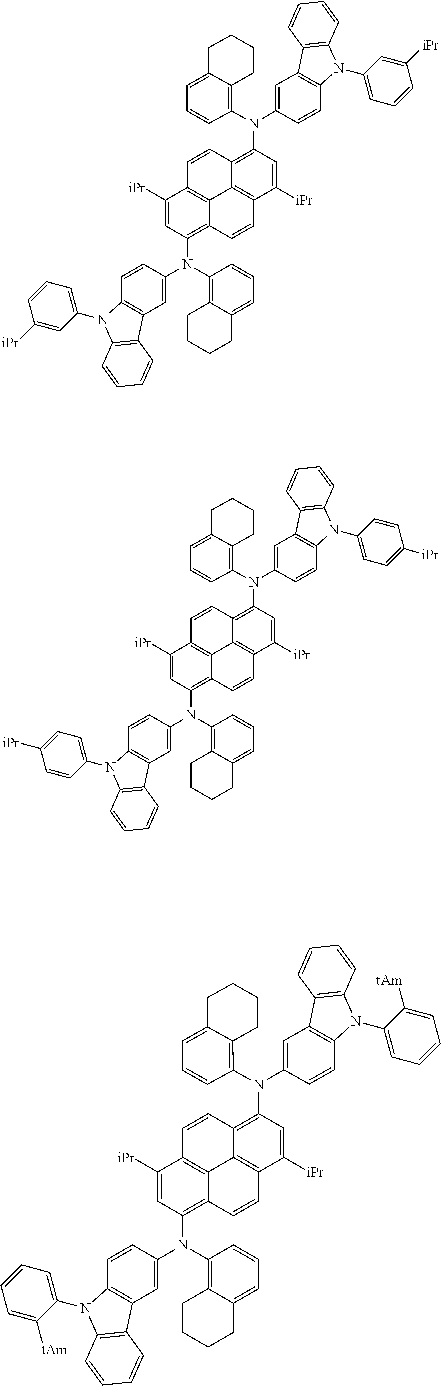

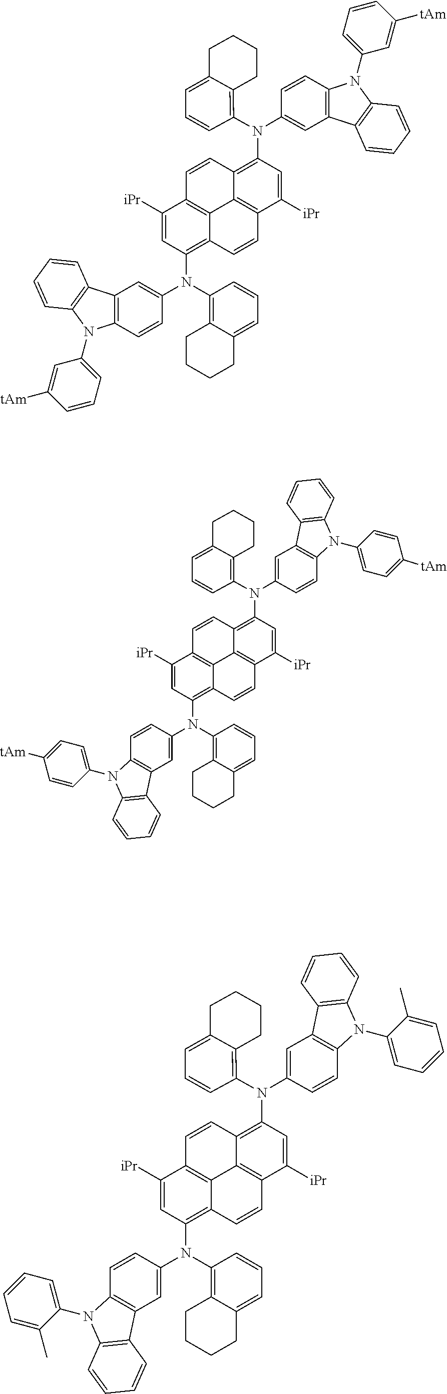

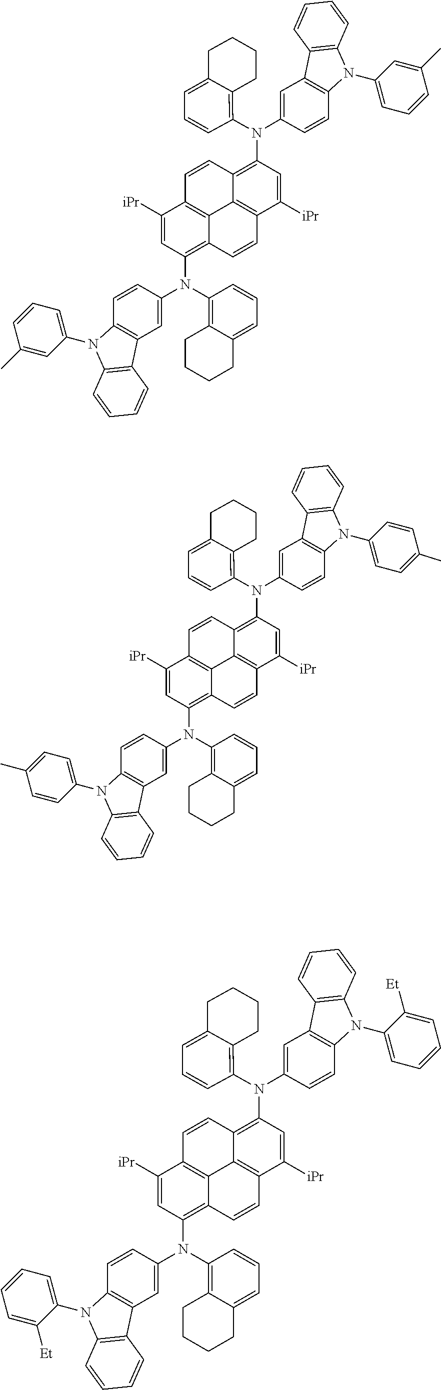

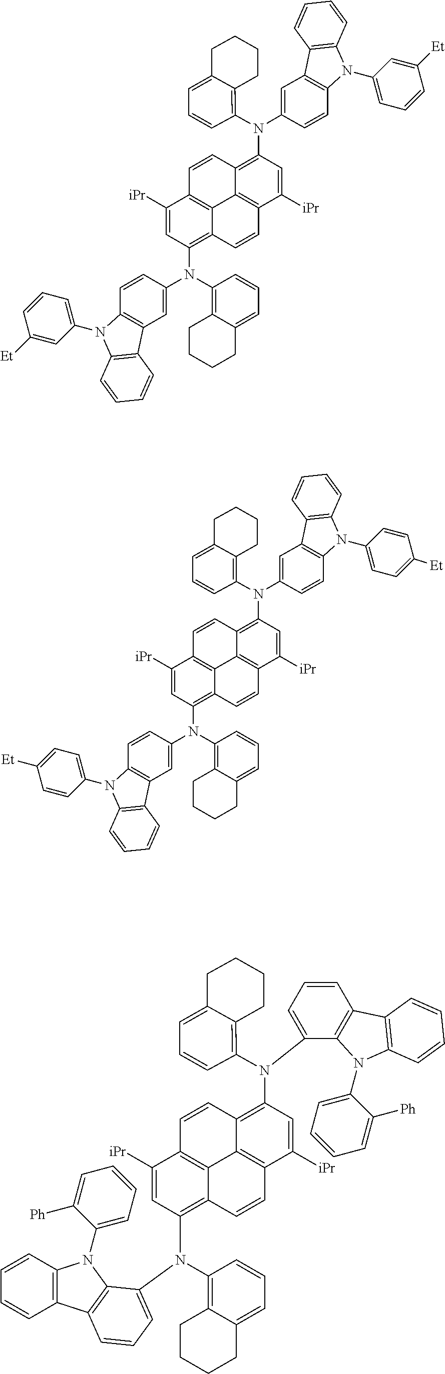 [SMILES: CCc1cccc(-n2c3ccccc3c3cc(N(c4cccc5c4CCCC5)c4cc(C(C)C)c5ccc6c(N(c7ccc8c(c7)c7ccccc7n8-c7cccc(CC)c7)c7cccc8c7CCCC8)cc(C(C)C)c7ccc4c5c76)ccc32)c1 ; CCc1ccc(-n2c3ccccc3c3cc(N(c4cccc5c4CCCC5)c4cc(C(C)C)c5ccc6c(N(c7ccc8c(c7)c7ccccc7n8-c7ccc(CC)cc7)c7cccc8c7CCCC8)cc(C(C)C)c7ccc4c5c76)ccc32)cc1 ; CC(C)c1cc(N(c2cccc3c2CCCC3)c2cccc3c4ccccc4n(-c4ccccc4-c4ccccc4)c23)c2ccc3c(C(C)C)cc(N(c4cccc5c4CCCC5)c4cccc5c6ccccc6n(-c6ccccc6-c6ccccc6)c45)c4ccc1c2c34]

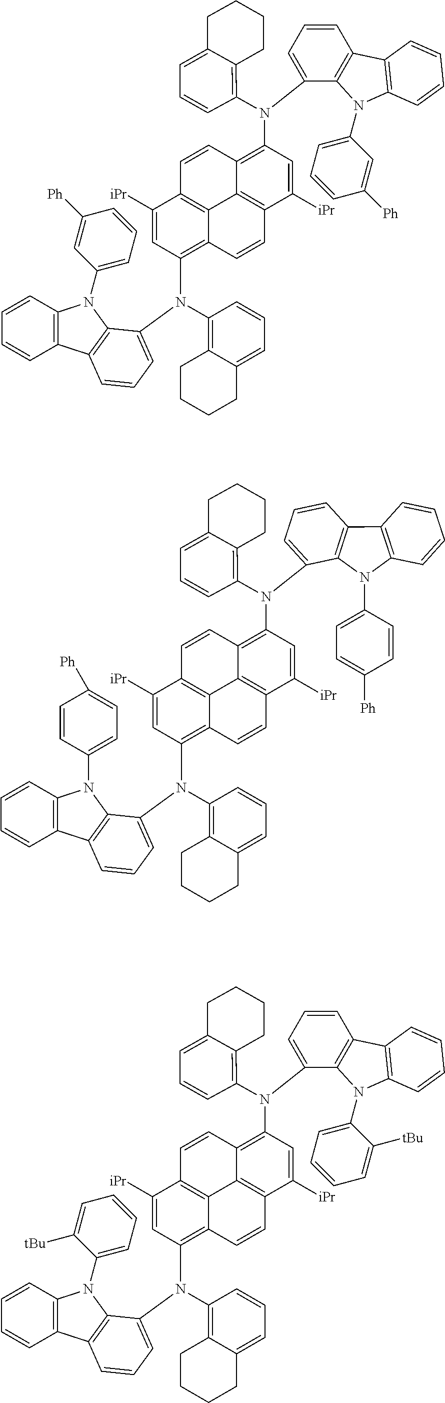 [SMILES: CC(C)c1cc(N(c2cccc3c2CCCC3)c2cccc3c4ccccc4n(-c4cccc(-c5ccccc5)c4)c23)c2ccc3c(C(C)C)cc(N(c4cccc5c4CCCC5)c4cccc5c6ccccc6n(-c6cccc(-c7ccccc7)c6)c45)c4ccc1c2c34 ; CC(C)c1cc(N(c2cccc3c2CCCC3)c2cccc3c4ccccc4n(-c4ccc(-c5ccccc5)cc4)c23)c2ccc3c(C(C)C)cc(N(c4cccc5c4CCCC5)c4cccc5c6ccccc6n(-c6ccc(-c7ccccc7)cc6)c45)c4ccc1c2c34 ; CC(C)c1cc(N(c2cccc3c2CCCC3)c2cccc3c4ccccc4n(-c4ccccc4C(C)(C)C)c23)c2ccc3c(C(C)C)cc(N(c4cccc5c4CCCC5)c4cccc5c6ccccc6n(-c6ccccc6C(C)(C)C)c45)c4ccc1c2c34]

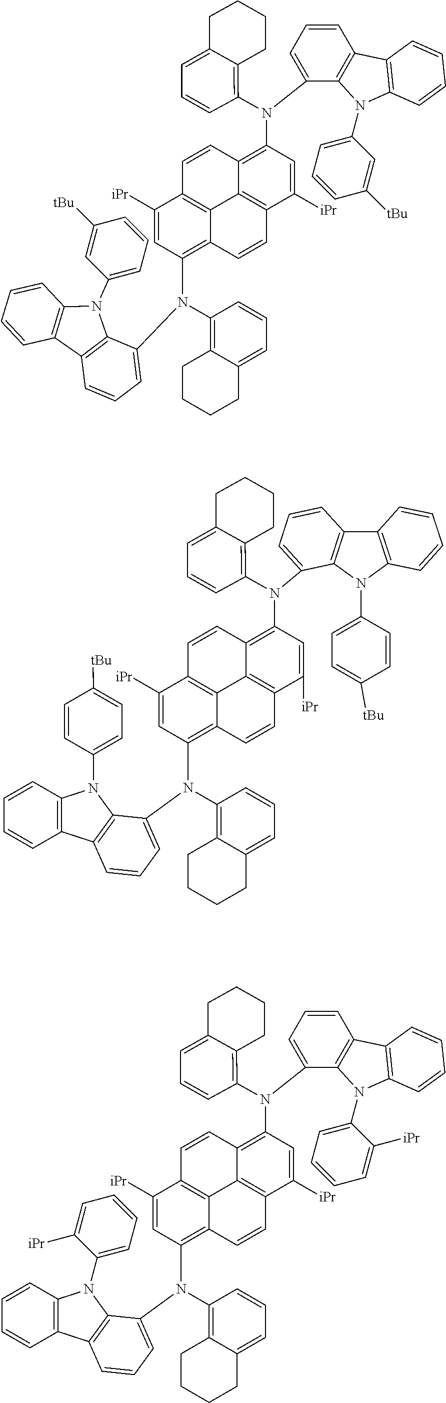 [SMILES: CC(C)c1cc(N(c2cccc3c2CCCC3)c2cccc3c4ccccc4n(-c4cccc(C(C)(C)C)c4)c23)c2ccc3c(C(C)C)cc(N(c4cccc5c4CCCC5)c4cccc5c6ccccc6n(-c6cccc(C(C)(C)C)c6)c45)c4ccc1c2c34 ; CC(C)c1cc(N(c2cccc3c2CCCC3)c2cccc3c4ccccc4n(-c4ccc(C(C)(C)C)cc4)c23)c2ccc3c(C(C)C)cc(N(c4cccc5c4CCCC5)c4cccc5c6ccccc6n(-c6ccc(C(C)(C)C)cc6)c45)c4ccc1c2c34 ; CC(C)c1ccccc1-n1c2ccccc2c2cccc(N(c3cccc4c3CCCC4)c3cc(C(C)C)c4ccc5c(N(c6cccc7c6CCCC7)c6cccc7c8ccccc8n(-c8ccccc8C(C)C)c67)cc(C(C)C)c6ccc3c4c65)c21]

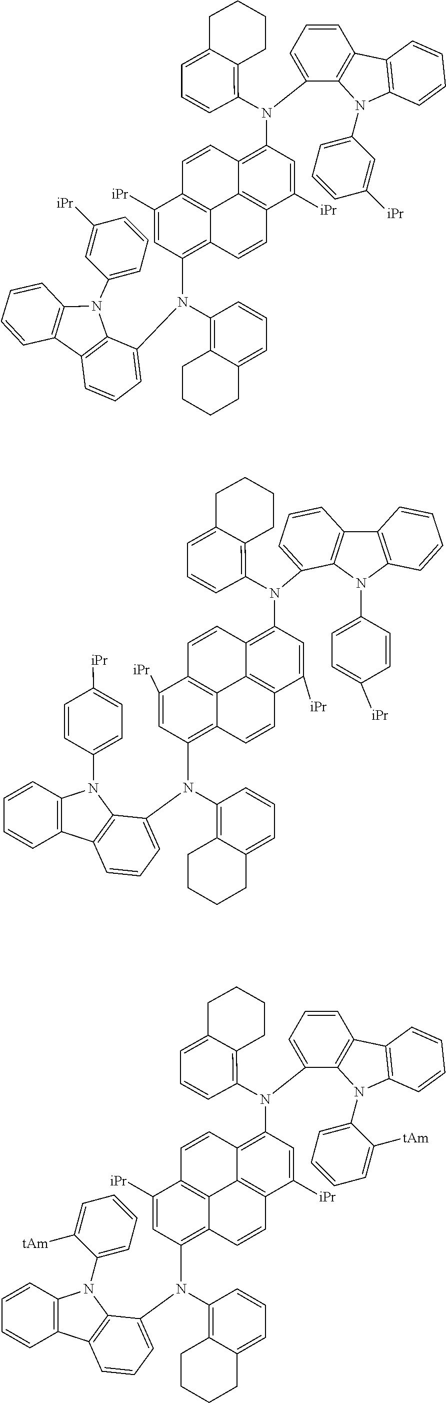

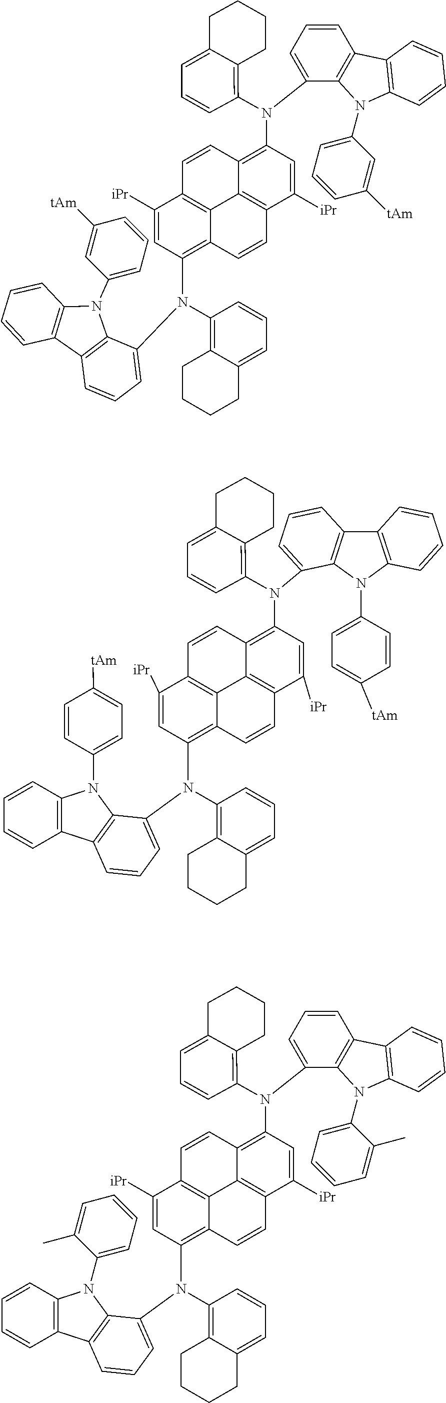

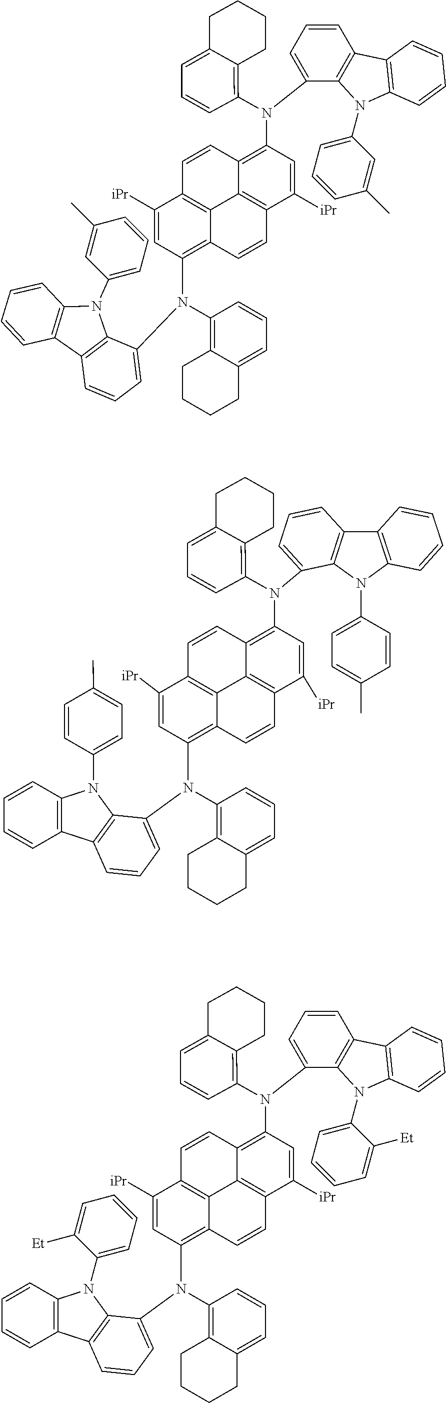 [SMILES: Cc1cccc(-n2c3ccccc3c3cccc(N(c4cccc5c4CCCC5)c4cc(C(C)C)c5ccc6c(N(c7cccc8c7CCCC8)c7cccc8c9ccccc9n(-c9cccc(C)c9)c78)cc(C(C)C)c7ccc4c5c76)c32)c1 ; Cc1ccc(-n2c3ccccc3c3cccc(N(c4cccc5c4CCCC5)c4cc(C(C)C)c5ccc6c(N(c7cccc8c7CCCC8)c7cccc8c9ccccc9n(-c9ccc(C)cc9)c78)cc(C(C)C)c7ccc4c5c76)c32)cc1 ; CCc1ccccc1-n1c2ccccc2c2cccc(N(c3cccc4c3CCCC4)c3cc(C(C)C)c4ccc5c(N(c6cccc7c6CCCC7)c6cccc7c8ccccc8n(-c8ccccc8CC)c67)cc(C(C)C)c6ccc3c4c65)c21]

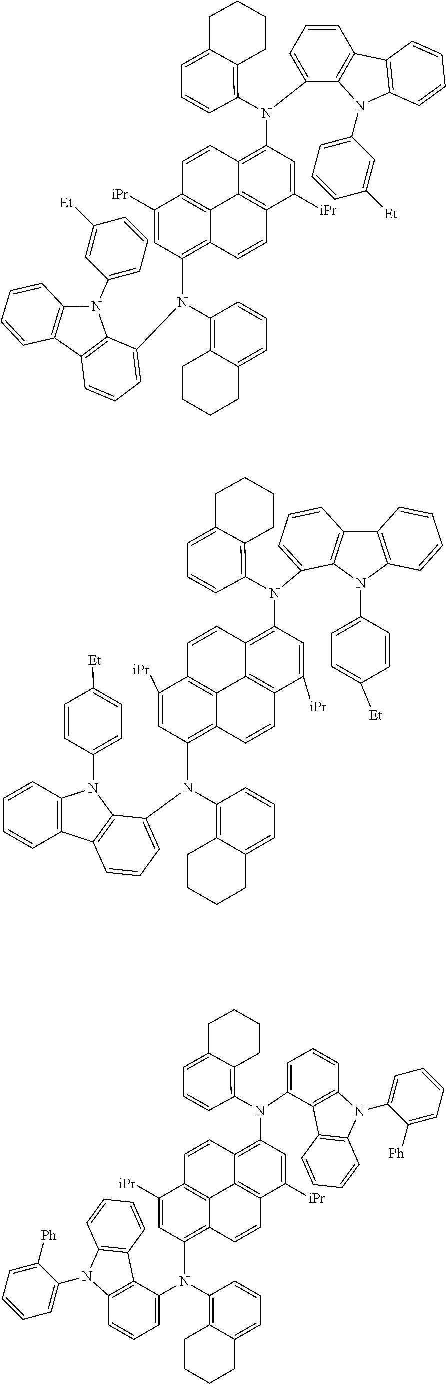 [SMILES: CCc1cccc(-n2c3ccccc3c3cccc(N(c4cccc5c4CCCC5)c4cc(C(C)C)c5ccc6c(N(c7cccc8c7CCCC8)c7cccc8c9ccccc9n(-c9cccc(CC)c9)c78)cc(C(C)C)c7ccc4c5c76)c32)c1 ; CCc1ccc(-n2c3ccccc3c3cccc(N(c4cccc5c4CCCC5)c4cc(C(C)C)c5ccc6c(N(c7cccc8c7CCCC8)c7cccc8c9ccccc9n(-c9ccc(CC)cc9)c78)cc(C(C)C)c7ccc4c5c76)c32)cc1 ; CC(C)c1cc(N(c2cccc3c2CCCC3)c2cccc3c2c2ccccc2n3-c2ccccc2-c2ccccc2)c2ccc3c(C(C)C)cc(N(c4cccc5c4CCCC5)c4cccc5c4c4ccccc4n5-c4ccccc4-c4ccccc4)c4ccc1c2c34]

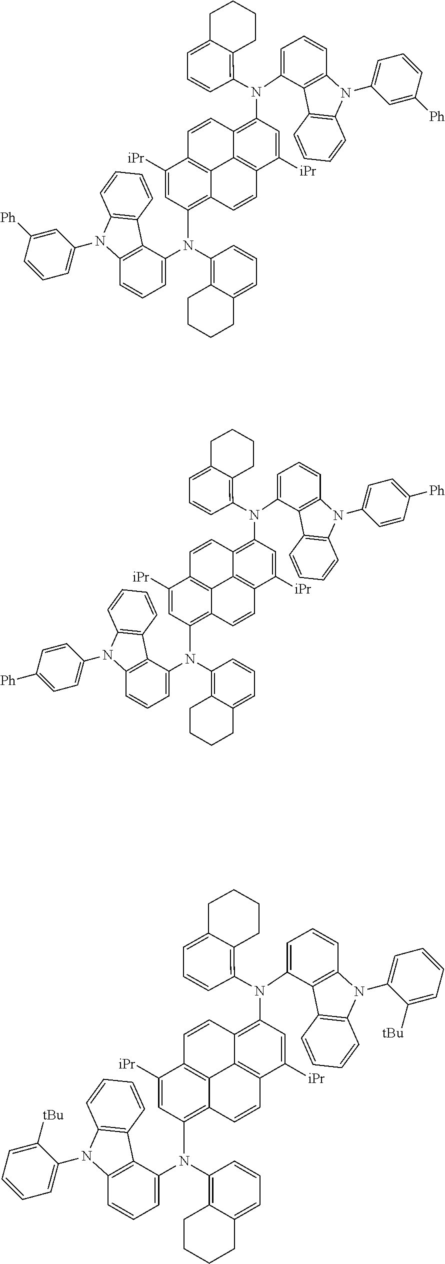 [SMILES: CC(C)c1cc(N(c2cccc3c2CCCC3)c2cccc3c2c2ccccc2n3-c2cccc(-c3ccccc3)c2)c2ccc3c(C(C)C)cc(N(c4cccc5c4CCCC5)c4cccc5c4c4ccccc4n5-c4cccc(-c5ccccc5)c4)c4ccc1c2c34 ; CC(C)c1cc(N(c2cccc3c2CCCC3)c2cccc3c2c2ccccc2n3-c2ccc(-c3ccccc3)cc2)c2ccc3c(C(C)C)cc(N(c4cccc5c4CCCC5)c4cccc5c4c4ccccc4n5-c4ccc(-c5ccccc5)cc4)c4ccc1c2c34 ; CC(C)c1cc(N(c2cccc3c2CCCC3)c2cccc3c2c2ccccc2n3-c2ccccc2C(C)(C)C)c2ccc3c(C(C)C)cc(N(c4cccc5c4CCCC5)c4cccc5c4c4ccccc4n5-c4ccccc4C(C)(C)C)c4ccc1c2c34]

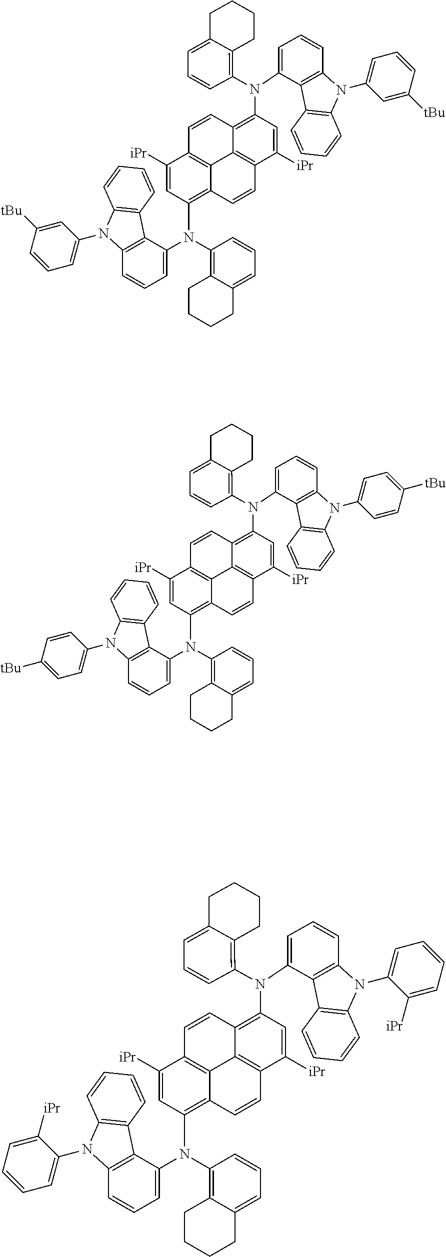 [SMILES: CC(C)c1cc(N(c2cccc3c2CCCC3)c2cccc3c2c2ccccc2n3-c2cccc(C(C)(C)C)c2)c2ccc3c(C(C)C)cc(N(c4cccc5c4CCCC5)c4cccc5c4c4ccccc4n5-c4cccc(C(C)(C)C)c4)c4ccc1c2c34 ; CC(C)c1cc(N(c2cccc3c2CCCC3)c2cccc3c2c2ccccc2n3-c2ccc(C(C)(C)C)cc2)c2ccc3c(C(C)C)cc(N(c4cccc5c4CCCC5)c4cccc5c4c4ccccc4n5-c4ccc(C(C)(C)C)cc4)c4ccc1c2c34 ; CC(C)c1ccccc1-n1c2ccccc2c2c(N(c3cccc4c3CCCC4)c3cc(C(C)C)c4ccc5c(N(c6cccc7c6CCCC7)c6cccc7c6c6ccccc6n7-c6ccccc6C(C)C)cc(C(C)C)c6ccc3c4c65)cccc21]

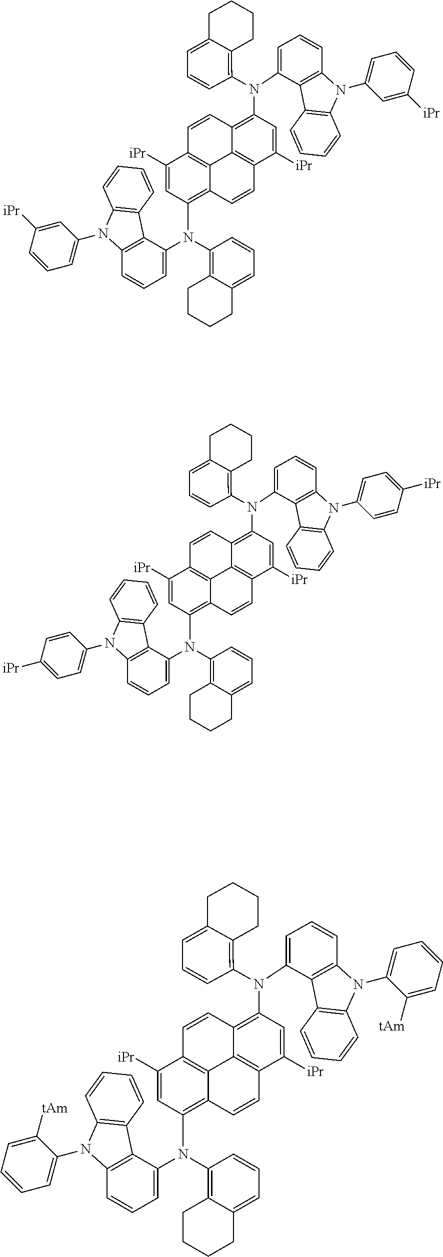

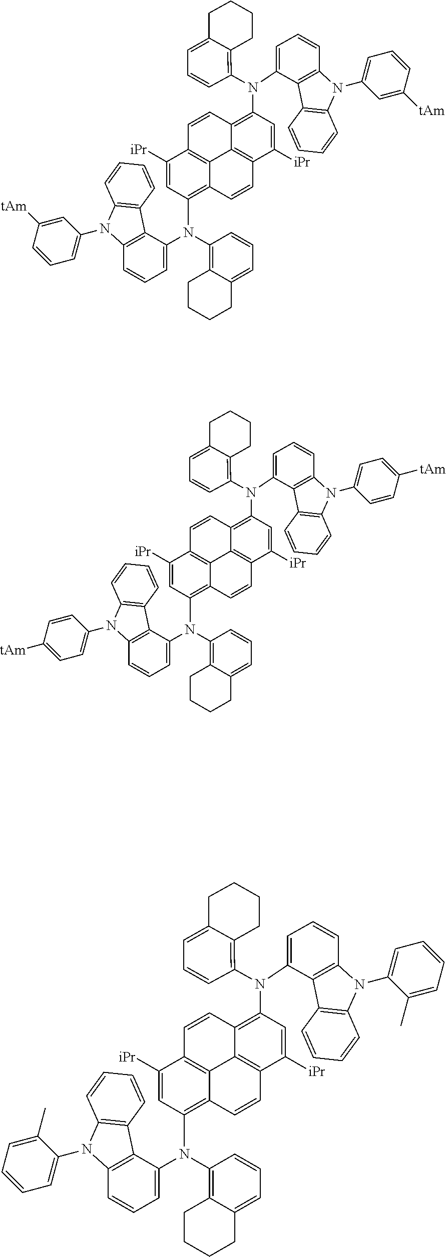

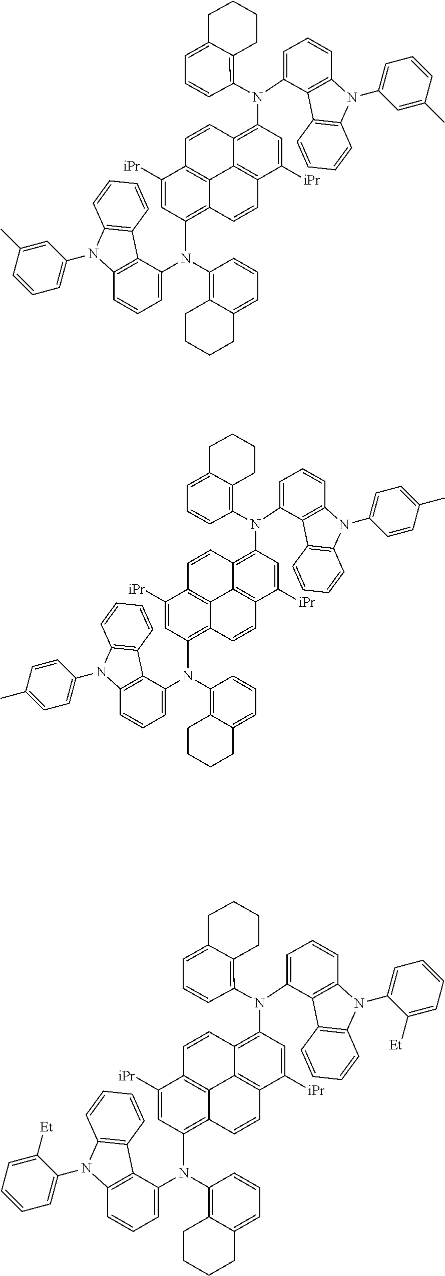

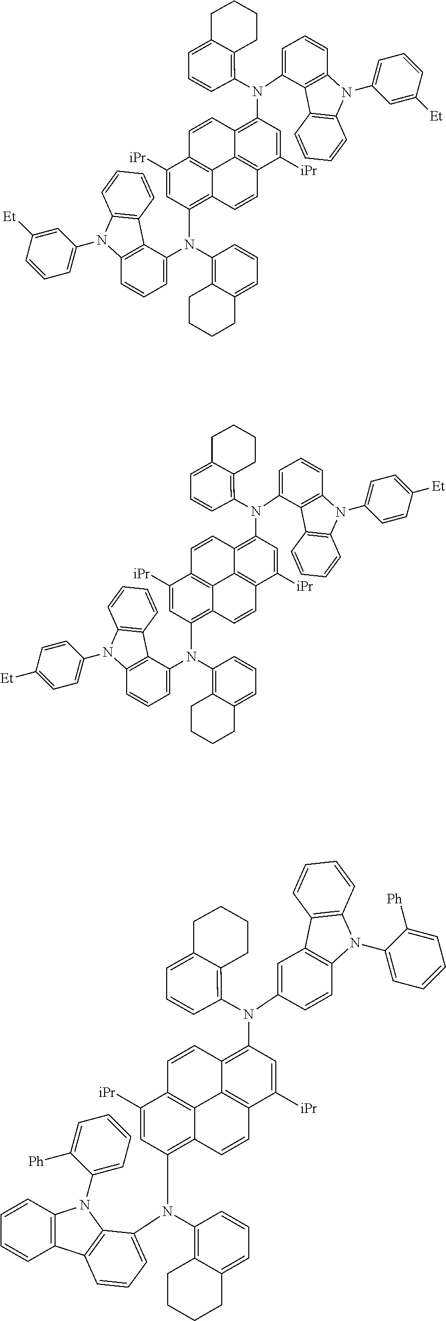 [SMILES: CCc1cccc(-n2c3ccccc3c3c(N(c4cccc5c4CCCC5)c4cc(C(C)C)c5ccc6c(N(c7cccc8c7CCCC8)c7cccc8c7c7ccccc7n8-c7cccc(CC)c7)cc(C(C)C)c7ccc4c5c76)cccc32)c1 ; CCc1ccc(-n2c3ccccc3c3c(N(c4cccc5c4CCCC5)c4cc(C(C)C)c5ccc6c(N(c7cccc8c7CCCC8)c7cccc8c7c7ccccc7n8-c7ccc(CC)cc7)cc(C(C)C)c7ccc4c5c76)cccc32)cc1 ; CC(C)c1cc(N(c2ccc3c(c2)c2ccccc2n3-c2ccccc2-c2ccccc2)c2cccc3c2CCCC3)c2ccc3c(C(C)C)cc(N(c4cccc5c4CCCC5)c4cccc5c6ccccc6n(-c6ccccc6-c6ccccc6)c45)c4ccc1c2c34]

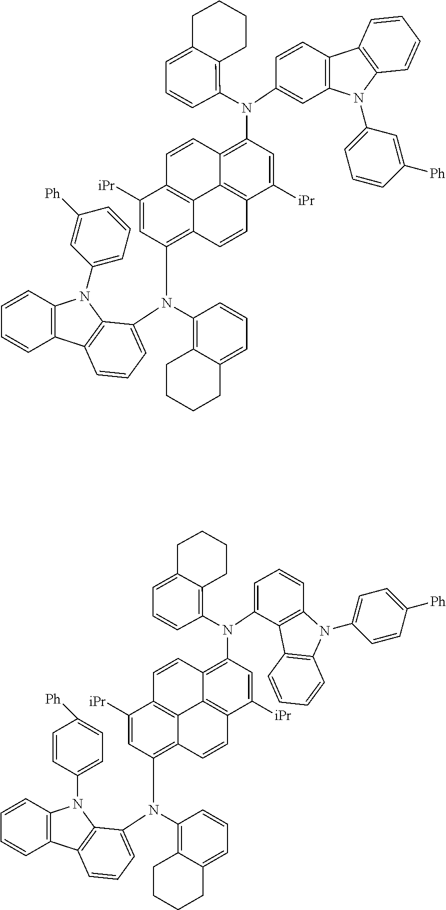 [SMILES: CC(C)c1cc(N(c2ccc3c4ccccc4n(-c4cccc(-c5ccccc5)c4)c3c2)c2cccc3c2CCCC3)c2ccc3c(C(C)C)cc(N(c4cccc5c4CCCC5)c4cccc5c6ccccc6n(-c6cccc(-c7ccccc7)c6)c45)c4ccc1c2c34 ; CC(C)c1cc(N(c2cccc3c2CCCC3)c2cccc3c2c2ccccc2n3-c2ccc(-c3ccccc3)cc2)c2ccc3c(C(C)C)cc(N(c4cccc5c4CCCC5)c4cccc5c6ccccc6n(-c6ccc(-c7ccccc7)cc6)c45)c4ccc1c2c34]

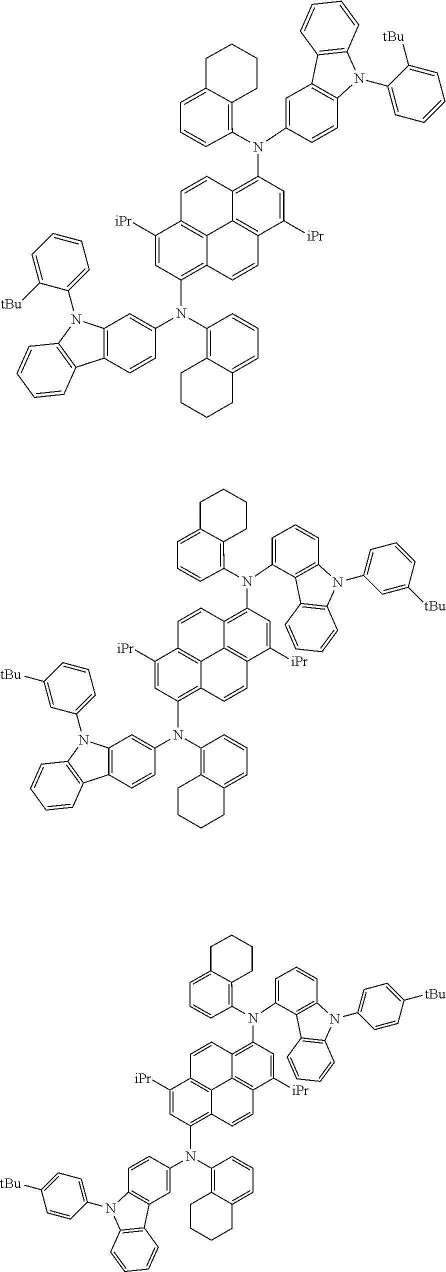 [SMILES: CC(C)c1cc(N(c2ccc3c(c2)c2ccccc2n3-c2ccccc2C(C)(C)C)c2cccc3c2CCCC3)c2ccc3c(C(C)C)cc(N(c4ccc5c6ccccc6n(-c6ccccc6C(C)(C)C)c5c4)c4cccc5c4CCCC5)c4ccc1c2c34 ; CC(C)c1cc(N(c2ccc3c4ccccc4n(-c4cccc(C(C)(C)C)c4)c3c2)c2cccc3c2CCCC3)c2ccc3c(C(C)C)cc(N(c4cccc5c4CCCC5)c4cccc5c4c4ccccc4n5-c4cccc(C(C)(C)C)c4)c4ccc1c2c34 ; CC(C)c1cc(N(c2ccc3c(c2)c2ccccc2n3-c2ccc(C(C)(C)C)cc2)c2cccc3c2CCCC3)c2ccc3c(C(C)C)cc(N(c4cccc5c4CCCC5)c4cccc5c4c4ccccc4n5-c4ccc(C(C)(C)C)cc4)c4ccc1c2c34]

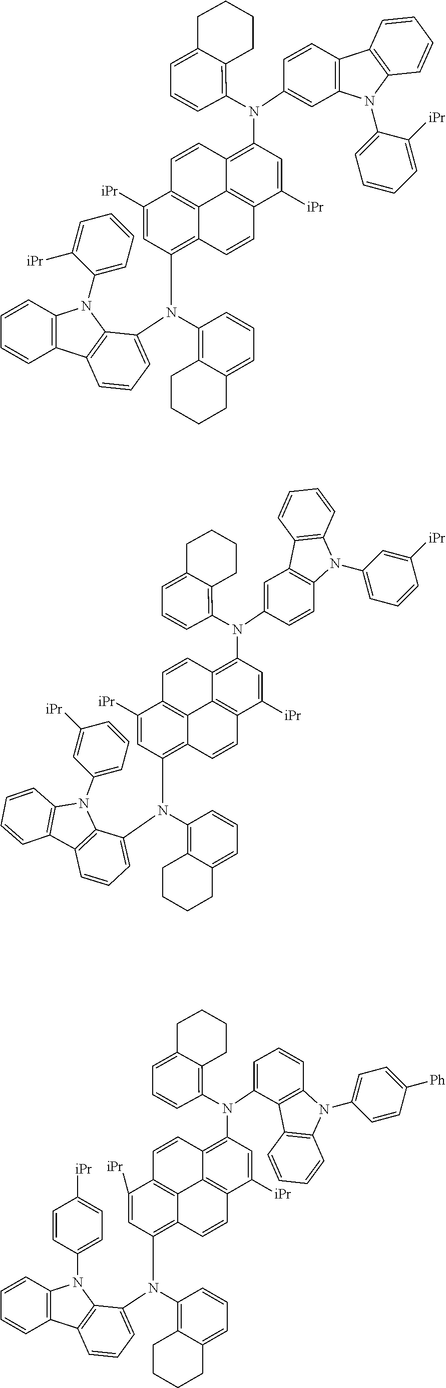 [SMILES: CC(C)c1ccccc1-n1c2ccccc2c2ccc(N(c3cccc4c3CCCC4)c3cc(C(C)C)c4ccc5c(N(c6cccc7c6CCCC7)c6cccc7c8ccccc8n(-c8ccccc8C(C)C)c67)cc(C(C)C)c6ccc3c4c65)cc21 ; CC(C)c1cccc(-n2c3ccccc3c3cc(N(c4cccc5c4CCCC5)c4cc(C(C)C)c5ccc6c(N(c7cccc8c7CCCC8)c7cccc8c9ccccc9n(-c9cccc(C(C)C)c9)c78)cc(C(C)C)c7ccc4c5c76)ccc32)c1 ; CC(C)c1ccc(-n2c3ccccc3c3cccc(N(c4cccc5c4CCCC5)c4cc(C(C)C)c5ccc6c(N(c7cccc8c7CCCC8)c7cccc8c7c7ccccc7n8-c7ccc(-c8ccccc8)cc7)cc(C(C)C)c7ccc4c5c76)c32)cc1]

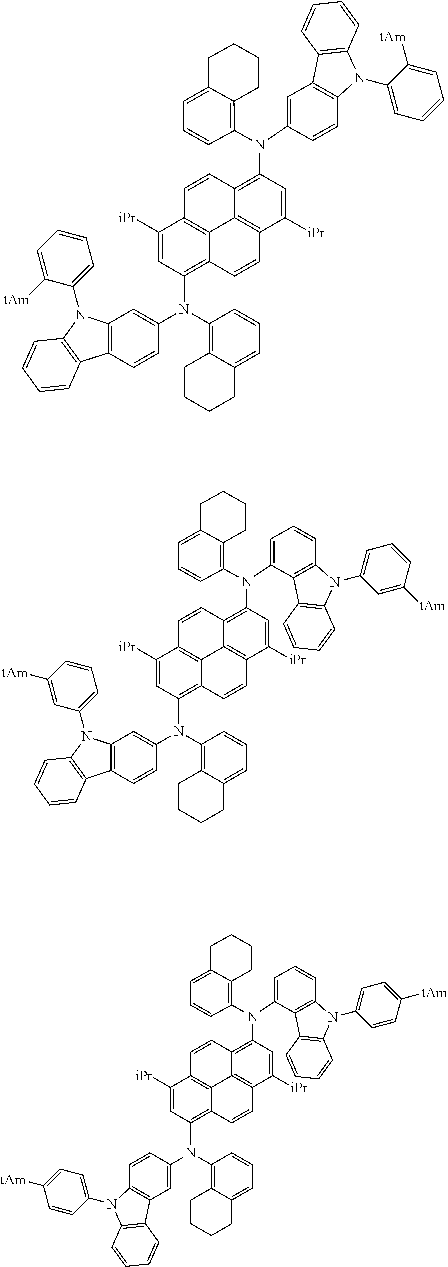

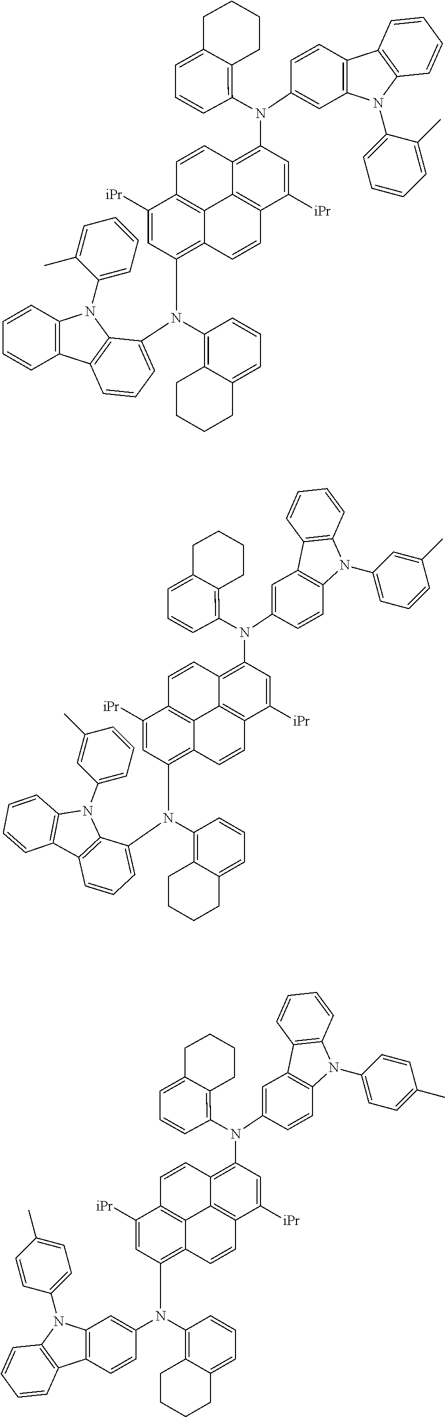 [SMILES: Cc1ccccc1-n1c2ccccc2c2ccc(N(c3cccc4c3CCCC4)c3cc(C(C)C)c4ccc5c(N(c6cccc7c6CCCC7)c6cccc7c8ccccc8n(-c8ccccc8C)c67)cc(C(C)C)c6ccc3c4c65)cc21 ; Cc1cccc(-n2c3ccccc3c3cc(N(c4cccc5c4CCCC5)c4cc(C(C)C)c5ccc6c(N(c7cccc8c7CCCC8)c7cccc8c9ccccc9n(-c9cccc(C)c9)c78)cc(C(C)C)c7ccc4c5c76)ccc32)c1 ; Cc1ccc(-n2c3ccccc3c3cc(N(c4cccc5c4CCCC5)c4cc(C(C)C)c5ccc6c(N(c7ccc8c9ccccc9n(-c9ccc(C)cc9)c8c7)c7cccc8c7CCCC8)cc(C(C)C)c7ccc4c5c76)ccc32)cc1]

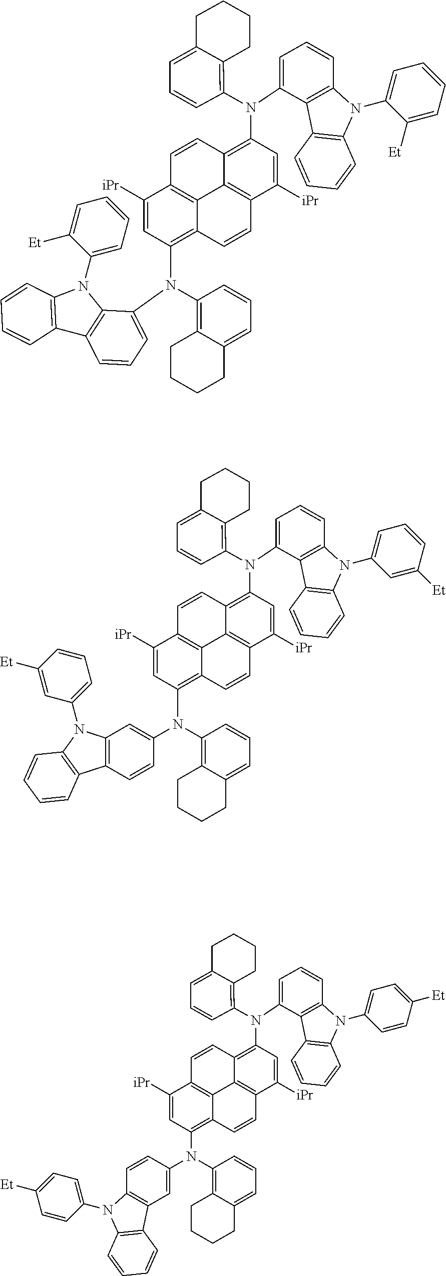 [SMILES: CCc1ccccc1-n1c2ccccc2c2c(N(c3cccc4c3CCCC4)c3cc(C(C)C)c4ccc5c(N(c6cccc7c6CCCC7)c6cccc7c8ccccc8n(-c8ccccc8CC)c67)cc(C(C)C)c6ccc3c4c65)cccc21 ; CCc1cccc(-n2c3ccccc3c3ccc(N(c4cccc5c4CCCC5)c4cc(C(C)C)c5ccc6c(N(c7cccc8c7CCCC8)c7cccc8c7c7ccccc7n8-c7cccc(CC)c7)cc(C(C)C)c7ccc4c5c76)cc32)c1 ; CCc1ccc(-n2c3ccccc3c3cc(N(c4cccc5c4CCCC5)c4cc(C(C)C)c5ccc6c(N(c7cccc8c7CCCC8)c7cccc8c7c7ccccc7n8-c7ccc(CC)cc7)cc(C(C)C)c7ccc4c5c76)ccc32)cc1]

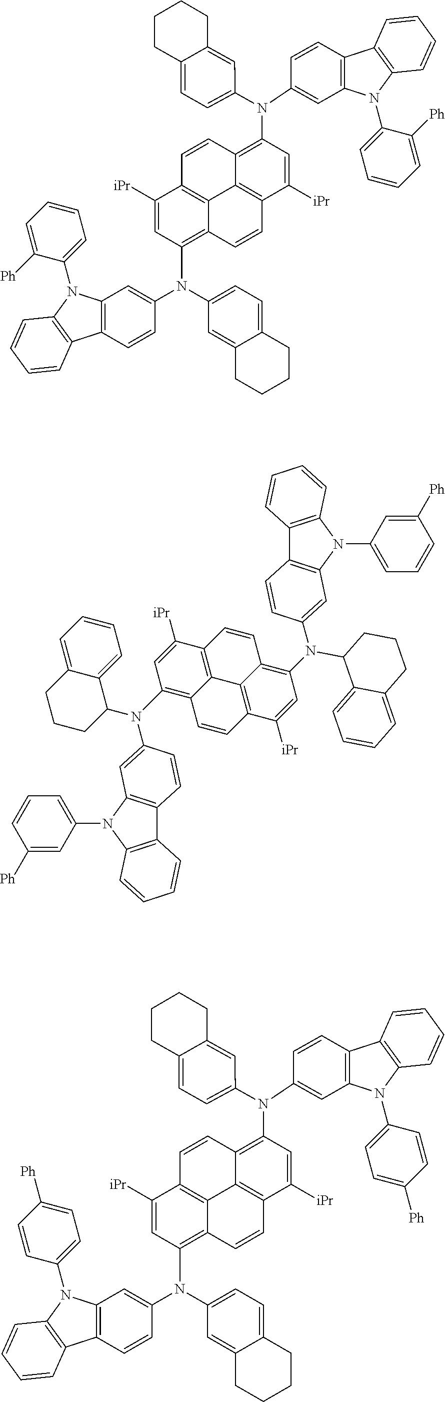 [SMILES: CC(C)c1cc(N(c2ccc3c(c2)CCCC3)c2ccc3c4ccccc4n(-c4ccccc4-c4ccccc4)c3c2)c2ccc3c(C(C)C)cc(N(c4ccc5c(c4)CCCC5)c4ccc5c6ccccc6n(-c6ccccc6-c6ccccc6)c5c4)c4ccc1c2c34 ; CC(C)c1cc(N(c2ccc3c4ccccc4n(-c4cccc(-c5ccccc5)c4)c3c2)C2CCCc3ccccc32)c2ccc3c(C(C)C)cc(N(c4ccc5c6ccccc6n(-c6cccc(-c7ccccc7)c6)c5c4)C4CCCc5ccccc54)c4ccc1c2c34 ; CC(C)c1cc(N(c2ccc3c(c2)CCCC3)c2ccc3c4ccccc4n(-c4ccc(-c5ccccc5)cc4)c3c2)c2ccc3c(C(C)C)cc(N(c4ccc5c(c4)CCCC5)c4ccc5c6ccccc6n(-c6ccc(-c7ccccc7)cc6)c5c4)c4ccc1c2c34]

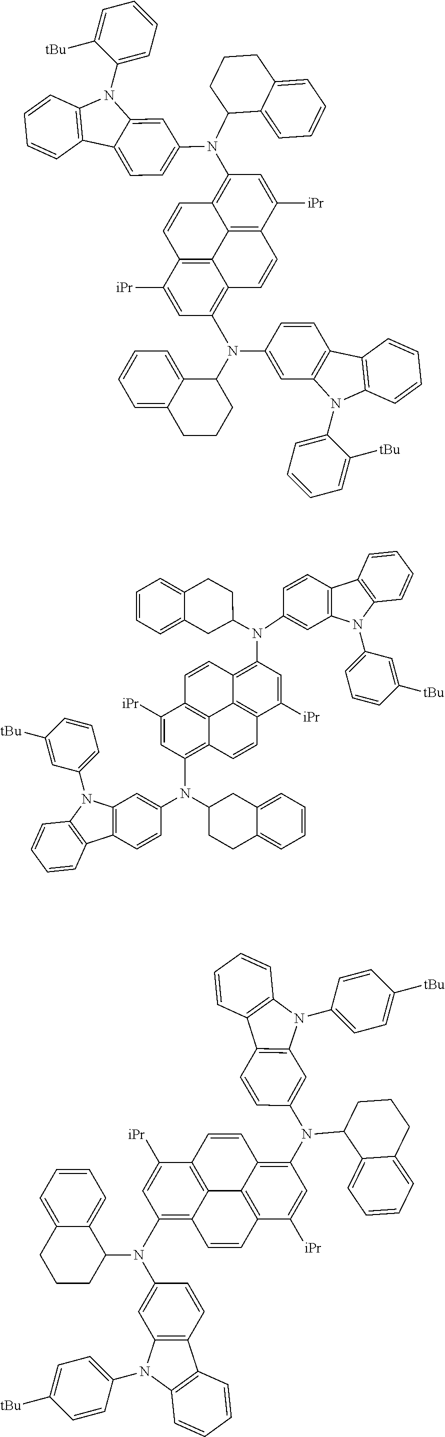 [SMILES: CC(C)c1cc(N(c2ccc3c4ccccc4n(-c4ccccc4C(C)(C)C)c3c2)C2CCCc3ccccc32)c2ccc3c(C(C)C)cc(N(c4ccc5c6ccccc6n(-c6ccccc6C(C)(C)C)c5c4)C4CCCc5ccccc54)c4ccc1c2c34 ; CC(C)c1cc(N(c2ccc3c4ccccc4n(-c4cccc(C(C)(C)C)c4)c3c2)C2CCc3ccccc3C2)c2ccc3c(C(C)C)cc(N(c4ccc5c6ccccc6n(-c6cccc(C(C)(C)C)c6)c5c4)C4CCc5ccccc5C4)c4ccc1c2c34 ; CC(C)c1cc(N(c2ccc3c4ccccc4n(-c4ccc(C(C)(C)C)cc4)c3c2)C2CCCc3ccccc32)c2ccc3c(C(C)C)cc(N(c4ccc5c6ccccc6n(-c6ccc(C(C)(C)C)cc6)c5c4)C4CCCc5ccccc54)c4ccc1c2c34]

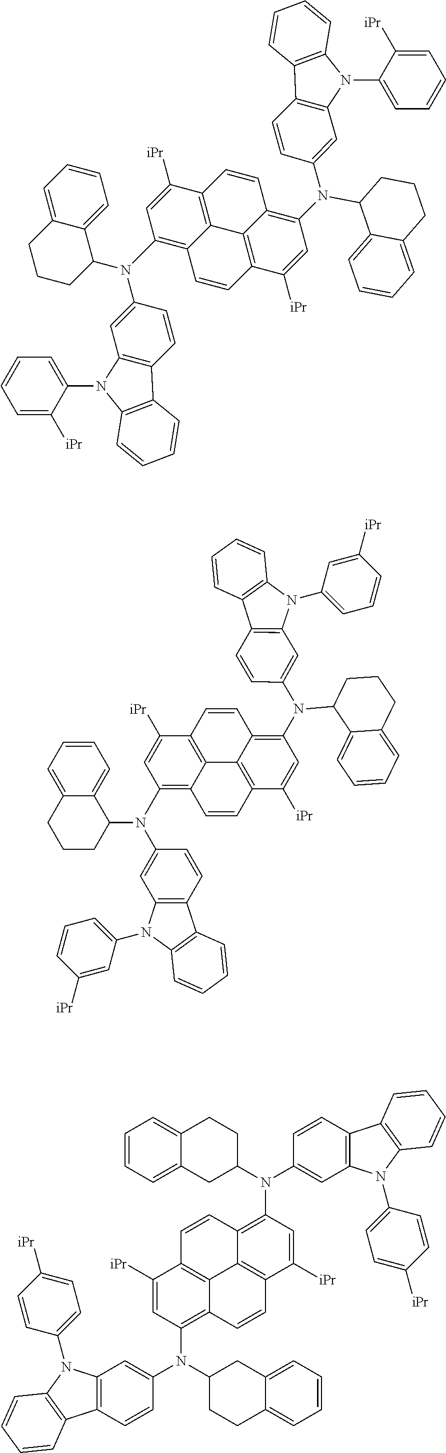 [SMILES: CC(C)c1ccccc1-n1c2ccccc2c2ccc(N(c3cc(C(C)C)c4ccc5c(N(c6ccc7c8ccccc8n(-c8ccccc8C(C)C)c7c6)C6CCCc7ccccc76)cc(C(C)C)c6ccc3c4c65)C3CCCc4ccccc43)cc21 ; CC(C)c1cccc(-n2c3ccccc3c3ccc(N(c4cc(C(C)C)c5ccc6c(N(c7ccc8c9ccccc9n(-c9cccc(C(C)C)c9)c8c7)C7CCCc8ccccc87)cc(C(C)C)c7ccc4c5c76)C4CCCc5ccccc54)cc32)c1 ; CC(C)c1ccc(-n2c3ccccc3c3ccc(N(c4cc(C(C)C)c5ccc6c(N(c7ccc8c9ccccc9n(-c9ccc(C(C)C)cc9)c8c7)C7CCc8ccccc8C7)cc(C(C)C)c7ccc4c5c76)C4CCc5ccccc5C4)cc32)cc1]

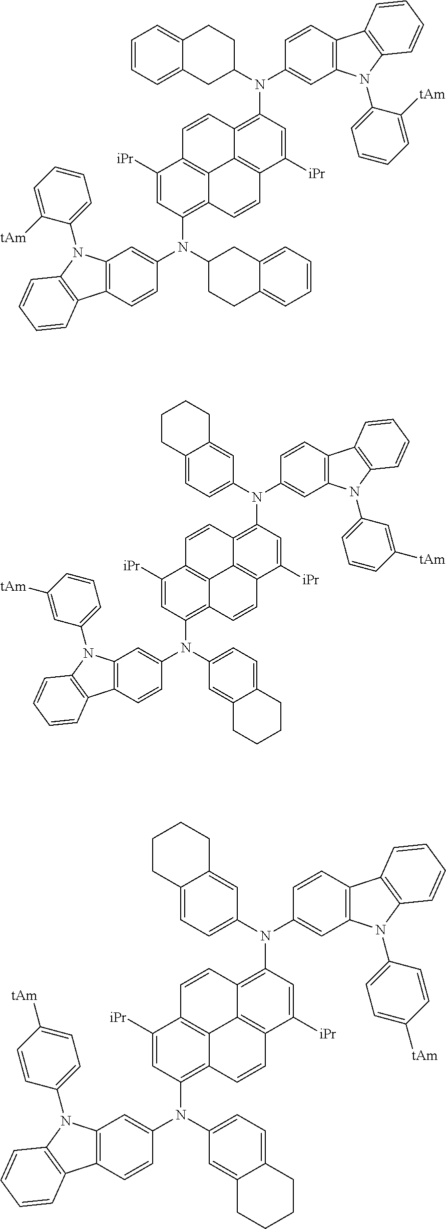

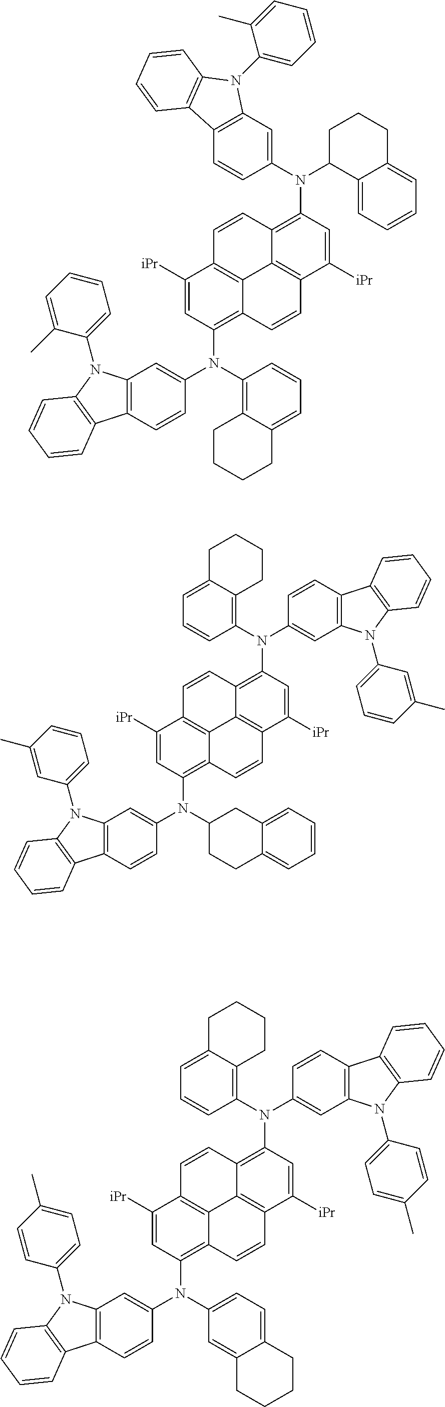

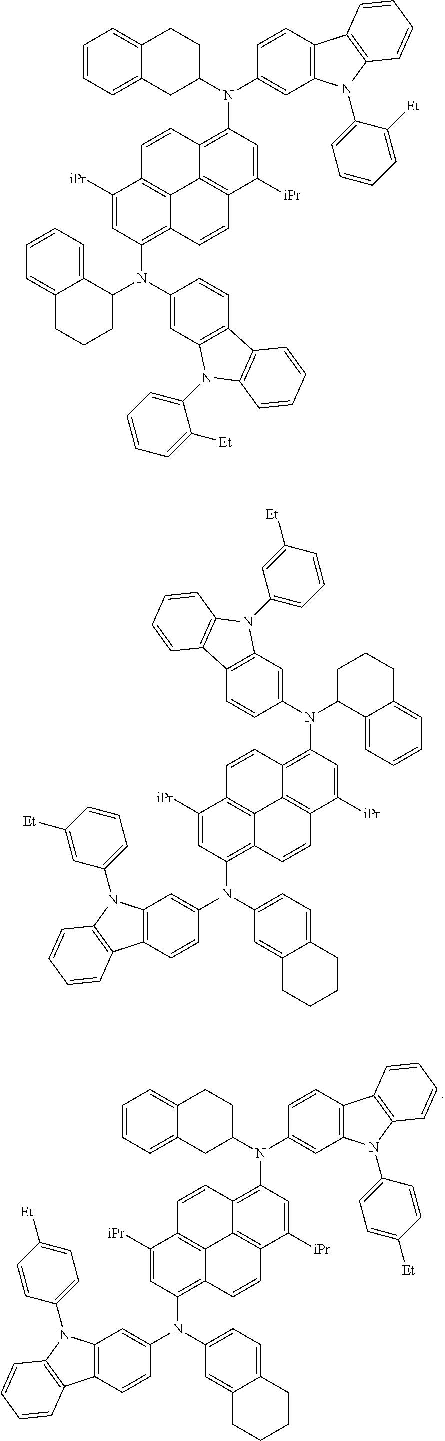 [SMILES: CCc1ccccc1-n1c2ccccc2c2ccc(N(c3cc(C(C)C)c4ccc5c(N(c6ccc7c8ccccc8n(-c8ccccc8CC)c7c6)C6CCCc7ccccc76)cc(C(C)C)c6ccc3c4c65)C3CCc4ccccc4C3)cc21 ; CCc1cccc(-n2c3ccccc3c3ccc(N(c4ccc5c(c4)CCCC5)c4cc(C(C)C)c5ccc6c(N(c7ccc8c9ccccc9n(-c9cccc(CC)c9)c8c7)C7CCCc8ccccc87)cc(C(C)C)c7ccc4c5c76)cc32)c1 ; CCc1ccc(-n2c3ccccc3c3ccc(N(c4ccc5c(c4)CCCC5)c4cc(C(C)C)c5ccc6c(N(c7ccc8c9ccccc9n(-c9ccc(CC)cc9)c8c7)C7CCc8ccccc8C7)cc(C(C)C)c7ccc4c5c76)cc32)cc1]

7. A composition comprising the tetrahydronaphthalene-based organic compound according to claim 1, and at least one organic solvent.

8. An organic electronic device comprising at least the tetrahydronaphthalene-based organic compound according to claim 1.

9. The organic electronic device according to claim 8, wherein the organic electronic device comprises at least a light-emitting layer comprising a tetrahydronaphthalene-based organic compound having a structure shown in general formula (1): 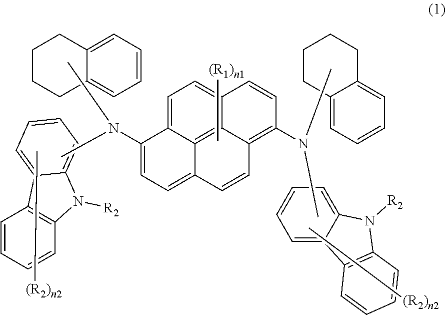
wherein, n1 is an integer selected from 1 to 8; n2 is an integer selected from 1 to 7;
R₁ and R₂ at each occurence is independently selected from the group consisting of —H, deuterium, a linear alkyl group containing 1 to 20 carbon atoms, a linear alkoxy containing 1 to 20 carbon atoms, a linear thioalkoxy group containing 1 to 20 carbon atoms, a branched or cyclic alkyl containing 3 to 20 carbon atoms, a branched or cyclic alkoxy containing 3 to 20 carbon atoms, a branched or cyclic thioalkoxy group containing 3 to 20 carbon atoms, a silyl group, a ketone group containing 1 to 20 carbon atoms, an alkoxycarbonyl group containing 2 to 20 carbon atoms, an aryloxycarbonyl group containing 7 to 20 carbon atoms, a substituted or unsubstituted aromatic or heteroaromatic group containing 5 to 60 ring atoms, an aryloxy or heteroaryloxy group containing 5 to 60 ring atoms, a cyano group, a carbamoyl group, a haloformyl group, a formyl group, an isocyano group, an isocyanate group, a thiocyanate group, an isothiocyanate group, a hydroxyl group, a nitro group, an amino group, —CF₃, —Cl, —Br, —F, —I, and combinations thereof.
